(19) United States
(12) Patent Application Publication
(10) Pub. No.: US 2026/0146286 A1
(43) Pub. Date: May 28, 2026

(54) SMALL RNA-BASED PROGNOSTIC SIGNATURES AND THERAPEUTIC COMPOSITIONS FOR IDIOPATHIC PULMONARY FIBROSIS

(71) Applicant: GATEHOUSE BIO INC., Natick, MA (US)

(72) Inventors: David W. SALZMAN, Natick, MA (US); Neal C. FOSTER, Natick, MA (US); Molly SROUR, Natick, MA (US); Matthew R. LONG, Natick, MA (US); Christian BRION, Natick, MA (US); Guangliang WANG, Natick, MA (US); Nathan RAY, Natick, MA (US)

(21) Appl. No.: 19/121,632

(22) PCT Filed: Oct. 17, 2023
(86) PCT No.: PCT/US2023/077089

§ 371 Date: Apr. 16, 2025

Related U.S. Application Data
(60) Provisional application No. 63/416,662, filed on Oct. 17, 2022.
(60) Provisional application No. 63/539,640, filed on Sep. 21, 2023.

(51) Int. Cl. C12Q 1/6883; A61K 9/00; A61K 9/127; A61K 31/7115; A61K 31/712; A61K 31/7125; A61K 31/713; A61P 11/00; C12N 15/10; C12N 15/113; C12N 15/88; C12Q 1/6823; C12Q 1/6851; C12Q 1/6869

(52) U.S. Cl. CPC C12Q 1/6883; A61K 9/0073; A61K 9/127; A61K 31/7115; A61K 31/712; A61K 31/7125; A61K 31/713; A61P 11/00; C12N 15/1096; C12N 15/113; C12N 15/88; C12Q 1/6823; C12Q 1/6851; C12Q 1/6869; C12N 2310/11; C12N 2310/14; C12N 2310/313; C12N 2310/315; C12N 2310/3181; C12N 2310/321; C12N 2310/322; C12N 2310/3231; C12N 2310/3233; C12N 2310/3341; C12Q 2600/118; C12Q 2600/158

(57) ABSTRACT

The present disclosure provides methods, kits, and therapeutic molecules for evaluating and treating patients with IPF and subtypes of IPF. Specifically, the present disclosure provides a method for diagnosing a patient with IPF and subtypes of IPF based on the expression profile of small, non-coding RNA biomarkers. The present disclosure also provides therapeutic molecules that target the dysregulated sRNAs of the expression profile. In some aspects and embodiments, the disclosure provides miR-92a-3p isoforms and compounds that mimic the action of miR-92a-3p or its isoforms for use in therapy, including but not limited to therapy for IPF.

### PRIORITY

This application claims the benefit of, and priority to, U.S. Provisional Application No. 63/539,640, filed Sep. 21, 2023 and U.S. Provisional Application No. 63/416,662, filed Oct. 17, 2022, the contents of which are hereby incorporated by reference in its entirety.

### INCORPORATION BY REFERENCE OF SEQUENCE LISTING

The present application is being filed with a Sequence Listing in electronic format. The Sequence Listing is provided as a file SRN-007PC_115987-5007_SequenceListing_ST26, created on Oct. 16, 2023, and is 797,594 bytes in size. The information in electronic format of the Sequence Listing is incorporated by reference in its entirety.

### BACKGROUND

Idiopathic pulmonary fibrosis (IPF) is a heterogeneous interstitial lung disease (ILD) with varied etiology and disease course. In the absence of effective treatments, the prognosis of IPF is poor, with a median survival after diagnosis ranging from 2 to 5 years based on several longitudinal studies. Some patients decline in an accelerated manner and die within months of diagnosis, while others experience a slow, gradual progression over many years. This inherent uncertainty in disease course prohibits physicians from providing patients with timely care, including life planning, treatment, and referral to lung transplant centers. With the advent of antifibrotic agents such as nintedanib and pirfenidone, IPF disease course can be slowed, but not cured. This disparate disease course presents a major challenge for both clinicians and drug developers.

There is a need for diagnostic tests and therapies, including therapies that utilize a precision medicine-based approach for IPF and other fibrotic disorders. In the various aspects and embodiments, this disclosure meets these and other objectives.

### SUMMARY OF THE DISCLOSURE

The present disclosure provides methods and kits for evaluating and risk stratifying subjects with Idiopathic Pulmonary Fibrosis (IPF). Specifically, the present disclosure provides methods and kits for determining an expression profile of small, non-coding RNA biomarkers that can predict risk of mortality in subjects with IPF. In other aspects, the present disclosure provides therapeutic compositions for IPF and other conditions based on small, non-coding RNAs, for example, with the potential to correct dysregulation of several messenger RNA targets with an sRNA mimetic, or with an antisense oligonucleotide targeting the sRNA. In embodiments, the sRNA is a sRNA isoform (e.g., a miR-92a-3p isoform) that is dysregulated in its expression in the disease state.

In one aspect, the present disclosure provides a method for risk stratifying a subject diagnosed with Idiopathic Pulmonary Fibrosis (IPF), comprising, providing a blood, serum, or plasma sample from the subject, generating an expression profile of at least five small RNAs listed in Table 2, and determining a risk of mortality as a function of the expression profile, thereby risk stratifying the subject. In embodiments, the method further comprises determining a GAP stage of the subject, and determining risk of mortality based on the expression profile and the GAP stage. In embodiments, the subject with a high-risk stratification is selected or prioritized for surgical or pharmaceutical intervention.

In one aspect, the present disclosure provides a method for evaluating Idiopathic Pulmonary Fibrosis in a subject, comprising, providing a blood, serum, or plasma sample from the subject, generating an expression profile of at least five small RNAs listed in Table 6, and based on the expression profile identifying the subject as having a subtype of Idiopathic Pulmonary Fibrosis that correlates to overall risk of mortality. In embodiments, the subject with a high-risk subtype is selected or prioritized for surgical or pharmaceutical intervention.

In another aspect, the present disclosure provides a kit for evaluating samples for risk stratifying IPF, e.g., in accordance with the methods described herein. In some embodiments, the kit comprises sRNA-specific probes and/or primers configured for detecting a plurality of sRNAs listed in Table 2 (SEQ ID NOS: 1-44). In some embodiments, the kit comprises sRNA-specific stem loop RT primers. Exemplary stem loop primers are listed in Table 3. In embodiments, the kit comprises forward and reverse primers to amplify the reverse transcripts (i.e., resulting from reverse transcription with the stem loop primer). In some embodiments, the reverse primer can be a universal primer. In some embodiments, forward primers are listed in Table 3. A universal reverse primer is also listed in Table 3. In embodiments, the kit comprises sRNA-specific probes that are fluorescent-labeled, for detecting amplicons in real time. In some embodiments, the probe further comprises a quencher moiety.

In another aspect, the present disclosure provides a kit for evaluating samples for a high risk subtype of IPF, e.g., in accordance with the methods described herein. In some embodiments, the kit comprises sRNA-specific probes and/or primers configured for detecting a plurality of sRNAs listed in Table 6 (SEQ ID NOS: 45-115). In some embodiments, the kit comprises sRNA-specific stem loop RT primers. Exemplary stem loop primers are listed in Table 7. In embodiments, the kit comprises forward and reverse primers to amplify the reverse transcripts (i.e., resulting from reverse transcription with the stem loop primer). In some embodiments, the reverse primer can be a universal primer. In some embodiments, forward primers are listed in Table 7. A universal reverse primer is also listed in Table 7. In embodiments, the kit comprises sRNA-specific probes that are fluorescent-labeled, for detecting amplicons in real time. In some embodiments, the probe further comprises a quencher moiety.

In other aspects, the present disclosure provides therapeutic molecules based on the dysregulated sRNAs of Table 2. Specifically, the present disclosure provides pharmaceutical compositions that comprise molecules designed to mimic the action of sRNAs that are down regulated in subjects that have IPF, or IPF with high risk of mortality. Such molecules in some embodiments induce RNA interference of target RNAs (e.g., mRNAs that are targeted by a particular sRNA) in a cell. In some embodiments, the present disclosure provides pharmaceutical compositions that comprise antisense oligonucleotides designed to reduce the expression of sRNAs that are upregulated in subjects that have IPF or IPF with high risk of mortality. Such antisense oligonucleotides can induce degradation of or hinder the action of the target sRNA.

In other aspects, the present disclosure provides therapeutic molecules based on the dysregulated sRNAs of Table 6. Specifically, the present disclosure provides pharmaceutical compositions that comprise molecules designed to mimic the action of sRNAs that are down regulated in subjects that have IPF or IPF with high risk of mortality. Such molecules in some embodiments induce RNA interference of target RNAs (e.g., mRNAs that are targeted by a particular sRNA) in a cell. In some embodiments, the present disclosure provides pharmaceutical compositions that comprise antisense oligonucleotides designed to reduce the expression of sRNAs that are upregulated in subjects that have IPF or IPF with high risk of mortality. Such antisense oligonucleotides can induce degradation of or hinder the action of the target sRNA.

In embodiments, the siRNA is a mimetic for miR-92a-3p or an isoform thereof (such as an isoform listed in Table 6). In various embodiments, the siRNA induces degradation of one or a plurality of mRNA targets of miR-92a-3p. In some embodiments, the siRNA induces degradation of one or a plurality of mRNA targets of an isoform of miR-92a-3p that is substantially less abundant in high risk IPF.

In some embodiments, the siRNA mimics the action of miR-92a-3p or an isoform thereof, such as an isoform listed in Table 10 (SEQ ID NOS: 734-762). These siRNAs comprise an antisense sequence or strand having the nucleobase sequence of the sRNA isoform (optionally with a 3′ overhang, such as dTdT), and which can be chemically modified according to known techniques. Exemplary nucleobase sequences for antisense strands are shown in Table 10 (SEQ ID NOS: 763-791). Exemplary sense or passenger strands are also shown (SEQ ID NOS: 792-820).

The siRNA, when introduced into cells either in vivo or ex vivo, induces the degradation of one or more target RNAs (e.g., target mRNAs) in cells. In some embodiments, the siRNA induces the degradation of a plurality of mRNAs in target cells. In some embodiments, one or more mRNAs are reduced in expression in several pro-fibrotic pathways (WNT, TGF-beta, and focal adhesion). In some embodiments, one or more mRNAs are reduced in expression in pathways such as ECM, Collagen, and interleukin-mediated inflammation pathways. In some embodiments, target cells are pulmonary epithelial cells. In embodiments, the siRNA is formulated for systemic delivery or for local delivery to the lungs. In embodiments the siRNA is encapsulated in a lipid nanoparticle, polymeric nanoparticle, or liposome. In embodiments, the siRNA is delivered by inhalation and is optionally delivered in aerosol (e.g., solution or powder aerosol) form.

In other aspects, the present disclosure provides a method (or use of composition) for treating a subject having IPF, comprising administering an effective amount of a composition sufficient for decreasing the expression of a small RNA selected from Table 2 (SEQ ID No 1 to 44). In various embodiments, the composition is effective for decreasing the expression of a small RNA from Table 2 that is demonstrated herein to be higher in abundance in IPF subjects having a high risk of mortality.

In other aspects, the present disclosure provides a method (or use of composition) for treating a subject having a subtype of IPF, comprising administering an effective amount of a composition sufficient for decreasing the expression of a small RNA selected from Table 6 (SEQ ID No 45 to 115). In various embodiments, the composition is effective for decreasing the expression of a small RNA from Table 6 that is demonstrated herein to be higher in abundance in IPF subjects having a high risk of mortality.

In other aspects, the present disclosure provides a method (or use of composition) for treating a subject having IPF, comprising administering an effective amount of a composition sufficient to mimic the action of a small RNA selected from Table 2 (SEQ ID No: 1 to 44). In various embodiments, the composition is effective for mimicking the action of a small RNA from Table 2 that is demonstrated herein to be lower in abundance in IPF subjects having a high risk of mortality. In various embodiments, the composition comprises an siRNA or shRNA, or another suitable format for inducing RNAi as described above.

In other aspects, the present disclosure provides a method (or use of composition) for treating a subject having IPF, comprising administering an effective amount of a composition sufficient to mimic the action of a small RNA selected from Table 6 (SEQ ID No 45 to 115). In various embodiments, the composition is effective for mimicking the action of a small RNA from Table 6 that is demonstrated herein to be lower in abundance in IPF subjects having a high risk of mortality. In various embodiments, the composition comprises an siRNA or shRNA, or another suitable format for inducing RNAi as described above.

In some embodiments, the disclosure provides a method for treating IPF, or IPF having a high risk of mortality, or a subtype of IPF (e.g., having a high risk of mortality), by administering a therapeutic agent that mimics the action or miR-92a-3p or an isoform thereof (such as an isoform described herein). In some embodiments, the isoform is described in Table 10. In various embodiments, the agent is an siRNA that induces degradation of one or a plurality of mRNA targets of miR-92a-3p. In some embodiments, the therapeutic agent is miR-92a-3p or an isoform thereof. In some embodiments, the therapeutic agent induces degradation of one or a plurality of mRNA targets of an isoform of miR-92a-3p that is substantially less abundant in high risk IPF.

In some embodiments, the subject is demonstrated as having IPF with a high risk of mortality as described herein, or is otherwise shown to have low or undetectable circulating levels of one or more miR-92a-3p isoforms that are less abundant (or not detected) in IPF with high risk of mortality.

In still other aspects and embodiments, the present disclosure provides a method (and use of a composition) for treating an inflammatory or fibrotic disorder. In various embodiments, the disorder is characterized by dysregulation of the WNT/TGF-b/ITGA/FAK regulatory axis. In various aspects and embodiments, the method or use involves administering a composition that comprises miR-92a-3p or an isoform thereof, or a mimic of miR-92a-3p or an isoform thereof. In some embodiments, the compound that mimics miR-92a-3p or an isoform thereof is an siRNA, where the antisense strand or sequence comprises the sequence of the microRNA or isoform (including as described herein). In various embodiments, the isoform will be an isoform that is downregulated in the disorder or disease of interest (e.g., downregulated in the tissue of interest).

Other aspects and embodiments of the present disclosure will be apparent from the following detailed description.

### DESCRIPTION OF FIGURES

FIG. 1 shows a summary of the study design.

FIG. 2 is a chart showing the regression coefficient (with 95% confidence intervals) for sRNA features in the DATASET 1 cohort model.

FIG. 3 is a chart showing the regression coefficient (with 95% confidence intervals) for sRNA features in the DATASET 2 cohort model.

FIG. 4 shows a graph of comparative regression coefficients for statistically significant features in the DATASET 1 and DATASET 2 Cox Regression models.

FIG. 5 shows a decision curve analysis for the DATASET 1 cohort with the following models applied: Cox Regression with sRNA features, Cox Regression with GAP features, and Cox Regression with GAP+sRNA features.

FIG. 6 shows a decision curve analysis for the DATASET 2 cohort with the following models applied: Cox Regression with sRNA features, Cox Regression with GAP features, and Cox Regression with GAP+sRNA features.

FIG. 7A-7C are graphs showing DATASET 1 Kaplan-Meier Curves with HR and Log-Rank test p-value for Cox Regression with (A) sRNA features, (B) GAP stage, and (C) GAP+sRNA features. Risk thresholds for sRNA and GAP+sRNA models are as follows: Low: 0-25% probability of death within 3 years; Moderate: 25-50% probability of death within 3 years; High: >50% probability of death within 3 years.

FIG. 8A-8C are graphs showing DATASET 1 Cumulative Mortality Curves for Cox Regression with (A) sRNA features, (B) GAP stage, and (C) GAP+sRNA features. Risk thresholds for sRNA and GAP+sRNA models are as follows: Low: 0-25% probability of death within 3 years; Moderate: 25-50% probability of death within 3 years; High: >50% probability of death within 3 years.

FIG. 9A-9C are graphs showing DATASET 1 Kaplan-Meier Curves with HR and Log-Rank test p-value for Cox Regression with (A) sRNA features, (B) GAP stage, and (C) GAP+sRNA features. Risk thresholds are as follows: Low: 0-40% probability of death within 3 years; High: >40% probability of death within 3 years.

FIG. 10A-10C are graphs showing DATASET 1 Cumulative Mortality Curves for Cox Regression with (A) sRNA features, (B) GAP stage, and (C) GAP+sRNA features. Risk thresholds are as follows: Low: 0-40% probability of death within 3 years; High: >40% probability of death within 3 years.

FIG. 11A-11C are graphs showing DATASET 2 IPF Kaplan-Meier Curves with HR and Log-Rank test p-value for Cox Regression with (A) sRNA features, (B) GAP stage, and (C) GAP+sRNA features. Risk thresholds for sRNA and GAP+sRNA models are as follows: Low: 0-25% probability of death within 3 years; Moderate: 25-50% probability of death within 3 years; High: >50% probability of death within 3 years.

FIG. 12A-12C are graphs showing DATASET 2 IPF Cumulative Mortality Curves for Cox Regression with (A) sRNA features, (B) GAP stage, and (C) GAP+sRNA features. Risk thresholds for sRNA and GAP+sRNA models are as follows: Low: 0-25% probability of death within 3 years; Moderate: 25-50% probability of death within 3 years; High: >50% probability of death within 3 years.

FIG. 13A-13C are graphs showing DATASET 2 IPF Kaplan-Meier Curves with HR and Log-Rank test p-value for Cox Regression with (A) sRNA features, (B) GAP stage, and (C) GAP+sRNA features. Risk thresholds are as follows: Low: 0-35% probability of death within 3 years; High: >35% probability of death within 3 years.

FIG. 14A-14C are graphs showing DATASET 2 IPF Cumulative Mortality Curves for Cox Regression with (A) sRNA features, (B) GAP stage, and (C) GAP+sRNA features. Risk thresholds are as follows: Low: 0-35% probability of death within 3 years; High: >35% probability of death within 3 years.

FIG. 15A-15C are graphs showing DATASET 2 non-IPF Kaplan-Meier Curves with HR and Log-Rank test p-value for Cox Regression with (A) sRNA features, (B) GAP stage, and (C) GAP+sRNA features. Risk thresholds for sRNA and GAP+sRNA models are as follows: Low: 0-25% probability of death within 3 years; Moderate: 25-50% probability of death within 3 years; High: >50% probability of death within 3 years.

FIG. 16A-16C are graphs showing DATASET 2 non-IPF Cumulative Mortality Curves for Cox Regression with (A) sRNA features, (B) GAP stage, and (C) GAP+sRNA features. Risk thresholds for sRNA and GAP+sRNA models are as follows: Low: 0-25% probability of death within 3 years; Moderate: 25-50% probability of death within 3 years; High: >50% probability of death within 3 years.

FIG. 17A-17C are graphs showing DATASET 2 IPF Kaplan-Meier Curves with HR and Log-Rank test p-value for Cox Regression with (A) sRNA features, (B) GAP stage, and (C) GAP+sRNA features. Risk thresholds are as follows: Low: 0-35% probability of death within 3 years; High: >35% probability of death within 3 years.

FIG. 18A-18C are graphs showing DATASET 2 non-IPF Cumulative Mortality Curves for Cox Regression with (A) sRNA features, (B) GAP stage, and (C) GAP+sRNA features. Risk thresholds are as follows: Low: 0-35% probability of death within 3 years; High: >35% probability of death within 3 years.

FIG. 19 shows a chart of the principal component analysis stratification of discovery cohort.

FIG. 20 shows a graph of Kaplan Meier Curve of four patient subtypes that correlate to overall survival.

FIG. 21A-B shows characterization of small RNA isoforms landscape in IPF. (A) Schematic diagram of sRNA biogenesis depicting the mechanism for templated 5′ and 3′ isoforms generation. (B) sRNA read distribution by isoform type at each end, grouped by sample matrix and originating pre-sRNA arm.

FIG. 22A-E show that miR-92a-3p isoform expression correlates to IPF and collagen expression. miR-92a-3p isoform expression was quantified in 4 independent datasets from 7 different clinical sites: microarray data from Patient Lung Biopsies (A), Whole Blood Samples (B), Patient Primary Fibroblasts (C), and Mouse Lung Tissue (D). For mouse data, bleomycin-induced pulmonary fibrosis was designated IPF, and corresponding saline controls were designated “Control”. All comparisons are statistically significant (t-test, p<0.05). (E) A negative correlation between miR-92a-3p expression and COL1A1 expression in primary fibroblast cell lines indicates the regulatory impact of miR-92a-3p in collagen deposition.

FIG. 23A-B shows that miR-92a-3p isoforms are a powerful feature for predicting 3-year survival in IPF patients. (A) Kaplan Meier plot with hazard ratios using sRNA sequencing data from IPF whole blood samples binned into GAP/GAPS+Stage I (Low risk: 0-25%), II (Moderate risk: 25-50%), and III (High risk: >50%), according to the GAP Index scoring criterion with or without blood-based sRNAs, where risk % represents probability of mortality within 3 years. (B) Hazard ratios for each sRNA isoform and component of GAP Index for GAPS+ model implicate miR-92a-3p isoform (highlighted) as significant contributor of 3-year transplant-free survival.

FIG. 24A-E show that deficiency of miR-92a-3p isoforms enhances bleomycin-induced lung fibrosis. (A) Percentage loss of body weight from Day 0. The data of percentage loss of body weight was ANCOVA analysis. Data are presented as mean±SEM. *P<0.05, **P<0.01. Black dotted line indicates 5% (expected). Grey dotted line indicates 15% and the threshold for required euthanasia according to wellness guidelines. (B) Kaplan Meier plot of overall survival. One Wild Type animal was removed due to a non-bleomycin related death event. (C) Representative images of picrosirius red (PSR) staining on Wild Type (top) and MIR92A-1+/−(bottom) animals. (D) Quantification of PSR staining in Wild Type (n=8) and MIR92A-1+/−(n=9) animals. (E) Sum miR-92-3p isoform expression in lung tissue collected on Day 21 (TRPM; mean±SEM).

FIG. 25A-F show that miR-92a-3p rescued bleomycin-induced pulmonary fibrosis in mice. (A) Schematic showing study design for in vivo efficacy. (B) Percentage change in body weight. (C) Plasma blood glucose levels in animals at Day 21. (D) Ratio of lung (mg) to body weight (g). (E) Quantitative analysis of hydroxyproline in lung homogenates. (F) Representative images of picrosirius red (PSR) staining of lung sections showing percentage fibrosis lesions in mice subjected to saline (n=8), bleomycin (n=10) and miR-92a mimic treated bleomycin (n=8). Data is presented as mean±SEM. *P<0.05, **P<0.01.

FIG. 26A-D shows activity screening of miR-92a-3p mimetics in patient primary IPF fibroblasts. (A) Heatmap showing the effect of cell growth on primary IPF fibroblasts treated with miR-92a-3p mimetics (100 nM). (B) Heatmap showing the effect of collagen type 1 alpha 1 (COL1A1) expression in primary IPF fibroblasts treated with either scrambled control or miR-92a-3p mimetics (100 nM). (C) The average collagen inhibition of Oligo-006 was highlighted. Immunofluorescence images of representative IPF cells and treatment condition stained for COL1A1, filamentous actin and Hoechst staining. The data for COL1A1 level and cell growth is normalized to the scrambled control. (D) Inhibition of cell growth using Oligo-006 was highlighted. Brightfield images of cells at 96-hours was shown.

FIG. 27A-E shows ex vivo efficacy of miR-92a-3p isoforms using precision cut lung slices. (A) Schematic representation of the experimental design. PCLS were treated in duplicate. (B) Collagen expression was significantly reduced (p<0.05) in PCLSs treated with Oligo-006 (GHB1589). Results are the mean (±) standard deviation (n=3). (C) Inflammation scoring of interstitial tissue was completed on H&E stained PCLSs (n=2). (D) Fibrosis scoring of interstitial tissue was completed on H&E stained PCLSs (n=2). (E) Proteomic analysis of inflammatory and fibrotic biomarkers using Luminex. Results are shown as the percent change relative to scrambled control (n=1).

FIG. 28A-C shows identification of miR-92a-3p targets and mechanism of action. (A) Volcano plot of the differential expression analysis of IPF fibroblast transfected with oligo-006 versus scrambled oligo (BH p-adjust threshold=0.05). (B) Pathway and transcription factor (TF) targets enrichment analysis (*: p-adjust<0.1, **: p-adjust<0.01, ***: p-adjust<0.001). (C) Binding site activity tested using luciferase reporters after transfection of Oligo-006, Highlighted significant genes are pro-fibrotic (*: p-adjust<0.05, **: p-adjust<0.01).

FIG. 29 shows validated mechanism of action for Oligo-006, and shows the regulatory feedback loop involved in the propagation of fibrosis. The color and value of the genes indicates the differential expression after transfection of oligo-006 in fibroblast. Underlined genes have a miR-92a-3p binding site, highlighted genes have been confirmed as physical target of the mimetic through reporter assay.

### DETAILED DESCRIPTION

The present disclosure provides methods and kits for evaluating and risk stratifying subjects with Idiopathic Pulmonary Fibrosis (IPF). Specifically, the present disclosure provides methods and kits for determining an expression profile of small, non-coding RNA biomarkers that can predict risk of mortality in subjects with IPF. In other aspects, the present disclosure provides therapeutic compositions for IPF and other conditions based on small, non-coding RNAs, for example, with the potential to correct dysregulation of several messenger RNA targets with an sRNA mimetic, or with an antisense oligonucleotide targeting the sRNA. In embodiments, the sRNA is a sRNA isoform (e.g., a miR-92a-3p isoform) that is dysregulated in its expression in the disease state.

Idiopathic pulmonary fibrosis is a chronic, progressive lung disease. This condition causes scar tissue (fibrosis) to build up in the lungs, which makes the lungs unable to transport oxygen into the bloodstream effectively. Idiopathic pulmonary fibrosis belongs to a group of conditions called interstitial lung diseases (also known as ILD), which describes lung diseases that involve inflammation or scarring in the lung. Some people with idiopathic pulmonary fibrosis develop other serious lung conditions, including lung cancer, blood clots in the lungs (pulmonary emboli), pneumonia, or high blood pressure in the blood vessels that supply the lungs (pulmonary hypertension). Most affected individuals survive 3 to 5 years after their diagnosis. However, the course of the disease is highly variable; some affected people become seriously ill within a few months, while others may live with the disease for a decade or longer.

Conventionally, the GAP model is used as a risk assessment system to determine the risk of mortality of IPF patients. The baseline “GAP model” (named for the model variables: Gender, Age, and Physiology) consists of 2 prognostic tools that provide physicians with a framework for discussing prognosis and evaluating stage-specific management options with patients. The first GAP index and staging system provides a simple screening method to determine the average risk of mortality of patients with IPF by GAP stage. The GAP calculator, an expanded version of the GAP index, provides a more precise mortality risk estimation. The GAP index and GAP calculator estimate stage and/or mortality of IPF patients based on gender, age, Forced Vital Capacity (% FVC), and diffusion capacity for carbon monoxide (% DLCO).

In aspects and embodiments, the present disclosure provides a method of evaluating and risk stratifying a patient diagnosed with IPF using a blood biomarker panel. The biomarker panel can be used independently or together with the GAP model to more accurately assess IPF mortality.

In one aspect, the present disclosure provides a method for risk stratifying a subject diagnosed with Idiopathic Pulmonary Fibrosis (IPF), comprising, providing a blood, serum, or plasma sample from the subject, generating an expression profile of at least five small RNAs listed in Table 2, and determining a risk of mortality as a function of the expression profile, thereby risk stratifying the subject. In embodiments, the method further comprises determining a GAP stage of the subject, and determining risk of mortality based on the expression profile and the GAP stage. In embodiments, the GAP stage comprises three thresholds based on probability of death within 3 years. In embodiments, the subject is stratified into three groups based on risk of mortality. In embodiments, the three groups are low risk, moderate risk, and high risk of death within 3 years. In embodiments, the subject is stratified into two groups based on risk of mortality. In embodiments, the two groups are low risk and high risk of death within 3 years. For example, in some embodiments an expression profile is generated for subjects having a moderate and/or high risk of death by the GAP index. In some embodiments, an expression profile is generated for subjects having a low risk of death by the GAP index.

In embodiments, the subject with a high-risk stratification (according to this disclosure) is selected or prioritized for surgical or pharmaceutical intervention. In embodiments, the subject is selected or prioritized for lung transplant. Thus, in some embodiments, a subject determined as having a high risk of mortality according to the present disclosure undergoes a lung transplant (either single lung or two lungs) within about 2 years, or within about 1 year, or within about 6 months of the biomarker evaluation (e.g., as determined from the time of blood draw).

In one aspect, the present disclosure provides a method for evaluating Idiopathic Pulmonary Fibrosis in a subject, comprising, providing a blood, serum, or plasma sample from the subject, generating an expression profile of at least five small RNAs listed in Table 6, and based on the expression profile identifying the subject as having a subtype of Idiopathic Pulmonary Fibrosis that correlates to overall risk of mortality. In embodiments, the subject with a high-risk subtype is selected or prioritized for surgical or pharmaceutical intervention. In embodiments, the subject with a high-risk subtype is selected or prioritized for lung transplant. Thus, in some embodiments, a subject determined as having a high-risk subtype according to the present disclosure undergoes a lung transplant (either single lung or two lungs) within about 2 years, or within about 1 year, or within about 6 months of the biomarker evaluation (e.g., as determined from the time of blood draw).

Thus, in various aspects and embodiments, the present disclosure provides a small RNA panel whose expression correlates to IPF, or risk of mortality for an IPF patient. Small RNA (“sRNAs”) are non-coding RNAs less than 200 nucleotides in length and include microRNAs (miRNAs) (including iso-miRs), Piwi-interacting RNAs (piRNAs), small interfering RNAs (siRNAs), vault RNAs (vtRNAs), small nucleolar RNAs (snoRNAs), transfer RNA-derived small RNAs (tsRNAs), ribosomal RNA-derived small RNA fragments (rsRNAs), small rRNA-derived RNAs (srRNA), and small nuclear RNAs (U-RNAs), as well as novel uncharacterized RNA species. Generally, “isoforms” refer to those sequences that have variations with respect to a reference sequence (e.g., the human genome GRCh38/hg38 build, miRBase, piRNAdb, etc.). In miRBase, each miRNA is associated with a miRNA precursor and with one or two mature miRNA (-5p and -3p). Deep sequencing has detected a large amount of variability in small RNA biogenesis, meaning that from the same precursor RNA many different sequences can be generated. There are three main variations of isoforms: (1) templated variants, where the 5′ and 3′ end are upstream or downstream of the reference; (2) non-templated variants, where nucleotides are added to the 5′ and 3′ end that do not align to the reference; (3) nucleotide substitutions, where internal nucleotides do not align to the reference.

In various embodiments, the expression profile comprises the expression levels of a plurality of sRNAs in Table 2. Table 2 lists sRNA markers whose serum level correlates (positively or negatively) with mortality in IPF. Table 2 provides 44 sRNA sequences whose expression level is correlated to risk of mortality in IPF patients, and which can be used to prepare models for evaluating and risk stratifying IPF subjects. As shown in Table 2, the sRNAs include various types of RNA species including miRNAs, tRNA-derived sRNA, pre-microRNAs and other species.

Table 2 shows sRNA sequences in DNA format (e.g., the sequence obtained after RT-PCR). It is understood that where nucleotide sequences described herein are intended to be RNA or comprise RNA nucleotides, thymine (T) will be replaced with uracil (U) nucleobases.

In various embodiments, the expression profile comprises the expression level of at least 10 sRNAs from Table 2. In embodiments, the expression profile comprises the expression level of at least 20 sRNAs from Table 2. In embodiments, the expression profile comprises the expression level of at least 30 sRNAs from Table 2. In embodiments, the expression profile comprises the expression level of at least 40 sRNAs from Table 2. In embodiments, the expression profile comprises, consists essentially of, or consists of the expression levels of sRNAs from Table 2.

In various embodiments, the expression profile comprises the expression levels of a plurality of sRNAs in Table 6. Table 6 provides 71 sRNA sequences whose expression levels identify different subtypes of IPF that differ in their mortality risk. As shown in Table 6, the sRNAs include microRNA isoforms.

Table 6 shows sRNA sequences in DNA format (e.g., the sequence obtained after RT-PCR). It is understood that where nucleotide sequences described herein are intended to be RNA or comprise RNA nucleotides, thymine (T) will be replaced with uracil (U) nucleobases.

In various embodiments, the expression profile comprises the expression level of at least 10 sRNAs from Table 6. In embodiments, the expression profile comprises the expression level of at least 20 sRNAs from Table 6. In embodiments, the expression profile comprises the expression level of at least 30 sRNAs from Table 6. In embodiments, the expression profile comprises the expression level of at least 40 sRNAs from Table 6. In embodiments, the expression profile comprises, consists essentially of, or consists of the expression levels of sRNAs from Table 6.

In this context (with regard to the sRNAs of Table 2 and 6), the term “consists essentially of” means that additional sRNAs can also be measured as part of the expression profile, and that such sRNAs do not significantly impact (i.e., reduce) the correlation of the expression profile with IPF mortality, or are not included in the expression profile analysis. In some embodiments, the additional sRNAs can be used as expression level controls. Models can be developed using training cohorts and employing supervised, regression modeling of expression profiles determined for IPF subjects and control subjects randomized into training and test groups.

In some embodiments, a risk score is calculated for the expression profile (e.g., based on RT-qPCR of the markers in Table 2) according to the following equation (Equation 1):

$$ln \frac{h ( t )}{{h}_{0} ( t )} = 0.153 * ( {log}_{10} ( {U}_{1} + 1 ) ) + 0.613 * ( {log}_{10} ( {U}_{2} + 1 ) ) - 0.562 * ( {log}_{10} ( {U}_{3} + 1 ) ) + 0.058 * ( {log}_{10} ( {U}_{4} + 1 ) ) + 1.143 * ( {log}_{10} ( {U}_{5} + 1 ) ) + 0.136 * ( {log}_{10} ( {U}_{6} + 1 ) ) - 0.85 * ( {log}_{10} ( {U}_{7} + 1 ) ) + 0.028 * ( {log}_{10} ( {U}_{8} + 1 ) ) - 0.218 * ( {log}_{10} ( {U}_{9} + 1 ) ) - 0.42 * ( {log}_{10} ( {U}_{10} + 1 ) ) + 1.462 * ( {log}_{10} ( {U}_{11} + 1 ) ) - 1.3 \; 72 * ( {log}_{10} ( {U}_{12} + 1 ) ) + 0.829 * ( {log}_{10} ( {U}_{13} + 1 ) ) - 1.089 * ( {log}_{10} ( {U}_{14} + 1 ) ) - 0.131 * ( {log}_{10} ( {U}_{15} + 1 ) ) - 0.399 * ( {log}_{10} ( {U}_{16} + 1 ) ) - 1.988 * ( {log}_{10} ( {U}_{17} + 1 ) ) + 0.204 * ( {log}_{10} ( {U}_{18} + 1 ) ) - 0.012 * ( {log}_{10} ( {U}_{19} + 1 ) ) + 0.5 \; 28 * ( {log}_{10} ( {U}_{20} + 1 ) ) + 0.8 * ( {log}_{10} ( {U}_{21} + 1 ) ) - 0.323 * ( {log}_{10} ( {U}_{22} + 1 ) ) - 0.113 * ( {log}_{10} ( {U}_{23} + 1 ) ) - 0.387 * ( {log}_{10} ( {U}_{24} + 1 ) ) + 0.794 * ( {log}_{10} ( {U}_{25} + 1 ) ) - 0.213 * ( {log}_{10} ( {U}_{26} + 1 ) ) - 1.66 * ( {log}_{10} ( {U}_{27} + 1 ) ) - 1.1 \; 14 * ( {log}_{10} ( {U}_{28} + 1 ) ) + 0.237 * ( {log}_{10} ( {U}_{29} + 1 ) ) + 1.318 * ( {log}_{10} ( {U}_{30} + 1 ) ) + 1.485 * ( {log}_{10} ( {U}_{31} + 1 ) ) + 0.566 * ( {log}_{10} ( {U}_{32} + 1 ) ) - 0.336 * ( {log}_{10} ( {U}_{33} + 1 ) ) - 0.23 * ( {log}_{10} ( {U}_{34} + 1 ) ) - 0.582 * ( {log}_{10} ( {U}_{35} + 1 ) ) - 0.1 \; 08 * ( {log}_{10} ( {U}_{36} + 1 ) ) - 0.217 * ( {log}_{10} ( {U}_{37} + 1 ) ) - 0.521 * ( {log}_{10} ( {U}_{38} + 1 ) ) + 0.114 * ( {log}_{10} ( {U}_{39} + 1 ) ) - 0.889 * ( {log}_{10} ( {U}_{40} + 1 ) ) - 0.135 * ( {log}_{10} ( {U}_{41} + 1 ) ) - 0.347 * ( {log}_{10} ( {U}_{42} + 1 ) ) - 0.338 * ( {log}_{10} ( {U}_{43} + 1 ) ) - 0.3 \; 46 * ( {log}_{10} ( {U}_{44} + 1 ) )$$

RNA can be extracted from the sample prior to sRNA detection and quantification. RNA may be purified using a variety of standard procedures as described, for example, in RNA Methodologies, A laboratory guide for isolation and characterization, 2nd edition, 1998, Robert E. Farrell, Jr., Ed., Academic Press. In addition, there are various processes as well as products commercially available for isolation of small molecular weight RNAs, including mirVANA™ Paris miRNA Isolation Kit (Ambion), miRNeasy™ kits (Qiagen), MagMAX™ kits (Life Technologies), and Pure Link™ kits (Life Technologies). For example, small molecular weight RNA may be isolated by organic extraction followed by purification on a glass fiber filter. Alternative methods for isolating sRNAs include hybridization to magnetic beads. Alternatively, sRNA processing for detection (e.g., cDNA synthesis) may be conducted in the biofluid sample, that is, without an RNA extraction step.

In various embodiments, detection of the sRNAs in the expression profile involves one of various detection platforms, which can employ reverse-transcription and amplification. In some embodiments, the detection platform involves hybridization of a probe. In some embodiments, the detection platform involves reverse transcription and quantitative PCR (e.g., RT-qPCR). In some embodiments, the sRNAs are reverse transcribed using stem-loop RT primers. Exemplary stem loop primers are shown in Table 3 and Table 7. In embodiments, the reverse transcripts are amplified with forward and reverse primers. Exemplary forward and reverse primers are also shown in Table 3 and Table 7. The reverse primer can be a universal primer, based on a constant sequence of the stem loop primer. In various embodiments, the quantitative PCR assay employs a fluorescent dye or fluorescent-labeled probe. In embodiments, the quantitative PCR assay employs a fluorescent-labeled probe further comprising a quencher moiety (e.g., TAQMAN Probe).

Generally, real-time PCR monitors the amplification of a targeted DNA molecule during the PCR, i.e. in real-time. Real-time PCR can be used quantitatively, and semi-quantitatively. Two common methods for the detection of PCR products in real-time PCR are: (1) non-specific fluorescent dyes that intercalate with any double-stranded DNA (e.g., SYBR Green (I or II), or ethidium bromide), and (2) sequence-specific DNA probes consisting of oligonucleotides that are labelled with a fluorescent reporter which permits detection only after hybridization of the probe with its complementary sequence (e.g., TAQMAN).

In some embodiments, the assay format is TAQMAN real-time PCR. TAQMAN probes are hydrolysis probes that are designed to increase the specificity of quantitative PCR. The TAQMAN probe principle relies on the 5′ to 3′ exonuclease activity of Taq polymerase to cleave a dual-labeled probe during hybridization to the complementary target sequence, with fluorophore-based detection. TAQMAN probes are dual labeled with a fluorophore and a quencher, and when the fluorophore is cleaved from the oligonucleotide probe by the Taq exonuclease activity, the fluorophore signal is detected (e.g., the signal is no longer quenched by the proximity of the labels). As in other quantitative PCR methods, the resulting fluorescence signal permits quantitative measurements of the accumulation of the product during the exponential stages of the PCR. The TAQMAN probe format provides high sensitivity and specificity of the detection.

Accordingly, in some embodiments, sRNAs in the expression profile are converted to cDNA using specific primers, e.g., a stem-loop primer. Amplification of the cDNA may then be quantified in real time, for example, by detecting the signal from a fluorescent reporting molecule, where the signal intensity correlates with the level of DNA at each amplification cycle.

In embodiments, the expression profile is determined using a hybridization assay. In some embodiments, the hybridization assay employs a hybridization array comprising sRNA-specific probes. Exemplary platforms for detecting hybridization include surface plasmon resonance (SPR) and microarray technology. Detection platforms can use microfluidics in some embodiments, for convenient sample processing and sRNA detection.

In other embodiments, the expression profile is determined by nucleic acid sequencing, and sRNAs are identified in the sample by a process that comprises trimming 5′ and 3′ sequencing adaptors from sRNA sequences. See, U.S. Pat. Nos. 10,889,862 and 11,028,440 (the full contents of which are hereby incorporated by reference), which disclose a process that includes computational trimming of sequencing adapters from RNA sequencing data and sorting data according to unique sequence reads. In some embodiments, RNA from multiple samples is pooled for determining expression profiles by sRNA sequencing, with sequences from different samples containing an identifying sample tag sequence (which can be added by RT-PCR or by ligation). In embodiments, the expression profile further comprises the expression level of one or more expression normalization controls.

Generally, any method for determining the presence or level of sRNAs in samples can be employed. Such methods further include nucleic acid sequence based amplification (NASBA), flap endonuclease-based assays, as well as direct RNA capture with branched DNA (QuantiGene™), Hybrid Capture™ (Digene), or nCounter™ miRNA detection (nanostring). The assay format, in addition to determining the abundance of sRNAs may also provide for the control of, inter alia, intrinsic signal intensity variation. Such controls may include, for example, controls for background signal intensity and/or sample processing, and/or hybridization efficiency, as well as other desirable controls for detecting sRNAs in patient samples (e.g., collectively referred to as “normalization controls”).

In some embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In various embodiments, where the subject is determined to have a high risk of mortality due to IPF, the subject is treated with surgical or pharmaceutical intervention, which is optionally a pharmaceutical intervention described herein.

In embodiments, surgical intervention is lung transplantation. In embodiments, the subject is selected or prioritized for lung transplant. Thus, in some embodiments, a subject determined as having a high risk of mortality according to the present disclosure undergoes a lung transplant (either single lung or two lungs) within about 2 years, or within about 1 year, or within about 6 months of the biomarker evaluation (e.g., which can be calculated from the time of blood draw).

As used herein, the term “pharmaceutical intervention” means that the subject is prescribed (and administered) at least one additional drug (compared to any existing treatment prior to the expression profiling), or the subjects' drug regimen is altered by at least one drug (i.e., at least one active agent is replaced in an ongoing regimen with one or more other agents) or drug dose, based on the results of the sRNA expression profiling.

In embodiments where pharmaceutical interventions are desired, pharmaceuticals can be selected from, but not limited to, nintedanib (Ofev®) and pirfenidone (Esbriet®). In some embodiments, the subject is administered a therapeutic agent described herein for targeting an sRNA or mimicking the action of an sRNA, based on the results of the sRNA biomarker expression profile. In some embodiments, the pharmaceutical intervention is an siRNA that mimics a miR-92a-3p isoform, and which is optionally administered locally to the lungs by inhalation. An exemplary isoform is provided herein as SEQ ID NO: 739, e.g., which can be mimicked with an siRNA comprising an antisense sequence or strand having the nucleobase sequence of SEQ ID NO: 768 (or a derivative thereof described herein). Other miR-92a-3p isoforms and siRNAs for use with the disclosure are provided in Table 10 (which can be modified according to the present disclosure).

In various embodiments, the method is repeated at a frequency of at least once per year, or at least once every six months, or at least once every two months, to monitor the subject's disease progression.

In another aspect, the present disclosure provides a kit for evaluating samples for risk stratifying IPF, e.g., in accordance with the methods described herein. In some embodiments, the kit comprises sRNA-specific probes and/or primers configured for detecting a plurality of sRNAs listed in Table 2 (SEQ ID NOS: 1-44). In embodiments, the kit comprises sRNA-specific probes and/or primers configured for detecting at least 10 sRNAs listed in Table 2 (SEQ ID NOS: 1-44). In embodiments, the kit comprises sRNA-specific probes and/or primers configured for detecting at least 20 sRNAs listed in Table 2 (SEQ ID NOS: 1-44). In embodiments, the kit comprises sRNA-specific probes and/or primers configured for detecting at least 30 sRNAs listed in Table 2 (SEQ ID NOS: 1-44). In embodiments, the kit comprises sRNA-specific probes and/or primers configured for detecting at least 40 sRNAs listed in Table 2 (SEQ ID NOS: 1-44). In embodiments, the kit comprises sRNA-specific probes and/or primers configured for detecting the sRNAs listed in Table 2 (SEQ ID NOS: 1-44).

In some embodiments, the kit comprises sRNA-specific stem loop RT primers. Exemplary stem loop primers are listed in Table 3. The stem-loop primer comprises a constant region that forms a stem loop and a variable nucleotide extension (e.g., of about 5-8 nucleotides, such as 6 nucleotides). The constant region acts as a priming region for a reverse primer during amplification. The variable region of the stem-loop RT primer is configured to specifically reverse transcribe a sRNA of Table 2. In embodiments, the kit comprises forward and reverse primers to amplify the reverse transcripts (i.e., resulting from reverse transcription with the stem loop primer). In some embodiments, the reverse primer can be a universal primer. In some embodiments, forward primers are listed in Table 3. A universal reverse primer is also listed in Table 3. In embodiments, the kit comprises sRNA-specific probes that are fluorescent-labeled, for detecting amplicons in real time. In some embodiments, the probe further comprises a quencher moiety. The probe can be a TAQMAN probe.

In embodiments, the kit comprises an array of sRNA-specific hybridization probes, configured to detect sRNAs of Table 2 (e.g., at least 20, at least 30, at least 40, at least 50, or all sRNAs of Table 2.

In another aspect, the present disclosure provides a kit for evaluating samples for a high risk subtype of IPF, e.g., in accordance with the methods described herein. In some embodiments, the kit comprises sRNA-specific probes and/or primers configured for detecting a plurality of sRNAs listed in Table 6 (SEQ ID NOS: 45-115). In embodiments, the kit comprises sRNA-specific probes and/or primers configured for detecting at least 10 sRNAs listed in Table 6 (SEQ ID NOS: 45-115). In embodiments, the kit comprises sRNA-specific probes and/or primers configured for detecting at least 20 sRNAs listed in Table 6 (SEQ ID NOS: 45-115). In embodiments, the kit comprises sRNA-specific probes and/or primers configured for detecting at least 30 sRNAs listed in Table 6 (SEQ ID NOS: 45-115). In embodiments, the kit comprises sRNA-specific probes and/or primers configured for detecting at least 40 sRNAs listed in Table 6 (SEQ ID NOS: 45-115). In embodiments, the kit comprises sRNA-specific probes and/or primers configured for detecting at least 50, at least 60, or all sRNAs listed in Table 6 (SEQ ID NOS: 45-115).

In some embodiments, the kit comprises sRNA-specific stem loop RT primers. Exemplary stem loop primers are listed in Table 7. The stem-loop primer comprises a constant region that forms a stem loop and a variable nucleotide extension (e.g., of about 5-8 nucleotides, such as 6 nucleotides). The constant region acts as a priming region for a reverse primer during amplification. The variable region of the stem-loop RT primer is configured to specifically reverse transcribe a sRNA of Table 6. In embodiments, the kit comprises forward and reverse primers to amplify the reverse transcripts (i.e., resulting from reverse transcription with the stem loop primer). In some embodiments, the reverse primer can be a universal primer. In some embodiments, forward primers are listed in Table 7. A universal reverse primer is also listed in Table 7. In embodiments, the kit comprises sRNA-specific probes that are fluorescent-labeled, for detecting amplicons in real time. In some embodiments, the probe further comprises a quencher moiety. The probe can be a TAQMAN probe.

In embodiments, the kit comprises an array of sRNA-specific hybridization probes, configured to detect sRNAs of Table 6 (e.g., at least 20, at least 30, at least 40, at least 50, or all sRNAs of Table 6.

In other aspects, the present disclosure provides therapeutic molecules based on the dysregulated sRNAs of Table 2. Specifically, the present disclosure provides pharmaceutical compositions that comprise molecules designed to mimic the action of sRNAs that are down regulated in subjects that have IPF, or IPF with high risk of mortality. Such molecules in some embodiments induce RNA interference of target RNAs (e.g., mRNAs that are targeted by a particular sRNA) in a cell. In some embodiments, the present disclosure provides pharmaceutical compositions that comprise antisense oligonucleotides designed to reduce the expression of sRNAs that are upregulated in subjects that have IPF or IPF with high risk of mortality. Such antisense oligonucleotides can induce degradation of or hinder the action of the target sRNA.

In other aspects, the present disclosure provides therapeutic molecules based on the dysregulated sRNAs of Table 6. Specifically, the present disclosure provides pharmaceutical compositions that comprise molecules designed to mimic the action of sRNAs that are down regulated in subjects that have IPF or IPF with high risk of mortality. Such molecules in some embodiments induce RNA interference of target RNAs (e.g., mRNAs that are targeted by a particular sRNA) in a cell. In some embodiments, the present disclosure provides pharmaceutical compositions that comprise antisense oligonucleotides designed to reduce the expression of sRNAs that are upregulated in subjects that have IPF or IPF with high risk of mortality. Such antisense oligonucleotides can induce degradation of or hinder the action of the target sRNA.

RNA interference (RNAi) is a sequence-specific RNA degradation process to knockdown, or silence, theoretically any gene containing the homologous sequence. In naturally occurring RNAi, a double-stranded RNA (dsRNA) is cleaved by an RNase III/helicase protein (Dicer) into small interfering RNA (siRNA) molecules, which are dsRNAs of 19-27 nucleotides (nt) with 2-nt overhangs at the 3′ ends. Afterwards, the siRNAs are incorporated into a multicomponent-ribonuclease called RNA-induced-silencing-complex (RISC). One strand of siRNA remains associated with RISC to guide the complex towards a cognate RNA that has a sequence complementary to the guider ss-siRNA in RISC. This siRNA-directed endonuclease digests the RNA, resulting in truncation and inactivation of the targeted RNA.

In various embodiments, the present disclosure provides a composition comprising a small interfering RNA (siRNA) that comprises an antisense strand and a sense strand, where the antisense strand comprises a nucleotide sequence selected from Table 2 (or at least 12, 14, 16, 18, 20, 21 or 22 consecutive nucleotides of a sequence selected from Table 2).

In various embodiments, the present disclosure provides a composition comprising a small interfering RNA (siRNA) that comprises an antisense strand and a sense strand, where the antisense strand comprises a nucleotide sequence selected from Table 6 (or at least 12, 14, 16, 18, 20, 21, or 22 consecutive nucleotides of a sequence selected from Table 6). In embodiments, the siRNA is a mimetic for miR-92a-3p or an isoform thereof (such as an isoform listed in Table 6). In various embodiments, the siRNA induces degradation of one or a plurality of mRNA targets of miR-92a-3p. In some embodiments, the siRNA induces degradation of one or a plurality of mRNA targets of an isoform of miR-92a-3p that is substantially less abundant in high risk IPF. In embodiments, the siRNA comprises an antisense sequence of SEQ ID NO: 732 (or at least 20, 21, or 22 consecutive nucleotides of SEQ ID NO: 732). Sense strands (i.e., passenger strands) for inducing RNAi can be designed as known in the art or as described below. Exemplary sense strands include SEQ ID NO: 733 or at least 16, 17, 18, 19, 20, or 21 consecutive nucleotides of SEQ ID NO: 733 (with the antisense strand of SEQ ID NO: 732). In these embodiments, dTdT overhangs are optional.

In some embodiments, the siRNA mimics the action of miR-92a-3p or an isoform thereof, such as an isoform listed in Table 10 (SEQ ID NOS: 734-762). These siRNAs comprise an antisense sequence or strand having the nucleobase sequence of the sRNA isoform (optionally with a 3′ overhang, such as dTdT), and which can be chemically modified according to known techniques (including as described herein). Exemplary nucleobase sequence for antisense strands are shown in Table 10 (SEQ ID NOS: 763-791). Exemplary sense or passenger strands are also shown (SEQ ID NOS: 792-820).

The siRNA, when introduced into cells either in vivo or ex vivo, induces the degradation of one or more target RNAs (e.g., target mRNAs) in cells. In some embodiments, the siRNA induces the degradation of a plurality of mRNAs (e.g., at least 2, 3, 4, 5 or more) in target cells. In some embodiments, one or more mRNAs are reduced in expression in several pro-fibrotic pathways (WNT, TGF-beta, and focal adhesion). In some embodiments, one or more mRNAs are reduced in expression in pathways such as ECM, Collagen, and interleukin-mediated inflammation pathways. In some embodiments, target cells are pulmonary epithelial cells. In embodiments, the siRNA is formulated for systemic delivery (e.g., parenteral delivery) or for local delivery to the lungs, such as but not limited to the route of inhalation. In embodiments the siRNA is encapsulated in a lipid nanoparticle, polymeric nanoparticle, or liposome. In embodiments, the siRNA is delivered by inhalation and is optionally delivered in aerosol (e.g., solution or powder aerosol) form. These delivery forms are well known in the art.

In embodiments, the siRNA comprises a chemical modification, including any of the well-known chemical modifications for siRNA. In embodiments, the chemical modifications increase stability, reduce endonuclease degradation, reduce immunogenicity, and/or reduce Toll-like receptor recognition. In embodiments, the chemical modification is a nucleobase modification, a backbone modification, and/or a sugar modification.

In embodiments, the nucleobase modification suppresses RNA recognition by a Toll-like receptor (TLR). In embodiments, the nucleobase modification is selected from pseudouridine (ψ), N1-methyl-pseudouridine (N1mΨ), 5-methylcytidine (m5C), 2′-thiouridine (s2U), N6′-methyladenosine (m6A), and 5′-fluorouridine.

In embodiments, the siRNA may have one or more backbone modification(s) selected from phosphorothioate, phosphorodithioate, methylphosphonate, and methoxypropylphosphonate. For example, such modifications may be placed at and/or near the 3′ end of the antisense strand and/or the sense strand. Other modified linkages are described elsewhere herein.

In embodiments, the siRNA comprises one or more sugar modifications, such as those selected from 2′-methoxy (2′-Ome), 2′-O-methoxyethyl (2′-O-MOE), 2′-fluoro (2′-F), 2′-arabino-fluoro (2′-Ara-F), constrained ethyl (cEt), bridged nucleic acid (BNA) and locked nucleic acid (LNA). BNA and LNA nucleotides are described elsewhere herein. In embodiments, the antisense strand comprises a 5′ phosphate. Other modifications that are suitable at the 5′ end are known in the art.

In embodiments, the siRNA comprises a sense and antisense strain, each having a length of about 12 to about 40 nucleotides. In embodiments, the siRNA comprises two substantially complementary RNA strands with a duplex length of about 12 to about 40 base pairs (such as from 16 to 24 base pairs). In embodiments, the siRNA comprises a sense strand overhang and an antisense strand overhang at the 3′ ends. The overhangs may be RNA overhangs or may be deoxythymidine (dT-dT) overhangs. In embodiments, the siRNA is an asymmetric siRNA (asiRNA) having a blunt end corresponding to the 5′ end of the antisense strand. In various embodiments, the antisense strand or sequence corresponds to the sequence of the sRNA or isoform being mimicked in its action.

Other siRNA formats, including but not limited to short-hairpin RNAs (shRNAs) (e.g., comprising the sequence of the desired isoform), that may be used are described in US 2008/0188430, which is hereby incorporated by reference.

In various aspects and embodiments, the present disclosure provides a composition comprising an antisense oligonucleotide that targets an sRNA isoform that is upregulated in IPF or another fibrotic disease. For example, the antisense oligonucleotide is at least 10 linked nucleotides in length, and which has a sequence that is complementary to a nucleotide sequence selected from Table 2 (SEQ ID NOS: 1 to 44). In embodiments, the oligonucleotide is at least 10, at least 12, at least 15, or at least 20 nucleotides in length. In embodiments, the oligonucleotide is about 12 to about 40 nucleotides in length or about 12 to about 25 nucleotides in length. In embodiments, the oligonucleotide is 12, 13, 14, 15, 16, 17, 18, 19, 20, or 21 nucleotides in length. In various embodiments, the antisense oligonucleotide can reduce the expression levels of the target sRNAs in a cell (e.g., a pulmonary epithelial cell) either ex vivo or in vivo. In embodiments, the oligonucleotide consists of a nucleotide sequence that is complementary to a sequence selected from SEQ ID Nos: 1 to 44.

In various aspects and embodiments, the present disclosure provides a composition comprising an antisense oligonucleotide that is at least 10 linked nucleotides in length, and which has a sequence that is complementary to a nucleotide sequence selected from Table 6 (SEQ ID NOS: 45 to 115). In embodiments, the oligonucleotide is at least 10, at least 12, at least 15, or at least 20 nucleotides in length. In embodiments, the oligonucleotide is about 12 to about 40 nucleotides in length or about 12 to about 25 nucleotides in length. In embodiments, the oligonucleotide is 12, 13, 14, 15, 16, 17, 18, 19, 20, or 21 nucleotides in length. In various embodiments, the antisense oligonucleotide can reduce the expression levels of the target sRNAs in a cell (e.g., a pulmonary epithelial cell) either ex vivo or in vivo. In embodiments, the oligonucleotide consists of a nucleotide sequence that is complementary to a sequence selected from SEQ ID Nos: 45 to 115.

In some embodiments, the oligonucleotide has a contiguous sequence of at least six, or at least eight, or at least 10 DNA nucleotides sufficient to recruit RNaseH. RNaseH is a non-sequence-specific endonuclease enzyme that catalyzes the cleavage of RNA in a hybridized RNA/DNA substrate. In some embodiments, the oligonucleotide of the present disclosure is a “gapmer,” that is, the oligonucleotides comprises a central block of deoxynucleotides (also referred to herein as “DNA nucleotides”). In embodiments, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl. A gapmer will typically further comprise a 5′ segment and a 3′ segment, each of the 5′ and 3′ segments being from 2 to 6 nucleotides or from 2 to 4 nucleotides, and where the 5′ and 3′ segments do not contain DNA nucleotides. In embodiments, one or more nucleotides of the 5′ segment and the 3′ segment comprise 2′-O substituents, optionally where all of the nucleotides of the 5′ segment and the 3′ segment comprise 2′-O substituents. In embodiments, the 2′-O substituents are selected from 2′-0 alkyl (e.g., 2′-0 methyl, 2′-O ethyl), 2′-O methoxyethyl (MOE), and a bridged nucleotide having a 2′ to 4′ bridge. In embodiments, the bridged nucleotide has a methylene bridge (LNA) or a constrained ethyl bridge (cEt).

In some embodiments, the oligonucleotide comprises one or more locked or bi-cyclic nucleotides, e.g., bridging the 2′ and 4′ positions (“a bridged nucleotide”). Locked nucleic acid (LNA) or “locked nucleotides” are described, for example, in U.S. Pat. Nos. 6,268,490; 6,316,198; 6,403,566; 6,770,748; 6,998,484; 6,670,461; and 7,034,133, all of which are hereby incorporated by reference in their entireties. LNAs are modified nucleotides that contain a bridge between the 2′ and 4′ carbons of the sugar moiety resulting in a “locked” conformation, and/or bicyclic structure. Other suitable locked nucleotides that can be incorporated in the oligonucleotides of this disclosure include those described in U.S. Pat. Nos. 6,403,566 and 6,833,361, both of which are hereby incorporated by reference in their entireties. In exemplary embodiments, the locked nucleotides are independently selected from a 2′ to 4′ methylene bridge and a constrained ethyl (cEt) bridge (see U.S. Pat. Nos. 7,399,845 and 7,569,686, which are hereby incorporated by reference in their entireties).

In embodiments, the oligonucleotide has a modified polynucleotide backbone or modified internucleotide linkages. The term “internucleotide linkage” refers to the linkage between two adjacent nucleosides in a polynucleotide molecule. Naturally, the internucleotide linkage is a phosphodiester bond that forms between two oxygen atoms of the phosphate group and an oxygen atom of the sugar (either at 3′ or 5′ position) to form two ester bonds bridging between the two adjacent nucleosides. Modification of the internucleotide linkage may provide different characteristics, including but not limited to enhanced stability. For example, phosphorothioate or phosphorodithioate linkages increase the resistance of the internucleotide linkage to nucleases. Another example is phosphoacetate linkage (PACE), which improves transfection characteristics and enhances nuclease resistance. Internucleotide linkages and oligonucleotide backbone modifications which may be employed in the oligonucleotides of the present description include, but are not limited to, phosphodiester, phosphorothioate, phosphorodithioate, methylphosphonate, alkylphosphonate, alkylphosphonothioate, phosphotriester, phosphoramidate, phosphoramidite, phosphorodiamidate, siloxane, carbonate, carboalkoxy, acetamidate, carbamate, morpholino, peptide nucleic acid, borano, thioether, bridged phosphoramidate, bridged methylene phosphonate, bridged phosphorothioate, and sulfone internucleoside linkages.

In some embodiments, the oligonucleotide comprises one or more phosphorothioate or phosphorodithioate internucleotide linkages. These bonds substitute a sulfur atom for a non-bridging oxygen in the phosphate backbone of the oligonucleotide, and can be effective for reducing nuclease digestion. In some embodiments, phosphorothioate or phosphorodithioate bonds can be introduced between the last three to five nucleotides at the 5′- and/or 3′-end of the oligonucleotide to inhibit exonuclease degradation. In some embodiments, the oligonucleotides have a combination of phosphodiester and phosphorothioate/phosphorodithioate linkages. In some embodiments, the oligonucleotides contain at least two, at least three, at least four, at least five, at least six, at least seven, at least eight, at least nine, or at least ten phosphorothioate or phosphorodithioate internucleotide linkages. In some embodiments, the oligonucleotides comprise substantially alternating phosphodiester and phosphorothioate internucleotide linkages. In some embodiments, the oligonucleotides are fully phosphorothioate/phosphorodithioate linked (i.e., all bonds are either phosphorothioate or phosphorodithioate).

In some embodiments, particularly where RnaseH recruitment is not desired, the oligonucleotides have a morpholino backbone. Morpholino oligonucleotides do not trigger the degradation of their target RNA molecules, and can be effective for steric blocking of a target RNA sequence. Morpholino oligonucleotides and their synthesis are disclosed generally in U.S. Pat. Nos. 11,028,386, 10,947,533, and 10,927,378, each of which is hereby incorporated by reference in its entirety. Other backbones that may be used include thiomorpholino and peptide nucleic acid (PNA).

In embodiments, the melting temperature of the oligonucleotide hybridized to its target sequence is at least about 35° C. The Tm of an oligonucleotide is the temperature at which 50% of the oligonucleotide is duplexed with its perfect complement and 50% is free in solution. The Tm can be determined experimentally by measuring the absorbance change of the oligonucleotide with its complement as a function of temperature. The Tm can also be estimated using known publicly available Tm calculators. In some embodiments, the Tm of the oligonucleotide hybridized to its target sequence is at least about 40° C., or at least about 45° C., or at least about 50° C. In some embodiments, the Tm of the oligonucleotide hybridized to its target sequence is from about 35° C. to about 60° C. In some embodiments, the Tm of the oligonucleotide hybridized to its target sequence is from about 40° C. to about 60° C., or from about 50° C. to about 60° C.

In embodiments, the antisense oligonucleotide is formulated for systemic delivery (e.g., parenteral delivery) or local delivery to the lungs, such as but not limited to the route of inhalation. In embodiments the antisense oligonucleotide is encapsulated in a lipid nanoparticle, polymeric nanoparticle, or liposome. In embodiments, the antisense oligonucleotide is delivered by inhalation and is optionally delivered in aerosol (e.g., solution or powder aerosol) form.

In some embodiments, the siRNA or oligonucleotide further comprises a targeting or cell penetrating moiety that increases distribution or accumulation of the agent in certain cells or tissues (e.g., pulmonary epithelium). For example, such a targeting or cell penetrating moiety may be conjugated directly or indirectly to the 3′ end of the oligonucleotides, optionally though a linker which may be biologically cleavable. In some embodiments, the conjugate comprises a sterol conjugate (e.g., cholesterol conjugate) or fatty acid conjugate such as a palmitoyl or stearyl lipid conjugate. Other conjugates are well known.

In various embodiments, the targeting or cell penetrating moiety comprises an antibody or antigen-binding fragment thereof, an aptamer, a peptide, a biological ligand (e.g., including a glycoconjugate), lipid, sterol, cholesterol or derivative thereof, integrin, RGD peptide, or cell-penetrating peptide (CPP). More specifically, the targeting moiety may be selected from a single-domain antibody, a single chain antibody, a bi-specific antibody, a recombinant heavy-chain-only antibody (VHH), a single-chain antibody (scFv), a shark heavy-chain-only antibody (VNAR), a microprotein (cysteine knot protein, knottin), a DARPin, a Tetranectin, an Affibody; a Transbody, an Anticalin, an AdNectin, an Affilin, a Microbody, a phylomer, a stradobody, a maxibody, an evibody, a fynomer, an armadillo repeat protein, a Kunitz domain, an avimer, an atrimer, a probody, an immunobody, a triomab, a troybody, a pepbody, a vaccibody, a UniBody, a DuoBody, a Fv, a Fab, a Fab′, a F(ab′)2, and a peptide mimetic molecule. Various ligand-binding platforms are described in US Patent Nos. or Patent Publication Nos. U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties.

In still other embodiments, the siRNA or oligonucleotide are encapsulated in liposomes, polymeric nanoparticles, or lipid nanoparticles, as is known in the art. In various embodiments, the LNPs comprise a cationic or ionizable lipid, a neutral lipid, a cholesterol or cholesterol moiety, and a PEGylated lipid. Lipid particle formulations that find use with embodiments of the present disclosure include those described in U.S. Pat. Nos. 9,738,593; 10,221,127; 10,166,298, which are hereby incorporated by reference in their entirety. In some embodiments, the LNPs, liposomes, or nanoparticles further comprise a targeting moiety as described. Accordingly, the disclosure in some aspects provides a pharmaceutical composition comprising an effective amount of a composition described herein, and one or more pharmaceutically acceptable excipients or carriers, including LNP, liposome, and polymeric particle compositions.

In other aspects, the present disclosure provides a method (or use of composition) for treating a subject having IPF, comprising administering an effective amount of a composition sufficient for decreasing the expression of a small RNA selected from Table 2 (SEQ ID No 1 to 44). In various embodiments, the composition is effective for decreasing the expression of a small RNA from Table 2 that is demonstrated herein to be higher in abundance in IPF subjects having a high risk of mortality. In various embodiments, the composition comprises an oligonucleotide or composition as described herein. Exemplary oligonucleotide sequences are described in Table 4. These nucleotides can comprise chemical modification patterns as described herein.

In other aspects, the present disclosure provides a method (or use of composition) for treating a subject having a subtype of IPF, comprising administering an effective amount of a composition sufficient for decreasing the expression of a small RNA selected from Table 6 (SEQ ID No 45 to 115). In various embodiments, the composition is effective for decreasing the expression of a small RNA from Table 6 that is demonstrated herein to be higher in abundance in IPF subjects having a high risk of mortality. In various embodiments, the composition comprises an oligonucleotide or composition as described herein. Exemplary oligonucleotide sequences are described in Table 8. These nucleotides can comprise chemical modification patterns as described herein.

In other aspects, the present disclosure provides a method (or use of composition) for treating a subject having IPF, comprising administering an effective amount of a composition sufficient to mimic the action of a small RNA selected from Table 2 (SEQ ID No: 1 to 44). In various embodiments, the composition is effective for mimicking the action of a small RNA from Table 2 that is demonstrated herein to be lower in abundance in IPF subjects having a high risk of mortality. In various embodiments, the composition comprises an siRNA or shRNA, or another suitable format for inducing RNAi as described above. In some embodiments, the pharmaceutical composition comprising the siRNA or shRNA is as described herein. Exemplary siRNAs are described in Table 5. These nucleotides can comprise chemical modification patterns as described herein.

In other aspects, the present disclosure provides a method (or use of composition) for treating a subject having IPF, comprising administering an effective amount of a composition sufficient to mimic the action of a small RNA selected from Table 6 (SEQ ID No 45 to 115). In various embodiments, the composition is effective for mimicking the action of a small RNA from Table 6 that is demonstrated herein to be lower in abundance in IPF subjects having a high risk of mortality. In various embodiments, the composition comprises an siRNA or shRNA, or another suitable format for inducing RNAi as described above. In some embodiments, the pharmaceutical composition comprising the siRNA or shRNA is as described herein. Exemplary siRNAs are described in Table 9. These nucleotides can comprise chemical modification patterns as described herein.

In some embodiments, the subject is identified as having IPF, IPF with a high risk of mortality, or subtype of IPF, according to the methods described herein (e.g., sRNA expression profiling). Dosing and administration schedules can vary, depending on the condition of the patient, and the chemistry of the composition. In various embodiments, the compositions are administered one or more times per week, about weekly, about bimonthly (i.e., about every other week), about monthly, or about quarterly. Dosing and administration schedules can further include varying dosing and administration frequency based on the route or delivery (e.g., parenterally, or direct administration to target tissues) and the patient's response.

In some embodiments, the disclosure provides a method for treating IPF, or IPF having a high risk of mortality, or a subtype of IPF (e.g., having a high risk of mortality), by administering a therapeutic agent that mimics the action or miR-92a-3p or an isoform thereof (such as an isoform described herein). In some embodiments, the isoform is described in Table 10. In various embodiments, the agent is an siRNA that induces degradation of one or a plurality of mRNA targets of miR-92a-3p. In some embodiments, the therapeutic agent is miR-92a-3p or an isoform thereof. In some embodiments, the therapeutic agent induces degradation of one or a plurality of mRNA targets of an isoform of miR-92a-3p that is substantially less abundant in high risk IPF. In embodiments, the siRNA comprises an antisense sequence of SEQ ID NO: 732 or comprising at least 19, 20 or 21 consecutive nucleotides of SEQ ID NO: 732. Nucleobase sequences for sense strands (i.e., passenger strands) for inducing RNAi can be designed as known in the art or as described below. Exemplary passenger strands include SEQ ID NO: 733 or at least 16, 17, 18, 19, or 21 consecutive nucleotides of SEQ ID NO: 733. In some embodiments, the composition has an antisense sequence or strand that comprises the nucleobase sequence of SEQ ID NO: 739 (optionally with an overhang on the 3′ end, such as dTdT as shown in SEQ ID NO: 768). An exemplary passenger strand is provided herein as SEQ ID NO: 797. Known chemical modifications, such as a 5′ phosphate, 2′ modifications, and backbone modifications may be employed as described herein and as known in the art. An exemplary siRNA is shown by SEQ ID NO: 768 and SEQ ID NO: 797.

In some embodiments, the subject is demonstrated as having IPF with a high risk of mortality as described herein, or is otherwise shown to have low or undetectable circulating levels of one or more miR-92a-3p isoforms that are less abundant (or not detected) in IPF with high risk of mortality (as described herein). In some embodiments, the therapeutic agent is administered systemically (e.g., parenterally), or in other embodiments is administered directly to the lungs, for example by inhalation. The therapeutic agent can optionally be encapsulated in particles, such as LNPs, liposome, or polymeric nanoparticles as already described. Composition can be administered periodically, such as from once daily to about monthly, including from about once weekly to about monthly.

In still other aspects and embodiments, the present disclosure provides a method (and use of a composition) for treating an inflammatory or fibrotic disorder. In various embodiments, the disorder is characterized by dysregulation of the WNT/TGF-b/ITGA/FAK regulatory axis. In various embodiments, the disorder is selected from IPF, nonalcoholic steatohepatitis (NASH), heart failure, cardiomyopathy, Crohn's disease, ulcerative colitis, Alzheimer's disease, Parkinson's disease, Huntington's disease, amyotrophic lateral sclerosis (ALS), pre-eclampsia, psoriasis, Pompe's disease, sCID, breast cancer, and other cancers. In various aspects and embodiments, the method or use involves administering a composition that comprises miR-92a-3p or an isoform thereof, or a mimic of miR-92a-3p or an isoform thereof. In some embodiments, the compound that mimics miR-92a-3p or an isoform thereof is an siRNA, where the antisense strand or sequence comprises the sequence of the microRNA or isoform (including as described herein). In various embodiments, the isoform will be an isoform that is downregulated in the disorder or disease of interest (e.g., downregulated in the tissue of interest). In some embodiments, the isoform is set forth in Table 10. In some embodiments, the isoform is SEQ ID NO: 739.

In various embodiments, the composition is administered locally to affected tissues, or is administered systemically. In some embodiments, the compound is encapsulated in particles, including but not limited to lipid nanoparticles (as already described).

Other aspects and embodiments of the present disclosure will be apparent from the following Examples and claims.

As used herein, the term “about”, unless the context requires otherwise, means±10% of an associated value.

### REFERENCES

(1) Wilson M S, Wynn T A. Pulmonary fibrosis: pathogenesis, etiology and regulation. Mucosal Immunol 2009; 2: 103-21.
(2) Ley B, Collard H R, King T E. Clinical Course and Prediction of Survival in Idiopathic Pulmonary Fibrosis. Am J Respir Crit Care Med 2011; 183: 431-40.
(3) Nagai S, Kitaichi M, Hamada K, et al. Hospital-based historical cohort study of 234 histologically proven Japanese patients with IPF. Sarcoidosis Vasc Diffuse Lung Dis Off J WASOG 1999; 16: 209-14.
(4) Kaunisto J, Salomaa E-R, Hodgson U, et al. Demographics and survival of patients with idiopathic pulmonary fibrosis in the FinnishIPF registry. ERJ Open Res 2019; 5: 00170-2018.
(5) Quinn C, Wisse A, Manns S T. Clinical course and management of idiopathic pulmonary fibrosis. Multidiscip Respir Med 2019; 14: 35.
(6) Fraser E, Hoyles R K. Therapeutic advances in idiopathic pulmonary fibrosis. Clin Med Lond Engl 2016; 16: 42-51.
(7) Ley B, Ryerson C J, Vittinghoff E, et al. A Multidimensional Index and Staging System for Idiopathic Pulmonary Fibrosis. Ann Intern Med 2012; 156: 684.
(8) Herazo-Maya J D, Sun J, Molyneaux P L, et al. Validation of a 52-gene risk profile for outcome prediction in patients with idiopathic pulmonary fibrosis: an international, multicentre, cohort study. Lancet Respir Med 2017; 5: 857-68.
(9) Zhang N, Guo Y, Wu C, Jiang B, Wang Y. Identification of the Molecular Subgroups in Idiopathic Pulmonary Fibrosis by Gene Expression profiles. Comput Math Methods Med 2021; 2021: 7922594.
(10) He Y, Shang Y, Li Y, et al. An 8-ferroptosis-related genes signature from Bronchoalveolar Lavage Fluid for prognosis in patients with idiopathic pulmonary fibrosis. BMC Pulm Med 2022; 22: 15.
(11) Huang T, He W-Y. Construction and Validation of a Novel Prognostic Signature of Idiopathic Pulmonary Fibrosis by Identifying Subtypes Based on Genes Related to 7-Methylguanosine Modification. Front Genet 2022; 13: 890530.
(12) Zhang C. Novel functions for small RNA molecules. Curr Opin Mol Ther 2009; 11:641-51.
(13) Zelli V, Compagnoni C, Capelli R, et al. Circulating MicroRNAs as Prognostic and Therapeutic Biomarkers in Breast Cancer Molecular Subtypes. J Pers Med 2020; 10: 98.
(14) Israeli D, Poupiot J, Amor F, et al. Circulating miRNAs are generic and versatile therapeutic monitoring biomarkers in muscular dystrophies. Sci Rep 2016; 6: 28097.
(15) Gupta S K, Bang C, Thum T. Circulating MicroRNAs as Biomarkers and Potential Paracrine Mediators of Cardiovascular Disease. Circ Cardiovasc Genet 2010; 3: 484-8.
(16) Herrera-Rivero M, Zhang R, Heilmann-Heimbach S, et al. Circulating microRNAs are associated with Pulmonary Hypertension and Development of Chronic Lung Disease in Congenital Diaphragmatic Hernia. Sci Rep 2018; 8: 10735.
(17) Komatsu M, Yamamoto H, Kitaguchi Y, et al. Clinical characteristics of non-idiopathic pulmonary fibrosis, progressive fibrosing interstitial lung diseases: A single-center retrospective study. Medicine (Baltimore) 2021; 100: e25322.
(18) Maher™, Oballa E, Simpson J K, et al. An epithelial biomarker signature for idiopathic pulmonary fibrosis: an analysis from the multicentre PROFILE cohort study. Lancet Respir Med 2017; 5: 946-55.
(19) Bommert A, Welchowski T, Schmid M, Rahnenfuhrer J. Benchmark of filter methods for feature selection in high-dimensional gene expression survival data. Brief Bioinform 2022; 23: bbab354.
(20) Longato E, Vettoretti M, Di Camillo B. A practical perspective on the concordance index for the evaluation and selection of prognostic time-to-event models. J Biomed Inform 2020; 108: 103496.
(21) Harrell F E. Regression modeling strategies: with applications to linear models, logistic and ordinal regression, and survival analysis, 2. ed. Cham: Springer, 2015.
(22) Vickers A J, Elkin E B. Decision Curve Analysis: A Novel Method for Evaluating Prediction Models. Med Decis Making 2006; 26: 565-74.
(23) Clark T G, Bradburn M J, Love S B, Altman D G. Survival Analysis Part I: Basic concepts and first analyses. Br J Cancer 2003; 89: 232-8.
(24) Blondal T, Brunetto M R, Cavallone D, et al. Genome-Wide Comparison of Next-Generation Sequencing and qPCR Platforms for microRNA Profiling in Serum. In: Dalmay T, ed. MicroRNA Detection and Target Identification. New York, N Y: Springer New York, 2017: 21-44.
(25) Glass D S, Grossfeld D, Renna H A, et al. Idiopathic pulmonary fibrosis: Current and future treatment. Clin Respir J 2022; 16: 84-96.
(26) Amor M S, Rosengarten D, Shitenberg D, Pertzov B, Shostak Y, Kramer M R. Lung Transplantation in Idiopathic Pulmonary Fibrosis: Risk Factors and Outcome. Isr Med Assoc J IMAJ 2020; 22: 741-6.
(27) Cottin V, Hirani N A, Hotchkin D L, et al. Presentation, diagnosis and clinical course of the spectrum of progressive-fibrosing interstitial lung diseases. Eur Respir Rev 2018; 27: 180076.

### EXAMPLES

### Example 1: Small RNA Biomarkers Predict 3-Year, Transplant-Free Survival in Idiopathic Pulmonary Fibrosis

### Participants

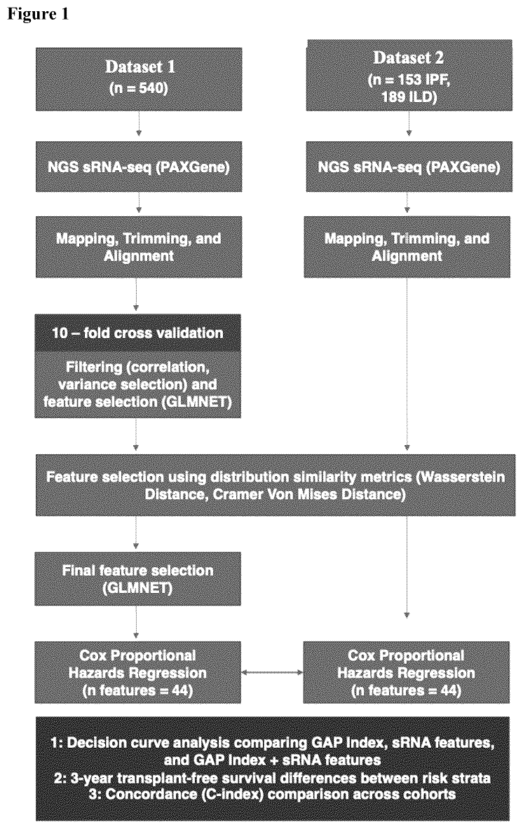

Study design is summarized in FIG. 1. PAXgene blood RNA tubes were collected from patients enrolled in the Prospective Study of Fibrosis In The Lung Endpoints (DATASET 1) (n=540) and patients participating in a long-term cohort study associated with DATASET 2 (n=153 IPF patients and n=189 fibrotic ILD patients).

Demographic information and spirometry results including forced vital capacity (FVC) and diffusion capacity of the lung for carbon monoxide (DLCO) were collected at the time of blood draw for the DATASET 1 cohort, and within 6 months of blood draw for the DATASET 2 cohort. Transplant-free survival in decimal years from blood draw is censored at 3 years for both datasets.

### Data Preparation and NGS Sequencing

Total RNA was extracted using the PAXgene Blood RNA Extraction Kit (Qiagen) in batches of 24. RNA quality was assessed using the RNA Pico Sensitivity Kit (Perkin Elmer) on a LabChip GX Touch (Perkin Elmer). Samples had an average RIN Score of 8.6 (±1.1). Small RNA libraries were prepared from 250 ng of input RNA, in batches of 96 using the NextFlex Small RNA-Seq Kit v3 (Perkin Elmer) using 20 PCR cycles. Resulting libraries were pooled and sequencing at a target depth of 20 million reads per sample using an S4, 300 cycle flow cell on a NovaSeq 6000 (Illumina).

### Adapter Trimming and Short Read Alignment

Sequencing reads were processed by trimming adaptor sequence using a Regex-based search and trim algorithm, where 5′ TGGAATTCCTCGGGTGCCAAGG 3′ (SEQ ID NO: 342) (containing up to a 15 nucleotide 3′-end truncation) was input to identify the 3′ adaptor, and a Levenshtein Distance of 2 or Hamming Distance of 5. Parameters for Regex searching required that the 1^{st} nucleotide of the 3′-adaptor be unaltered with respect to nucleotide insertions, deletions, and/or swaps; 5′-adaptor 5′ TCTTTCCCTACACGACGCTCTTCCGATCT 3′ (SEQ ID NO: 343) (containing up to a 15 nucleotide 5′-end truncation) was input to identify the 5′ cloning adaptor sequence, and a Levenshtein Distance of 2 or Hamming Distance of 5. Parameters for Regex searching required that the 29th nucleotide of the user-specified search term be unaltered with respect to nucleotide insertions, deletions, and/or swaps. Paired-end reads were removed if they were not an exact match. A 4-nucleotide NNNN prefix and NNNN postfix were used as a Unique Molecular Index (UMI) to quantify reads of unique small RNAs. The UMIs were removed after quantification. Reads were aligned to a 17-95 nucleotide tiled array of the human genome (hG38) with a Levenshtein distance of 2. The number of trimmed reads per million was calculated for each unique small RNA cloned in the dataset.

### Feature Selection

sRNA feature expression was calculated using trimmed reads per million (TRPM). A feature selection pipeline using 10-fold cross validation on the DATASET 1 cohort incorporated filtering methods and Cox proportional hazards regression with an elastic net penalty (GLMNET) filtered to a candidate pool of features most likely to predict survival. Two filtering methods for sRNA feature selection were chosen from recommendations by Bommert et. al., for high-dimensional gene expression survival data: (1) correlation to Martingale residuals, and (2) variance selection. Martingale residuals were calculated through fitting a Cox proportional hazards regression model without covariates to predict 3-year transplant-free survival. Variance selection referred to filtering features to those with the highest variance, following from the assumption that features with high variance are likely to show signal.

For each fold, miRNA, pre-miRNA and tRNA features with presence in at least 40% of the training set were included for consideration in the panel if the absolute value of Spearman's rho between TRPM feature expression and Martingale residuals was ≥0.01, with a p value<0.05. The feature pool was filtered to the top 300 small RNAs with the highest variance. Finally, a Cox proportional hazards regression with an elastic net penalty (GLMNET) was fit to the 300 sRNA features using the training set with a cutoff of lambda=0.01. The resulting candidate feature set included 504 unique small RNAs from the aforementioned Cox proportional hazards models for each fold split.

Wasserstein and Cramer Von Mises distances were calculated for the 504—feature candidate set, comparing the DATASET 1 and DATASET 2 studies. Features were limited to those Cramer Von Mises and Wasserstein distances below the median across the 504 features. The final 44-sRNA panel was chosen through fitting a Cox proportional hazards regression with an elastic net penalty (GLMNET) on the full DATASET 1 dataset for the remaining 235 features with a cutoff of lambda=0.04.

### Time to Event Analysis

To determine whether the 44-sRNA panel has utility when compared to or combined with the GAP index, three Cox proportional hazards regression models were fit using the following features: (1) sRNA data alone, (2) numeric features based on age, gender, FVC and DLCO that sum to create the GAP index and (3) sRNA data and the aforementioned GAP features. All models containing sRNA features utilize log 10-transformed TRPM expression for the 44 features in the final panel. Cox regression models were fit separately to the DATASET 1 dataset and the DATASET 2 dataset (both IPF and non-IPF). The concordance index, or C-index, which summarizes how well a predicted risk score described time-to-event data, was utilized to compare performance of the three types of models. C-indices were compared both retrospectively and with confidence intervals using 50 bootstraps (using R's rms package).

Decision curve analysis was utilized to compare stratification performance at a range of thresholds. Thresholds were chosen using the probability of mortality within 3 years, extracted from the fitted survival models. Kaplan Meier curves and cumulative incidence plots were utilized to evaluate differences in transplant-free survival and mortality across risk strata and compared to GAP stage. Hazard ratios compared risk strata in each model.

### Dataset Comparison and Model Coefficients

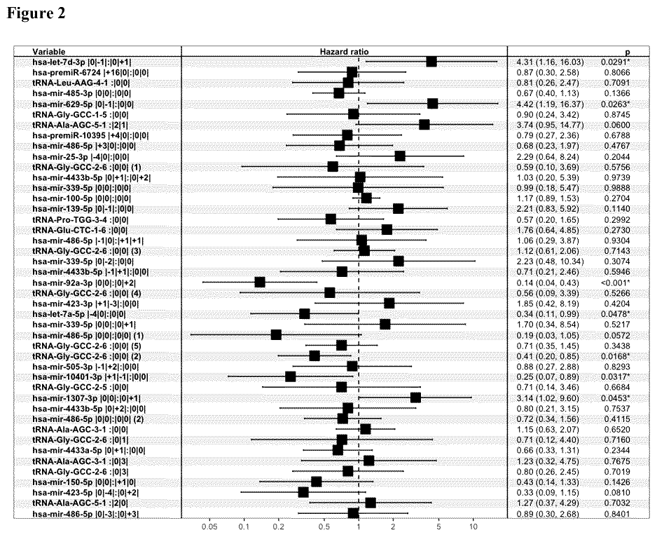

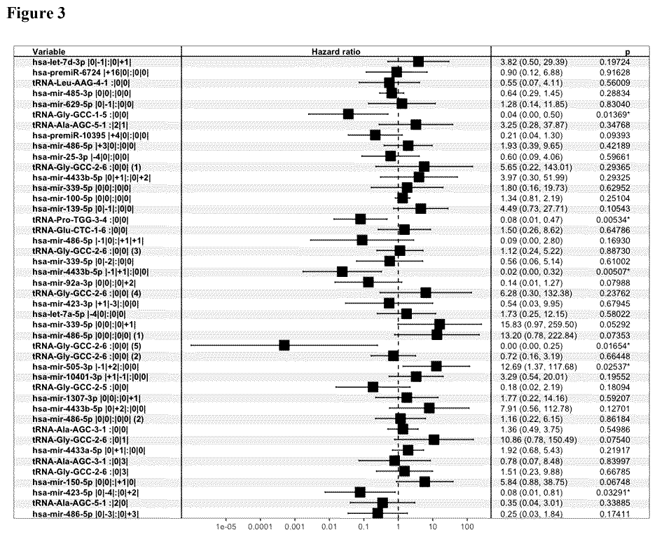

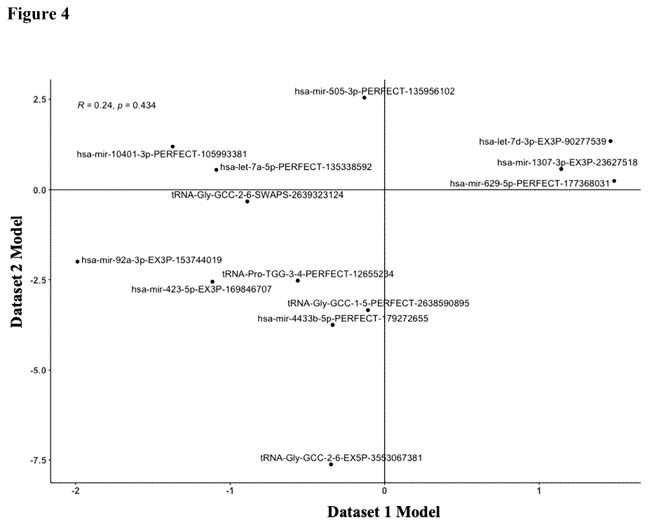

Regression coefficients for the sRNA-alone Cox regression models in each dataset (DATASET 1 IPF and DATASET 2 pan-ILD) can be found in FIGS. 2 and 3, respectively. Of the 13 sRNAs with statistically significant coefficients in either model, including miR-92a-3p (3′ extension isoform), tRNA-Gly-GCC-2-6 (multiple swaps isoform), miR-10401-3p (perfect mapping), let-7a-5p (perfect mapping), mir-629-5p (perfect mapping), mir-1307-3p (3′ extension isoform), let-7d-3p (3′ extension isoform), tRNA-Pro-TGG-3-4 (perfect mapping), miR-4433b-5p (perfect mapping), miR-423-5p (3′ extension isoform), tRNA-Gly-GCC-1-5 (perfect mapping), miR-595-3p (perfect mapping), and tRNA-Gly-GCC-2-8 (5′ extension isoform), 10 of 13 have consistent directionality across the two models, indicating overall similarity across models (FIG. 4).

### Comparative Concordance Indices

For the DATASET 1 cohort, the sRNA-alone Cox regression model achieved a retrospective C-index of 0.746, bootstrapped training C-index of 0.772 (95% CI 0.769-0.777) and bootstrapped testing C-index of 0.714 (95% CI 0.713-0.716). The GAP components—alone Cox regression model achieved a retrospective C-index of 0.725, bootstrapped training C-index of 0.728 (95% CI 0.723-0.732), and bootstrapped testing C-index of 0.722 (0.720-0.723). The sRNA+GAP Cox regression model achieved a retrospective C-index of 0.792, bootstrapped training C-index of 0.815 (95% CI 0.810-0.818) and bootstrapped testing C-index of 0.767 (95% CI 0.764-0.769).

For the DATASET 2 pan-ILD cohort, the sRNA-alone Cox regression model achieved a retrospective C-index of 0.757, bootstrapped training C-index of 0.818 (95% CI 0.810-0.825) and bootstrapped testing C-index of 0.705 (95% CI 0.700-0.711). The GAP components—alone Cox regression model achieved a retrospective C-index of 0.729, bootstrapped training C-index of 0.731 (95% CI 0.718-0.722), and bootstrapped testing C-index of 0.720 (0.718-0.722). The sRNA+GAP Cox regression model achieved a retrospective C-index of 0.801, bootstrapped training C-index of 0.854 (95% CI 0.850-0.861) and bootstrapped overfitting-corrected C-index of 0.744 (95% CI 0.738-0.751).

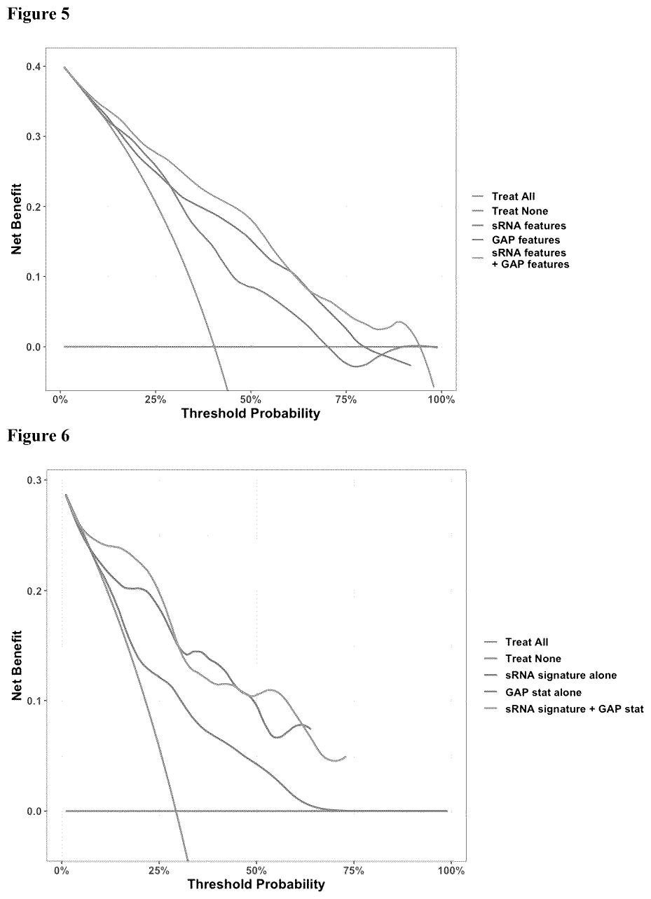

Decision Curve Analysis Probability of mortality within 3 years was extracted from Cox regression model fit for each model. Net benefit of stratification at varying probability thresholds was calculated using methods described by Vickers et al (2008). For the DATASET 1 cohort, results of decision curve analysis plotting net benefit vs. threshold to treat are presented in FIG. 5, comparing net benefit of the three Cox regression models in addition to the case where all are treated and where none are treated. The benefit of the sRNA features alone model is greater than that of the GAP model at all thresholds greater than −0.25, while the sRNA+GAP features model has the greatest net benefit at nearly all thresholds.

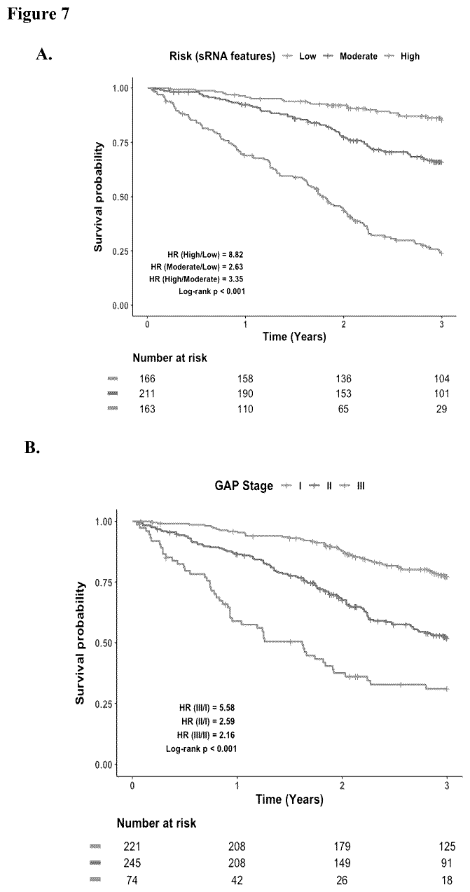

For the DATASET 2 pan-ILD cohort, results of decision curve analysis plotting net benefit vs. threshold to treat are presented in FIG. 6, comparing net benefit of the three Cox regression models in addition to the case where all are treated and where none are treated. The benefit of both the sRNA model alone and the sRNA+GAP model is greater than the GAP model at nearly all thresholds, with the sRNA alone model and the sRNA+GAP model alternating in highest benefit at different thresholds.

### Time to Event Analysis

Two stratification methodologies were considered to designate high risk individuals in need of treatment or transplant: three-strata and two-strata binning. For both the DATASET 1 IPF and DATASET 2 pan-ILD datasets, the three—strata thresholds were as follows: 0-25% probability of mortality within 3 years (Low Risk), 25-50% probability of mortality within 3 years (Moderate Risk), and >50% probability of mortality within 3 years (High Risk). These strata were applied to the sRNA models and the sRNA+GAP models, while the GAP alone equivalent was represented by stratification using GAP stage. The two-strata thresholds were as follows for the sRNA, GAP, and sRNA+GAP models: 40% probability of mortality within 3 years for the DATASET 1 dataset and 35% probability of mortality within 3 years for the DATASET 2 dataset.

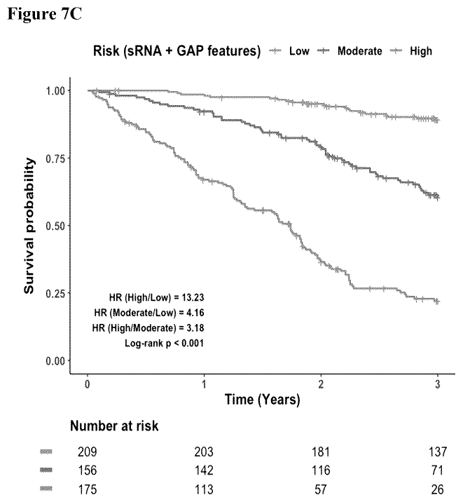

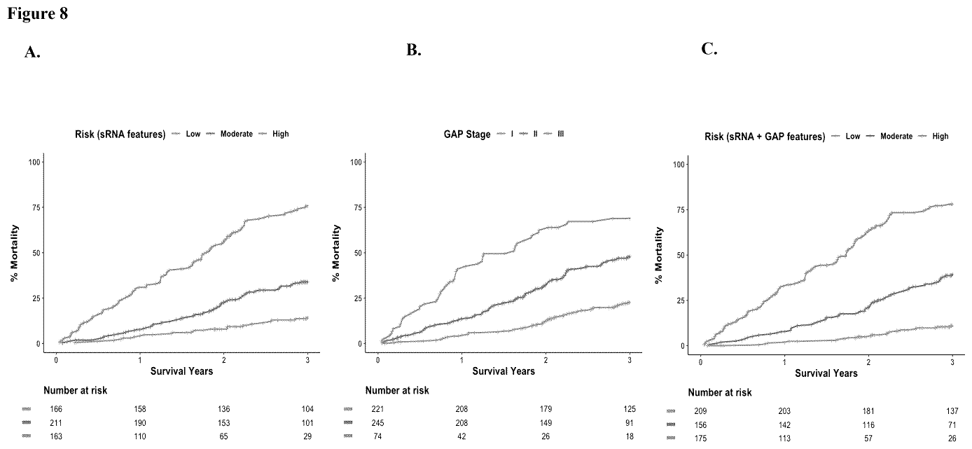

Clinical metadata by risk strata for the 3—strata model using the sRNA model, GAP stage, and sRNA+GAP model for the DATASET 1 cohort was evaluated. The sRNA model stratification has no significant differences in sex (p=0.401) or age (0.063), while both GAP stage and sRNA+GAP stratification are highly differentiated by sex and age. Additionally, the high—risk group in the sRNA model stratification has a greater proportion of members dead within 3 years with similar overall survival to the GAP stage III group, while identifying over twice the population (163 vs. 74). Kaplan-Meier curves with hazard ratios for the 3-class models in DATASET 1 are shown in FIG. 7, with cumulative mortality curves in FIG. 8. Both the sRNA and sRNA+GAP models' resulting three classes were significantly different with respect to transplant-free survival, with log-rank p-value<0.001, as were the three GAP stages. Hazard ratios for the sRNA model alone were significantly greater than the GAP stage, especially that of High/Low risk (8.82 vs. 5.58), while the sRNA+GAP model outperformed both the sRNA and GAP stage models (13.23 HR for High/Low).

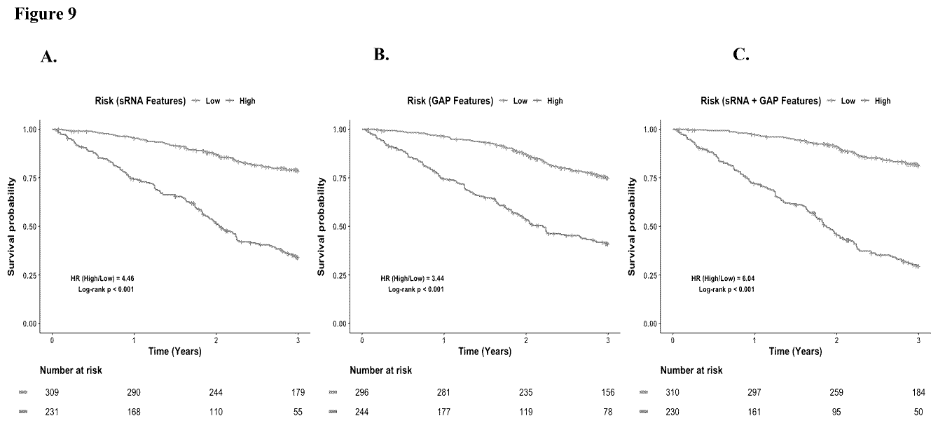

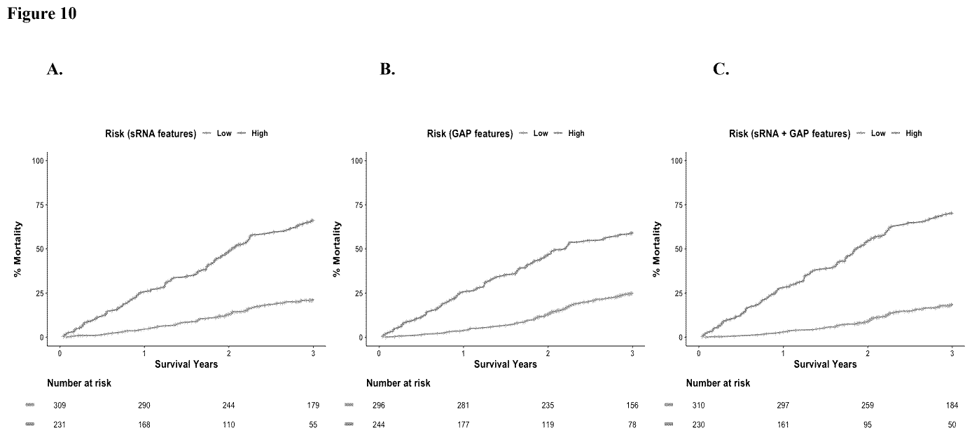

Clinical metadata by risk strata for the 2—strata model using the sRNA model, GAP model, and sRNA+GAP model for the DATASET 1 cohort was evaluated. While there exist differences in GAP stage distribution by strata in the sRNA and GAP+sRNA models, members of all three GAP stages appear in all strata. The sRNA model and the sRNA+GAP model stratifications have no significant differences in sex (p=0.59 and p=0.078, respectively), while the GAP stratification is highly differentiated by sex. All 2—class stratification models were partially differentiated by age. Additionally, the high—risk group in the sRNA model stratification has a greater proportion of members dead within 3 years with similar overall survival to the high-risk GAP group. Kaplan-Meier curves with hazard ratios for the 2-class models in DATASET 1 are shown in FIG. 9, with cumulative mortality curves in FIG. 10. Both the sRNA and sRNA+GAP models' resulting two classes were significantly different with respect to transplant-free survival, with log-rank p-value<0.001, as were the three GAP stages. The hazard ratio for the sRNA model alone was significantly greater than the GAP stage, (4.46 vs. 3.44), while the sRNA+GAP model outperformed both the sRNA and GAP stage models (HR=6.04).

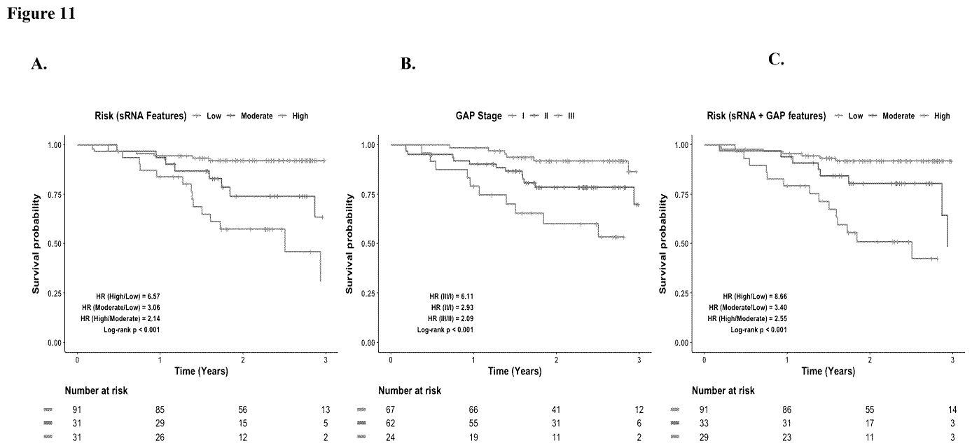

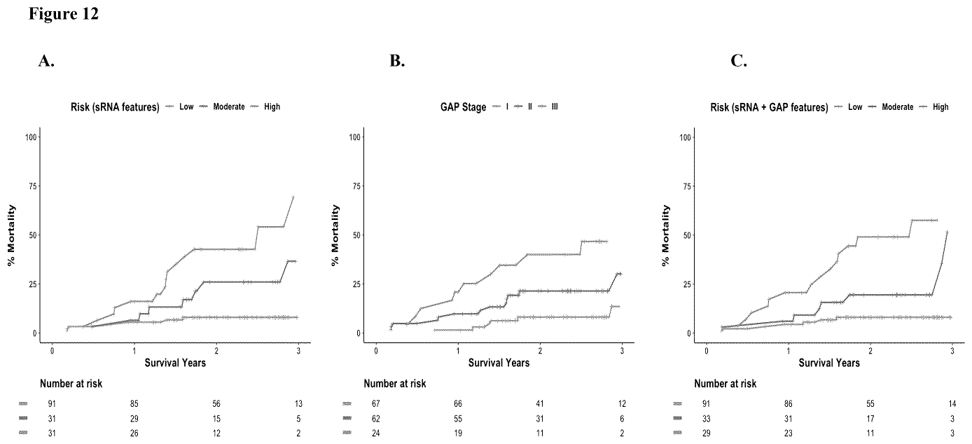

Clinical metadata by risk strata for the 3—strata model using the sRNA model, GAP stage, and sRNA+GAP model for the DATASET 2 IPF cohort was evaluated. While there exist differences in GAP stage distribution by strata in the sRNA and GAP+sRNA models, members of all three GAP stages appear in all strata. The sRNA model stratification has no significant differences in sex (p=0.32) or age (p=0.754). The GAP stage stratification is differentiated by sex (p=0.002) but not age (p=0.118). The sRNA+GAP stratification has no significant differences in sex (p=0.29) or age (p=0.823). Kaplan-Meier curves with hazard ratios for the 3-class models in DATASET 2 IPF are shown in FIG. 11, with cumulative mortality curves in FIG. 12. Both the sRNA and sRNA+GAP models' resulting three classes were significantly different with respect to transplant-free survival, with log-rank p-value<0.001, as were the three GAP stages. Hazard ratios for the sRNA model alone were moderately greater than the GAP stage, especially that of High/Low risk (6.57 vs. 6.11), while the sRNA+GAP model outperformed both the sRNA and GAP stage models (8.66 HR for High/Low).

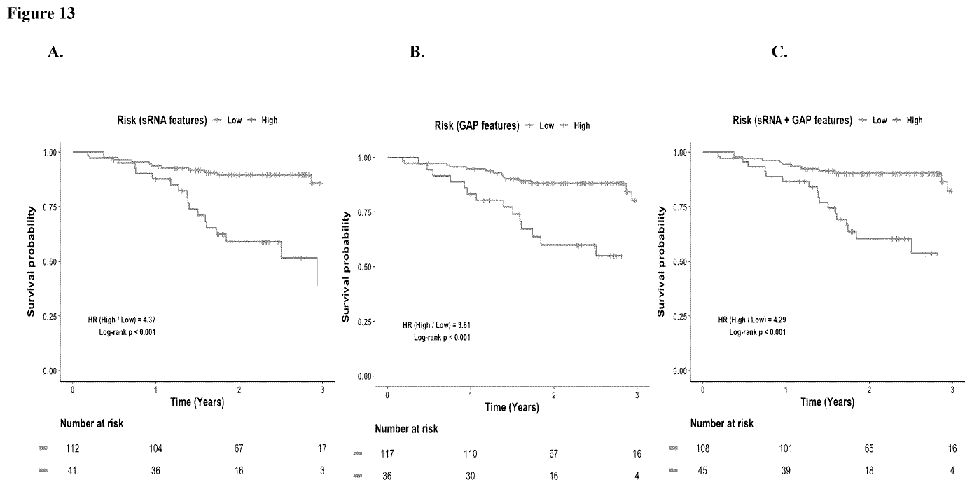

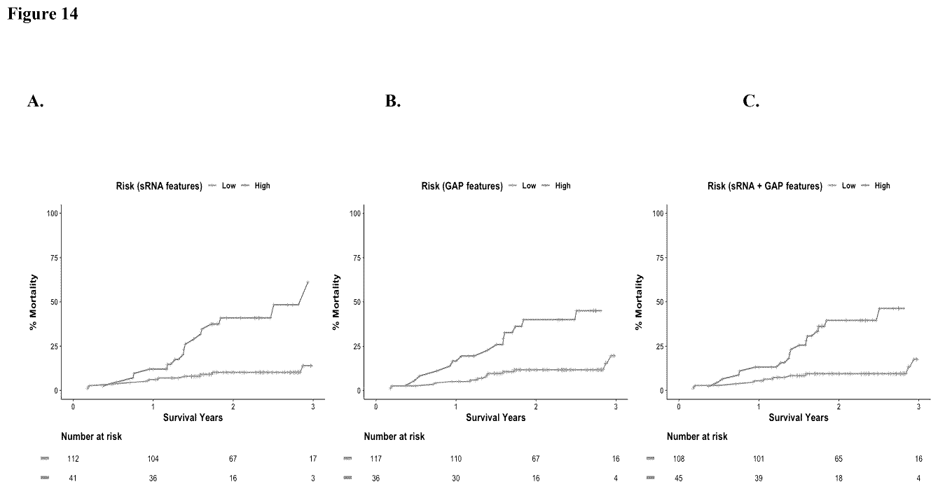

Clinical metadata by risk strata for the 2—strata model using the sRNA model, GAP model, and sRNA+GAP model for the DATASET 2 IPF cohort was evaluated. No models are differentiated by sex or age in this case, potentially due to low sample size. Additionally, high and low risk groups have a similar proportion of members dead within 3 years across models, with the sRNA and sRNA+GAP models identifying modestly more high-risk members. Kaplan-Meier curves with hazard ratios for the 2-class models in DATASET 2 IPF are shown in FIG. 13, with cumulative mortality curves in FIG. 14. All models' resulting two classes were significantly different with respect to transplant-free survival, with log-rank p-value<0.001. The hazard ratio for the sRNA model alone (4.37) was greater than the GAP model, (HR=3.81) and the sRNA+GAP model (HR=4.29).

Clinical metadata by risk strata for the 3—strata model using the sRNA model, GAP stage, and sRNA+GAP model for the DATASET 2 non-IPF cohort was evaluated. While there exist differences in GAP stage distribution by strata in the sRNA and GAP+sRNA models, members of all three GAP stages appear in all strata. The sRNA model stratification has no significant differences in sex (p=0.76) or age (p=0.57). The GAP stage stratification is differentiated by sex (p=0.003) and age (p=0.004). The sRNA+GAP stratification has no significant differences in sex (p=0.237) or age (p=0.313). No stratification appears to have any differences in diagnosis. Additionally, the high—risk group in the sRNA model stratification has a greater proportion of members dead within 3 years with similar overall survival to the GAP stage III group, while identifying nearly twice the population (42 vs. 22).

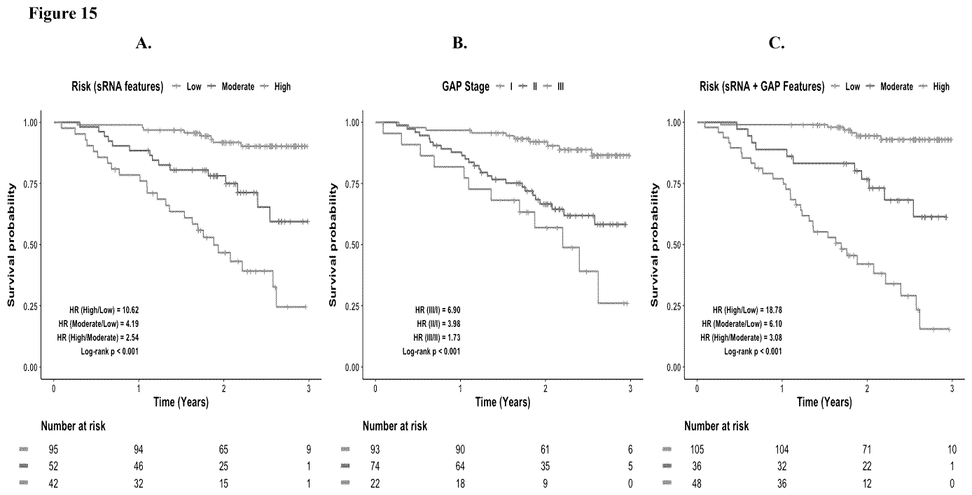

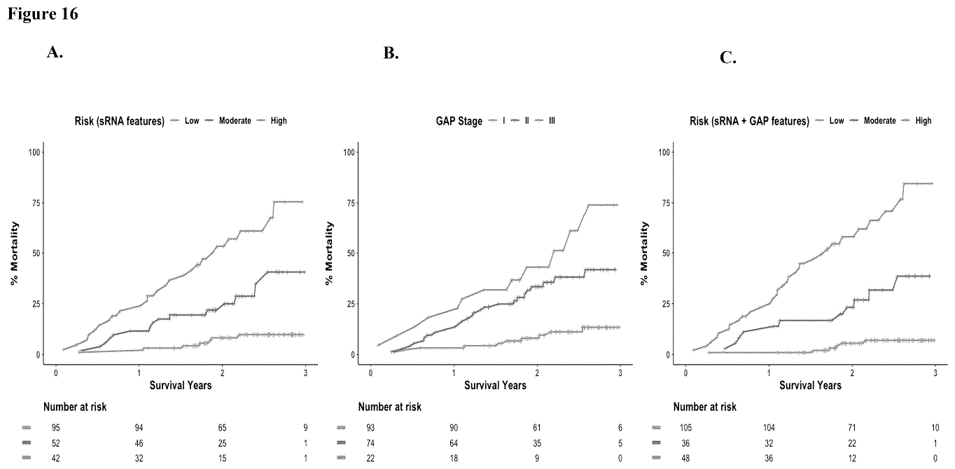

Kaplan-Meier curves with hazard ratios for the 3-class models in DATASET 2 non-IPF are shown in FIG. 15, with cumulative mortality curves in FIG. 16. Both the sRNA and sRNA+GAP models' resulting three classes were significantly different with respect to transplant-free survival, with log-rank p-value<0.001, as were the three GAP stages. Hazard ratios for the sRNA model alone were moderately greater than the GAP stage, especially that of High/Low risk (10.62 vs. 6.90), while the sRNA+GAP model outperformed both the sRNA and GAP stage models (18.78 HR for High/Low).

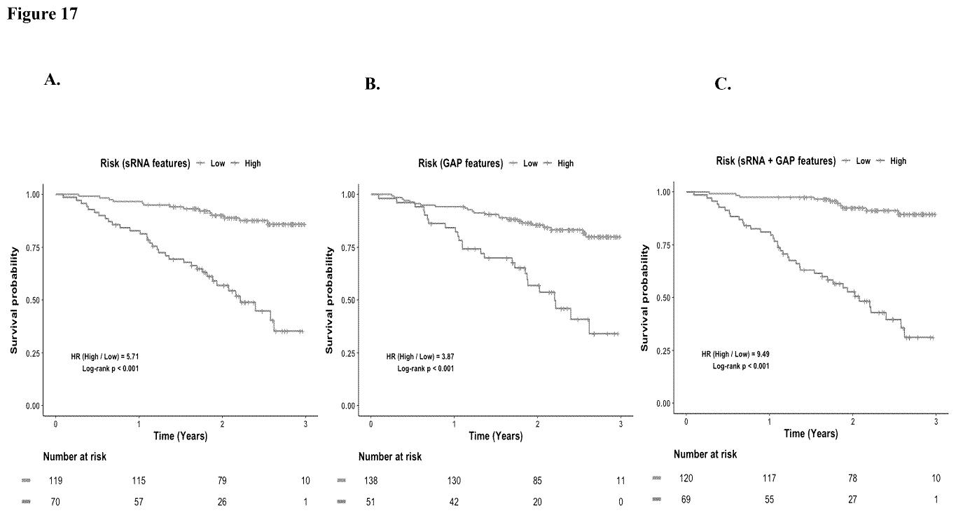

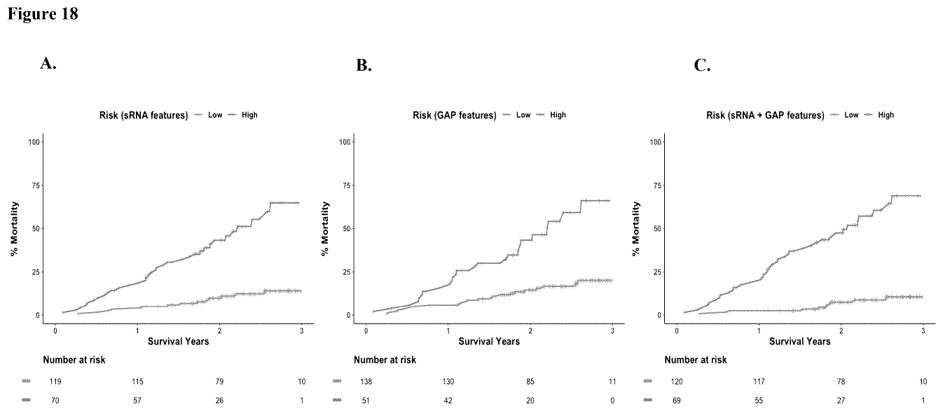

Clinical metadata by risk strata for the 2—strata model using the sRNA model, GAP model, and sRNA+GAP model for the DATASET 2 non-IPF cohort was evaluated. No models are differentiated by sex or age in this case, potentially due to low sample size. Additionally, the high risk group in the sRNA+GAP model has a modestly higher proportion of members dead within 3 years compared to the other two models, while identifying modestly more high-risk members than the GAP model. Kaplan-Meier curves with hazard ratios for the 2-class models in DATASET 2 IPF are shown in FIG. 17, with cumulative mortality curves in FIG. 18. All models' resulting two classes were significantly different with respect to transplant-free survival, with log-rank p-value<0.001. The hazard ratio for the sRNA model alone (5.37) was greater than the GAP model, (HR=3.87), with the sRNA+GAP model outperforming both the sRNA and GAP models (HR=9.49).

A signature of 44 small RNAs as predictive of 3-year transplant free survival was identified in the discovery cohort (DATASET 1). Filtering criteria identified features that are statistically significantly associated with transplant-free survival using correlation and variance selection methods. Martingale residuals were used as uncensored continuous survival outcome variables, which enabled filtering based on correlation. Cox regression with elastic net penalty using cross validation created a more robust predictive model. Using these 44 features, the data was fitted into a separate Cox regression model to the validation cohort (DATASET 2). Decision curve analysis indicated that models containing sRNA features performed better than the GAP index, with a model containing GAP and sRNA features performing the best. Both two and three-strata risk groupings were created in each dataset, separating IPF and non-IPF in DATASET 2. The justification for each was as follows: three strata allows for direct comparison to the GAP staging model, while two strata allows for efficient decision making among physicians, where a high risk patient would receive a treatment or transplant, while a low risk patient would not. The combined sRNA and clinical prediction model including TRPM features in addition to age, gender, FVC % and DLCO % provided better performance than the GAP features alone.

### Example 2: Idiopathic Pulmonary Fibrosis Subtyping sRNA Panel

49 IPF PAXgene Blood RNA samples were used as a discovery cohort. RNA was extracted using the PAXgene Blood RNA Extraction Kit (QIAGEN) in batches of 24. The extracted RNA was (1) quantified in triplicate using the quant-IT RNA Assay Kit (ThermoScientific) on a Fluoroskan fluorometer (ThermoScientific) to calculate concentration, and (2) RNA quality was assessed using the RNA Pico Sensitivity Kit (PerkinElmer) on a LabChip GX Touch (PerkinElmer). Small RNA sequencing was performed on the purified RNA samples using the Next generation sequencing (NGS) libraries prepared from 250 ng of input RNA, in batches of 96 using the NextFlex Small RNA-Seq Kit v3 for Sciclone Automation on a Sciclone iQ NGS Workstation (PerkinElmer).

To facilitate sample pooling, i7/i5 indexes were incorporated using the NextFlex Small RNA-Seq Kit v3 for Sciclone Automation (BIOO). Libraries were quantified in triplicate using the quant-IT dsDNA HS Assay Kit (Thermo) on a Fluoroskan fluorometer (Thermo). Library quality analysis was assessed using the LabChip DNA 3K NGS Assay Kit (PerkinElmer) to verify proper library size generation.

Libraries were pooled and re-quantified using the KAPA NGS Library Quantification Kit (Kapa Biosciences) and were diluted to a final concentration of 1.6 nM using a coefficient of 200 bp as the mean calculated insert size. To target 20 million reads per sample, libraries were individually prepared using the XP 4-Lane Kit (Illumina). Libraries were sequenced using S4, 300 cycle Flow Cell Kits (Illumina) on a NovaSeq 6000 Sequencing System (Illumina).

Sequencing reads were processed by trimming adaptor sequence using a Regex-based search and trim algorithm, where 5′ TGGAATTCCTCGGGTGCCAAGG 3′ (SEQ ID NO: 342) (containing up to a 15 nucleotide 3′-end truncation) was input to identify the 3′ adaptor, and a Levenshtein Distance of 2 or Hamming Distance of 5. Parameters for Regex searching required that the 1^{st} nucleotide of the 3′-adaptor be unaltered with respect to nucleotide insertions, deletions, and/or swaps; 5′-adaptor 5′ TCTTTCCCTACACGACGCTCTTCCGATCT 3′ (SEQ ID NO: 342) (containing up to a 15 nucleotide 5′-end truncation) was input to identify the 5′ cloning adaptor sequence, and a Levenshtein Distance of 2 or Hamming Distance of 5. Parameters for Regex searching required that the 29^{th} nucleotide of the user-specified search term be unaltered with respect to nucleotide insertions, deletions, and/or swaps. Paired-end reads were removed if they were not an exact match. A 4-nucleotide NNNN prefix and NNNN postfix were used as a Unique Molecular Index (UMI) to quantify reads of unique small RNAs. The UMIs were removed after quantification. Reads were aligned to a 17-95 nucleotide tiled array of the human genome (hG38) with a Levenshtein distance of 2. The number of trimmed reads per million was calculated for each unique small RNA cloned in the dataset.

Corresponding control PAXgene blood RNA sRNA NGS data were sourced from GSE100467 and GSE46579 and downloaded from the GEO database. Additional control samples were sourced from healthy volunteers. The total control cohort consisted of 170 samples, either healthy or diagnosed with a neurogenerative disorder, 57% male with a mean age of 69.31 (±7.85). The 49 PAXgene IPF samples were 87.8% male with a mean age of 64.67 (±8.71). An initial sRNA-signature of 86 sRNA features was identified that differentiated IPF from non-IPF control. Features were selected based on filtering criteria that maximize separation between classes, then filtered to features with high classification importance based on coefficients from a GLMnet ridge classification model. Finally, a support vector machine (SVM) was fit using a linear kernel. SVM model testing resulted in an AUC=0.999 (p<0.001) with an accuracy of 99.6%. This sRNA signature was used to identify 4 putative subtypes within the discovery cohort. These subtypes were identified using Hierarchical Clustering on Principal Components, which combines Principal Component Analysis (PCA) and two clustering algorithms: hierarchical clustering, and k-means clustering. PCA reduces dimensionality in high-dimensional datasets by geometrically projecting the feature set onto lower-dimensional summaries known as principal components. The principal components are uncorrelated and minimize the distance between the data and its projection. The input variables for PCA included log 10 transformed sRNA expression data, measured in trimmed reads per million (TRPM), scaled using unit variance scaling. Using goodness of fit (R²) and predictability (Q²) measures, the first five principal components, calculated using singular value decomposition, were chosen as the optimum number to capture variation in the data. A scree plot confirmed the choice of 5 principal components to explain the majority of variation in the data.

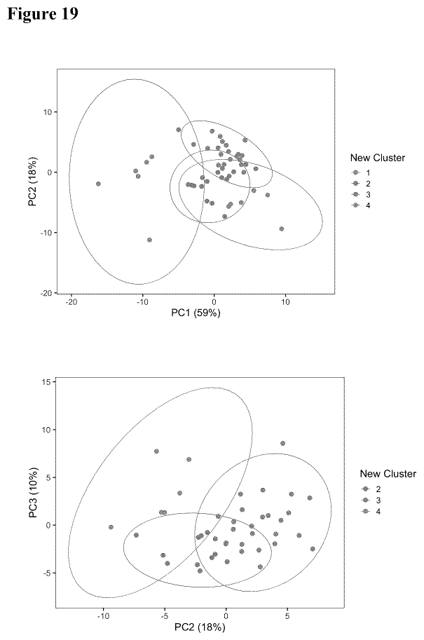

Hierarchical clustering using Ward's linkage and k-means clustering consolidated the partitions using a predetermined number of 4 clusters. The data's projection onto the first two principal components is shown in the score plot in FIG. 19. Principal Component 1 (PC1) and Principal Component 2 (PC2) accounted for 59% and 18% of the variation in the data, respectively.

A sRNA signature of 719 sRNA features was identified that classified the four subtypes in the discovery cohort resulting from two repetitions of ten-fold cross validation. SVM model testing resulted in an AUC=0.917 (p<0.001) with a minimum per-class accuracy of 88%.

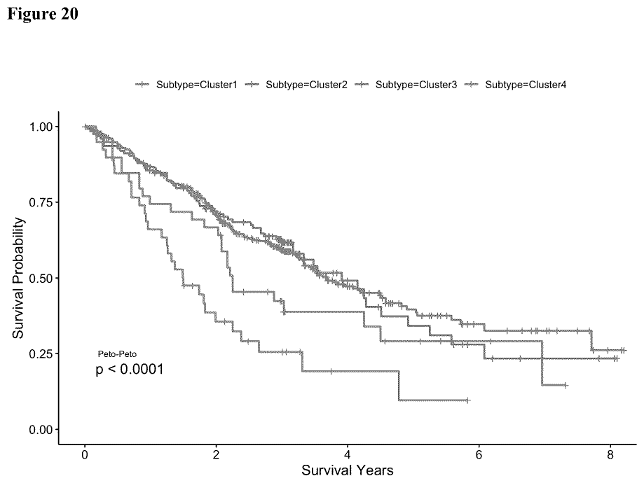

Following the initial discovery of putative subtypes, an additional 546 samples were sourced from the DATASET 1 study as a validation cohort. Wasserstein distance and Cramer Von Mises distance were computed for three comparative pairs: the discovery cohort to the two experimental batches in the validation cohort, and the two validation batches with each other, for each sRNA in the 719-sRNA feature set. sRNA expression was measured by log-10 transformed trimmed reads per million, scaled to the mean and standard deviation of each marker. A subset of 71 sRNA features were identified using the filtering criteria of both Cramer Von Mises and Wasserstein distances below the median for each pair of cohorts filtering to only miRNAs. Applying this SVM model to the validation cohort resulted in a stratified cohort for the entire DATASET 1 study, with four distinct subtypes that correlate to overall survival (FIG. 20).

### Example 3: Therapeutic Potential of mir92a Small RNA Isoforms to Treat Idiopathic Pulmonary Fibrosis

sRNAs play critical roles in controlling regulatory pathways associated with IPF progression. This example evaluates the IPF sRNA expression landscape in lung tissue, whole blood, and primary lung fibroblast samples collected from IPF patients and bleomycin-induced pulmonary fibrosis (PF) mice. The results identify genomic hotspots of sRNA isoforms with significant gain or loss-of-expression in IPF subjects. Loss-of-sRNA isoforms mapping to the MIR92A loci was observed in IPF subjects across all datasets and was a significant predictor of 3-year survival. MIR92-1+/− mice had an accelerated and more severe disease course following bleomycin-induction with respect to wild type mice, recapitulating findings from the human population and validating the protective role of miR-92a-3p isoforms in IPF. Further, inhaled miR-92a-3p mimetics rescued bleomycin-induced PF in mice. Twenty two sRNA mimetics were synthesized, representing the most significantly down-regulated miR-92a-3p isoforms identified by the computational models. One lead (oligo-006, Table 10) was the most effective at reducing collagen deposition and IPF fibroblast growth. Additionally, oligo-006 reduced collagen, fibrotic, and inflammatory biomarkers in precision cut lung slices (hPCLS) from IPF patients. These results elucidate a key role for sRNA isoforms mapping to the MTR92A loci in disease and demonstrate the novel therapeutic potential of miR-92a-3p and its isoforms in IPF.

### Introduction

Idiopathic pulmonary fibrosis (IPF) is a chronic, progressively fatal disorder. Patients with IPF exhibit heterogenous outcomes due to a combination of environmental and genetic factors that impact disease severity and clinical course. Lung transplant is the only curative option for IPF, however, the number of healthy lung donors is rate-limiting, making pharmaceutical intervention imperative for most patients¹. Two FDA approved antifibrotic therapeutics (pirfenidone and nintedanib) are available, but do not significantly improve outcomes. Moreover, severe adverse side effects from these medications often lead to a lack of adherence by IPF patients. Therefore, new treatment options are needed to improve patient outcomes and quality of life.²⁻³

IPF is characterized by progressively reduced pulmonary function, reduced blood oxygen levels, and exercise-induced exertion. Pathologically, IPF is defined by the presence of fibrotic foci concomitant with trans-differentiation of type 2 alveolar cells to (myo)fibroblasts, as well as leukocyte infiltration and structural abnormalities. Numerous signaling pathways have been implicated in IPF, including transforming growth factor beta (TGFß), α and ß integrins (ITGAs/ITGBs), Wingless-related integration pathway (WNT), extracellular matrix (ECM), and cytokine signaling.⁴⁻⁶

New therapeutics targeting these proteins and signaling pathways are currently in various stages of clinical development. However, current investigational drugs have two major challenges. First, they rely on a mono-therapeutic mechanism of action which implies a single disease driver. However, given the complexity of regulatory networks in IPF, therapeutics must target multiple pathways to be successful.⁷ Second, their oral administration poses risks for drug-induced side-effects, particularly in the gastrointestinal tract, where these same regulatory networks are disproportionately activated.⁸ This creates opportunities to develop polypharmacologic inhaled therapies that address current challenges.

Recent evidence showed the safe and efficacious direct-to-lung delivery of small interfering RNAs (siRNAs) with durability lasting over 50-days.^{9,10} siRNAs are a powerful modality that selectively target and inhibit messenger RNAs (mRNAs). siRNA backbones can also be used to deliver mimetics that reconstitute the activity of endogenous sRNA genes. sRNAs are master regulators of gene expression that play critical roles in controlling stress response, growth, and differentiation pathways.¹¹ sRNAs are 18-26 nucleotide RNAs that are bound by an Argonaute protein creating the effector RNA Induced Silencing Complex (or RISC).^{12,13}

RISC is guided to mRNAs that have complementary target sites to the loaded sRNA. Three major functional motifs within a sRNA sequence have been characterized. The 5′-terminal nucleotide is required for RISC loading, nucleotides 2-8 are required for mRNA targeting, and nucleotides 9-12 determine the mechanism for gene silencing. Complementarity at the 3′-end is also important, but less defined. Because the functional motifs are so short, a single sRNA can target many mRNAs, making them pleiotropic (or polypharmacologic) effectors.¹⁴ Previous studies have implicated the role of sRNAs in IPF and IPF-associated regulatory pathways.¹⁵ However, a growing body of evidence suggests that sRNAs are expressed as arrays of sequences called isoforms that impact analysis and biological modeling.^{16,17} Isoforms can have higher expression levels and greater differential gene expression changes when compared to the annotated sRNA, indicating biological importance.¹⁸ Isoforms can have nucleotide additions and subtractions at the 5′ and 3′ end as well as internal swaps which impact their function.¹⁹

Therefore, in accordance with aspects of this disclosure, sRNA isoforms could provide novel biomarkers for stratifying IPF patients and may provide a reservoir of new polypharmacologic therapeutic targets that could be delivered directly to the lungs for the treatment of IPF. To this end, we analyzed sRNA sequencing data generated from samples spanning tissue and blood, human and animal, and in vivo and in vitro sources. This dataset permitted the identification of novel, translatable sRNA drug targets.

The expression of unique sRNA isoforms was characterized across genomic loci. Loci that have an extraordinary number of unique sRNAs are referenced as ‘isoform hotspots’. Interrogation illuminated uniformly down-regulated isoforms from a hotspot on chromosome 13, coincident with the MIR92A1 locus, which enhanced the prediction of 3-year, transplant-free mortality. MIR92-1+/− mice displayed fast progressing, severe bleomycin-induced pulmonary fibrosis compared to Wild Type animals, indicating the protective role of this locus in disease. Replacement of miR-92a-3p isoforms with therapeutic mimetics inhibited collagen deposition and cell growth in primary IPF fibroblasts and precision cut lung slices. Inhaled delivery of miR-92a-3p mimetics reversed bleomycin-induced PF in mice, and improved overall wellness. Additionally, we identified a large set of miR-92a-3p mRNA targets that were successfully down-regulated in primary IPF fibroblasts following treatment with miR-92a-3p mimetics. Many of these genes are involved in ECM production and regulation (including COL5A1, ITGAV, ITGA5, and FBN1),^{20,21} which demonstrate the pleiotropic effect of these isoforms. These results provide identification and validation of oligonucleotide therapeutics that can be used to treat IPF patients with reduced expression of miR-92a-3p isoforms, thereby unlocking the potential for precision medicine in patients with the most severe outcomes.

Discovery of sRNA Isoform Drug Targets in Human and Mouse IPF Datasets

Therapeutic drug programs that integrate biomarkers into early-stage R&D have a 5-times greater likelihood of success for FDA Approval resulting from the ability to select patients during clinical trial enrollment.^{22,23} sRNAs offer a unique opportunity for early-stage biomarker integration because up or down dysregulation is quantifiable in samples, and because dysregulated sRNAs can be targeted directly by oligonucleotide therapies.²⁴ For a biomarker to be useful in early-stage R&D it must correlate to a clinically relevant endpoint and translate across tissue and blood, human and animal, and in vivo and in vitro systems.

To enable the discovery of translatable small RNA drug targets, samples were aggregated from human and animal, tissue and blood, and in vitro and in vivo system (Table 1). sRNA sequencing data was generated from PAXgene Blood RNA samples collected at 3 independent study sites. Matched mRNA sequencing data was generated from 176 samples. Matched sRNA and mRNA sequencing data was generated from primary IPF patient fibroblast cell lines (BioIVT and Lonza). Matched sRNA and mRNA sequencing data was generated from mouse lung tissue that was collected from a longitudinal study of saline or bleomycin-induced C57Bl/6 animals on Day 3, 5, 7, 14 and 21 post-treatment. Saline-treated mice were considered Controls, and bleomycin-treated mice were considered IPF. Additionally, matched sRNA and mRNA microarray data from human lung biopsies was incorporated from GSE3253925.

TABLE 1
Overview of samples used for small RNA isoform drug target discovery.

| Category | | Blood Samples | | GSE32539 | | Lung Fibroblasts | | Mouse Lungs | |
|---|---|---|---|---|---|---|---|---|---|
| N | | 716 | | 156 | | 11 | | 70 | |
| Sample Matrix | | Whole Blood | | Lung Tissue | | Lung fibroblasts | | Lung Tissue | |
| Species | | Homo sapiens | | Homo sapiens | | Homo sapiens | | Mus musculus | |
| Sex, n (%) | Male | 545 | (76%) | 94 | (60%) | 10 | (91%) | 0 | (0%) |
| | Female | 171 | (24%) | 62 | (40%) | 1 | (9%) | 70 | (100%) |
| Age, mean ± SD | | 71.2 ± 8.5 | | 57.7 ± 14.9 | | 62.1 ± 10.4 | | 11 weeks | |
| Diagnosis, n (%) | Control | 19 | (3%) | 106 | (68%) | 9 | (82%) | 30 | (43%) |
| | IPF | 697 | (97%) | 50 | (32%) | 2 | (18%) | 40 | (57%) |

| FVC %, mean ± SD | | 77.3 ± 18.5 | | 59.8 ± 17.0 | NA | NA |
|---|---|---|---|---|---|---|
| DLCO %, mean ± SD | | 48.3 ± 17.1 | | 43.6 ± 19.1 | NA | NA |
| GAP Index, mean ± SD | | 3.9 ± 1.4 | | 3.39 ± 1.72 | NA | NA |
| 3 Year Transplant-Free | | 2.3 ± 0.9 | | NA | NA | NA |
| Survival, mean ± SD | | | | | | |
| Vital Status, n (%) | Dead | 235 | (33.7%) | NA | NA | NA |
| | Alive | 481 | (66.3%) | NA | NA | NA |

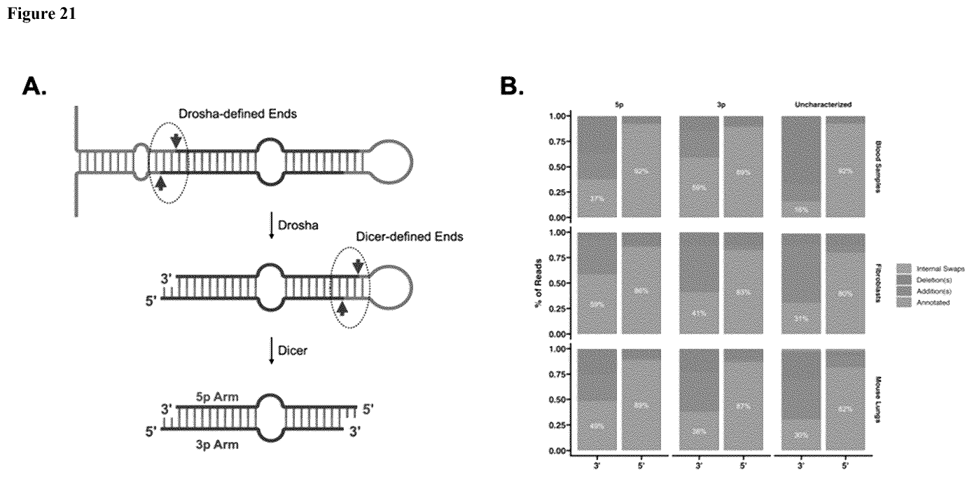

sRNAs originate from either the 3 prime (3p) or 5p (5p) arm of the pre-miRNA and are cleaved by two ribonuclease III enzymes, Drosha and Dicer. The 5′ end of the 3p arm and the 3′ end of the 5p arm are cleaved by Drosha, and the 3′ end of the 3p arm and the 5′ end of the 5p arm are cleaved by Dicer.^{26,27} Therefore, isoforms can be characterized by 3′ or 5′ end additions or deletions and split up by those from a mapped sRNAs originating from the 3p or the 5p arm (FIG. 21A). Isoforms were considered valid if they had a prevalence of at least 5% with a minimum expression of 1 in trimmed reads per million (TRPM). With these filters, 5,687 unique isoforms were identified for 404 annotated sRNAs in PAXgene Blood RNA samples, 11,805 unique isoforms were identified for 605 annotated sRNAs in primary fibroblasts, and 4,867 unique isoforms were identified for 234 annotated sRNAs in lung tissue from bleomycin-induced mice. The percentage of reads attributed to an annotated sRNA were compared across sample types, originating arms, and end (3′ or 5′) using a test of two proportions, of which all comparisons were statistically significant (p<0.05) (FIG. 21B). 1,798 isoforms mapping to 225 annotated sRNAs were conserved across all 3 cohorts and provided a reservoir of novel, translatable, and druggable sRNA targets.

Discovery of miR-92a-3p Hotspot: A Regulator of Progressive IPF Biology

Prior research showed that hotspots of genetic variation and RNA editing create sRNA isoforms that can potentially drive disease biology.^{28,29} Therefore, sRNA hotspots were identified where a disproportionate number of isoforms were expressed. Hotspot were defined as a genomic locus with at least 100 unique isoforms with a TRPM≥1 and a frequency ≥5% within each respective dataset. These parameters corresponded to an FDR threshold of 0.1% for the negative binomial distribution of valid isoforms per genomic locus.

Analysis of sRNA hotspots in human PAXgene Blood RNA and primary fibroblast cell lines revealed that miR-92a-3p was a hotspot with 108 isoforms shared between the two datasets. Isoforms from this hotspot mapped indistinctly to the MIR92A-1 (chromosome 13) and MIR92A-2 (chromosome X) loci, due to sequence identity. The miR-92a-3p hotspot was not observed in bleomycin-induced mice, which may be attributed to homogeneity of mice. Nevertheless, twenty-five miR-92a-3p isoforms were conserved between human and mice.

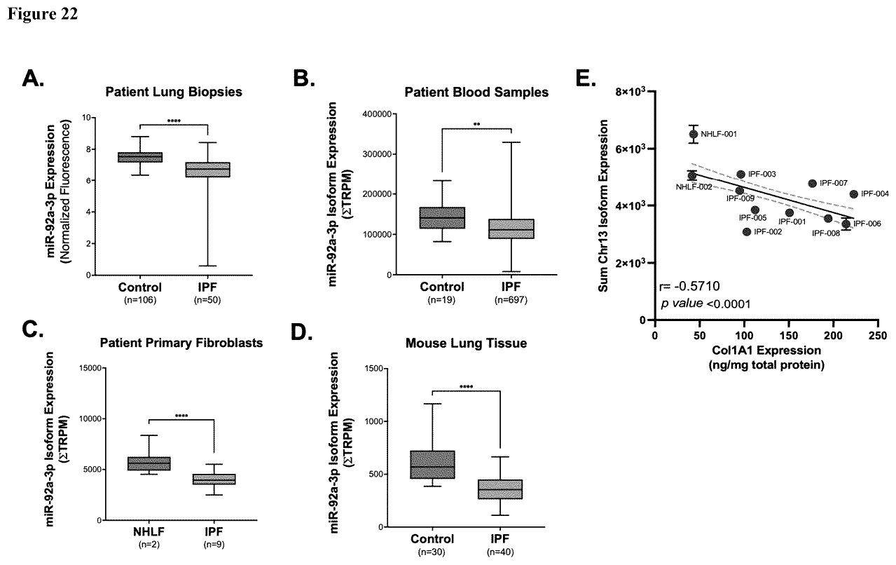

The sum expression of miR-92a-3p isoforms was calculated using trimmed reads per million (TRPM). T tests comparing the sum miR-92a-3p isoform count in IPF samples over Controls showed that miR-92a-3p was significantly down-regulated in PAXgene Blood RNA, IPF fibroblasts and lung tissue from bleomycin-induced mice (FIG. 22B, 22C, 22D). These findings were independently validated using sRNA microarray data from GSE32539, which showed significant down-regulation of miR-92a-3p in lung tissue collected from IPF patients compared to Controls (FIG. 22A). These results indicate that IPF is associated with loss-of-miR-92a-3p isoform expression.

Increased collagen deposition is a characteristic hallmark of IPF30. Therefore, we characterized collagen expression in the primary fibroblast cell lines. Results showed that collagen levels were significantly elevated in fibroblasts derived from IPF patients compared to fibroblasts derived from healthy donors (FIG. 22E). IPF fibroblasts were observed to have a wide range of collagen expression with a 5-fold differential between the highest and lowest expressing cell line (FIG. 22E). The sRNA sequencing data also suggested a wide range of miR-92a-3p isoform expression in the IPF fibroblasts with a 4-fold differential between the highest and lowest expressing cell line (FIG. 22E). miR-92a-3p has previously been implicated in regulating collagen expression.³¹ Therefore, the expression of miR-92a-3p isoform was correlated to collagen. Results showed that miR-92a-3p isoforms were negatively correlated to collagen (Spearman rho: −0.5710, p<0.001) (FIG. 22E). These results suggest that miR-92a-3p isoforms may play a conserved role in regulating pathways that control collagen deposition, and tightly linked miR-92a-3p isoform expression to IPF biology.

miR-92a-3p Isoforms are Predictors of 3-year Transplant-Free Survival in IPF

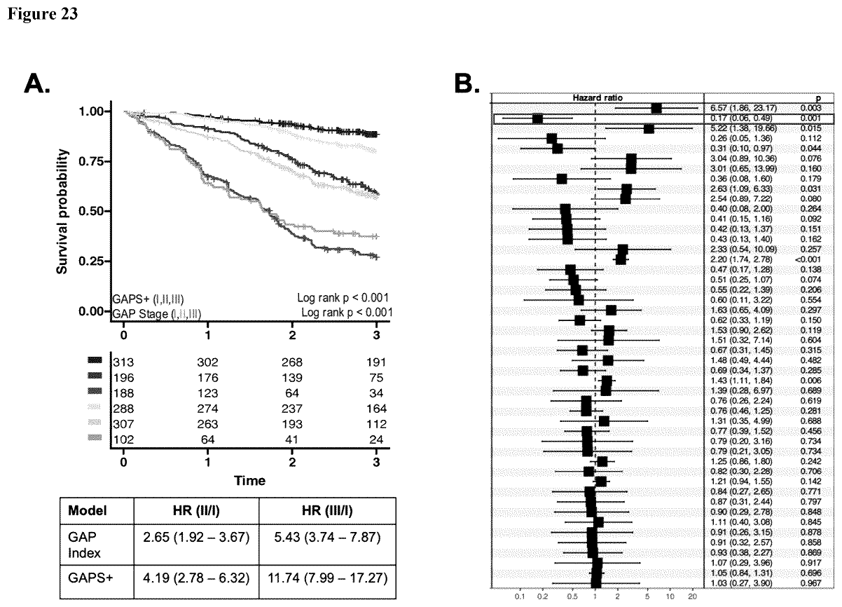

To assess the importance of miR-92a-3p isoforms, we compared a Cox proportional hazards regression model against stratification based on the IPF Clinical GAP Index.³² The Cox model was fit to IPF blood samples (n=697) with 44 small RNA features and included the 4 clinical components of the IPF GAP Index (Gender, Age, DLCO and FVC). Binning criteria of risk strata was based on the probability of mortality within 3 years extracted from the Cox regression model fit are as follows: 0-25% (I), 25-50% (II), and >50% (III) (FIG. 23A).

These three risk strata were compared to the clinical GAP Index, which was represented using GAP stage (I, II, or III). Both models result in three classes with significant differences in three-year transplant-free survival (log rank p<0.001). The incorporation of blood-based sRNAs identified over twice as many high-risk patients compared to the GAP Index alone (test of two proportions, p<0.05). Additionally, the hazard ratios between Stage II/I and Stage III/I were increased from 2.65 to 4.19 and 5.43 and 11.74, respectively, with the incorporation of blood-based sRNAs (FIG. 23A). One miR-92a-3p isoform is the 2nd most important feature for predicting survival, with a significant hazard ratio of 0.17 (p<0.001, log-rank test) (FIG. 23B). Additionally, 41% of patients from the IPF blood samples in the lowest quartile of miR-92a-3p hotspot expression have died within 3 years, as opposed to 31.7% of the patients in the top three quartiles of miR-92a-3p hotspot expression (test of two proportions, p<0.05).

miR-92a-3p Isoforms Protect Against Bleomycin-induced Pulmonary Fibrosis in Mice

Given the compendium of evidence implicating the loss of miR-92a-3p in IPF in multiple discovery cohorts, we hypothesized that miR-92a-3p isoforms play a protective role in IPF. To test this, we induced pulmonary fibrosis in MIR92-1 heterozygous knockout mice using 1.45 mg/kg of bleomycin via oropharyngeal (OP) aspiration. Loss of body weight from Day 0 and survival were tracked during the in-life portion of the observational study. Bleomycin-induced lung fibrosis and miR-92a-3p isoform expression were assessed on Day 21.

Loss of body weight from Day 0 through Day 21 is a characterized metric in the bleomycin mouse model.³³ Wild type mice display a maximum loss of body weight of 8-12% between Days 8-9 following bleomycin challenge compared to saline-treated animals. Following bleomycin challenge, wild type animals recover to their Day 0 body weight between Days 14-16 and plateau until study termination on Day 21. This is concomitant with increased respiratory rate, decreased activity, and histologically increased interstitial collagen deposition, inflammation, and fibrosis.

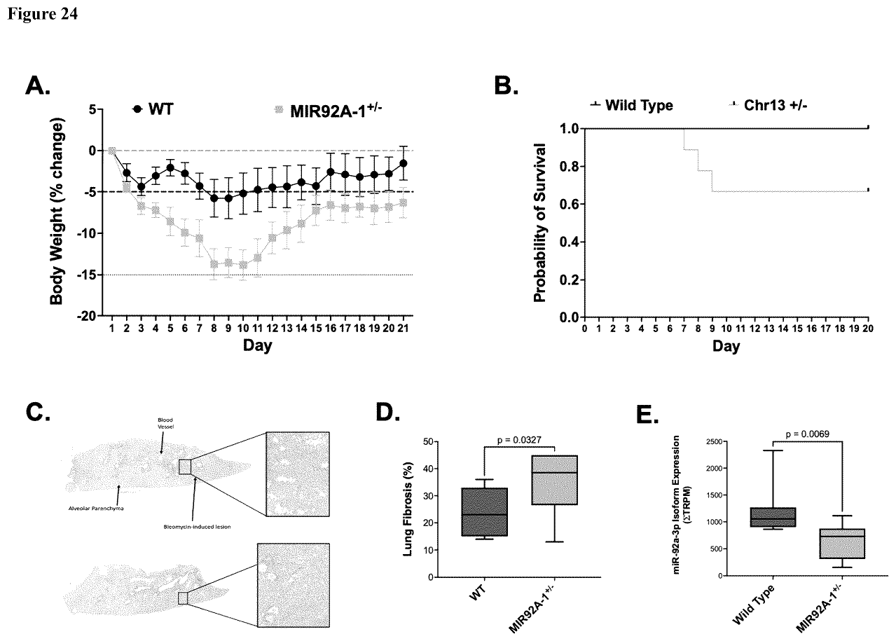

Our results showed that MIR92-1+/− animals experienced faster progression of bleomycin-induced pulmonary fibrosis with respect to wild type. Wild type animals lost 8-12% of their body weight by Day 8 of the study (p<0.001), while MIR92-1+/− animals lost 15-20% of their body weight at the same time point, indicating greater bleomycin-induced severity with respect to wild type (FIG. 24A). Additionally, 3 of the 9 MIR92-1+/− animals (30%) lost greater than 20% of their body weight from Day 0. This exceeded the wellness protocols of the study center, resulting in premature euthanasia of these animals. While differences in overall survival were not significant (log-rank test, p>0.05), these results modeled our observations in the human population (FIG. 24B).

On Day 21 of the study, animals were euthanized and lungs were perfused. The left lung was inflation fixed for histological analysis and the right lung was snap-frozen for molecular analysis. Left lungs were sectioned (3 per lung) and collagen was stained using Picrosirius red (PSR). Blinded scoring from board certified pathologists showed that MIR92-1+/− animals had significantly greater bleomycin-induced collagen deposition compared to Wild Type (p=0.033) (FIGS. 24C and 24D).

In mice, miR-92a-3p is expressed on 2 independent loci on chromosome 14 (MIR92-1) and chromosome X (MIR92-2). The knockout mice utilized in this study contained a heterozygous knockout of the chromosome 14 allele, indicating that miR-92a-3p isoform haploinsufficiency was sufficient to drive greater bleomycin-induced severity. The results from human PAXgene Blood RNA samples suggested that IPF patients have a 40% reduction in miR-92a-3p isoforms compared to healthy donors. Experiments were conducted to understand the extent to which the heterozygous knockout was contributing to haploinsufficiency and the resulting phenotype. sRNA sequencing data generated from the right lungs showed a significant reduction of miR-92a-3p isoforms (t-test, p=0.02), compared to wild type (C57BL/6) (FIG. 24E). These results establish a linkage between miR-92a-3p haploinsufficiency and IPF severity which translated between human and mouse subjects.

miR-92a-3p Mimetics Rescued Bleomycin-induced Pulmonary Fibrosis in Mice

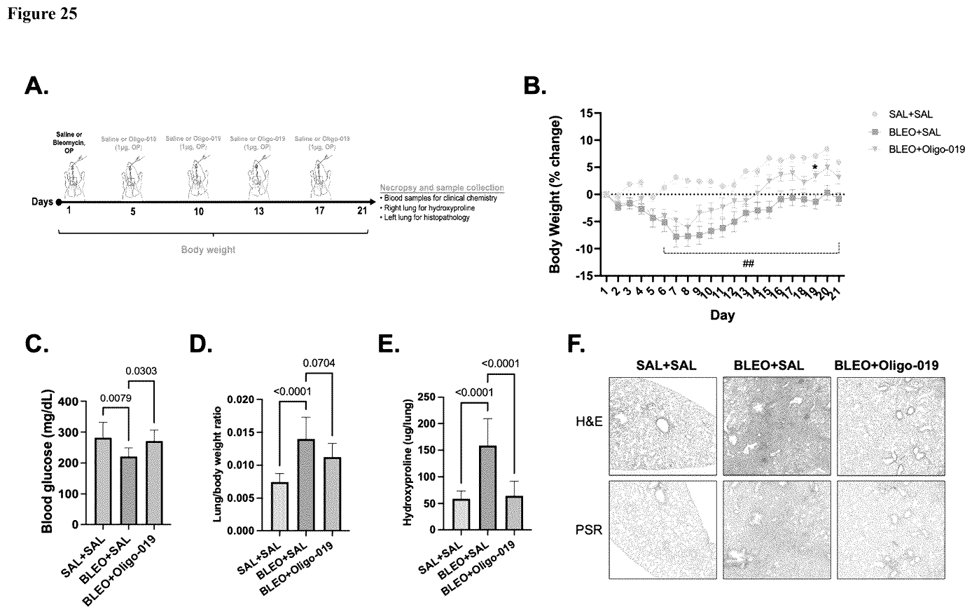

A miR-92a-3p mimetic (Oligo-019, Table 10) was synthesized to determine if over-expression of miR-92-3p was sufficient to rescue of bleomycin-induced pulmonary fibrosis. The mimetic was formulated in an LNP to facilitate delivery to the lung. Formulated compound (1 ug/mouse) or saline control was delivered directly to the lungs of bleomycin-induced animals via oropharyngeal (OP) inhalation on Day 5, 10, 13 and 17 following bleomycin-challenge. Body weights were measured daily starting from Day 1. Animals were sacrificed on day 21 at which time blood and lung tissue was collected (FIG. 25A).

Mice subjected to bleomycin (n=10) experienced a significant reduction in body weight starting from Day 5 as compared to saline control (n=8). Body weight was restored with miR-92a-3p (Oligo-019) in animals subjected to bleomycin (n=8; FIG. 25B). Restoration of body weight was concomitant with increased blood glucose levels in animals treated with miR-92a-3p (FIG. 25C). In addition, the pulmonary inhalation of bleomycin lead to a significant increase in lung weight (p<0.01) which was restored with miR-92a-3p (p<0.05; FIG. 25D).

Consistent with these results, mice treated with miR-92a-3p (Oligo-019) demonstrated protection against bleomycin-induced lung fibrosis, as indicated by a significant reduction in collagen content in bleomycin-challenged animals (p<0.01), measured by hydroxyproline (FIG. 25E). This result was reinforced by histopathological evaluation of PSR staining in lung tissues (FIG. 25F). Histopathological evaluation of H&E staining suggested that pulmonary inhalation of bleomycin induced inflammatory infiltration in lung tissues (inflammation score: Sal/Sal: 0.0±0.0, Bleo/Sal: 3.19±0.284 and Bleo/Oligo-019: 2.5±0.236); which was ameliorated with the delivery of miR-92a-3p in mice subjected with bleomycin (FIG. 25F).

In Vitro Screening of miR-92 Isoform Mimics

Nine primary fibroblast cell lines were selected to screen miR-92a-3p isoforms, and to give a broad spectrum of results that would best model the human population. The use of patient primary IPF fibroblasts enabled a screen of miR-92a-3p isoforms in a ‘native’ (non-induced) background. Cell growth was measured continuously every 4 hours for 96 hours. Collagen expression was measured at 48 hours.

IPF cells grow faster and produce more collagen than NHLF. To assess the effect of miR-92a-3p isoforms on their ability to slow down growth and/or decrease the production of COL1A1, an in vitro screen was performed in IPF human lung fibroblast from 9 IPF patients. The effect of the 22 different mimetics was measured on growth at 10 nM and 100 nM concentrations for 96 hours and the effect on COL1A1 in 100 nM mimetic after 48 hours of exposure.

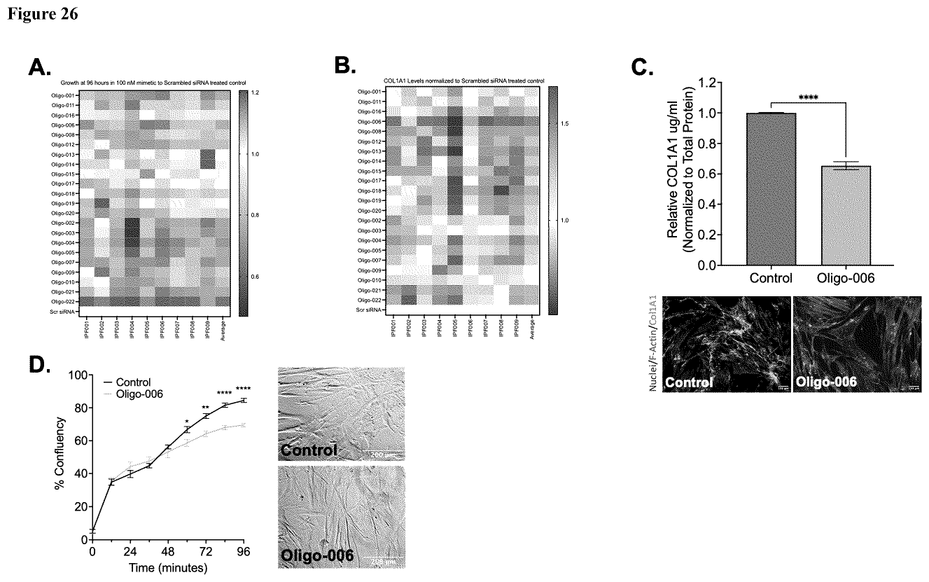

Results showed that 18 of 22 mimetics attenuated growth up to 60% of the scrambled control in all 9 fibroblast cell lines using 10 nM and 100 nM compounds (FIG. 26A). These differences were statistically significant (p<0.001) for all mimetics tested except for Oligos-013, 014, 015, 017, and 019 using an unpaired, two-tailed t-test with Welch's correction for unequal standard deviation. Visual analysis of the cells after 96-hours of exposure showed that there was no cell death or morphological changes following treatment. This indicated that miR-92-3p mimetics were in fact, inhibiting cell growth and not causing cell death or rounding, which could be mistaken for inhibiting cell growth.

Collagen was significantly reduced with 14 out of 22 mimetics in all 9 fibroblast cell lines to varying degrees, up to 50% of control (FIG. 26B). The initial screen revealed that the level of collagen inhibition varied considerably across fibroblast cell lines and isoform mimetics. All mimetics except Oligos-009 and 010 were able to alter collagen levels significantly (p<0.001 using an unpaired two-tailed t-test with Welch's correction for unequal standard deviation). These findings were independently confirmed by immunostaining, which showed decreased collagen expression for mimetic-treated fibroblast cell lines. Based on the initial screen, 6 mimetics with the highest average activity for collagen and fibroblast cell growth inhibition were selected (Oligos-006, 008, 013, 016, 018 and 019).

Mimetics were further assessed using dose response curves to evaluate their EC50 on inhibition of fibroblast cell growth and collagen using cell line IPF005, which was the most sensitive of the 9 cell lines. They were benchmarked against the current standard-of-care drugs (Pirfenidone and Nintedanib) and a competitor, Saracatinib (AstraZeneca) which is currently in clinical trial phase 1 and has obtained orphan drug designation. The mimetics are 6-1000 times more efficacious than the small molecule drugs.

Ex Vivo Efficacy of miR-92a-3p Isoforms Using Precision Cut Lung Slices

Next, the efficacy of miR-92a-3p in pulmonary fibrosis was tested, using precision cut lung slices (hPCLS) collected from pulmonary fibrosis donors. To do this, PCLSs were transfected in duplicate with the most active miR-92a-3p mimetics (10 uM). Mimetics were chemically modified at the 2′-hydroxyl, phosphodiester backbone, 3′-end, and 5′-end to increase stability. PCLSs were analyzed by molecular and histopathology after an acute (48-hour) or chronic (96-hour) exposure. Collagen expression was measured after acute exposure. Inflammatory and fibrotic markers were measured after chronic exposure using Luminex and histopathology. A scrambled mimetic was used as a negative control.

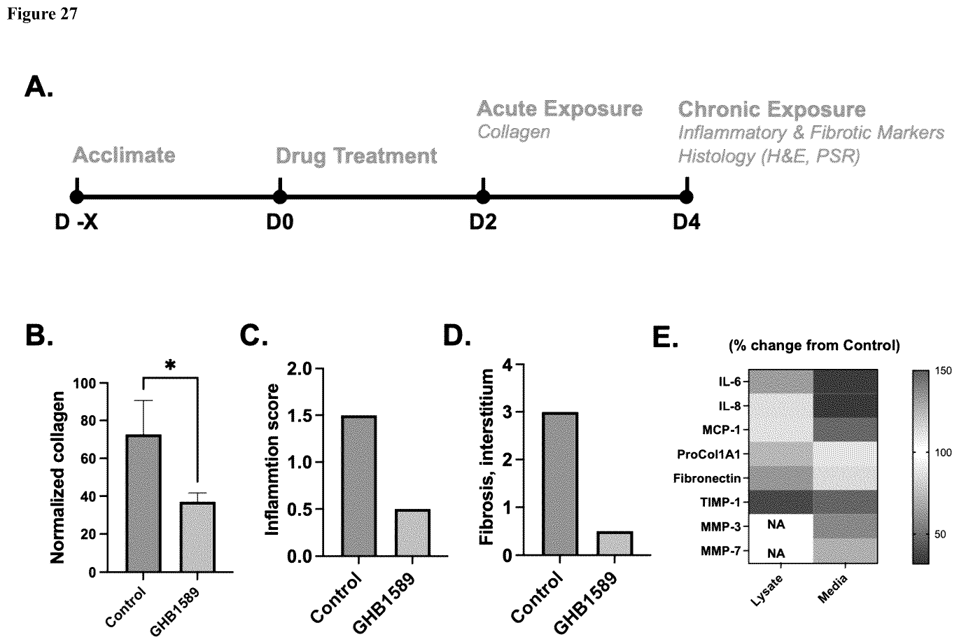

After 2 rounds of screening, Oligo-006 (also referred to as GHB1589) was determined to be the most efficacious mimetic for reducing collagen, inflammatory, and fibrotic markers. Oligo-006 reduced collagen greater than 50% after an acute 48-hour exposure (FIG. 27B). Scoring of H&E stained for inflammatory cell infiltration and fibrosis of the interstitium was completed on PCLS following chronic (96-hour) exposure. Results showed that Oligo-006 reduced the number of inflammatory cells and foci throughout the section (FIG. 27C). Additionally, Oligo-006 reduced fibrosis of alveolar septa over 40% and reduced alveolar septa thickness by 3-fold (FIG. 27D). Scoring was completed on 2-independent PCLSs and results show the mean (±) standard deviation. While the small sample size did not permit statistical analysis, the pathology results trended in the correct direction and covered 2-independent section per PCLS treated.

Molecular analysis showed a marked reduction of IL-6, IL-8, MCP-1, ProColA1, FN1, and TIMP-1 in media and lysates collected at 96-hours compared to the scrambled control. Reduced levels of MMP-3 and MMP-7 were observed in media collected at 96-hours compared to the scrambled control; these analytes were not detected in lysates (FIG. 27E). Together, these results show that Oligo-006 (a miR-92a-3p isoform) was sufficient to rescue pulmonary fibrosis in human derived PCLSs.

Pleotropic Repression of Profibrotic Genes by miR-92a-3p Isoforms

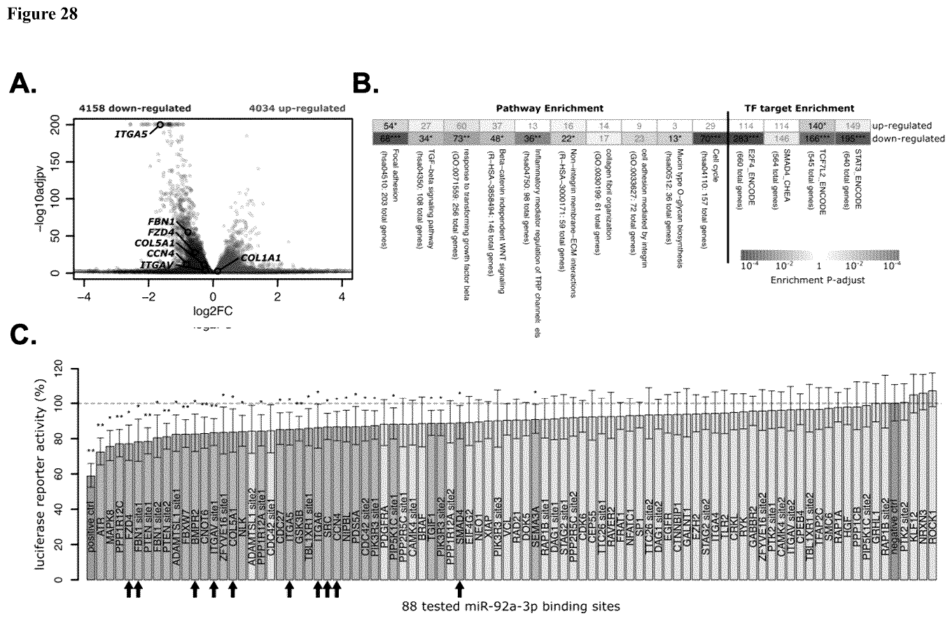

To identify the mechanism of action through which miR-92a-3p isoforms repress the development of fibrosis, mRNA sequencing (NGS) was performed on 4 replicates of one primary human lung fibroblast cell line (IPF005) 48 hours after transfection with a miR-92a-3p mimetic (oligo-006) or scrambled siRNA (control). Differential gene expression resulted in over 8,000 differentially expressed genes in treated cells with respect to the scrambled siRNA, indicating that the mimetic transfection had a wide impact on the mRNA landscape (FIG. 28A). Using an in-house sRNA target site identification method inspired by TargetScan,³⁵ potential targets of miR-92a-3p were identified. These targets were significantly enriched among genes down-regulated in treated cells (1,565 down-regulated genes among the 4,249 genes with binding sites, Chi-2 p-value<10⁻¹⁶)

Significant enrichment of down-regulated genes was also found in the following pathways related to fibrosis: Focal adhesion, TGF-beta pathway, WNT pathway, ECM, and Mucin production. Enrichment analysis among targets of transcription factors (TF) showed evidence that oligo-006 repressed the expression of genes downstream of the profibrotic pathway: SMAD4 and TCF7L2, as well as pro-inflammatory regulator STAT3.³⁶ Additionally, oligo-006 appears to repress genes involved in promoting the cell cycle (KEGG pathway hsa04110, adjusted p=<0.05), consistent with our survey of the isoform effect on cell growth and can be linked to repression of E2F4 TF (FIG. 28B). COL1A1 and other collagen genes, which lack a miR-92a-3p binding site, are not down-regulated with oligo-006, conflicting with the ELISA results. These results suggest that the down-regulation of COL1A1 protein by miR-92a-3p isoforms is indirect and is either translational or post-translational.

Over 200 genes carrying a miR-92a-3p binding site are involved in the three major pro-fibrotic pathways: WNT (FZD4, FZD6, WNT5A, CCN4), TGF-beta (TGFBR2, SMAD2, SMAD4, BMPR2) and Focal adhesion (ITGA5, ITGAV, SRC). Some genes carrying the miR-92a-3p binding site are also involved in the ECM and Collagen pathways (COL5A1, COL1A2, FBN1, FBN2) or interleukin mediated inflammation (IL1R1, STAT3).^{31,37} Many of these genes are down-regulated after transfection with oligo-006 (FIG. 28B).

To confirm the direct interaction of miR-92a-3p isoforms with the mRNA of these genes, we tested the activity of miR-92a-3p activity at their binding sites using a dual luciferase report assay. A total of 88 binding sites from 70 genes were introduced in the 3′ UTR of a luciferase reporter on a plasmid, and A549 cells carrying the plasmid were transfected with oligo-006. Significant down-regulation was observed for 34 binding sites (p-value<0.05), which confirms the activity of miR-92a-3p isoforms on their respective genes (FIG. 28C).

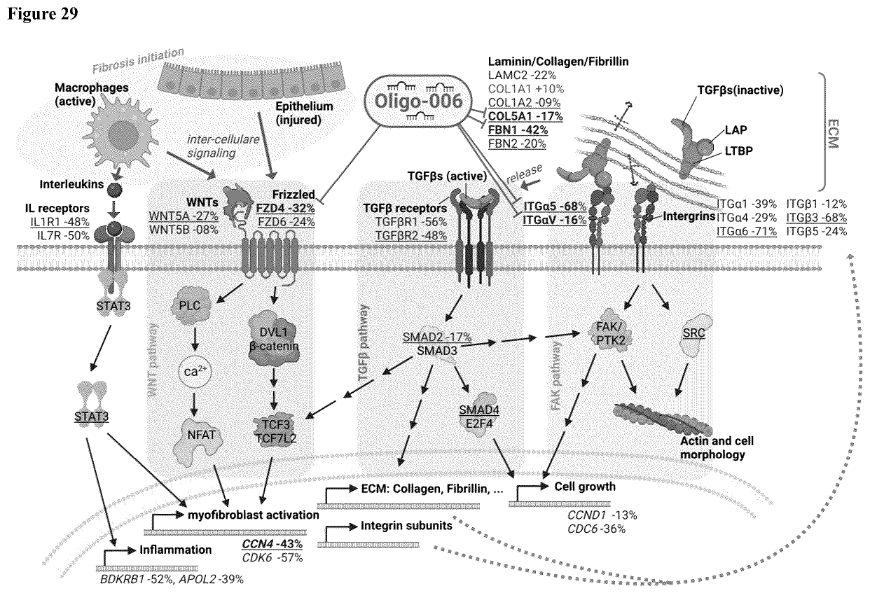

Among the most significant down-regulated genes with an active binding site are COL5A1, ITGAV, ITGA5, FZD4 and FBN1 (FIG. 28C, FIG. 29). Strikingly, these genes are involved in ECM sensing and trigger the pro-fibrotic pathways.³⁸ ITGAV is particularly important, as it is involved in releasing TGF-beta from the latency-associated peptide (LAP) and acts in both Focal Adhesion and TGF-beta signaling.^{39,40} These five genes highlight the pleiotropic effect of miR-92a-3p isoforms, but many other genes are also implicated in the anti-fibrotic effect of miR-92a-3p, with either an active binding site (SRC, BMPR2, CCN4, SMAD4) or down-regulation in our NGS data. From these results, the mechanism of action of Oligo-006 drug can be proposed. After external stress, collagen and other ECM and integrin compounds are produced, triggering focal adhesion, TGF-beta, cell proliferation, and the fibrotic circle of regulation.³⁸ Our findings suggest that the miR-92a-3p isoform represses this cycle at multiple nodes, consistent with the aggravated outcome for patients with lower levels of these miRNA isoforms. Re-introducing a mimetic of this miR-92a-3p isoform as a polypharmacologic therapy should rectify the progression of IPF through its action on integrins alpha, collagen, WNT, and TGF-beta pathways.

### Discussion

In this study, a disease-agnostic approach was used to identify the miR-92a-3p locus as a miRNA isoform hotspot using lung tissue samples, blood samples, and fibroblast cell lines from IPF patients. Leveraging multiple methods including NGS sequencing, ELISA, and hydroxyproline assays, we provide evidence that MIR92A is an important driver for IPF progression. Furthermore, we present evidence of the therapeutic effect of miR-92a-3p mimetic oligonucleotide therapeutics for IPF. These data provide strong evidence that mimetics of miR-92a-3p isoforms are equal or superior to the two FDA-approved drugs, Nintedanib and Pirfenidone, at inhibiting pulmonary fibrosis in both in vitro and ex vivo models derived from IPF donors.

IPF is a heterogeneous disease with varying rates of clinical progression, decline in lung function, and response to therapies.⁴¹ The identification of novel, effective, long-lasting anti-fibrotic agents that target the underlying mechanisms of IPF is an unmet clinical need. Individual sRNAs can modulate the expression of multiple mRNA targets and have a broad effect on multiple cellular pathways. For this reason, therapies targeting individual sRNAs can have a broader impact than traditional monotherapeutic approaches.

At the inception of the study, small RNA-sequencing was performed in human PAXGene blood samples and primary human lung fibroblasts. sRNA hotspots (i.e. specific genomic loci with at least 100 isoform variants) were identified with at least 1 trimmed read per million (TRPM) with a per-study prevalence of at least 5%. Of these hotspots, the miR-92a-3p hotspot, mapped indistinctly to chromosome 13 and chromosome X, was consistently downregulated in all IPF cohorts as compared to their respective controls. Additionally, a miR-92a-3p isoform is the 2nd highest weighted contributor to a Cox Proportional Hazards model predicting 3-year transplant-free survival in IPF.

These findings were confirmed in an open-source microarray dataset of lung biopsy samples obtained from IPF patients.²⁵ Analysis of RNA-sequencing data from human IPF cell lines also confirmed the down-regulation of the MIR92A hotspot as compared to normal human lung fibroblast (NHLF) cell lines. miR-92a-3p expression is also correlated with the up-regulation of collagen expression (p<0.05). sRNA NGS studies in the bleomycin-induced pulmonary fibrosis mouse model confirmed the consistent downregulation of the miR-92a-3p hotspot, implicating miR-92a-3p as a key driver of the murine IPF model. Taken together, data from IPF patients and the mouse model indicate that miR-92a-3p isoforms may be a potential therapeutic target for the regulation of pulmonary fibrosis.

MicroRNA biogenesis starts with pri-miRNA transcription by RNA polymerase II, which is processed in the nucleus by Drosha and assisted by DGCR.⁸ Drosha cleavage is consistent, but alterations in pri-miRNA structure, such as stem length or loop size, can influence the cleavage efficiency and accuracy, thus impacting sRNA biology.⁴² Compared to the canonical sequence, sRNA isoforms can be categorized according to variation of length, sequence, or both.¹⁷ To date, sRNA isoforms have been recognized, but their biological functions have been overlooked, thus leading to many contradictory conclusions in the sRNA field.¹⁹

Located in the miR-17/92 cluster, miR-92a-3p has been characterized in various diseases, with established fibrotic drivers including cancer, coronary artery disease, and pulmonary fibrosis.⁴³ However, the mechanisms of action remain elusive due to the diversity of sRNA isoforms. Therefore, we hypothesize that different miR-92a-3p isoforms could act differential functions thus impacting the progression of IPF disease. To test our hypothesis we ranked and selected 22 human miR-92a-3p isoforms and tested their anti-fibrotic capabilities using in vitro and ex vivo systems. In consideration of IPF heterogeneity, we measured the function of miR-92a-3p isoform in IPF fibroblasts collected from different donors. Consistent with our hypothesis, we observed that different isoforms from the same miR-92-3p family had varying degrees of anti-fibrotic activity. The mimetics identified from in vitro screening were further tested in fibrotic human precision-cut lung slices (hPCLS), which is an established ex vivo model system.⁴⁴ An important advantage of the hPCLS model is that the structural and functional heterogeneity of organs is conserved, partly due to the presence of cell-matrix and intercellular interactions.⁴⁵

According to our results, the miR-92a-3p isoform mimetic (Oligo-006, GLB1589), which showed the strongest repression of proliferation and collagen accumulation, also target numerous genes involved in the WNT/TGF-b/FAK circle of regulation. Importantly, in these pathways, ITGAV ITGA5, COL5A1, FBN1 and FDZ4 genes has been demonstrated to be physically targeted by the isoform, four of them are up-regulated in IPF (FBN1, COL5A1, ITGAV, FZD4). These show the anti-fibrotic efficacy of the mimetic through polypharmacologic mechanism of action.

While sRNAs are conserved across mice and humans, individual isoforms may not be conserved, and inherent genetic differences between humans and mice may restrict the target efficacy of a human miRNA sequence in a preclinical study.⁴⁶ Therefore, mouse surrogate siRNA sequences are commonly used in preclinical drug development for siRNAs.⁴⁷ sRNA sequencing was performed in a bleomycin-induced mouse model of pulmonary fibrosis at 3, 5, 7, 14, and 21 days post-bleomycin administration.⁴⁸ Consistent with human datasets, chromosome 13 mapped miR-92a-3p isoforms were downregulated in the bleomycin mouse model of pulmonary fibrosis. Of which, 6 miR-92a-3p isoforms with >100 TRPM and reduced expression at day 7 (at the initiation of fibrosis) of bleomycin were identified for the in vivo study. Most importantly, delivering a murine miR-92a-3p mimetic encapsulated in LNP to the lungs via oropharyngeal (OP) route restored the disease-related phenotypes in bleomycin induced IPF murine model, indicating the critical role of miR-92a-3p in the IPF treatment.

In conclusion, our study provides evidence of a disease-guided biomarker discovery and therapeutics using patient biofluid. Leveraging in vitro, ex vivo and in vivo model, we confirmed the function of miR-92a-3p isoforms in the IPF prognosis and therapeutics. Furthermore, computational analysis of 24 different small RNA sequencing datasets has also identified the miR-92a-3p isoform hotspot to be evident in 14 other disease conditions: NASH, Heart Failure, Cardiomyopathy, Huntington's disease, Parkinson's disease, sCID, ALS, Alzheimer's disease, Crohn's disease, Ulcerative colitis, Psoriasis, Pompe's disease, Breast Cancer, and pre-eclampsia. While there has been prior research that indicated the pathways included in the MOA individually, the role of miR-92a-3p isoforms in regulating the WNT/TGFb/ITGA/FAK regulatory axis is novel and surprising.

### REFERENCES

(1) Laporta Hernandez, R et al. Lung Transplantation in Idiopathic Pulmonary Fibrosis. Med Sci (Basel) 2018, 6 (3), 68.
(2) Richeldi, L, et al.; INPULSIS Trial Investigators. Efficacy and Safety of Nintedanib in Idiopathic Pulmonary Fibrosis. N Engl J Med 2014, 370 (22), 2071-2082.
(3) Noble, P. W., et al.; CAPACITY Study Group. Pirfenidone in Patients with Idiopathic Pulmonary Fibrosis (CAPACITY): Two Randomised Trials. Lancet 2011, 377 (9779), 1760-1769.
(4) Herrera, J.; Henke, C. A.; Bitterman, P. B. Extracellular Matrix as a Driver of Progressive Fibrosis. J Clin Invest 2018, 128 (1), 45-53.
(5) Willis, B. C., et al. Induction of Epithelial-Mesenchymal Transition in Alveolar Epithelial Cells by Transforming Growth Factor-Beta1: Potential Role in Idiopathic Pulmonary Fibrosis. Am J Pathol 2005, 166 (5), 1321-1332.
(6) Chilosi, M., et al. Aberrant Wnt/Beta-Catenin Pathway Activation in Idiopathic Pulmonary Fibrosis. Am J Pathol 2003, 162 (5), 1495-1502.
(7) Sontake, V., et al. New Therapeutics Based on Emerging Concepts in Pulmonary Fibrosis. Expert Opin Ther Targets 2019, 23 (1), 69-81.
(8) Spagnolo, P.; Tzouvelekis, A.; Bonella, F. The Management of Patients With Idiopathic Pulmonary Fibrosis. Front Med (Lausanne) 2018, 5, 148.
(9) Bumcrot, D, et al. RNAi Therapeutics: A Potential New Class of Pharmaceutical Drugs. Nat Chem Biol 2006, 2 (12), 711-719.
(10) Zoulikha, M., et al. Pulmonary Delivery of siRNA against Acute Lung Injury/Acute Respiratory Distress Syndrome. Acta Pharm Sin B 2022, 12 (2), 600-620.
(11) Kaikkonen, M. U.; Lam, M. T. Y.; Glass, C. K. Non-Coding RNAs as Regulators of Gene Expression and Epigenetics. Cardiovasc Res 2011, 90 (3), 430-440.
(12) Lee, Y., et al. The Nuclear RNase III Drosha Initiates microRNA Processing. Nature 2003, 425 (6956), 415-419.
(13) Bernstein, E.; Caudy, A. A.; Hammond, S. M.; Hannon, G. J. Role for a Bidentate Ribonuclease in the Initiation Step of RNA Interference. Nature 2001, 409 (6818), 363-366.
(14) Bartel, D. P. MicroRNA Target Recognition and Regulatory Functions. Cell 2009, 136 (2), 215-233.
(15) Gangwar, I., et al. Detecting the Molecular System Signatures of Idiopathic Pulmonary Fibrosis through Integrated Genomic Analysis. Sci Rep 2017, 7 (1), 1554.
(16) Londin, E, et al. Analysis of 13 Cell Types Reveals Evidence for the Expression of Numerous Novel Primate- and Tissue-Specific microRNAs. Proc Natl Acad Sci USA 2015, 112 (10), E1106-1115.
(17) Tomasello, L.; Distefano, R.; Nigita, G.; Croce, C. M. The MicroRNA Family Gets Wider: The IsomiRs Classification and Role. Front Cell Dev Biol 2021, 9, 668648.
(18) Karlsen, T. A.; Aae, T. F.; Brinchmann, J. E. Robust Profiling of microRNAs and isomiRs in Human Plasma Exosomes across 46 Individuals. Sci Rep 2019, 9 (1), 19999.
(19) Neilsen, C. T.; Goodall, G. J.; Bracken, C. P. IsomiRs—the Overlooked Repertoire in the Dynamic microRNAome. Trends Genet 2012, 28 (11), 544-549.
(20) Lei, G., et al. Regulation of Collagen V Expression and Epithelial-Mesenchymal Transition by miR-185 and miR-186 during Idiopathic Pulmonary Fibrosis. Am J Pathol 2016, 186 (9), 2310-2316.
(21) Shochet, G. E., et al. Integrin Alpha-5 Silencing Leads to Myofibroblastic Differentiation in IPF-Derived Human Lung Fibroblasts. Ther Adv Chronic Dis 2020, 11, 2040622320936023.
(22) Wong, C. H.; Siah, K. W.; Lo, A. W. Estimation of Clinical Trial Success Rates and Related Parameters. Biostatistics 2019, 20 (2), 273-286.
(23) Mohamed, L., et al. The Effect of Biomarker Use on the Speed and Duration of Clinical Trials for Cancer Drugs. Oncologist 2022, 27 (10), 849-856.
(24) Watson, C. N.; Belli, A.; Di Pietro, V. Small Non-Coding RNAs: New Class of Biomarkers and Potential Therapeutic Targets in Neurodegenerative Disease. Front Genet 2019, 10, 364.
(25) Yang, I. V., et al. Expression of Cilium-Associated Genes Defines Novel Molecular Subtypes of Idiopathic Pulmonary Fibrosis. Thorax 2013, 68 (12), 1114-1121.
(26) Kim, B.; Jeong, K.; Kim, V. N. Genome-Wide Mapping of DROSHA Cleavage Sites on Primary MicroRNAs and Noncanonical Substrates. Mol Cell 2017, 66 (2), 258-269.e5.
(27) Kim, Y.-K.; Kim, B.; Kim, V. N. Re-Evaluation of the Roles of DROSHA, Export in 5, and DICER in microRNA Biogenesis. Proc Natl Acad Sci USA 2016, 113 (13), E1881-1889.
(28) Wang, Y., et al. Systematic Characterization of A-to-I RNA Editing Hotspots in microRNAs across Human Cancers. Genome Res 2017, 27 (7), 1112-1125.
(29) Park, E., et al. Genetic Variation and microRNA Targeting of A-to-I RNA Editing Fine Tune Human Tissue Transcriptomes. Genome Biol 2021, 22 (1), 77.
(30) Mei, Q.; Liu, Z.; Zuo, H.; Yang, Z.; Qu, J. Idiopathic Pulmonary Fibrosis: An Update on Pathogenesis. Front Pharmacol 2021, 12, 797292.
(31) Mao, G., et al. Exosomes Derived from miR-92a-3p-Overexpressing Human Mesenchymal Stem Cells Enhance Chondrogenesis and Suppress Cartilage Degradation via Targeting WNT5A. Stem Cell Res Ther 2018, 9 (1), 247.
(32) Ley, B., et al. A Multidimensional Index and Staging System for Idiopathic Pulmonary Fibrosis. Ann Intern Med 2012, 156 (10), 684-691.
(33) Lai, R. et al. The Longitudinal and Regional Analysis of Bleomycin-Induced Pulmonary Fibrosis in Mice by Microcomputed Tomography. Heliyon 2023, 9 (5), e15681.
(34) Selman, M.; Pardo, A. Idiopathic Pulmonary Fibrosis: An Epithelial/Fibroblastic Cross-Talk Disorder. Respir Res 2002, 3 (1), 3.
(35) Agarwal, V., et al. Predicting Effective microRNA Target Sites in Mammalian mRNAs. Elife 2015, 4, e05005.
(36) Epstein Shochet, G, et al. TGF-β Pathway Activation by Idiopathic Pulmonary Fibrosis (IPF) Fibroblast Derived Soluble Factors Is Mediated by IL-6 Trans-Signaling. Respir Res 2020, 21 (1), 56.
(37) Zhang, G.-J., et al. MiR-92a Promotes Stem Cell-like Properties by Activating Wnt/0-Catenin Signaling in Colorectal Cancer. Oncotarget 2017, 8 (60), 101760-101770.
(38) Upagupta, C.; Shimbori, C.; Alsilmi, R.; Kolb, M. Matrix Abnormalities in Pulmonary Fibrosis. Eur Respir Rev 2018, 27 (148), 180033.
(39) Malenica, I., et al. Integrin-αV-Mediated Activation of TGF-β Regulates Anti-Tumour CD8 T Cell Immunity and Response to PD-1 Blockade. Nat Commun 2021, 12 (1), 5209.
(40) Henderson, N. C.; Sheppard, D. Integrin-Mediated Regulation of TGFβ in Fibrosis. Biochim Biophys Acta 2013, 1832 (7), 891-896.
(41) Clarke, D. L, et al. Is Personalised Medicine the Key to Heterogeneity in Idiopathic Pulmonary Fibrosis? Pharmacol Ther 2017, 169, 35-46.
(42) Han, J., et al. Molecular Basis for the Recognition of Primary microRNAs by the Drosha-DGCR8 Complex. Cell 2006, 125 (5), 887-901.
(43) Mogilyansky, E.; Rigoutsos, I. The miR-17/92 Cluster: A Comprehensive Update on Its Genomics, Genetics, Functions and Increasingly Important and Numerous Roles in Health and Disease. Cell Death Differ 2013, 20 (12), 1603-1614.
(44) Patel, V. S., et al. Cryopreserved Human Precision-Cut Lung Slices Provide an Immune Competent Pulmonary Test System for “on-Demand” Use and Long-Term Cultures. Toxicol Sci 2023, 191 (2), 253-265.
(45) Vazquez-Armendariz, A. I., et al. 3D In Vitro Models: Novel Insights into Idiopathic Pulmonary Fibrosis Pathophysiology and Drug Screening. Cells 2022, 11 (9), 1526.
(46) Langschied, F., et al. Efficient and Reliable Identification of miRNA Orthologs. Nucleic Acids Res 2023, 51 (13), e71.
(47) Humphreys, S. C., et al. Considerations and Recommendations for Assessment of Plasma Protein Binding and Drug-Drug Interactions for siRNA Therapeutics. Nucleic Acids Res 2022, 50 (11), 6020-6037.
(48) Moeller, A., et al. The Bleomycin Animal Model: A Useful Tool to Investigate Treatment Options for Idiopathic Pulmonary Fibrosis?Int J Biochem Cell Biol 2008, 40 (3), 362-382.
(49) White, E. S. Lung Extracellular Matrix and Fibroblast Function. Ann Am Thorac Soc 2015, 12 Suppl 1 (Suppl 1), S30-33.
(50) Maher, T. M., et al. An Epithelial Biomarker Signature for Idiopathic Pulmonary Fibrosis: An Analysis from the Multicentre PROFILE Cohort Study. Lancet Respir Med 2017, 5 (12), 946-955.

TABLE 2
Small RNA Features for Evaluating IPF

| SEQ | | | |
|---|---|---|---|
| ID | | Mapped | Class |
| NO. | Small RNA Sequence 5′ to 3′ | Name | Small RNA |
| 1 | AACCCGTAGATCCGAACTTGTG | hsa-mir- | MicroRNA |
| | | 100-5p | Isoform |
| 2 | AAGCTCGGTCTGAGGCCCCTC | hsa-mir- | MicroRNA |
| | | 423-3p | Isoform |
| 3 | AATCCCGGACGAGCCCC | tRNA-Pro- | tRNA-derived |
| | | TGG-3-4 | Fragment |
| 4 | ACCTGTACTGAGCTGCCCCGAGA | hsa-mir- | MicroRNA |
| | | 486-5p | Isoform |
| 5 | ACTCGGCGTGGCGTCGGTCGTGT | hsa-mir- | MicroRNA |
| | | 1307-3p | Isoform |
| 6 | ATCCCCGGCACCTCCAC | tRNA-Ala- | tRNA-derived |
| | | AGC-3-1 | Fragment |
| 7 | ATCTCCCAACCCTTGTACCAGTG | hsa-mir- | MicroRNA |
| | | 150-5p | Isoform |
| 8 | ATGTCCCACCCCCACTCCTGTTAA | hsa-mir- | MicroRNA |
| | | 4433b-5p | Isoform |
| 9 | ATGTCCCACCCCCACTCCTGTTT | hsa-mir- | MicroRNA |
| | | 4433b-5p | Isoform |
| 10 | CGTCCCACCCCCCACTCCTGTT | hsa-mir- | MicroRNA |
| | | 4433a-5p | Isoform |
| 11 | CTATACGACCTGCTGCCTTTCG | hsa-let-7d- | MicroRNA |
| | | 3p | Isoform |
| 12 | GACCTCGCCGTCCCGCCCGCC | hsa-mir- | MicroRNA |
| | | 10401-3p | Isoform |
| 13 | GCACTTGTCTCGGTCTGA | hsa-mir-25- | MicroRNA |
| | | 3p | Isoform |
| 14 | GTAGTAGGTTGTATAGTT | hsa-let-7a- | MicroRNA |
| | | 5p | Isoform |
| 15 | GTCAACACTTGCTGGTTTCCTCT | hsa-mir- | MicroRNA |
| | | 505-3p | Isoform |
| 16 | GTCATACACGGCTCTCCTCTCT | hsa-mir- | MicroRNA |
| | | 485-3p | Isoform |
| 17 | TATTGCACTTGTCCCGGCCTGTGA | hsa-mir- | MicroRNA |
| | | 92a-3p | Isoform |
| 18 | TCCCCGGCATCTCCACCAAAA | tRNA-Ala- | tRNA-derived |
| | | AGC-3-1 | Fragment |
| 19 | TCCCTGTCCTCCAGGAGCTCACG | hsa-mir- | MicroRNA |
| | | 339-5p | Isoform |
| 20 | TCCCTGTCCTCCAGGAGCTCACGA | hsa-mir- | MicroRNA |
| | | 339-5p | Isoform |
| 21 | TCCCTGTCCTCCAGGAGCTCA | hsa-mir- | MicroRNA |
| | | 339-5p | Isoform |
| 22 | TCCTGTACTGAGCTACCCCGAG | hsa-mir- | MicroRNA |
| | | 486-5p | Isoform |
| 23 | TCCTGTACTGAGCTGCCCCAGA | hsa-mir- | MicroRNA |
| | | 486-5p | Isoform |
| 24 | TCCTGTACTGAGCTGCCCCGAGGCC | hsa-mir- | MicroRNA |
| | | 486-5p | Isoform |
| 25 | TCTACAGTGCACGTGTCTCCAG | hsa-mir- | MicroRNA |
| | | 139-5p | Isoform |
| 26 | TCTTCGGGGGCGTGGGT | tRNA-Leu- | tRNA-derived |
| | | AAG-4-1 | Fragment |
| 27 | TCTTGTACTGAGCTGCCCCGAG | hsa-mir- | MicroRNA |
| | | 486-5p | Isoform |
| 28 | TGAGGGGCAGAGAGCGAGAAA | hsa-mir- | MicroRNA |
| | | 423-5p | Isoform |
| 29 | TGGCATGCACGAGGCCCCGGGTT | tRNA-Ala- | tRNA-derived |
| | | AGC-5-1 | Fragment |
| 30 | TGGCATGCACGAGGCCCCGGGTTT | tRNA-Ala- | tRNA-derived |
| | | AGC-5-1 | Fragment |
| 31 | TGGGTTTACGTTGGGAGAAC | hsa-mir- | MicroRNA |
| | | 629-5p | Isoform |
| 32 | TGGTTAGGATTCGGCGCTCTCA | tRNA-Glu- | tRNA-derived |
| | | CTC-1-6 | Fragment |
| 33 | TGTCCCACCCCCACTCCTGTT | hsa-mir- | MicroRNA |
| | | 4433b-5p | Isoform |
| 34 | TCAAGTGATGGAGAGCAATACCCGGGGATGACTGTGACCACAT | hsa-mir- | Pre-MicroRNA |
| | TGGGATGTATTCGTACTGTCTGATG | 10395-3p | Isoform |
| 35 | GCATTGGTGGTTCAGTGGTAGAATTCTCGCCTGCCACGCGGGA | tRNA-Gly- | tRNA-derived |
| | GGCCCGGG | GCC-2-6 | Fragment |
| 36 | GCATGGGTGGTTCAGTGGTAGAATTCTCGCCTGCCACGCGGGA | tRNA-Gly- | tRNA-derived |
| | GGCCCGGG | GCC-1-5 | Fragment |
| 37 | GCATTGGTGGTTCAGTGGTAGAATTCTCGCCTGCCACGCGGGA | tRNA-Gly- | tRNA-derived |
| | GGCCCGGGTTCGTTT | GCC-2-6 | Fragment |
| 38 | TTGGTGGTTCAGTGGTAGAATTCTCGCCTGCCACGCGGGAGGC | tRNA-Gly- | tRNA-derived |
| | CCGGGT | GCC-2-6 | Fragment |
| 39 | GCATTGGTGGTTCAGTGGTAGAATTCTCGCCTGCCACGCGGGA | tRNA-Gly- | tRNA-derived |
| | GACCCGGGT | GCC-2-6 | Fragment |
| 40 | GCATTGGTGGTTCAGTGGTAGAATTCTCGCCTGCCACGCGGGA | tRNA-Gly- | tRNA-derived |
| | GGCCCGGGTTCGTTTCCCGGCC | GCC-2-6 | Fragment |
| 41 | GTGGGGGAGAGGCTGTCGCTGCGCTTCTGGGCCCGCGGCGGGC | hsa-mir- | Pre-MicroRNA |
| | GTGGGG | 6724-5p | Isoform |
| 42 | GCATTAGTGGTTCAGTGGTAGAATTCTCGCCTGCCACGCGGGA | tRNA-Gly- | tRNA-derived |
| | GGCCCGGGT | GCC-2-5 | Fragment |
| 43 | GCATTGGTGGTTCAGTGGTAGAATTCTCGCCTGCCACGCGGGA | tRNA-Gly- | tRNA-derived |
| | GGCCCGGGTA | GCC-2-6 | Fragment |
| 44 | GCATTGGTGGTTTAGTGGTAGAATTCTCGCCTGCCACGCGGGA | tRNA-Gly- | tRNA-derived |
| | GGCCCGGGT | GCC-2-6 | Fragment |

| | | Spearman | |
|---|---|---|---|
| | | Correlation | |
| SEQ | | to | |
| ID | Population | Martingale | |
| NO. | Frequency | Residuals | P-value |
| 1 | 86% | −0.379 | 0.043 |
| 2 | 91% | −0.373 | 0.261 |
| 3 | 91% | −0.900 | 0.083 |
| 4 | 91% | −0.393 | 0.165 |
| 5 | 90% | 0.348 | 0.016 |
| 6 | 90% | 0.257 | 0.374 |
| 7 | 89% | −0.503 | 0.079 |
| 8 | 88% | 0.125 | 0.172 |
| 9 | 90% | 0.146 | 0.469 |
| 10 | 82% | 0.325 | 0.219 |
| 11 | 91% | 0.301 | 0.342 |
| 12 | 91% | −0.261 | 0.466 |
| 13 | 87% | 0.657 | 0.175 |
| 14 | 87% | 0.079 | 0.035 |
| 15 | 90% | 0.102 | 0.010 |
| 16 | 90% | −0.097 | 0.010 |
| 17 | 91% | −0.116 | 0.003 |
| 18 | 91% | 0.052 | 0.124 |
| 19 | 91% | −0.171 | 0.000 |
| 20 | 90% | −0.176 | 0.000 |
| 21 | 90% | −0.092 | 0.020 |
| 22 | 91% | −0.103 | 0.010 |
| 23 | 91% | −0.058 | 0.092 |
| 24 | 90% | 0.115 | 0.003 |
| 25 | 90% | −0.107 | 0.005 |
| 26 | 91% | 0.144 | 0.001 |
| 27 | 91% | 0.072 | 0.056 |
| 28 | 91% | 0.143 | 0.001 |
| 29 | 91% | −0.039 | 0.177 |
| 30 | 90% | 0.072 | 0.052 |
| 31 | 90% | −0.092 | 0.018 |
| 32 | 91% | 0.160 | 0.000 |
| 33 | 91% | 0.151 | 0.000 |
| 34 | 91% | 0.155 | 0.000 |
| 35 | 91% | −0.092 | 0.018 |
| 36 | 90% | −0.085 | 0.018 |
| 37 | 90% | −0.122 | 0.003 |
| 38 | 91% | 0.142 | 0.001 |
| 39 | 82% | −0.102 | 0.010 |
| 40 | 89% | −0.121 | 0.003 |
| 41 | 90% | −0.076 | 0.032 |
| 42 | 90% | 0.096 | 0.010 |
| 43 | 91% | 0.106 | 0.005 |
| 44 | 77% | 0.105 | 0.010 |

TABLE 3 Primers and Probes For RT-qPCR Analysis of Small RNA Features
Correlated to IPF Risk of Mortality

| Targets | | | | |
|---|---|---|---|---|
| SRNA | | | | |
| Feature | | | | |
| of | SEQ | | SEQ | |
| SEQ ID | ID | | ID | Gene Specific |
| NO: | NO. | RT-primer 5′ to 3 ′ | NO. | Primer 5′ to 3′ |
| 1 | 342 | GTCGTATCCAGTGCAGGGTCCGAGGTATTCGCACTGGATACGACCACAAG | 386 | AACCCGTAGATCCGAACTTG |
| 2 | 343 | GTCGTATCCAGTGCAGGGTCCGAGGTATTCGCACTGGATACGACGAGGGG | 387 | AAGCTCGGTCTGAGGCCCCT |
| 3 | 344 | GTCGTATCCAGTGCAGGGTCCGAGGTATTCGCACTGGATACGACGGGGCT | 388 | AATCCCGGACGAGCCCC |
| 4 | 345 | GTCGTATCCAGTGCAGGGTCCGAGGTATTCGCACTGGATACGACTCTCGG | 389 | ACCTGTACTGAGCTGCCCCG |
| 5 | 346 | GTCGTATCCAGTGCAGGGTCCGAGGTATTCGCACTGGATACGACACACGA | 390 | ACTCGGCGTGGCGTCGGTCG |
| 6 | 347 | GTCGTATCCAGTGCAGGGTCCGAGGTATTCGCACTGGATACGACGTGGAG | 391 | ATCCCCGGCACCTCCAC |
| 7 | 348 | GTCGTATCCAGTGCAGGGTCCGAGGTATTCGCACTGGATACGACCACTGG | 392 | ATCTCCCAACCCTTGTACCA |
| 8 | 349 | GTCGTATCCAGTGCAGGGTCCGAGGTATTCGCACTGGATACGACTTAACA | 393 | ATGTCCCACCCCCACTCCTG |
| 9 | 350 | GTCGTATCCAGTGCAGGGTCCGAGGTATTCGCACTGGATACGACAAACAG | 394 | ATGTCCCACCCCCACTCCTG |
| 10 | 351 | GTCGTATCCAGTGCAGGGTCCGAGGTATTCGCACTGGATACGACAACAGG | 395 | CGTCCCACCCCCCACTCCTG |
| 11 | 352 | GTCGTATCCAGTGCAGGGTCCGAGGTATTCGCACTGGATACGACCGAAAG | 396 | CTATACGACCTGCTGCCTTT |
| 12 | 353 | GTCGTATCCAGTGCAGGGTCCGAGGTATTCGCACTGGATACGACGGCGGG | 397 | GACCTCGCCGTCCCGCCCGC |
| 13 | 354 | GTCGTATCCAGTGCAGGGTCCGAGGTATTCGCACTGGATACGACTCAGAC | 398 | GCACTTGTCTCGGTCTGA |
| 14 | 355 | GTCGTATCCAGTGCAGGGTCCGAGGTATTCGCACTGGATACGACAACTAT | 399 | GTAGTAGGTTGTATAGTT |
| 15 | 356 | GTCGTATCCAGTGCAGGGTCCGAGGTATTCGCACTGGATACGACAGAGGA | 400 | GTCAACACTTGCTGGTTTCC |
| 16 | 357 | GTCGTATCCAGTGCAGGGTCCGAGGTATTCGCACTGGATACGACAGAGAG | 401 | GTCATACACGGCTCTCCTCT |
| 17 | 358 | GTCGTATCCAGTGCAGGGTCCGAGGTATTCGCACTGGATACGACTCACAG | 402 | TATTGCACTTGTCCCGGCCT |
| 18 | 359 | GTCGTATCCAGTGCAGGGTCCGAGGTATTCGCACTGGATACGACTTTTGG | 403 | TCCCCGGCATCTCCACCAAA |
| 19 | 360 | GTCGTATCCAGTGCAGGGTCCGAGGTATTCGCACTGGATACGACCGTGAG | 404 | TCCCTGTCCTCCAGGAGCTC |
| 20 | 361 | GTCGTATCCAGTGCAGGGTCCGAGGTATTCGCACTGGATACGACTCGTGA | 405 | TCCCTGTCCTCCAGGAGCTC |
| 21 | 362 | GTCGTATCCAGTGCAGGGTCCGAGGTATTCGCACTGGATACGACTGAGCT | 406 | TCCCTGTCCTCCAGGAGCTC |
| 22 | 363 | GTCGTATCCAGTGCAGGGTCCGAGGTATTCGCACTGGATACGACCTCGGG | 407 | TCCTGTACTGAGCTACCCCG |
| 23 | 364 | GTCGTATCCAGTGCAGGGTCCGAGGTATTCGCACTGGATACGACTCTGGG | 408 | TCCTGTACTGAGCTGCCCCA |
| 24 | 365 | GTCGTATCCAGTGCAGGGTCCGAGGTATTCGCACTGGATACGACGGCCTC | 409 | TCCTGTACTGAGCTGCCCCG |
| 25 | 366 | GTCGTATCCAGTGCAGGGTCCGAGGTATTCGCACTGGATACGACCTGGAG | 410 | TCTACAGTGCACGTGTCTCC |
| 26 | 367 | GTCGTATCCAGTGCAGGGTCCGAGGTATTCGCACTGGATACGACACCCAC | 41. | TCTTCGGGGGCGTGGGT |
| 27 | 368 | GTCGTATCCAGTGCAGGGTCCGAGGTATTCGCACTGGATACGACCTCGGG | 412 | TCTTGTACTGAGCTGCCCCG |
| 28 | 369 | GTCGTATCCAGTGCAGGGTCCGAGGTATTCGCACTGGATACGACTTTCTC | 413 | TGAGGGGCAGAGAGCGAGAA |
| 29 | 370 | GTCGTATCCAGTGCAGGGTCCGAGGTATTCGCACTGGATACGACAACCCG | 414 | TGGCATGCACGAGGCCCCGG |
| 30 | 371 | GTCGTATCCAGTGCAGGGTCCGAGGTATTCGCACTGGATACGACAAACCC | 415 | TGGCATGCACGAGGCCCCGG |
| 31 | 372 | GTCGTATCCAGTGCAGGGTCCGAGGTATTCGCACTGGATACGACGTTCTC | 416 | TGGGTTTACGTTGGGAGAAC |
| 32 | 373 | GTCGTATCCAGTGCAGGGTCCGAGGTATTCGCACTGGATACGACTGAGAG | 417 | TGGTTAGGATTCGGCGCTCT |
| 33 | 374 | GTCGTATCCAGTGCAGGGTCCGAGGTATTCGCACTGGATACGACAACAGG | 418 | TGTCCCACCCCCACTCCTGT |
| 34 | 375 | GTCGTATCCAGTGCAGGGTCCGAGGTATTCGCACTGGATACGACCATCAG | 419 | TCAAGTGATGGAGAGCAATA |
| 35 | 376 | GTCGTATCCAGTGCAGGGTCCGAGGTATTCGCACTGGATACGACCCCGGG | 420 | GCATTGGTGGTTCAGTGGTA |
| 36 | 377 | GTCGTATCCAGTGCAGGGTCCGAGGTATTCGCACTGGATACGACCCCGGG | 421 | GCATGGGTGGTTCAGTGGTA |
| 37 | 378 | GTCGTATCCAGTGCAGGGTCCGAGGTATTCGCACTGGATACGACAAACGA | 422 | GCATTGGTGGTTCAGTGGTA |
| 38 | 379 | GTCGTATCCAGTGCAGGGTCCGAGGTATTCGCACTGGATACGACACCCGG | 423 | TTGGTGGTTCAGTGGTAGAA |
| 39 | 380 | GTCGTATCCAGTGCAGGGTCCGAGGTATTCGCACTGGATACGACACCCGG | 424 | GCATTGGTGGTTCAGTGGTA |
| 40 | 381 | GTCGTATCCAGTGCAGGGTCCGAGGTATTCGCACTGGATACGACGGCCGG | 425 | GCATTGGTGGTTCAGTGGTA |
| 41 | 382 | GTCGTATCCAGTGCAGGGTCCGAGGTATTCGCACTGGATACGACCCCCAC | 426 | GTGGGGGAGAGGCTGTCGCT |
| 42 | 383 | GTCGTATCCAGTGCAGGGTCCGAGGTATTCGCACTGGATACGACACCCGG | 427 | GCATTAGTGGTTCAGTGGTA |
| 43 | 384 | GTCGTATCCAGTGCAGGGTCCGAGGTATTCGCACTGGATACGACTACCCG | 428 | GCATTGGTGGTTCAGTGGTA |
| 44 | 385 | GTCGTATCCAGTGCAGGGTCCGAGGTATTCGCACTGGATACGACACCCGG | 429 | GCATTGGTGGTTTAGTGGTA |
| Targets | | | | |
| SRNA | | | | |
| Feature | | | | |
| of | SEQ | | SEQ | |
| SEQ ID | ID | | ID | TaqMan Probe |
| NO: | NO. | Reverse Primer 5′ to 3′ | NO. | 5′ to 3′ |
| 1 | 430 | GTGCAGGGTCCGAGGT | 431 | CTTGAACACCAGCAT |
| 2 | 430 | GTGCAGGGTCCGAGGT | 432 | TCCGGGGAGCAGCAT |
| 3 | 430 | GTGCAGGGTCCGAGGT | 433 | TGCTCGGGGCAGCAT |
| 4 | 430 | GTGCAGGGTCCGAGGT | 434 | CGGGGCTCTCAGCAT |
| 5 | 430 | GTGCAGGGTCCGAGGT | 435 | GCCAGCACACAGCAT |
| 6 | 430 | GTGCAGGGTCCGAGGT | 436 | GTGGAGGTGCAGCAT |
| 7 | 430 | GTGCAGGGTCCGAGGT | 437 | CATGGTCACCAGCAT |
| 8 | 430 | GTGCAGGGTCCGAGGT | 438 | AGGACAATTCAGCAT |
| 9 | 430 | GTGCAGGGTCCGAGGT | 439 | GAGGACAAACAGCAT |
| 10 | 430 | GTGCAGGGTCCGAGGT | 440 | TGAGGACAACAGCAT |
| 11 | 430 | GTGCAGGGTCCGAGGT | 441 | ACGGAAAGCCAGCAT |
| 12 | 430 | GTGCAGGGTCCGAGGT | 442 | GGCGGGCGGCAGCAT |
| 13 | 430 | GTGCAGGGTCCGAGGT | 443 | AGCCAGACTCAGCAT |
| 14 | 430 | GTGCAGGGTCCGAGGT | 444 | ACATATCAACAGCAT |
| 15 | 430 | GTGCAGGGTCCGAGGT | 445 | CAAAGGAGACAGCAT |
| 16 | 430 | GTGCAGGGTCCGAGGT | 446 | GAGGAGAGACAGCAT |
| 17 | 430 | GTGCAGGGTCCGAGGT | 447 | CCGGACACTCAGCAT |
| 18 | 430 | GTGCAGGGTCCGAGGT | 448 | GGTGGTTTTCAGCAT |
| 19 | 430 | GTGCAGGGTCCGAGGT | 449 | CTCGAGTGCCAGCAT |
| 20 | 430 | GTGCAGGGTCCGAGGT | 450 | TCGAGTGCTCAGCAT |
| 21 | 430 | GTGCAGGGTCCGAGGT | 451 | TCCTCGAGTCAGCAT |
| 22 | 430 | GTGCAGGGTCCGAGGT | 452 | ATGGGGCTCCAGCAT |
| 23 | 430 | GTGCAGGGTCCGAGGT | 453 | ACGGGGTCTCAGCAT |
| 24 | 430 | GTGCAGGGTCCGAGGT | 454 | GGGCTCCGGCAGCAT |
| 25 | 430 | GTGCAGGGTCCGAGGT | 455 | ACAGAGGTCCAGCAT |
| 26 | 430 | GTGCAGGGTCCGAGGT | 456 | CCGCACCCACAGCAT |
| 27 | 430 | GTGCAGGGTCCGAGGT | 457 | ACGGGGCTCCAGCAT |
| 28 | 430 | GTGCAGGGTCCGAGGT | 458 | TCGCTCTTTCAGCAT |
| 29 | 430 | GTGCAGGGTCCGAGGT | 459 | GGGGCCCAACAGCAT |
| 30 | 430 | GTGCAGGGTCCGAGGT | 460 | GGGCCCAAACAGCAT |
| 31 | 430 | GTGCAGGGTCCGAGGT | 461 | ACCCTCTTGCAGCAT |
| 32 | 430 | GTGCAGGGTCCGAGGT | 462 | CGCGAGAGTCAGCAT |
| 33 | 430 | GTGCAGGGTCCGAGGT | 463 | TGAGGACAACAGCAT |
| 34 | 430 | GTGCAGGGTCCGAGGT | 464 | ACAGACTACCAGCAT |
| 35 | 430 | GTGCAGGGTCCGAGGT | 465 | TCCGGGCCCCAGCAT |
| 36 | 430 | GTGCAGGGTCCGAGGT | 466 | TCCGGGCCCCAGCAT |
| 37 | 430 | GTGCAGGGTCCGAGGT | 467 | CCAAGCAAACAGCAT |
| 38 | 430 | GTGCAGGGTCCGAGGT | 468 | CCGGGCCCACAGCAT |
| 39 | 430 | GTGCAGGGTCCGAGGT | 469 | CTGGGCCCACAGCAT |
| 40 | 430 | GTGCAGGGTCCGAGGT | 470 | AAGGGCCGGCAGCAT |
| 41 | 430 | GTGCAGGGTCCGAGGT | 471 | CCGCACCCCCAGCAT |
| 42 | 430 | GTGCAGGGTCCGAGGT | 472 | CCGGGCCCACAGCAT |
| 43 | 430 | GTGCAGGGTCCGAGGT | 473 | CGGGCCCATCAGCAT |
| 44 | 430 | GTGCAGGGTCCGAGGT | 474 | CCGGGCCCACAGCAT |

TABLE 4
Antisense Molecules Targeting Small RNA Features Correlated
to IPF Risk of Mortality

| Targets | | |
|---|---|---|
| SRNA | SEQ | |
| Feature of | ID | |
| SEQ ID NO: | NO. | Antisense 5′ to 3′ |
| 1 | 475 | CACAAGTTCGGATCTACGGGTT |
| 2 | 476 | GAGGGGCCTCAGACCGAGCTT |
| 3 | 477 | GGGGCTCGTCCGGGATT |
| 4 | 478 | TCTCGGGGCAGCTCAGTACAGGT |
| 5 | 479 | ACACGACCGACGCCACGCCGAGT |
| 6 | 480 | GTGGAGGTGCCGGGGAT |
| 7 | 481 | CACTGGTACAAGGGTTGGGAGAT |
| 8 | 482 | TTAACAGGAGTGGGGGTGGGACAT |
| 9 | 483 | AAACAGGAGTGGGGGTGGGACAT |
| 10 | 484 | AACAGGAGTGGGGGGTGGGACG |
| 11 | 485 | CGAAAGGCAGCAGGTCGTATAG |
| 12 | 486 | GGCGGGCGGGACGGCGAGGTC |
| 13 | 487 | TCAGACCGAGACAAGTGC |
| 14 | 488 | AACTATACAACCTACTAC |
| 15 | 489 | AGAGGAAACCAGCAAGTGTTGAC |
| 16 | 490 | AGAGAGGAGAGCCGTGTATGAC |
| 17 | 491 | TCACAGGCCGGGACAAGTGCAATA |
| 18 | 492 | TTTTGGTGGAGATGCCGGGGA |
| 19 | 493 | CGTGAGCTCCTGGAGGACAGGGA |
| 20 | 494 | TCGTGAGCTCCTGGAGGACAGGGA |
| 21 | 495 | TGAGCTCCTGGAGGACAGGGA |
| 22 | 496 | CTCGGGGTAGCTCAGTACAGGA |
| 23 | 497 | TCTGGGGCAGCTCAGTACAGGA |
| 24 | 498 | GGCCTCGGGGCAGCTCAGTACAGGA |
| 25 | 499 | CTGGAGACACGTGCACTGTAGA |
| 26 | 500 | ACCCACGCCCCCGAAGA |
| 27 | 501 | CTCGGGGCAGCTCAGTACAAGA |
| 28 | 502 | TTTCTCGCTCTCTGCCCCTCA |
| 29 | 503 | AACCCGGGGCCTCGTGCATGCCA |
| 30 | 504 | AAACCCGGGGCCTCGTGCATGCCA |
| 31 | 505 | GTTCTCCCAACGTAAACCCA |
| 32 | 506 | TGAGAGCGCCGAATCCTAACCA |
| 33 | 507 | AACAGGAGTGGGGGTGGGACA |
| 34 | 508 | CATCAGACAGTACGAATACATCCCAATGTGGTCACAGTCATCCCCGGGTATTGCTCTCCATCACTTGA |
| 35 | 509 | CCCGGGCCTCCCGCGTGGCAGGCGAGAATTCTACCACTGAACCACCAATGC |
| 36 | 510 | CCCGGGCCTCCCGCGTGGCAGGCGAGAATTCTACCACTGAACCACCCATGC |
| 37 | 511 | AAACGAACCCGGGCCTCCCGCGTGGCAGGCGAGAATTCTACCACTGAACCACCAATGC |
| 38 | 512 | ACCCGGGCCTCCCGCGTGGCAGGCGAGAATTCTACCACTGAACCACCAA |
| 39 | 513 | ACCCGGGTCTCCCGCGTGGCAGGCGAGAATTCTACCACTGAACCACCAATGC |
| 40 | 514 | GGCCGGGAAACGAACCCGGGCCTCCCGCGTGGCAGGCGAGAATTCTACCACTGAACCACCAATGC |
| 41 | 515 | CCCCACGCCCGCCGCGGGCCCAGAAGCGCAGCGACAGCCTCTCCCCCAC |
| 42 | 516 | ACCCGGGCCTCCCGCGTGGCAGGCGAGAATTCTACCACTGAACCACTAATGC |
| 43 | 517 | TACCCGGGCCTCCCGCGTGGCAGGCGAGAATTCTACCACTGAACCACCAATGC |
| 44 | 518 | ACCCGGGCCTCCCGCGTGGCAGGCGAGAATTCTACCACTAAACCACCAATGC |

TABLE 5
Therapeutic Molecules Based on Small RNA Features Correlated to IPF Risk of Mortality

| SEQ | |
|---|---|
| ID NO | siRNA Mimetic |
| 116 | AACCCGUAGAUCCGAACUUGUGdTdT |
| | \|\|\|\|\|\|\|\|\|\|\|\|\|\|\|\|\|\|\|\|\| |
| 117 | dTdTAUGGGCUUCUAGGCUUGUACAC |
| 118 | AAGCUCGGUCUGAGGCCCCUCdTdT |
| | \|\|\|\|\|\|\|\|\|\|\|\|\|\|\|\|\|\|\|\| |
| 119 | dTdTAUCGAGCCAGACUCCGGGGAG |
| 822 | AAUCCCGGACGAGCCCCdTdT |
| | \|\|\|\|\|\|\|\|\|\|\|\|\|\|\|\| |
| 823 | dTdTAUAGGGCCUGCUCGGGG |
| 120 | ACCUGUACUGAGCUGCCCCGAGAdTdT |
| | \|\|\|\|\|\|\|\|\|\|\|\|\|\|\|\|\|\|\|\|\|\| |
| 121 | dTdTAGGACAUGACUCGACGGGGCUCU |
| 122 | ACUCGGCGUGGCGUCGGUCGUGUdTdT |
| | \|\|\|\|\|\|\|\|\|\|\|\|\|\|\|\|\|\|\|\|\|\| |
| 123 | dTdTAGAGCCGCACCGCAGCCUGCACA |
| 124 | AUCCCCGGCACCUCCACdTdT |
| | \|\|\|\|\|\|\|\|\|\|\|\|\|\|\|\| |
| 125 | dTdTAAGGGGCCGUGGAGGUG |
| 126 | AUCUCCCAACCCUUGUACCAGUGdTdT |
| | \|\|\|\|\|\|\|\|\|\|\|\|\|\|\|\|\|\|\|\|\|\| |
| 127 | dTdTAAGAGGGUUGGGAACAUGGUCAC |
| 128 | AUGUCCCACCCCCACUCCUGUUAAdTdT |
| | \|\|\|\|\|\|\|\|\|\|\|\|\|\|\|\|\|\|\|\|\|\|\| |
| 129 | dTdTAACAGGGUGGGGGUGAGGACAAUU |
| 130 | AUGUCCCACCCCCACUCCUGUUUdTdT |
| | \|\|\|\|\|\|\|\|\|\|\|\|\|\|\|\|\|\|\|\|\|\| |
| 131 | dTdTAACAGGGUGGGGGUGAGGUCAAA |
| 132 | CGUCCCACCCCCCACUCCUGUUdTdT |
| | \|\|\|\|\|\|\|\|\|\|\|\|\|\|\|\|\|\|\|\|\| |
| 133 | dTdTCCAGGGUGGGGGGUGAGGACAA |
| 134 | CUAUACGACCUGCUGCCUUUCGdTdT |
| | \|\|\|\|\|\|\|\|\|\|\|\|\|\|\|\|\|\|\|\|\| |
| 135 | dTdTCAUAUGCUGGACGACGGAAAGC |
| 136 | GACCUCGCCGUCCCGCCCGCCdTdT |
| | \|\|\|\|\|\|\|\|\|\|\|\|\|\|\|\|\|\|\|\|\| |
| 137 | dTdTGUGGAGCGGCAGGGCGGGCGG |
| 138 | GCACUUGUCUCGGUCUGAdTdT |
| | \|\|\|\|\|\|\|\|\|\|\|\|\|\|\|\|\| |
| 139 | dTdTGGUGAACAGUGCCAGACU |
| 140 | GUAGUAGGUUGUAUAGUUdTdT |
| | \|\|\|\|\|\|\|\|\|\|\|\|\|\|\|\|\| |
| 141 | dTdTGAUCAUCCAACAUAUCAA |
| 142 | GUCAACACUUGCUGGUUUCCUCUdTdT |
| | \|\|\|\|\|\|\|\|\|\|\|\|\|\|\|\|\|\|\|\|\|\| |
| 143 | dTdTGAGUUGUGAACGACCAAAGGAGA |
| 144 | GUCAUACACGGCUCUCCUCUCUdTdT |
| | \|\|\|\|\|\|\|\|\|\|\|\|\|\|\|\|\|\|\|\|\| |
| 145 | dTdTGAGUAUGUGCCGAGAGGAGAGA |
| 146 | UAUUGCACUUGUCCCGGCCUGUGAdTdT |
| | \|\|\|\|\|\|\|\|\|\|\|\|\|\|\|\|\|\|\|\|\|\|\| |
| 147 | dTdTUUAACGUGAACAGGGCCGGACACU |
| 148 | UCCCCGGCAUCUCCACCAAAAdTdT |
| | \|\|\|\|\|\|\|\|\|\|\|\|\|\|\|\|\|\|\|\| |
| 149 | dTdTUGGGGCCGUAGAGGUGGUUUU |
| 150 | UCCCUGUCCUCCAGGAGCUCACGdTdT |
| | \|\|\|\|\|\|\|\|\|\|\|\|\|\|\|\|\|\|\|\|\|\| |
| 151 | dTdTUGGGACAGGAGGUCCUCGAGUGC |
| 152 | UCCCUGUCCUCCAGGAGCUCACGAdTdT |
| | \|\|\|\|\|\|\|\|\|\|\|\|\|\|\|\|\|\|\|\|\|\|\| |
| 153 | dTdTUGGGUCUGGUGGUCCUCGUGUGCU |
| 154 | UCCCUGUCCUCCAGGAGCUCAdTdT |
| | \|\|\|\|\|\|\|\|\|\|\|\|\|\|\|\|\|\|\|\| |
| 155 | dTdTUGGGACAGGAGGUCCUCGAGU |
| 156 | UCCUGUACUGAGCUACCCCGAGdTdT |
| | \|\|\|\|\|\|\|\|\|\|\|\|\|\|\|\|\|\|\|\|\| |
| 157 | dTdTUGGACAUGACUCGAUGGGGCUC |
| 158 | UCCUGUACUGAGCUGCCCCAGAdTdT |
| | \|\|\|\|\|\|\|\|\|\|\|\|\|\|\|\|\|\|\|\|\| |
| 159 | dTdTUGGACAUGACUCGACGGGGUCU |
| 160 | UCCUGUACUGAGCUGCCCCGAGGCCdTdT |
| | \|\|\|\|\|\|\|\|\|\|\|\|\|\|\|\|\|\|\|\|\|\|\|\| |
| 161 | dTdTUGGACAUGACUCGACGGGGCUCCGG |
| 162 | UCUACAGUGCACGUGUCUCCAGdTdT |
| | \|\|\|\|\|\|\|\|\|\|\|\|\|\|\|\|\|\|\|\|\| |
| 163 | dTdTUGAUGUCACGUGCACAGUGGUC |
| 164 | UCUUCGGGGGCGUGGGUdTdT |
| | \|\|\|\|\|\|\|\|\|\|\|\|\|\|\|\| |
| 165 | dTdTUGAAGCCCCCGCACCCA |
| 166 | UCUUGUACUGAGCUGCCCCGAGdTdT |
| | \|\|\|\|\|\|\|\|\|\|\|\|\|\|\|\|\|\|\|\|\| |
| 167 | dTdTUGAACAUGACUCGACGGGGCUC |
| 168 | UGAGGGGCAGAGAGCGAGAAAdTdT |
| | \|\|\|\|\|\|\|\|\|\|\|\|\|\|\|\|\|\|\|\| |
| 169 | dTdTUCUCCCCGUCUCUCGCUCUUU |
| 170 | UGGCAUGCACGAGGCCCCGGGUUdTdT |
| | \|\|\|\|\|\|\|\|\|\|\|\|\|\|\|\|\|\|\|\|\|\| |
| 171 | dTdTUCCGUACGUGCUCCGGGGCCCAA |
| 172 | UGGCAUGCACGAGGCCCCGGGUUUdTdT |
| | \|\|\|\|\|\|\|\|\|\|\|\|\|\|\|\|\|\|\|\|\|\|\| |
| 173 | dTdTUCCGUUCGUGCUCCGGGGCCCAAA |
| 174 | UGGGUUUACGUUGGGAGAACdTdT |
| | \|\|\|\|\|\|\|\|\|\|\|\|\|\|\|\|\|\|\| |
| 175 | dTdTUCCCAAAUGCAACCCUCUUG |
| 176 | UGGUUAGGAUUCGGCGCUCUCAdTdT |
| | \|\|\|\|\|\|\|\|\|\|\|\|\|\|\|\|\|\|\|\|\| |
| 177 | dTdTUCCAAUCCUAAGCCGCGAGAGU |
| 178 | UGUCCCACCCCCACUCCUGUUdTdT |
| | \|\|\|\|\|\|\|\|\|\|\|\|\|\|\|\|\|\|\|\| |
| 179 | dTdTUCAGGGUGGGGGUGAGGACAA |
| 180 | UCAAGUGAUGGAGAGCAAUACCCGGGGAUGACUGUGACCACAUUGGGAUGUAUUCGUACUGUCUGAUGdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 181 | dTdTUGUUCACUACCUCUCGUUAUGGGCCCCUACUGACACUGGUGUAACCCUACAUAAGCAUGACAGACUAC |
| 896 | GCAUUGGUGGUUCAGUGGUAGAAUUCUCGCCUGCCACGCGGGAGGCCCGGGdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 182 | dTdTGGUAACCACCAAGUCACCAUCUUAAGAGCGGACGGUGCGCCCUCCGGGCCC |
| 183 | GCAUGGGUGGUUCAGUGGUAGAAUUCUCGCCUGCCACGCGGGAGGCCCGGGdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 184 | dTdTGGUACCCACCAAGUCACCAUCUUAAGAGCGGACGGUGCGCCCUCCGGGCCC |
| 185 | GCAUUGGUGGUUCAGUGGUAGAAUUCUCGCCUGCCACGCGGGAGGCCCGGGUUCGUUUdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 186 | dTdTGGUAACCACCAAGUCACCAUCUUAAGAGCGGACGGUGCGCCCUCCGGGCCCAAGCAAA |
| 187 | UUGGUGGUUCAGUGGUAGAAUUCUCGCCUGCCACGCGGGAGGCCCGGGUdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 188 | dTdTUACCACCAAGUCACCAUCUUAAGAGCGGACGGUGCGCCCUCCGGGCCCA |
| 189 | GCAUUGGUGGUUCAGUGGUAGAAUUCUCGCCUGCCACGCGGGAGACCCGGGUdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 190 | dTdTGGUAACCACCAAGUCACCAUCUUAAGAGCGGACGGUGCGCCCUCUGGGCCCA |
| 191 | GCAUUGGUGGUUCAGUGGUAGAAUUCUCGCCUGCCACGCGGGAGGCCCGGGUUCGUUUCCCGGCCdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 192 | dTdTGGUAACCACCAAGUCACCAUCUUAAGAGCGGACGGUGCGCCCUCCGGGCCCAAGCAAAGGGCCGG |
| 193 | GUGGGGGAGAGGCUGUCGCUGCGCUUCUGGGCCCGCGGCGGGCGUGGGGdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 194 | dTdTGACCCCCUCUCCGACAGCGACGCGAAGACCCGGGCGCCGCCCGCACCCC |
| 195 | GCAUUAGUGGUUCAGUGGUAGAAUUCUCGCCUGCCACGCGGGAGGCCCGGGUdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 196 | dTdTGGUAAUCACCAAGUCACCAUCUUAAGAGCGGACGGUGCGCCCUCCGGGCCCA |
| 197 | GCAUUGGUGGUUCAGUGGUAGAAUUCUCGCCUGCCACGCGGGAGGCCCGGGUAdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 198 | dTdTGGUAACCUCCAAGUCUCCAUCUUAAGAGCGGACGGUGCGCCCUCCGGGCCCAU |
| 199 | GCAUUGGUGGUUUAGUGGUAGAAUUCUCGCCUGCCACGCGGGAGGCCCGGGUdTdT |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| 200 | dTdTGGUAACCACCAAAUCUCCAUCUUAAGAGCGGACGGUGCGCCCUCCGGGCCCA |

TABLE 6A
Small RNA Features for Evaluating Subtype of IPF

| SEQ ID | | | | Population |
|---|---|---|---|---|
| NO: | Small RNA Sequence 5′ to 3′ | Mapped Name | Small RNA Class | Frequency |
| 45 | TATTGCACTTGTCCCGGCCTATA | hsa-mir-92a-3p | MicroRNA Isoform | 100% |
| 46 | TATTGCACTTGTCCCGGCCTGT | hsa-mir-92a-3p | MicroRNA Isoform | 100% |
| 47 | TATGGCACTGGTAGAATTCACT | hsa-mir-183-5p | MicroRNA Isoform | 100% |
| 48 | ATGGCACTGGTAGAATTCACTGT | hsa-mir-183-5p | MicroRNA Isoform | 96% |
| 49 | AATATTGCACTTGTCCCGGCCTGTA | hsa-mir-92b-3p | MicroRNA Isoform | 98% |
| 50 | CCAAAACTGCAGTTACTTTTG | hsa-mir-5480-3p | MicroRNA Isoform | 97% |
| 51 | TATTACACTTGTCCCGGCCTGT | hsa-mir-92a-3p | MicroRNA Isoform | 100% |
| 52 | CAAAGTGCTCATAGTGCAGGTAGA | hsa-mir-20b-5p | MicroRNA Isoform | 93% |
| 53 | GGGAGCCAGGAAGTATTGATGTT | hsa-mir-505-5p | MicroRNA Isoform | 100% |
| 54 | TATTGCACTTGTCCCGGCCTGTTAA | hsa-mir-92a-3p | MicroRNA Isoform | 98% |
| 55 | GATTGCACTTGTCCCGGCCTGT | hsa-mir-92a-3p | MicroRNA Isoform | 99% |
| 56 | GTGTCAGTTTGTCAAATACCCCAAT | hsa-mir-223-3p | MicroRNA Isoform | 50% |
| 57 | AGCTCGGTCTGAGGCCCCTCAGTTAT | hsa-mir-423-3p | MicroRNA Isoform | 99% |
| 58 | ACAAACCGTTACCATTACTGAGTT | hsa-mir-451a | MicroRNA Isoform | 26% |
| 59 | CCTGTACTGAGCTGCCCCTAG | hsa-mir-486-5p | MicroRNA Isoform | 96% |
| 60 | AATGCACCCGGGCAAGGATT | hsa-mir-501-3p | MicroRNA Isoform | 100% |
| 61 | TTAGCAGCACATCATGGTTTA | hsa-mir-15b-5p | MicroRNA Isoform | 87% |
| 62 | AATGGCGCCACTAGGGTTGTGAA | hsa-mir-652-3p | MicroRNA Isoform | 100% |
| 63 | TTCTGGAATTCTGTGTGAGGGA | hsa-mir-1299 | MicroRNA Isoform | 66% |
| 64 | ATATTGCACTTGTCCCGGCCTGA | hsa-mir-92b-3p | MicroRNA Isoform | 100% |
| 65 | AAACTCTACTTGTCCTTCTGAG | hsa-mir-618 | MicroRNA Isoform | 97% |
| 66 | TTATTGCACTTGTCCCGGCCTG | hsa-mir-92a-3p | MicroRNA Isoform | 99% |
| 67 | CTCGTCCTGTACTGAGCTGCCCCGAG | hsa-mir-486-5p | MicroRNA Isoform | 92% |
| 68 | CCTGTACTGAGCTGCCCCGAGTTT | hsa-mir-486-5p | MicroRNA Isoform | 99% |
| 69 | GAACTCACCCTCTGCTCCCAGT | hsa-mir-6750-3p | MicroRNA Isoform | 100% |
| 70 | TGGAGAGAAAGGCAGTTCCTGT | hsa-mir-185-5p | MicroRNA Isoform | 99% |
| 71 | TATTGCACTTGTCCCGGCCAAAA | hsa-mir-92a-3p | MicroRNA Isoform | 99% |
| 72 | TTTTAGAGACGGGGTCTTGCT | hsa-mir-1303 | MicroRNA Isoform | 99% |
| 73 | TTTGGCAATGGTAGAACTCACACTGGA | hsa-mir-182-5p | MicroRNA Isoform | 98% |
| 74 | TGTAGAGCAGGGAGCAGGAAG | hsa-mir-4732-5p | MicroRNA Isoform | 100% |
| 75 | GTATTGCACTTGTCCCGGCCTGT | hsa-mir-92a-3p | MicroRNA Isoform | 100% |
| 76 | TGTGAGGTTGGCATTGTTGTCT | hsa-mir-1294 | MicroRNA Isoform | 90% |
| 77 | GTATTGCACTTGTCCCGGCCTGTA | hsa-mir-92a-3p | MicroRNA Isoform | 100% |
| 78 | TGTGAGGTTGGCATTGTTGT | hsa-mir-1294 | MicroRNA Isoform | 100% |
| 79 | TCCACTCTCCTGGCCCCCAGTT | hsa-mir-6777-3p | MicroRNA Isoform | 98% |
| 80 | TCCTGTACTGATCTGCCCCGAGT | hsa-mir-486-5p | MicroRNA Isoform | 99% |
| 81 | AAACATTCAACGCTGTCGGTGAG | hsa-mir-181a-5p | MicroRNA Isoform | 96% |
| 82 | TAAAGTGCTGACAGTGCAGATAGA | hsa-mir-106b-5p | MicroRNA Isoform | 66% |
| 83 | TCCTGTACTGAGCTGCCCCGAGTTC | hsa-mir-486-5p | MicroRNA Isoform | 88% |
| 84 | TCCTGTACTGAGCTGCCCCGTTT | hsa-mir-486-5p | MicroRNA Isoform | 91% |
| 85 | TCTCTCGGCTCCTCGCGGCTCGTA | hsa-mir-3615 | MicroRNA Isoform | 99% |
| 86 | CCTGTACTGAGCTGCGCCGAG | hsa-mir-486-5p | MicroRNA Isoform | 89% |
| 87 | ATGGCACTGGTAGAATTCA | hsa-mir-183-5p | MicroRNA Isoform | 97% |
| 88 | ATATTGCACTTGTCCCGGCCTGTA | hsa-mir-92b-3p | MicroRNA Isoform | 100% |
| 89 | ATATTGCACTTGTCCCGGCCTGT | hsa-mir-92b-3p | MicroRNA Isoform | 100% |
| 90 | TATTGCACTTGTCTCGGCCTGTA | hsa-mir-92a-3p | MicroRNA Isoform | 100% |
| 91 | TATTGCACTTGTCCCGGCCTGATT | hsa-mir-92a-3p | MicroRNA Isoform | 99% |
| 92 | ATATTGCACTTGTCCCGGCCTG | hsa-mir-92b-3p | MicroRNA Isoform | 100% |
| 93 | TGTCCTGTACTGAGCTGCCCCGAGT | hsa-mir-486-5p | MicroRNA Isoform | 96% |
| 94 | TGTGTCCGGGAAGTGGAGGAG | hsa-mir-4669 | MicroRNA Isoform | 48% |
| 95 | TTATTGCACTTGTCCCGGCCTGT | hsa-mir-92a-3p | MicroRNA Isoform | 100% |
| 96 | CTCCTGTACTGAGCTGCCCCGAGAA | hsa-mir-486-5p | MicroRNA Isoform | 99% |
| 97 | AAAGTGCTGTTCGTGCAGGTAGT | hsa-mir-93-5p | MicroRNA Isoform | 86% |
| 98 | TATTGCACTTATCCCGGCCTGT | hsa-mir-92a-3p | MicroRNA Isoform | 100% |
| 99 | TATTGCACTTGTCCCGGCCTGTCA | hsa-mir-92a-3p | MicroRNA Isoform | 94% |
| 100 | TCAGGCTCAGTCCCCTCCCGGT | hsa-mir-484 | MicroRNA Isoform | 99% |
| 101 | TATTGCACTTGTCCCGGCCTGTACG | hsa-mir-92a-3p | MicroRNA Isoform | 44% |
| 102 | CTATTGCACTTGTCCCGGCCTGTA | hsa-mir-92a-3p | MicroRNA Isoform | 100% |
| 103 | CCTGTACTGAGCTGCCCCGAGTGA | hsa-mir-486-5p | MicroRNA Isoform | 92% |
| 104 | CTGTACTGAGCTGCCCCGAAG | hsa-mir-486-5p | MicroRNA Isoform | 98% |
| 105 | TACTGCCCTAAATGCCCCTTC | hsa-mir-18b-3p | MicroRNA Isoform | 98% |
| 106 | CAAAGTGCTTACAGTGCAGGTAGAA | hsa-mir-17-5p | MicroRNA Isoform | 71% |
| 107 | TGTAGAGCAGGGAGCAGGAAGCT | hsa-mir-4732-5p | MicroRNA Isoform | 100% |
| 108 | TGTCAGTTTGTCAAATACCCCATAT | hsa-mir-223-3p | MicroRNA Isoform | 37% |
| 109 | CCGCACTGTGGGTACTTGCTGAT | hsa-mir-106b-3p | MicroRNA Isoform | 98% |
| 110 | ATCCTGTACTGAGCTGCCCCGTT | hsa-mir-486-5p | MicroRNA Isoform | 98% |
| 111 | TCACCCTGCATCCCGCACCCAGT | hsa-mir-3620-3p | MicroRNA Isoform | 100% |
| 112 | CATTGCACTTGTCCCGGCCTGTA | hsa-mir-92a-3p | MicroRNA Isoform | 100% |
| 113 | TTGGCAATGGTAGAACTCACACTG | hsa-mir-182-5p | MicroRNA Isoform | 83% |
| 114 | AAACCGTTACCATTACTGAGTTTAGTA | hsa-mir-451a | MicroRNA Isoform | 44% |
| 115 | AACCGTTACCATTACTGAGTTTAGA | hsa-mir-451a | MicroRNA Isoform | 21% |

TABLE 6B
Expression profiles for IPF Subtypes

| SEQ. ID. NO | Cluster1 | Cluster2 | Cluster3 | Cluster4 |
|---|---|---|---|---|
| 45 | Low | High | Low | High |
| 46 | Low | High | Low | High |
| 47 | Low | High | Low | High |
| 48 | High | High | Low | High |
| 49 | Low | High | Low | High |
| 50 | Low | High | Low | High |
| 51 | Low | High | Low | High |
| 52 | Low | High | Low | High |
| 53 | Low | High | Low | High |
| 54 | Low | High | Low | High |
| 55 | Low | High | Low | High |
| 56 | Low | High | Low | High |
| 57 | Low | High | Low | High |
| 58 | Low | High | Low | High |
| 59 | Low | Low | High | Low |
| 60 | Low | High | Low | High |
| 61 | Low | High | Low | High |
| 62 | Low | High | Low | High |
| 63 | Low | High | Low | High |
| 64 | Low | High | Low | High |
| 65 | Low | High | Low | High |
| 66 | Low | High | Low | High |
| 67 | Low | Low | High | Low |
| 68 | Low | Low | High | High |
| 69 | Low | High | High | Low |
| 70 | Low | High | Low | High |
| 71 | Low | High | Low | High |
| 72 | Low | High | Low | High |
| 73 | Low | High | Low | High |
| 74 | Low | High | Low | Low |
| 75 | Low | High | Low | High |
| 76 | Low | High | Low | High |
| 77 | Low | High | Low | High |
| 78 | High | High | Low | High |
| 79 | Low | High | Low | High |
| 80 | Low | Low | High | High |
| 81 | Low | High | Low | High |
| 82 | Low | High | Low | High |
| 83 | Low | Low | High | High |
| 84 | Low | Low | High | High |
| 85 | Low | High | Low | High |
| 86 | Low | Low | High | Low |
| 87 | Low | High | Low | High |
| 88 | Low | High | Low | High |
| 89 | Low | High | Low | High |
| 90 | Low | High | Low | High |
| 91 | Low | High | Low | High |
| 92 | Low | High | Low | High |
| 93 | Low | Low | High | High |
| 94 | High | High | Low | Low |
| 95 | Low | High | Low | High |
| 96 | Low | Low | High | Low |
| 97 | Low | High | Low | High |
| 98 | Low | High | Low | High |
| 99 | Low | High | Low | High |
| 100 | Low | High | Low | High |
| 101 | Low | Low | Low | High |
| 102 | Low | High | Low | High |
| 103 | Low | Low | High | High |
| 104 | Low | Low | High | High |
| 105 | Low | High | Low | High |
| 106 | Low | High | Low | High |
| 107 | Low | High | Low | High |
| 108 | Low | High | Low | High |
| 109 | Low | High | Low | High |
| 110 | Low | Low | High | Low |
| 111 | Low | High | High | High |
| 112 | Low | High | Low | High |
| 113 | Low | High | Low | High |
| 114 | Low | High | Low | High |
| 115 | Low | High | Low | High |

TABLE 7
Primers and Probes For RT-qPCR Analysis of Small RNA Features Correlated to Subtype of IPF

| Targets | | | | |
|---|---|---|---|---|
| sRNA | | | | |
| Feature | SEQ | | SEQ | |
| of SEQ | ID | | ID | Gene Specific Primer |
| ID NO: | NO: | RT-primer 5′ to 3′ | NO: | 5′ to 3′ |
| 45 | 519 | GTCGTATCCAGTGCAGGGTCCGAGGTATTCGCACTGGATACGACTATAGG | 590 | TATTGCACTTGTCCCGGCCT |
| 46 | 520 | GTCGTATCCAGTGCAGGGTCCGAGGTATTCGCACTGGATACGACACAGGC | 591 | TATTGCACTTGTCCCGGCCT |
| 47 | 521 | GTCGTATCCAGTGCAGGGTCCGAGGTATTCGCACTGGATACGACAGTGAA | 592 | TATGGCACTGGTAGAATTCA |
| 48 | 522 | GTCGTATCCAGTGCAGGGTCCGAGGTATTCGCACTGGATACGACACAGTG | 593 | ATGGCACTGGTAGAATTCAC |
| 49 | 523 | GTCGTATCCAGTGCAGGGTCCGAGGTATTCGCACTGGATACGACTACAGG | 594 | AATATTGCACTTGTCCCGGC |
| 50 | 524 | GTCGTATCCAGTGCAGGGTCCGAGGTATTCGCACTGGATACGACCAAAAG | 595 | CCAAAACTGCAGTTACTTTT |
| 51 | 525 | GTCGTATCCAGTGCAGGGTCCGAGGTATTCGCACTGGATACGACACAGGC | 596 | TATTACACTTGTCCCGGCCT |
| 52 | 526 | GTCGTATCCAGTGCAGGGTCCGAGGTATTCGCACTGGATACGACTCTACC | 597 | CAAAGTGCTCATAGTGCAGG |
| 53 | 527 | GTCGTATCCAGTGCAGGGTCCGAGGTATTCGCACTGGATACGACAACATC | 598 | GGGAGCCAGGAAGTATTGAT |
| 54 | 528 | GTCGTATCCAGTGCAGGGTCCGAGGTATTCGCACTGGATACGACTTAACA | 599 | TATTGCACTTGTCCCGGCCT |
| 55 | 529 | GTCGTATCCAGTGCAGGGTCCGAGGTATTCGCACTGGATACGACACAGGC | 600 | GATTGCACTTGTCCCGGCCT |
| 56 | 530 | GTCGTATCCAGTGCAGGGTCCGAGGTATTCGCACTGGATACGACATTGGG | 601 | GTGTCAGTTTGTCAAATACC |
| 57 | 531 | GTCGTATCCAGTGCAGGGTCCGAGGTATTCGCACTGGATACGACATAACT | 602 | AGCTCGGTCTGAGGCCCCTC |
| 58 | 532 | GTCGTATCCAGTGCAGGGTCCGAGGTATTCGCACTGGATACGACAACTCA | 603 | ACAAACCGTTACCATTACTG |
| 59 | 533 | GTCGTATCCAGTGCAGGGTCCGAGGTATTCGCACTGGATACGACCTAGGG | 604 | CCTGTACTGAGCTGCCCCTA |
| 60 | 534 | GTCGTATCCAGTGCAGGGTCCGAGGTATTCGCACTGGATACGACAATCCT | 605 | AATGCACCCGGGCAAGGATT |
| 61 | 535 | GTCGTATCCAGTGCAGGGTCCGAGGTATTCGCACTGGATACGACTAAACC | 606 | TTAGCAGCACATCATGGTTT |
| 62 | 536 | GTCGTATCCAGTGCAGGGTCCGAGGTATTCGCACTGGATACGACTTCACA | 607 | AATGGCGCCACTAGGGTTGT |
| 63 | 537 | GTCGTATCCAGTGCAGGGTCCGAGGTATTCGCACTGGATACGACTCCCTC | 608 | TTCTGGAATTCTGTGTGAGG |
| 64 | 538 | GTCGTATCCAGTGCAGGGTCCGAGGTATTCGCACTGGATACGACTCAGGC | 609 | ATATTGCACTTGTCCCGGCC |
| 65 | 539 | GTCGTATCCAGTGCAGGGTCCGAGGTATTCGCACTGGATACGACCTCAGA | 610 | AAACTCTACTTGTCCTTCTG |
| 66 | 540 | GTCGTATCCAGTGCAGGGTCCGAGGTATTCGCACTGGATACGACCAGGCC | 611 | TTATTGCACTTGTCCCGGCC |
| 67 | 541 | GTCGTATCCAGTGCAGGGTCCGAGGTATTCGCACTGGATACGACCTCGGG | 612 | CTCGTCCTGTACTGAGCTGC |
| 68 | 542 | GTCGTATCCAGTGCAGGGTCCGAGGTATTCGCACTGGATACGACAAACTC | 613 | CCTGTACTGAGCTGCCCCGA |
| 69 | 543 | GTCGTATCCAGTGCAGGGTCCGAGGTATTCGCACTGGATACGACACTGGG | 614 | GAACTCACCCTCTGCTCCCA |
| 70 | 544 | GTCGTATCCAGTGCAGGGTCCGAGGTATTCGCACTGGATACGACACAGGA | 615 | TGGAGAGAAAGGCAGTTCCT |
| 71 | 545 | GTCGTATCCAGTGCAGGGTCCGAGGTATTCGCACTGGATACGACTTTTGG | 616 | TATTGCACTTGTCCCGGCCA |
| 72 | 546 | GTCGTATCCAGTGCAGGGTCCGAGGTATTCGCACTGGATACGACAGCAAG | 617 | TTTTAGAGACGGGGTCTTGC |
| 73 | 547 | GTCGTATCCAGTGCAGGGTCCGAGGTATTCGCACTGGATACGACTCCAGT | 618 | TTTGGCAATGGTAGAACTCA |
| 74 | 548 | GTCGTATCCAGTGCAGGGTCCGAGGTATTCGCACTGGATACGACCTTCCT | 619 | TGTAGAGCAGGGAGCAGGAA |
| 75 | 549 | GTCGTATCCAGTGCAGGGTCCGAGGTATTCGCACTGGATACGACACAGGC | 620 | GTATTGCACTTGTCCCGGCC |
| 76 | 550 | GTCGTATCCAGTGCAGGGTCCGAGGTATTCGCACTGGATACGACAGACAA | 621 | TGTGAGGTTGGCATTGTTGT |
| 77 | 551 | GTCGTATCCAGTGCAGGGTCCGAGGTATTCGCACTGGATACGACTACAGG | 622 | GTATTGCACTTGTCCCGGCC |
| 78 | 552 | GTCGTATCCAGTGCAGGGTCCGAGGTATTCGCACTGGATACGACACAACA | 623 | TGTGAGGTTGGCATTGTTGT |
| 79 | 553 | GTCGTATCCAGTGCAGGGTCCGAGGTATTCGCACTGGATACGACAACTGG | 624 | TCCACTCTCCTGGCCCCCAG |
| 80 | 554 | GTCGTATCCAGTGCAGGGTCCGAGGTATTCGCACTGGATACGACACTCGG | 625 | TCCTGTACTGATCTGCCCCG |
| 81 | 555 | GTCGTATCCAGTGCAGGGTCCGAGGTATTCGCACTGGATACGACCTCACC | 626 | AAACATTCAACGCTGTCGGT |
| 82 | 556 | GTCGTATCCAGTGCAGGGTCCGAGGTATTCGCACTGGATACGACTCTATC | 627 | TAAAGTGCTGACAGTGCAGA |
| 83 | 557 | GTCGTATCCAGTGCAGGGTCCGAGGTATTCGCACTGGATACGACGAACTC | 628 | TCCTGTACTGAGCTGCCCCG |
| 84 | 558 | GTCGTATCCAGTGCAGGGTCCGAGGTATTCGCACTGGATACGACAAACGG | 629 | TCCTGTACTGAGCTGCCCCG |
| 85 | 559 | GTCGTATCCAGTGCAGGGTCCGAGGTATTCGCACTGGATACGACTACGAG | 630 | TCTCTCGGCTCCTCGCGGCT |
| 86 | 560 | GTCGTATCCAGTGCAGGGTCCGAGGTATTCGCACTGGATACGACCTCGGC | 631 | CCTGTACTGAGCTGCGCCGA |
| 87 | 561 | GTCGTATCCAGTGCAGGGTCCGAGGTATTCGCACTGGATACGACTGAATT | 632 | ATGGCACTGGTAGAATTCA |
| 88 | 562 | GTCGTATCCAGTGCAGGGTCCGAGGTATTCGCACTGGATACGACTACAGG | 633 | ATATTGCACTTGTCCCGGCC |
| 89 | 563 | GTCGTATCCAGTGCAGGGTCCGAGGTATTCGCACTGGATACGACACAGGC | 634 | ATATTGCACTTGTCCCGGCC |
| 90 | 564 | GTCGTATCCAGTGCAGGGTCCGAGGTATTCGCACTGGATACGACTACAGG | 635 | TATTGCACTTGTCTCGGCCT |
| 91 | 565 | GTCGTATCCAGTGCAGGGTCCGAGGTATTCGCACTGGATACGACAATCAG | 636 | TATTGCACTTGTCCCGGCCT |
| 92 | 566 | GTCGTATCCAGTGCAGGGTCCGAGGTATTCGCACTGGATACGACCAGGCC | 637 | ATATTGCACTTGTCCCGGCC |
| 93 | 567 | GTCGTATCCAGTGCAGGGTCCGAGGTATTCGCACTGGATACGACACTCGG | 638 | TGTCCTGTACTGAGCTGCCC |
| 94 | 568 | GTCGTATCCAGTGCAGGGTCCGAGGTATTCGCACTGGATACGACCTCCTC | 639 | TGTGTCCGGGAAGTGGAGGA |
| 95 | 569 | GTCGTATCCAGTGCAGGGTCCGAGGTATTCGCACTGGATACGACACAGGC | 640 | TTATTGCACTTGTCCCGGCC |
| 96 | 570 | GTCGTATCCAGTGCAGGGTCCGAGGTATTCGCACTGGATACGACTTCTCG | 641 | CTCCTGTACTGAGCTGCCCC |
| 97 | 571 | GTCGTATCCAGTGCAGGGTCCGAGGTATTCGCACTGGATACGACACTACC | 642 | AAAGTGCTGTTCGTGCAGGT |
| 98 | 572 | GTCGTATCCAGTGCAGGGTCCGAGGTATTCGCACTGGATACGACACAGGC | 643 | TATTGCACTTATCCCGGCCT |
| 99 | 573 | GTCGTATCCAGTGCAGGGTCCGAGGTATTCGCACTGGATACGACTGACAG | 644 | TATTGCACTTGTCCCGGCCT |
| 100 | 574 | GTCGTATCCAGTGCAGGGTCCGAGGTATTCGCACTGGATACGACACCGGG | 645 | TCAGGCTCAGTCCCCTCCCG |
| 101 | 575 | GTCGTATCCAGTGCAGGGTCCGAGGTATTCGCACTGGATACGACCGTACA | 646 | TATTGCACTTGTCCCGGCCT |
| 102 | 576 | GTCGTATCCAGTGCAGGGTCCGAGGTATTCGCACTGGATACGACTACAGG | 647 | CTATTGCACTTGTCCCGGCC |
| 103 | 577 | GTCGTATCCAGTGCAGGGTCCGAGGTATTCGCACTGGATACGACTCACTC | 648 | CCTGTACTGAGCTGCCCCGA |
| 104 | 578 | GTCGTATCCAGTGCAGGGTCCGAGGTATTCGCACTGGATACGACCTTCGG | 649 | CTGTACTGAGCTGCCCCGAA |
| 105 | 579 | GTCGTATCCAGTGCAGGGTCCGAGGTATTCGCACTGGATACGACGAAGGG | 650 | TACTGCCCTAAATGCCCCTT |
| 106 | 580 | GTCGTATCCAGTGCAGGGTCCGAGGTATTCGCACTGGATACGACTTCTAC | 651 | CAAAGTGCTTACAGTGCAGG |
| 107 | 581 | GTCGTATCCAGTGCAGGGTCCGAGGTATTCGCACTGGATACGACAGCTTC | 652 | TGTAGAGCAGGGAGCAGGAA |
| 108 | 582 | GTCGTATCCAGTGCAGGGTCCGAGGTATTCGCACTGGATACGACATATGG | 653 | TGTCAGTTTGTCAAATACCC |
| 109 | 583 | GTCGTATCCAGTGCAGGGTCCGAGGTATTCGCACTGGATACGACATCAGC | 654 | CCGCACTGTGGGTACTTGCT |
| 110 | 584 | GTCGTATCCAGTGCAGGGTCCGAGGTATTCGCACTGGATACGACAACGGG | 655 | ATCCTGTACTGAGCTGCCCC |
| 111 | 585 | GTCGTATCCAGTGCAGGGTCCGAGGTATTCGCACTGGATACGACACTGGG | 656 | TCACCCTGCATCCCGCACCC |
| 112 | 586 | GTCGTATCCAGTGCAGGGTCCGAGGTATTCGCACTGGATACGACTACAGG | 657 | CATTGCACTTGTCCCGGCCT |
| 113 | 587 | GTCGTATCCAGTGCAGGGTCCGAGGTATTCGCACTGGATACGACCAGTGT | 658 | TTGGCAATGGTAGAACTCAC |
| 114 | 588 | GTCGTATCCAGTGCAGGGTCCGAGGTATTCGCACTGGATACGACTACTAA | 659 | AAACCGTTACCATTACTGAG |
| 115 | 589 | GTCGTATCCAGTGCAGGGTCCGAGGTATTCGCACTGGATACGACTCTAAA | 660 | AACCGTTACCATTACTGAGT |
| Targets | | | | |
| SRNA | | | | |
| Feature | SEQ | | SEQ | |
| of SEQ | ID | | ID | TaqMan Probe |
| ID NO: | NO: | Reverse Primer 5′ to 3′ | NO: | 5′ to 3′ |
| 45 | 661 | GTGCAGGGTCCGAGGT | 662 | GCCGGATATCAGCAT |
| 46 | 661 | GTGCAGGGTCCGAGGT | 663 | GGCCGGACACAGCAT |
| 47 | 661 | GTGCAGGGTCCGAGGT | 664 | CTTAAGTGACAGCAT |
| 48 | 661 | GTGCAGGGTCCGAGGT | 665 | TAAGTGACACAGCAT |
| 49 | 661 | GTGCAGGGTCCGAGGT | 666 | GCCGGACATCAGCAT |
| 50 | 661 | GTGCAGGGTCCGAGGT | 667 | AATGAAAACCAGCAT |
| 51 | 661 | GTGCAGGGTCCGAGGT | 668 | GGCCGGACACAGCAT |
| 52 | 661 | GTGCAGGGTCCGAGGT | 669 | CGTCCATCTCAGCAT |
| 53 | 661 | GTGCAGGGTCCGAGGT | 670 | TAACTACAACAGCAT |
| 54 | 661 | GTGCAGGGTCCGAGGT | 671 | CGGACAATTCAGCAT |
| 55 | 661 | GTGCAGGGTCCGAGGT | 672 | GGCCGGACACAGCAT |
| 56 | 661 | GTGCAGGGTCCGAGGT | 673 | ATGGGGTTACAGCAT |
| 57 | 661 | GTGCAGGGTCCGAGGT | 674 | GAGTCAATACAGCAT |
| 58 | 661 | GTGCAGGGTCCGAGGT | 675 | ATGACTCAACAGCAT |
| 59 | 661 | GTGCAGGGTCCGAGGT | 676 | ACGGGGATCCAGCAT |
| 60 | 661 | GTGCAGGGTCCGAGGT | 677 | CGTTCCTAACAGCAT |
| 61 | 661 | GTGCAGGGTCCGAGGT | 678 | GTACCAAATCAGCAT |
| 62 | 661 | GTGCAGGGTCCGAGGT | 679 | CCAACACTTCAGCAT |
| 63 | 661 | GTGCAGGGTCCGAGGT | 680 | ACACTCCCTCAGCAT |
| 64 | 661 | GTGCAGGGTCCGAGGT | 681 | GGCCGGACTCAGCAT |
| 65 | 661 | GTGCAGGGTCCGAGGT | 682 | GGAAGACTCCAGCAT |
| 66 | 661 | GTGCAGGGTCCGAGGT | 683 | GGGCCGGACCAGCAT |
| 67 | 661 | GTGCAGGGTCCGAGGT | 684 | ACGGGGCTCCAGCAT |
| 68 | 661 | GTGCAGGGTCCGAGGT | 685 | GGGCTCAAACAGCAT |
| 69 | 661 | GTGCAGGGTCCGAGGT | 686 | CGAGGGTCACAGCAT |
| 70 | 661 | GTGCAGGGTCCGAGGT | 687 | TCAAGGACACAGCAT |
| 71 | 661 | GTGCAGGGTCCGAGGT | 688 | GCCGGTTTTCAGCAT |
| 72 | 661 | GTGCAGGGTCCGAGGT | 689 | CCAGAACGACAGCAT |
| 73 | 661 | GTGCAGGGTCCGAGGT | 690 | GTGTGACCTCAGCAT |
| 74 | 661 | GTGCAGGGTCCGAGGT | 691 | TCGTCCTTCCAGCAT |
| 75 | 661 | GTGCAGGGTCCGAGGT | 692 | GGCCGGACACAGCAT |
| 76 | 661 | GTGCAGGGTCCGAGGT | 693 | AACAACAGACAGCAT |
| 77 | 661 | GTGCAGGGTCCGAGGT | 694 | GCCGGACATCAGCAT |
| 78 | 661 | GTGCAGGGTCCGAGGT | 695 | GTAACAACACAGCAT |
| 79 | 661 | GTGCAGGGTCCGAGGT | 696 | GGGGGTCAACAGCAT |
| 80 | 661 | GTGCAGGGTCCGAGGT | 697 | CGGGGCTCACAGCAT |
| 81 | 661 | GTGCAGGGTCCGAGGT | 698 | CAGCCACTCCAGCAT |
| 82 | 661 | GTGCAGGGTCCGAGGT | 699 | CGTCTATCTCAGCAT |
| 83 | 661 | GTGCAGGGTCCGAGGT | 700 | GGGCTCAAGCAGCAT |
| 84 | 661 | GTGCAGGGTCCGAGGT | 701 | CGGGGCAAACAGCAT |
| 85 | 661 | GTGCAGGGTCCGAGGT | 702 | GCCGAGCATCAGCAT |
| 86 | 661 | GTGCAGGGTCCGAGGT | 703 | ACGCGGCTCCAGCAT |
| 87 | 661 | GTGCAGGGTCCGAGGT | 704 | ATCTTAAGTCAGCAT |
| 88 | 661 | GTGCAGGGTCCGAGGT | 705 | GCCGGACATCAGCAT |
| 89 | 661 | GTGCAGGGTCCGAGGT | 706 | GGCCGGACACAGCAT |
| 90 | 661 | GTGCAGGGTCCGAGGT | 707 | GCCGGACATCAGCAT |
| 91 | 661 | GTGCAGGGTCCGAGGT | 708 | CCGGACTAACAGCAT |
| 92 | 661 | GTGCAGGGTCCGAGGT | 709 | GGGCCGGACCAGCAT |
| 93 | 661 | GTGCAGGGTCCGAGGT | 710 | CGGGGCTCACAGCAT |
| 94 | 661 | GTGCAGGGTCCGAGGT | 711 | CACCTCCTCCAGCAT |
| 95 | 661 | GTGCAGGGTCCGAGGT | 712 | GGCCGGACACAGCAT |
| 96 | 661 | GTGCAGGGTCCGAGGT | 713 | GGGGCTCTTCAGCAT |
| 97 | 661 | GTGCAGGGTCCGAGGT | 714 | CGTCCATCACAGCAT |
| 98 | 661 | GTGCAGGGTCCGAGGT | 715 | GGCCGGACACAGCAT |
| 99 | 661 | GTGCAGGGTCCGAGGT | 716 | CCGGACAGTCAGCAT |
| 100 | 661 | GTGCAGGGTCCGAGGT | 717 | GGAGGGCCACAGCAT |
| 101 | 661 | GTGCAGGGTCCGAGGT | 718 | CGGACATGCCAGCAT |
| 102 | 661 | GTGCAGGGTCCGAGGT | 719 | GCCGGACATCAGCAT |
| 103 | 661 | GTGCAGGGTCCGAGGT | 720 | GGGCTCACTCAGCAT |
| 104 | 661 | GTGCAGGGTCCGAGGT | 721 | CGGGGCTTCCAGCAT |
| 105 | 661 | GTGCAGGGTCCGAGGT | 722 | ACGGGGAAGCAGCAT |
| 106 | 661 | GTGCAGGGTCCGAGGT | 723 | GTCCATCTTCAGCAT |
| 107 | 661 | GTGCAGGGTCCGAGGT | 724 | GTCCTTCGACAGCAT |
| 108 | 661 | GTGCAGGGTCCGAGGT | 725 | TGGGGTATACAGCAT |
| 109 | 661 | GTGCAGGGTCCGAGGT | 726 | GAACGACTACAGCAT |
| 110 | 661 | GTGCAGGGTCCGAGGT | 727 | ACGGGGCAACAGCAT |
| 111 | 661 | GTGCAGGGTCCGAGGT | 728 | CGTGGGTCACAGCAT |
| 112 | 661 | GTGCAGGGTCCGAGGT | 729 | GCCGGACATCAGCAT |
| 113 | 661 | GTGCAGGGTCCGAGGT | 730 | GAGTGTGACCAGCAT |
| 114 | 661 | GTGCAGGGTCCGAGGT | 731 | TCAAATCATCAGCAT |
| 115 | 661 | GTGCAGGGTCCGAGGT | 732 | CTCAAATCTCAGCAT |

TABLE 8
Therapeutic Molecules Targeting Small RNA Features
Correlated to Subtype of IPF

| Targets | | |
|---|---|---|
| SRNA | | |
| Feature | | |
| of SEQ | SEQ ID | Antisense Oligonucleotide |
| ID NO: | NO. | 5′ to 3′ |
| 45 | 825 | TATAGGCCGGGACAAGTGCAATA |
| 46 | 826 | ACAGGCCGGGACAAGTGCAATA |
| 47 | 827 | AGTGAATTCTACCAGTGCCATA |
| 48 | 828 | ACAGTGAATTCTACCAGTGCCAT |
| 49 | 829 | TACAGGCCGGGACAAGTGCAATATT |
| 50 | 830 | CAAAAGTAACTGCAGTTTTGG |
| 51 | 831 | ACAGGCCGGGACAAGTGTAATA |
| 52 | 832 | TCTACCTGCACTATGAGCACTTTG |
| 53 | 833 | AACATCAATACTTCCTGGCTCCC |
| 54 | 834 | TTAACAGGCCGGGACAAGTGCAATA |
| 55 | 835 | ACAGGCCGGGACAAGTGCAATC |
| 56 | 836 | ATTGGGGTATTTGACAAACTGACAC |
| 57 | 837 | ATAACTGAGGGGCCTCAGACCGAGCT |
| 58 | 838 | AACTCAGTAATGGTAACGGTTTGT |
| 59 | 839 | CTAGGGGCAGCTCAGTACAGG |
| 60 | 840 | AATCCTTGCCCGGGTGCATT |
| 61 | 841 | TAAACCATGATGTGCTGCTAA |
| 62 | 842 | TTCACAACCCTAGTGGCGCCATT |
| 63 | 843 | TCCCTCACACAGAATTCCAGAA |
| 64 | 844 | TCAGGCCGGGACAAGTGCAATAT |
| 65 | 845 | CTCAGAAGGACAAGTAGAGTTT |
| 66 | 846 | CAGGCCGGGACAAGTGCAATAA |
| 67 | 847 | CTCGGGGCAGCTCAGTACAGGACGAG |
| 68 | 848 | AAACTCGGGGCAGCTCAGTACAGG |
| 69 | 849 | ACTGGGAGCAGAGGGTGAGTTC |
| 70 | 850 | ACAGGAACTGCCTTTCTCTCCA |
| 71 | 851 | TTTTGGCCGGGACAAGTGCAATA |
| 72 | 852 | AGCAAGACCCCGTCTCTAAAA |
| 73 | 853 | TCCAGTGTGAGTTCTACCATTGCCAAA |
| 74 | 854 | CTTCCTGCTCCCTGCTCTACA |
| 75 | 855 | ACAGGCCGGGACAAGTGCAATAC |
| 76 | 856 | AGACAACAATGCCAACCTCACA |
| 77 | 857 | TACAGGCCGGGACAAGTGCAATAC |
| 78 | 858 | ACAACAATGCCAACCTCACA |
| 79 | 859 | AACTGGGGGCCAGGAGAGTGGA |
| 80 | 860 | ACTCGGGGCAGATCAGTACAGGA |
| 81 | 861 | CTCACCGACAGCGTTGAATGTTT |
| 82 | 862 | TCTATCTGCACTGTCAGCACTTTA |
| 83 | 863 | GAACTCGGGGCAGCTCAGTACAGGA |
| 84 | 864 | AAACGGGGCAGCTCAGTACAGGA |
| 85 | 865 | TACGAGCCGCGAGGAGCCGAGAGA |
| 86 | 866 | CTCGGCGCAGCTCAGTACAGG |
| 87 | 867 | TGAATTCTACCAGTGCCAT |
| 88 | 868 | TACAGGCCGGGACAAGTGCAATAT |
| 89 | 869 | ACAGGCCGGGACAAGTGCAATAT |
| 90 | 870 | TACAGGCCGAGACAAGTGCAATA |
| 91 | 871 | AATCAGGCCGGGACAAGTGCAATA |
| 92 | 872 | CAGGCCGGGACAAGTGCAATAT |
| 93 | 873 | ACTCGGGGCAGCTCAGTACAGGACA |
| 94 | 874 | CTCCTCCACTTCCCGGACACA |
| 95 | 875 | ACAGGCCGGGACAAGTGCAATAA |
| 96 | 876 | TTCTCGGGGCAGCTCAGTACAGGAG |
| 97 | 877 | ACTACCTGCACGAACAGCACTTT |
| 98 | 878 | ACAGGCCGGGATAAGTGCAATA |
| 99 | 879 | TGACAGGCCGGGACAAGTGCAATA |
| 100 | 880 | ACCGGGAGGGGACTGAGCCTGA |
| 101 | 881 | CGTACAGGCCGGGACAAGTGCAATA |
| 102 | 882 | TACAGGCCGGGACAAGTGCAATAG |
| 103 | 883 | TCACTCGGGGCAGCTCAGTACAGG |
| 104 | 884 | CTTCGGGGCAGCTCAGTACAG |
| 105 | 885 | GAAGGGGCATTTAGGGCAGTA |
| 106 | 886 | TTCTACCTGCACTGTAAGCACTTTG |
| 107 | 887 | AGCTTCCTGCTCCCTGCTCTACA |
| 108 | 888 | ATATGGGGTATTTGACAAACTGACA |
| 109 | 889 | ATCAGCAAGTACCCACAGTGCGG |
| 110 | 890 | AACGGGGCAGCTCAGTACAGGAT |
| 111 | 891 | ACTGGGTGCGGGATGCAGGGTGA |
| 112 | 892 | TACAGGCCGGGACAAGTGCAATG |
| 113 | 893 | CAGTGTGAGTTCTACCATTGCCAA |
| 114 | 894 | TACTAAACTCAGTAATGGTAACGGTTT |
| 115 | 895 | TCTAAACTCAGTAATGGTAACGGTT |

TABLE 9
Therapeutic Molecules Based on Small RNA Features
Correlated to Subtype of IPF

| SEQ | |
|---|---|
| ID NO | Small RNA Mimetic Design |
| 821 | UAUUGCACUUGUCCCGGCCUAUAdTdT |
| | \|\|\|\|\|\|\|\|\|\|\|\|\|\|\|\|\|\|\|\|\|\| |
| 201 | dTdTTUAACGUGAACAGGGCCGGAUAU |
| 202 | UAUUGCACUUGUCCCGGCCUGUdTdT |
| | \|\|\|\|\|\|\|\|\|\|\|\|\|\|\|\|\|\|\|\|\| |
| 203 | dTdTTUAACGUGAACAGGGCCGGACA |
| 204 | UAUGGCACUGGUAGAAUUCACUdTdT |
| | \|\|\|\|\|\|\|\|\|\|\|\|\|\|\|\|\|\|\|\|\| |
| 205 | dTdTTUACCGUGACCAUCUUAAGUGA |
| 206 | AUGGCACUGGUAGAAUUCACUGUdTdT |
| | \|\|\|\|\|\|\|\|\|\|\|\|\|\|\|\|\|\|\|\|\|\| |
| 207 | dTdTAACCGUGACCAUCUUAAGUGACA |
| 208 | AAUAUUGCACUUGUCCCGGCCUGUAdTdT |
| | \|\|\|\|\|\|\|\|\|\|\|\|\|\|\|\|\|\|\|\|\|\|\|\| |
| 209 | dTdTAUAUAACGUGAACAGGGCCGGACAU |
| 210 | CCAAAACUGCAGUUACUUUUGdTdT |
| | \|\|\|\|\|\|\|\|\|\|\|\|\|\|\|\|\|\|\|\| |
| 211 | dTdTCGUUUUGACGUCAAUGAAAAC |
| 212 | UAUUACACUUGUCCCGGCCUGUdTdT |
| | \|\|\|\|\|\|\|\|\|\|\|\|\|\|\|\|\|\|\|\|\| |
| 213 | dTdTTUAAUGUGAACAGGGCCGGACA |
| 214 | CAAAGUGCUCAUAGUGCAGGUAGAdTdT |
| | \|\|\|\|\|\|\|\|\|\|\|\|\|\|\|\|\|\|\|\|\|\|\| |
| 215 | dTdTCUUUCACGAGUAUCACGUCCAUCU |
| 216 | GGGAGCCAGGAAGUAUUGAUGUUdTdT |
| | \|\|\|\|\|\|\|\|\|\|\|\|\|\|\|\|\|\|\|\|\|\| |
| 217 | dTdTGCCUCGGUCCUUCAUAACUACAA |
| 218 | UAUUGCACUUGUCCCGGCCUGUUAAdTdT |
| | \|\|\|\|\|\|\|\|\|\|\|\|\|\|\|\|\|\|\|\|\|\|\|\| |
| 219 | dTdTTUAACGUGAACAGGGCCGGACAAUU |
| 220 | GAUUGCACUUGUCCCGGCCUGUdTdT |
| | \|\|\|\|\|\|\|\|\|\|\|\|\|\|\|\|\|\|\|\|\| |
| 221 | dTdTGUAACGUGAACAGGGCCGGACA |
| 222 | GUGUCAGUUUGUCAAAUACCCCAAUdTdT |
| | \|\|\|\|\|\|\|\|\|\|\|\|\|\|\|\|\|\|\|\|\|\|\|\| |
| 223 | dTdTGACAGUCAAACAGUUUAUGGGGUUA |
| 224 | AGCUCGGUCUGAGGCCCCUCAGUUAUdTdT |
| | \|\|\|\|\|\|\|\|\|\|\|\|\|\|\|\|\|\|\|\|\|\|\|\|\| |
| 225 | dTdTACGAGCCAGACUCCGGGGAGUCAAUA |
| 226 | ACAAACCGUUACCAUUACUGAGUUdTdT |
| | \|\|\|\|\|\|\|\|\|\|\|\|\|\|\|\|\|\|\|\|\|\|\| |
| 227 | dTdTAGUUUGGCAAUGGUAAUGACUCAA |
| 228 | CCUGUACUGAGCUGCCCCUAGdTdT |
| | \|\|\|\|\|\|\|\|\|\|\|\|\|\|\|\|\|\|\|\| |
| 229 | dTdTCGACAUGACUCGACGGGGAUC |
| 230 | AAUGCACCCGGGCAAGGAUUdTdT |
| | \|\|\|\|\|\|\|\|\|\|\|\|\|\|\|\|\|\|\| |
| 231 | dTdTAUACGUGGGCCCGUUCCUAA |
| 232 | UUAGCAGCACAUCAUGGUUUAdTdT |
| | \|\|\|\|\|\|\|\|\|\|\|\|\|\|\|\|\|\|\|\| |
| 233 | dTdTTAUCGUCGUGUAGUACCAAAU |
| 234 | AAUGGCGCCACUAGGGUUGUGAAdTdT |
| | \|\|\|\|\|\|\|\|\|\|\|\|\|\|\|\|\|\|\|\|\|\| |
| 235 | dTdTAUACCGCGGUGAUCCCAACACUU |
| 236 | UUCUGGAAUUCUGUGUGAGGGAdTdT |
| | \|\|\|\|\|\|\|\|\|\|\|\|\|\|\|\|\|\|\|\|\| |
| 237 | dTdTTAGACCUUAAGACACACUCCCU |
| 238 | AUAUUGCACUUGUCCCGGCCUGAdTdT |
| | \|\|\|\|\|\|\|\|\|\|\|\|\|\|\|\|\|\|\|\|\|\| |
| 239 | dTdTAAUAACGUGAACAGGGCCGGACU |
| 240 | AAACUCUACUUGUCCUUCUGAGdTdT |
| | \|\|\|\|\|\|\|\|\|\|\|\|\|\|\|\|\|\|\|\|\| |
| 241 | dTdTAUUGAGAUGAACAGGAAGACUC |
| 242 | UUAUUGCACUUGUCCCGGCCUGdTdT |
| | \|\|\|\|\|\|\|\|\|\|\|\|\|\|\|\|\|\|\|\|\| |
| 243 | dTdTTAUAACGUGAACAGGGCCGGAC |
| 244 | CUCGUCCUGUACUGAGCUGCCCCGAGdTdT |
| | \|\|\|\|\|\|\|\|\|\|\|\|\|\|\|\|\|\|\|\|\|\|\|\|\| |
| 245 | dTdTCAGCAGGACAUGACUCGACGGGGCUC |
| 246 | CCUGUACUGAGCUGCCCCGAGUUUdTdT |
| | \|\|\|\|\|\|\|\|\|\|\|\|\|\|\|\|\|\|\|\|\|\|\| |
| 247 | dTdTCGACAUGACUCGACGGGGCUCAAA |
| 248 | GAACUCACCCUCUGCUCCCAGUdTdT |
| | \|\|\|\|\|\|\|\|\|\|\|\|\|\|\|\|\|\|\|\|\| |
| 249 | dTdTGUUGAGUGGGAGACGAGGGUCA |
| 250 | UGGAGAGAAAGGCAGUUCCUGUdTdT |
| | \|\|\|\|\|\|\|\|\|\|\|\|\|\|\|\|\|\|\|\|\| |
| 251 | dTdTTCCUCUCUUUCCGUCAAGGACA |
| 252 | UAUUGCACUUGUCCCGGCCAAAAdTdT |
| | \|\|\|\|\|\|\|\|\|\|\|\|\|\|\|\|\|\|\|\|\|\| |
| 253 | dTdTTUAACGUGAACAGGGCCGGUUUU |
| 254 | UUUUAGAGACGGGGUCUUGCUdTdT |
| | \|\|\|\|\|\|\|\|\|\|\|\|\|\|\|\|\|\|\|\| |
| 255 | dTdTTAAAUCUCUGCCCCAGAACGA |
| 256 | UUUGGCAAUGGUAGAACUCACACUGGAdTdT |
| | \|\|\|\|\|\|\|\|\|\|\|\|\|\|\|\|\|\|\|\|\|\|\|\|\|\| |
| 257 | dTdTTAACCGUUACCAUCUUGAGUGUGACCU |
| 258 | UGUAGAGCAGGGAGCAGGAAGdTdT |
| | \|\|\|\|\|\|\|\|\|\|\|\|\|\|\|\|\|\|\|\| |
| 259 | dTdTTCAUCUCGUCCCUCGUCCUUC |
| 260 | GUAUUGCACUUGUCCCGGCCUGUdTdT |
| | \|\|\|\|\|\|\|\|\|\|\|\|\|\|\|\|\|\|\|\|\|\| |
| 261 | dTdTGAUAACGUGAACAGGGCCGGACA |
| 262 | UGUGAGGUUGGCAUUGUUGUCUdTdT |
| | \|\|\|\|\|\|\|\|\|\|\|\|\|\|\|\|\|\|\|\|\| |
| 263 | dTdTTCACUCCAACCGUAACAACAGA |
| 264 | GUAUUGCACUUGUCCCGGCCUGUAdTdT |
| | \|\|\|\|\|\|\|\|\|\|\|\|\|\|\|\|\|\|\|\|\|\|\| |
| 265 | dTdTGAUAACGUGAACAGGGCCGGACAU |
| 266 | UGUGAGGUUGGCAUUGUUGUdTdT |
| | \|\|\|\|\|\|\|\|\|\|\|\|\|\|\|\|\|\|\| |
| 267 | dTdTTCACUCCAACCGUAACAACA |
| 268 | UCCACUCUCCUGGCCCCCAGUUdTdT |
| | \|\|\|\|\|\|\|\|\|\|\|\|\|\|\|\|\|\|\|\|\| |
| 269 | dTdTTGGUGAGAGGACCGGGGGUCAA |
| 270 | UCCUGUACUGAUCUGCCCCGAGUdTdT |
| | \|\|\|\|\|\|\|\|\|\|\|\|\|\|\|\|\|\|\|\|\|\| |
| 271 | dTdTTGGACAUGACUAGACGGGGCUCA |
| 272 | AAACAUUCAACGCUGUCGGUGAGdTdT |
| | \|\|\|\|\|\|\|\|\|\|\|\|\|\|\|\|\|\|\|\|\|\| |
| 273 | dTdTAUUGUAAGUUGCGACAGCCACUC |
| 274 | UAAAGUGCUGACAGUGCAGAUAGAdTdT |
| | \|\|\|\|\|\|\|\|\|\|\|\|\|\|\|\|\|\|\|\|\|\|\| |
| 275 | dTdTTUUUCACGACUGUCACGUCUAUCU |
| 276 | UCCUGUACUGAGCUGCCCCGAGUUCdTdT |
| | \|\|\|\|\|\|\|\|\|\|\|\|\|\|\|\|\|\|\|\|\|\|\|\| |
| 277 | dTdTTGGACAUGACUCGACGGGGCUCAAG |
| 278 | UCCUGUACUGAGCUGCCCCGUUUdTdT |
| | \|\|\|\|\|\|\|\|\|\|\|\|\|\|\|\|\|\|\|\|\|\| |
| 279 | dTdTTGGACAUGACUCGACGGGGCAAA |
| 280 | UCUCUCGGCUCCUCGCGGCUCGUAdTdT |
| | \|\|\|\|\|\|\|\|\|\|\|\|\|\|\|\|\|\|\|\|\|\|\| |
| 281 | dTdTTGAGAGCCGAGGAGCGCCGAGCAU |
| 282 | CCUGUACUGAGCUGCGCCGAGdTdT |
| | \|\|\|\|\|\|\|\|\|\|\|\|\|\|\|\|\|\|\|\| |
| 283 | dTdTCGACAUGACUCGACGCGGCUC |
| 284 | AUGGCACUGGUAGAAUUCAdTdT |
| | \|\|\|\|\|\|\|\|\|\|\|\|\|\|\|\|\|\| |
| 285 | dTdTAACCGUGACCAUCUUAAGU |
| 286 | AUAUUGCACUUGUCCCGGCCUGUAdTdT |
| | \|\|\|\|\|\|\|\|\|\|\|\|\|\|\|\|\|\|\|\|\|\|\| |
| 287 | dTdTAAUAACGUGAACAGGGCCGGACAU |
| 288 | AUAUUGCACUUGUCCCGGCCUGUdTdT |
| | \|\|\|\|\|\|\|\|\|\|\|\|\|\|\|\|\|\|\|\|\|\| |
| 289 | dTdTAAUAACGUGAACAGGGCCGGACA |
| 290 | UAUUGCACUUGUCUCGGCCUGUAdTdT |
| | \|\|\|\|\|\|\|\|\|\|\|\|\|\|\|\|\|\|\|\|\|\| |
| 291 | dTdTTUAACGUGAACAGAGCCGGACAU |
| 292 | UAUUGCACUUGUCCCGGCCUGAUUdTdT |
| | \|\|\|\|\|\|\|\|\|\|\|\|\|\|\|\|\|\|\|\|\|\|\| |
| 293 | dTdTTUAACGUGAACAGGGCCGGACUAA |
| 294 | AUAUUGCACUUGUCCCGGCCUGdTdT |
| | \|\|\|\|\|\|\|\|\|\|\|\|\|\|\|\|\|\|\|\|\| |
| 295 | dTdTAAUAACGUGAACAGGGCCGGAC |
| 296 | UGUCCUGUACUGAGCUGCCCCGAGUdTdT |
| | \|\|\|\|\|\|\|\|\|\|\|\|\|\|\|\|\|\|\|\|\|\|\|\| |
| 297 | dTdTTCAGGACAUGACUCGACGGGGCUCA |
| 298 | UGUGUCCGGGAAGUGGAGGAGdTdT |
| | \|\|\|\|\|\|\|\|\|\|\|\|\|\|\|\|\|\|\|\| |
| 299 | dTdTTCACAGGCCCUUCACCUCCUC |
| 300 | UUAUUGCACUUGUCCCGGCCUGUdTdT |
| | \|\|\|\|\|\|\|\|\|\|\|\|\|\|\|\|\|\|\|\|\|\| |
| 301 | dTdTTAUAACGUGAACAGGGCCGGACA |
| 302 | CUCCUGUACUGAGCUGCCCCGAGAAdTdT |
| | \|\|\|\|\|\|\|\|\|\|\|\|\|\|\|\|\|\|\|\|\|\|\|\| |
| 303 | dTdTCAGGACAUGACUCGACGGGGCUCUU |
| 304 | AAAGUGCUGUUCGUGCAGGUAGUdTdT |
| | \|\|\|\|\|\|\|\|\|\|\|\|\|\|\|\|\|\|\|\|\|\| |
| 305 | dTdTAUUCACGACAAGCACGUCCAUCA |
| 306 | UAUUGCACUUAUCCCGGCCUGUdTdT |
| | \|\|\|\|\|\|\|\|\|\|\|\|\|\|\|\|\|\|\|\|\| |
| 307 | dTdTTUAACGUGAAUAGGGCCGGACA |
| 308 | UAUUGCACUUGUCCCGGCCUGUCAdTdT |
| | \|\|\|\|\|\|\|\|\|\|\|\|\|\|\|\|\|\|\|\|\|\|\| |
| 309 | dTdTTUAACGUGAACAGGGCCGGACAGU |
| 310 | UCAGGCUCAGUCCCCUCCCGGUdTdT |
| | \|\|\|\|\|\|\|\|\|\|\|\|\|\|\|\|\|\|\|\|\| |
| 311 | dTdTTGUCCGAGUCAGGGGAGGGCCA |
| 312 | UAUUGCACUUGUCCCGGCCUGUACGdTdT |
| | \|\|\|\|\|\|\|\|\|\|\|\|\|\|\|\|\|\|\|\|\|\|\|\| |
| 313 | dTdTTUAACGUGAACAGGGCCGGACAUGC |
| 314 | CUAUUGCACUUGUCCCGGCCUGUAdTdT |
| | \|\|\|\|\|\|\|\|\|\|\|\|\|\|\|\|\|\|\|\|\|\|\| |
| 315 | dTdTCAUAACGUGAACAGGGCCGGACAU |
| 316 | CCUGUACUGAGCUGCCCCGAGUGAdTdT |
| | \|\|\|\|\|\|\|\|\|\|\|\|\|\|\|\|\|\|\|\|\|\|\| |
| 317 | dTdTCGACAUGACUCGACGGGGCUCACU |
| 318 | CUGUACUGAGCUGCCCCGAAGdTdT |
| | \|\|\|\|\|\|\|\|\|\|\|\|\|\|\|\|\|\|\|\| |
| 319 | dTdTCACAUGACUCGACGGGGCUUC |
| 320 | UACUGCCCUAAAUGCCCCUUCdTdT |
| | \|\|\|\|\|\|\|\|\|\|\|\|\|\|\|\|\|\|\|\| |
| 321 | dTdTTUGACGGGAUUUACGGGGAAG |
| 322 | CAAAGUGCUUACAGUGCAGGUAGAAdTdT |
| | \|\|\|\|\|\|\|\|\|\|\|\|\|\|\|\|\|\|\|\|\|\|\|\| |
| 323 | dTdTCUUUCACGAAUGUCACGUCCAUCUU |
| 324 | UGUAGAGCAGGGAGCAGGAAGCUdTdT |
| | \|\|\|\|\|\|\|\|\|\|\|\|\|\|\|\|\|\|\|\|\|\| |
| 325 | dTdTTCAUCUCGUCCCUCGUCCUUCGA |
| 326 | UGUCAGUUUGUCAAAUACCCCAUAUdTdT |
| | \|\|\|\|\|\|\|\|\|\|\|\|\|\|\|\|\|\|\|\|\|\|\|\| |
| 327 | dTdTTCAGUCAAACAGUUUAUGGGGUAUA |
| 328 | CCGCACUGUGGGUACUUGCUGAUdTdT |
| | \|\|\|\|\|\|\|\|\|\|\|\|\|\|\|\|\|\|\|\|\|\| |
| 329 | dTdTCGCGUGACACCCAUGAACGACUA |
| 330 | AUCCUGUACUGAGCUGCCCCGUUdTdT |
| | \|\|\|\|\|\|\|\|\|\|\|\|\|\|\|\|\|\|\|\|\|\| |
| 331 | dTdTAAGGACAUGACUCGACGGGGCAA |
| 332 | UCACCCUGCAUCCCGCACCCAGUdTdT |
| | \|\|\|\|\|\|\|\|\|\|\|\|\|\|\|\|\|\|\|\|\|\| |
| 333 | dTdTTGUGGGACGUAGGGCGUGGGUCA |
| 334 | CAUUGCACUUGUCCCGGCCUGUAdTdT |
| | \|\|\|\|\|\|\|\|\|\|\|\|\|\|\|\|\|\|\|\|\|\| |
| 335 | dTdTCUAACGUGAACAGGGCCGGACAU |
| 336 | UUGGCAAUGGUAGAACUCACACUGdTdT |
| | \|\|\|\|\|\|\|\|\|\|\|\|\|\|\|\|\|\|\|\|\|\|\| |
| 337 | dTdTTACCGUUACCAUCUUGAGUGUGAC |
| 338 | AAACCGUUACCAUUACUGAGUUUAGUAdTdT |
| | \|\|\|\|\|\|\|\|\|\|\|\|\|\|\|\|\|\|\|\|\|\|\|\|\|\| |
| 339 | dTdTAUUGGCAAUGGUAAUGACUCAAAUCAU |
| 340 | AACCGUUACCAUUACUGAGUUUAGAdTdT |
| | \|\|\|\|\|\|\|\|\|\|\|\|\|\|\|\|\|\|\|\|\|\|\|\| |
| 341 | dTdTAUGGCAAUGGUAAUGACUCAAAUCU |
| 824 | AUAUUGCACUUGUCCCGGCCUGdTdT |
| | \|\|\|\|\|\|\|\|\|\|\|\|\|\|\|\|\|\|\|\|\| |
| 733 | dTdTCAUAACGUGAACAGGGCCGGAC |

TABLE 10
miR-92a-3p isoforms and siRNA mimics

| Oligo | SRNA Sequence | Oligo Antisense (5′→3′) (modified) | Oligo Sense (5′→3′) (modified) |
|---|---|---|---|
| 001 | AATATTGCACTTGTCCCGGCCTGTA | /5′Phos/AAUAUUGCACUUGUCCCGGCCUGUAdTdT | UACAGGCCGGGACAAGUGCAAUAUAdTdT |
| | (SEQ ID NO: 734) | (SEQ ID NO: 763) | (SEQ ID NO: 792) |
| 002 | ATATTGCACTTGTCCCGGCCTGA | /5′Phos/AUAUUGCACUUGUCCCGGCCUGAdTdT | UCAGGCCGGGACAAGUGCAAUAAdTdT |
| | (SEQ ID NO: 735) | (SEQ ID NO: 764) | (SEQ ID NO: 793) |
| 003 | ATATTGCACTTGTCCCGGCCTGTA | /5′Phos/AUAUUGCACUUGUCCCGGCCUGUAdTdT | UACAGGCCGGGACAAGUGCAAUAAdTdT |
| | (SEQ ID NO: 736) | (SEQ ID NO: 765) | (SEQ ID NO: 794) |
| 004 | ATATTGCACTTGTCCCGGCCTG | /5′Phos/AUAUUGCACUUGUCCCGGCCUGdTdT | CAGGCCGGGACAAGUGCAAUAAdTdT |
| | (SEQ ID NO: 737) | (SEQ ID NO: 766) | (SEQ ID NO: 795) |
| 005 | ATATTGCACTTGTCCCGGCCTGT | /5′Phos/AUAUUGCACUUGUCCCGGCCUGUdTdT | ACAGGCCGGGACAAGUGCAAUAAdTdT |
| | (SEQ ID NO: 738) | (SEQ ID NO: 767) | (SEQ ID NO: 796) |
| 006 | CATTGCACTTGTCCCGGCCTGTA | /5′Phos/CAUUGCACUUGUCCCGGCCUGUAdTdT | UACAGGCCGGGACAAGUGCAAUCdTdT |
| | (SEQ ID NO: 739) | (SEQ ID NO: 768) | (SEQ ID NO: 797) |
| 007 | CTATTGCACTTGTCCCGGCCTGTA | /5′Phos/CUAUUGCACUUGUCCCGGCCUGUAdTdT | UACAGGCCGGGACAAGUGCAAUACdTdT |
| | (SEQ ID NO: 740) | (SEQ ID NO: 769) | (SEQ ID NO: 798) |
| 008 | GATTGCACTTGTCCCGGCCTGT | /5′Phos/GAUUGCACUUGUCCCGGCCUGUdTdT | ACAGGCCGGGACAAGUGCAAUGdTdT |
| | (SEQ ID NO: 741) | (SEQ ID NO: 770) | (SEQ ID NO: 799) |
| 009 | GTATTGCACTTGTCCCGGCCTGTA | /5′Phos/GUAUUGCACUUGUCCCGGCCUGUAdTdT | UACAGGCCGGGACAAGUGCAAUAGdTdT |
| | (SEQ ID NO: 742) | (SEQ ID NO: 771) | (SEQ ID NO: 800) |
| 010 | GTATTGCACTTGTCCCGGCCTGT | /5′Phos/GUAUUGCACUUGUCCCGGCCUGUdTdT | ACAGGCCGGGACAAGUGCAAUAGdTdT |
| | (SEQ ID NO: 743) | (SEQ ID NO: 772) | (SEQ ID NO: 801) |
| 011 | TATTACACTTGTCCCGGCCTGT | /5′Phos/UAUUACACUUGUCCCGGCCUGUdTdT | ACAGGCCGGGACAAGUGUAAUUdTdT |
| | (SEQ ID NO: 744) | (SEQ ID NO: 773) | (SEQ ID NO: 802) |
| 012 | TATTGCACTTGTCCCGGCCTATA | /5′Phos/UAUUGCACUUGUCCCGGCCUAUAdTdT | UAUAGGCCGGGACAAGUGCAAUUdTdT |
| | (SEQ ID NO: 745) | (SEQ ID NO: 774) | (SEQ ID NO: 803) |
| 013 | TATTGCACTTGTCCCGGCCTGTACG | /5′Phos/UAUUGCACUUGUCCCGGCCUGUACGdTdT | CGUACAGGCCGGGACAAGUGCAAUUdTdT |
| | (SEQ ID NO: 746) | (SEQ ID NO: 775) | (SEQ ID NO: 804) |
| 014 | TATTGCACTTGTCCCGGCCAAAA | /5′Phos/UAUUGCACUUGUCCCGGCCAAAAdTdT | UUUUGGCCGGGACAAGUGCAAUUdTdT |
| | (SEQ ID NO: 747) | (SEQ ID NO: 776) | (SEQ ID NO: 805) |
| 015 | TATTGCACTTGTCCCGGCCTGATT | /5′Phos/UAUUGCACUUGUCCCGGCCUGAUUdTdT | AAUCAGGCCGGGACAAGUGCAAUUdTdT |
| | (SEQ ID NO: 748) | (SEQ ID NO: 777) | (SEQ ID NO: 806) |
| 016 | TATTGCACTTATCCCGGCCTGT | /5′Phos/UAUUGCACUUAUCCCGGCCUGUdTdT | ACAGGCCGGGAUAAGUGCAAUUdTdT |
| | (SEQ ID NO: 749) | (SEQ ID NO: 778) | (SEQ ID NO: 807) |
| 017 | TATTGCACTTGTCCCGGCCTGTCA | /5′Phos/UAUUGCACUUGUCCCGGCCUGUCAdTdT | UGACAGGCCGGGACAAGUGCAAUUdTdT |
| | (SEQ ID NO: 750) | (SEQ ID NO: 779) | (SEQ ID NO: 808) |
| 018 | TATTGCACTTGTCTCGGCCTGTA | /5′Phos/UAUUGCACUUGUCUCGGCCUGUAdTdT | UACAGGCCGAGACAAGUGCAAUUdTdT |
| | (SEQ ID NO: 751) | (SEQ ID NO: 780) | (SEQ ID NO: 809) |
| 019 | TATTGCACTTGTCCCGGCCTGT | /5′Phos/UAUUGCACUUGUCCCGGCCUGUdTdT | ACAGGCCGGGACAAGUGCAAUUdTdT |
| | (SEQ ID NO: 752) | (SEQ ID NO: 781) | (SEQ ID NO: 810) |
| 020 | TATTGCACTTGTCCCGGCCTGTTAA | /5′Phos/UAUUGCACUUGUCCCGGCCUGUUAAdTdT | UUAACAGGCCGGGACAAGUGCAAUUdTdT |
| | (SEQ ID NO: 753) | (SEQ ID NO: 782) | (SEQ ID NO: 811) |
| 021 | TTATTGCACTTGTCCCGGCCTGT | /5′Phos/UUAUUGCACUUGUCCCGGCCUGUdTdT | ACAGGCCGGGACAAGUGCAAUAUdTdT |
| | (SEQ ID NO: 754) | (SEQ ID NO: 783) | (SEQ ID NO: 812) |
| 022 | TTATTGCACTTGTCCCGGCCTG | /5′Phos/UUAUUGCACUUGUCCCGGCCUGdTdT | CAGGCCGGGACAAGUGCAAUAUdTdT |
| | (SEQ ID NO: 755) | (SEQ ID NO: 784) | (SEQ ID NO: 813) |
| 023 | CATTGCACTTGTCCCGGCCTGTA | /5′Phos/CmAUUmGCmACUUmGUCCCmGmGCCUmGUAdTdT | UAmCAGGmCmCGGGAmCAAGmUGmCAAmUCdTdT |
| | (SEQ ID NO: 756) | (SEQ ID NO: 785) | (SEQ ID NO: 814) |
| 024 | GATTGCACTTGTCCCGGCCTGT | /5′Phos/GmAUUmGCmACUUmGUCCCmGmGCCUmGUdTdT | AmCAGGmCmCGGGAmCAAGmUGmCAAmUGdTdT |
| | (SEQ ID NO: 757) | (SEQ ID NO: 786) | (SEQ ID NO: 815) |
| 025 | TATTGCACTTGTCCCGGCCTGTACG | /5′Phos/UmAUUmGCmACUUmGUCCCmGmGCCUmGUACGdTdT | CGUAmCAGGmCmCGGGAmCAAGmUGmCAAmUUdTdT |
| | (SEQ ID NO: 758) | (SEQ ID NO: 787) | (SEQ ID NO: 816) |
| 026 | TATTGCACTTATCCCGGCCTGT | /5′Phos/UmAUUmGCmACUUAUCCCmGmGCCUmGUdTdT | AmCAGGmCmCGGGAUAAGmUGmCAAmUUdTdT |
| | (SEQ ID NO: 759) | (SEQ ID NO: 788) | (SEQ ID NO: 817) |
| 027 | TATTGCACTTGTCTCGGCCTGTA | /5′Phos/UmAUUmGCmACUUmGUCUCmGmGCCUmGUAdTdT | UAmCAGGmCmCGAGAmCAAGmUGmCAAmUUdTdT |
| | (SEQ ID NO: 760) | (SEQ ID NO: 789) | (SEQ ID NO: 818) |
| 028 | TATTGCACTTGTCCCGGCCTGT | /5′Phos/UmAUUmGCmACUUmGUCCCmGmGCCUmGUdTdT | AmCAGGmCmCGGGAmCAAGmUGmCAAmUUdTdT |
| | (SEQ ID NO: 761) | (SEQ ID NO: 790) | (SEQ ID NO: 819) |
| 029 | TATTGCACTTGTCCCGGCCTGT | /5′Phos/UmAUUmGCmACUUmGUCCCmGmGCCUmGUdTdT | AmCAGGmCmCGGGAmCAAGmUGmCAAmUUdTdT |
| | (SEQ ID NO: 762) | (SEQ ID NO: 791) | (SEQ ID NO: 820) |

1. A method for risk stratifying a subject diagnosed with Idiopathic Pulmonary Fibrosis (IPF), comprising:
providing a blood, serum, or plasma sample from the subject,
generating an expression profile of at least five small RNAs listed in Table 2, and
determining a risk of mortality as a function of the expression profile, thereby risk stratifying the subject.

2. The method of claim 1, further comprising determining a GAP stage of the subject, and determining risk of mortality based on the expression profile and the GAP stage.

3. The method of claim 2, wherein the GAP stage comprises three thresholds based on probability of death within 3 years.

4. The method of claims 1 to 3, wherein the subject is stratified into three groups based on risk of mortality.

5. The method of claims 1 to 4, wherein the subject with a high-risk stratification is selected for surgical or pharmaceutical intervention.

6. The method of claim 5, wherein the surgical intervention is lung transplant.

7. The method of claim any one of claims 1 to 6, wherein the expression profile comprises the expression level of at least 10 sRNAs from Table 2.

8. The method of claim 7, wherein the expression profile comprises the expression level of at least 20 sRNAs from Table 2.

9. The method of claim 7, wherein the expression profile comprises the expression level of at least 30 sRNAs from Table 2.

10. The method of claim 7, wherein the expression profile comprises the expression level of at least 40 sRNAs from Table 2.

11. The method of claim 7, wherein the expression profile comprises, consists essentially of, or consists of the expression levels of sRNAs from Table 2.

12. The method of any one of claims 1 to 11, wherein the expression profile is determined by a quantitative PCR assay.

13. The method of claim 12, wherein the sRNAs are reverse transcribed using stem-loop RT primer.

14. The method of claim 13, wherein reverse transcripts are amplified with forward and reverse primers.

15. The method of any one of claims 12 to 14, wherein the quantitative PCR assay employs a fluorescent dye or fluorescent-labeled probe.

16. The method of claim 15, wherein the quantitative PCR assay employs a fluorescent-labeled probe, the probe further comprising a quencher moiety.

17. The method of any one of claims 1 to 11, wherein the expression profile is determined using a hybridization assay.

18. The method of claim 17, wherein the hybridization assay employs a hybridization array comprising sRNA-specific probes.

19. The method of any one of claims 1 to 11, wherein the expression profile is determined by nucleic acid sequencing, and sRNAs are identified in the sample by a process that comprises trimming 5′ and 3′ sequencing adaptors from sRNA sequences.

20. The method of claim 19, wherein RNA from multiple samples are pooled for determining expression profiles, with sequences from different samples containing an identifying sample tag sequence.

21. The method of any one of claims 1 to 20, wherein the expression profile further comprises the expression level of one or more expression normalization controls.

22. The method of any one of claims 1 to 21, wherein the subject is risk stratified using a risk score calculated from Equation 1.

23. The method of any one of claims 1 to 22, wherein the method is repeated at a frequency of at least once per year, or at least once every six months, or at least once every two months.

24. A kit for evaluating samples for Idiopathic Pulmonary Fibrosis (IPF), comprising:
sRNA-specific probes and/or primers configured for detecting a plurality of sRNAs listed in Table 2 (SEQ ID NOS: 1 to 44).

25. The kit of claim 24, comprising: sRNA-specific probes and/or primers configured for detecting at least 10 sRNAs listed in Table 2 (SEQ ID NOS: 1 to 44), and wherein the probes and/or primers are listed in Table 3.

26. The kit of claim 24, comprising: sRNA-specific probes and/or primers configured for detecting at least 20 sRNAs listed in Table 2 (SEQ ID NOS: 1 to 44), and wherein the probes and/or primers are listed in Table 3.

27. The kit of claim 24, comprising: sRNA-specific probes and/or primers configured for detecting at least 30 sRNAs listed in Table 2 (SEQ ID NOS: 1 to 44), and wherein the probes and/or primers are listed in Table 3.

28. The kit of claim 24, comprising: sRNA-specific probes and/or primers configured for detecting at least 40 sRNAs listed in Table 2 (SEQ ID NOS: 1 to 44), and wherein the probes and/or primers are listed in Table 3.

29. The kit of claim 24, comprising sRNA-specific probes and/or primers configured for detecting at least 50 sRNAs listed in Table 2 (SEQ ID NOS: 1 to 44), and wherein the probes and/or primers are listed in Table 3.

30. The kit of any one of claims 24 to 29, comprising sRNA-specific stem loop RT primers, which are optionally from Table 3.

31. The kit of any one of claims 24 to 30, comprising forward and reverse primer pairs for amplifying sRNA reverse transcripts, which are optionally as shown in Table 3.

32. The kit of claim any one of claims 24 to 31, comprising sRNA-specific probes for detecting amplicons, and which are optionally listed in Table 3.

33. The kit of claim 32, wherein the sRNA-specific probes are fluorescent-labeled probes.

34. The kit of claim 33, wherein the probe further comprising a quencher moiety.

35. The kit of any one of claims 24 to 34, comprising an array of sRNA-specific hybridization probes.

36. A small interfering RNA (siRNA) comprising an antisense strand and a sense strand, the antisense strand comprising at least 12 consecutive nucleotides of a sequence of Table 2.

37. The siRNA of claim 36, wherein the siRNA comprises a chemical modification.

38. The siRNA of 37, wherein the chemical modification increases stability, reduces endonuclease degradation, reduces immunogenicity, and/or reduces Toll-like receptor recognition.

39. The siRNA of claim 37 or 38, wherein the chemical modification is a nucleobase modification, a backbone modification, and/or a sugar modification.

40. The siRNA of claim 39, wherein the nucleobase modification suppresses RNA recognition by a Toll-like receptor (TLR).

41. The siRNA of claim 39 or 40, wherein the backbone modification is selected from phosphorothioate, phosphorodithioate, methylphosphonate, and methoxypropylphosphonate.

42. The siRNA of any one of claims 39 to 41, wherein the sugar modification is selected from 2′-methoxy (2′-OMe), 2′-O-methoxyethyl (2′-O-MOE), 2′-fluoro (2′-F), constrained ethyl (cEt), bridged nucleic acid (BNA) and locked nucleic acid (LNA).

43. The siRNA of any one of claims 36 to 42, wherein the sense and antisense strain each have a length of about 12 to about 40 nucleotides.

44. The siRNA of any one of claims 36 to 43, wherein the siRNA comprises two substantially complementary RNA strands with a duplex length of about 12 to about 40 base pairs.

45. The siRNA of any one of claims 36 to 44, wherein the siRNA comprises one or two 3′ end overhangs.

46. The siRNA of claim 45, wherein the siRNA comprises a sense strand overhang and an antisense strand overhang.

47. The siRNA of claim 45 or 46, wherein the overhangs are deoxythymidine (dT-dT) overhangs.

48. The siRNA of claim 47, wherein the siRNA is in a 19+2 format.

49. The siRNA of claim 44 or 45, wherein the siRNA is an asymmetric siRNA (asiRNA) having a blunt end corresponding to the 5′ end of the antisense strand.

50. The siRNA of claim 49, wherein the asiRNA comprises a 19-24 nucleotide antisense strand and a 15-21 nucleotide sense strand.

51. The siRNA of any one of claims 36 to 50, wherein the asiRNA has an antisense strand with a nucleotide at its 5′ end that is not base paired with the sense strand.

52. The siRNA of any one of claims 36 to 51, wherein the composition is formulated for parenteral delivery or for local delivery to the lungs.

53. The siRNA of claim 52, wherein the siRNA is encapsulated in a lipid nanoparticle, polymeric nanoparticle, or liposome.

54. The siRNA of claim 52 or 53, wherein the siRNA is formulated for delivered by inhalation, and is optionally in aerosol form.

55. An antisense oligonucleotide that is at least 10 linked nucleotides in length and having a sequence that is complementary to a nucleotide sequence selected from Table 2 (SEQ ID NOS: 1 to 44).

56. The antisense oligonucleotide of claim 55, wherein the oligonucleotide is fully complementary to a nucleotide sequence selected from Table 2 (SEQ ID NOS: 1 to 44).

57. The antisense oligonucleotide of any one of claims 55 or 56, wherein the oligonucleotide is about 12 to about 40 nucleotides in length.

58. The antisense oligonucleotide of claim 57, wherein the oligonucleotide is 12, 13, 14, 15, 16, 17, 18, 19, or 20 nucleotides in length.

59. The antisense oligonucleotide of any one of claims 55 to 58, wherein the oligonucleotide consists of a nucleotide sequence that is complementary to a sequence selected from SEQ ID NOs: 1 to 44.

60. The antisense oligonucleotide of any one of claims 55 to 59, wherein the oligonucleotide has a contiguous sequence of at least 10 DNA nucleotides sufficient to recruit RNaseH.

61. The antisense oligonucleotide of claim 60, wherein the oligonucleotide is a gapmer having a 5′ segment, a 3′ segment, and a middle segment recruiting RNaseH; each of the 5′ and 3′ segments being from 2 to 6 nucleotides or from 2 to 4 nucleotides in length, and where the 5′ and 3′ segments do not contain DNA nucleotides.

62. The antisense oligonucleotide of claim 61, wherein one or more nucleotides of the 5′ segment and the 3′ segment comprise 2′-O substituents, optionally wherein all of the nucleotides of the 5′ segment and the 3′ segment comprise 2′-O substituents.

63. The antisense oligonucleotide of any one of claims 55 to 62, wherein the oligonucleotide comprises one or more 2′ chemical modifications independently selected from 2′-Fluoro, 2′-Methyl, and 2′-Ethyl.

64. The antisense oligonucleotide of any one of claims 55 to 63, wherein the oligonucleotide comprises one or more 2′-O substituents are selected from 2′-O methyl, 2′-O ethyl, 2′-O methoxyethyl (MOE), and a bridged nucleotide having a 2′ to 4′ bridge.

65. The antisense oligonucleotide of claim 64, wherein the bridged nucleotide has a methylene bridge (LNA) or a constrained ethyl bridge (cEt).

66. The antisense oligonucleotide of any one of claims 55 to 65, wherein the oligonucleotide has a modified backbone.

67. The antisense oligonucleotide of claim 66, wherein the oligonucleotide comprises one or more phosphorothioate or phosphorodithioate nucleotides.

68. The antisense oligonucleotide of claim 67, wherein the oligonucleotide is fully phosphorothioate or phosphorodithioate linked.

69. The antisense oligonucleotide of any one of claims 55 to 68, wherein cytidine nucleobases are 5-methyl cytidine.

70. The antisense oligonucleotide of any one of claims 55 to 59, wherein the oligonucleotide has a morpholino or thiomorpholino backbone.

71. The antisense oligonucleotide of any one of claims 55 to 59, wherein the oligonucleotide has a PNA backbone.

72. The antisense oligonucleotide of any one of claims 55 to 71, wherein the Tm of the oligonucleotide hybridized to its target sequence is at least about 35° C.

73. The antisense oligonucleotide of claim 72, wherein the Tm of the oligonucleotide hybridized to its target sequence is at least about 40° C., or at least about 45° C., or at least about 50° C.

74. The antisense oligonucleotide of claim 72 or 73, wherein the Tm of the oligonucleotide hybridized to its target sequence is from about 35° C. to about 60° C., or from about 40° C. to about 60° C., or from about 50° C. to about 60° C.

75. The antisense oligonucleotide of any one of claims 55 to 74, wherein the composition further comprises a targeting or cell penetrating moiety.

76. The antisense oligonucleotide of any one of claims 55 to 75, wherein the oligonucleotide is formulated for parenteral delivery or for local delivery to the lungs.

77. The antisense oligonucleotide of claim 76, wherein the antisense oligonucleotide is encapsulated in a lipid nanoparticle, polymeric nanoparticle, or liposome.

78. The antisense oligonucleotide of claim 76 or 77, wherein the oligonucleotide is formulated for delivery by inhalation, and is optionally in aerosol form.

79. A pharmaceutical composition comprising an effective amount of an siRNA or antisense oligonucleotide of any one of claims 36 to 78 and one or more pharmaceutically acceptable excipients or carriers.

80. The pharmaceutical composition of claim 79, wherein the siRNA or antisense oligonucleotide is encapsulated in liposomes, polymeric nanoparticles, or lipid nanoparticles.

81. A method for treating a subject having Idiopathic Pulmonary Fibrosis, comprising administering an effective amount of a pharmaceutical composition of claim 79 or 80.

82. A method for evaluating Idiopathic Pulmonary Fibrosis in a subject, comprising
providing a blood, serum, or plasma sample from the subject,
generating an expression profile of at least five small RNAs listed in Table 6, and based on the expression profile identifying whether the subject as has a high risk of mortality.

83. The method of 82, wherein the subject is identified as having a subtype of IPF that correlates to high risk of mortality.

84. The method of claim 83, wherein the subject is selected for surgical or pharmaceutical intervention.

85. The method of claim 84, wherein the surgical intervention is lung transplant.

86. The method of claim any one of claims 82 to 85, wherein the expression profile comprises the expression level of at least 10 sRNAs from Table 6.

87. The method of claim 86, wherein the expression profile comprises the expression level of at least 20 sRNAs from Table 6.

88. The method of claim 86, wherein the expression profile comprises the expression level of at least 30 sRNAs from Table 6.

89. The method of claim 86, wherein the expression profile comprises the expression level of at least 40 sRNAs from Table 6.

90. The method of claim 86, wherein the expression profile comprises, consists essentially of, or consists of the expression levels of sRNAs from Table 6.

91. The method of any one of claims 82 to 90, wherein the expression profile is determined by a quantitative PCR assay.

92. The method of claim 91, wherein the sRNAs are reverse transcribed using stem-loop RT primer.

93. The method of claim 92, wherein reverse transcripts are amplified with forward and reverse primers.

94. The method of any one of claims 91 to 93, wherein the quantitative PCR assay employs a fluorescent dye or fluorescent-labeled probe.

95. The method of claim 94, wherein the quantitative PCR assay employs a fluorescent-labeled probe, the probe further comprising a quencher moiety.

96. The method of any one of claims 82 to 90, wherein the expression profile is determined using a hybridization assay.

97. The method of claim 96, wherein the hybridization assay employs a hybridization array comprising sRNA-specific probes.

98. The method of any one of claims 82 to 90, wherein the expression profile is determined by nucleic acid sequencing, and sRNAs are identified in the sample by a process that comprises trimming 5′ and 3′ sequencing adaptors from sRNA sequences.

99. The method of claim 98, wherein RNA from multiple samples are pooled for determining expression profiles, with sequences from different samples containing an identifying sample tag sequence.

100. The method of any one of claims 82 to 99, wherein the expression profile further comprises the expression level of one or more expression normalization controls.

101. A kit for evaluating samples for subtypes of Idiopathic Pulmonary Fibrosis (IPF), comprising:
sRNA-specific probes and/or primers configured for detecting a plurality of sRNAs listed in Table 6 (SEQ ID NOS: 45 to 115).

102. The kit of claim 101, comprising: sRNA-specific probes and/or primers configured for detecting at least 10 sRNAs listed in Table 6 (SEQ ID NOS: 45 to 115), and wherein the probes and/or primers are listed in Table 7.

103. The kit of claim 101, comprising: sRNA-specific probes and/or primers configured for detecting at least 20 sRNAs listed in Table 6 (SEQ ID NOS: 45 to 115), and wherein the probes and/or primers are listed in Table 7.

104. The kit of claim 101, comprising: sRNA-specific probes and/or primers configured for detecting at least 30 sRNAs listed in Table 6 (SEQ ID NOS: 45 to 115), and wherein the probes and/or primers are listed in Table 7.

105. The kit of claim 101, comprising: sRNA-specific probes and/or primers configured for detecting at least 40 sRNAs listed in Table 6 (SEQ ID NOS: 45 to 115), and wherein the probes and/or primers are listed in Table 7.

106. The kit of claim 101, comprising sRNA-specific probes and/or primers configured for detecting at least 50 sRNAs listed in Table 6 (SEQ ID NOS: 45 to 115), and wherein the probes and/or primers are listed in Table 7.

107. The kit of any one of claims 101 to 106, comprising sRNA-specific stem loop RT primers, which are optionally from Table 7.

108. The kit of any one of claims 101 to 107, comprising forward and reverse primer pairs for amplifying sRNA reverse transcripts, which are optionally as shown in Table 7.

109. The kit of claim any one of claims 101 to 108, comprising sRNA-specific probes for detecting amplicons, and which are optionally listed in Table 7.

110. The kit of claim 109, wherein the sRNA-specific probes are fluorescent-labeled probes.

111. The kit of claim 110, wherein the probe further comprising a quencher moiety.

112. The kit of any one of claims 101 to 106, comprising an array of sRNA-specific hybridization probes.

113. A small interfering RNA (siRNA) comprising an antisense strand and a sense strand, the antisense strand comprising at least 12 consecutive nucleotides of a sequence of Table 6.

114. The siRNA of claim 113, wherein the siRNA is a mimetic for miR-92a or an isoform thereof.

115. The siRNA of claim 113, wherein the antisense strand comprises the nucleotide sequence of SEQ ID NO: 732, and wherein the sense strand is optionally SEQ ID NO: 733.

116. The siRNA of any one of claims 113 to 115, wherein the siRNA comprises a chemical modification.

117. The siRNA of any of claim 116, wherein the chemical modification increases stability, reduces endonuclease degradation, reduces immunogenicity, and/or reduces Toll-like receptor recognition.

118. The siRNA of claim 117, wherein the chemical modification is a nucleobase modification, a backbone modification, and/or a sugar modification.

119. The siRNA of claim 118, wherein the nucleobase modification suppresses RNA recognition by a Toll-like receptor (TLR).

120. The siRNA of any one of claims 118 or 119, wherein the backbone modification is selected from phosphorothioate, phosphorodithioate, methylphosphonate, and methoxypropylphosphonate.

121. The siRNA of any one of claims 118 to 120, wherein the sugar modification is selected from 2′-methoxy (2′-OMe), 2′-O-methoxyethyl (2′-O-MOE), 2′-fluoro (2′-F), constrained ethyl (cEt), bridged nucleic acid (BNA) and locked nucleic acid (LNA).

122. The siRNA of any one of claims 113 to 121, wherein the siRNA comprises a sense and antisense strain, each having a length of about 12 to about 40 nucleotides.

123. The siRNA of any one of claims 113 to 122, wherein the siRNA comprises two substantially complementary RNA strands with a duplex length of about 12 to about 40 base pairs.

124. The siRNA of any one of claims 113 to 123, wherein the siRNA comprises one or two 3′ end overhangs.

125. The siRNA of claim 124, wherein the siRNA comprises a sense strand overhang and an antisense strand overhang.

126. The siRNA of claim 124 or 125, wherein the overhangs are deoxythymidine (dT-dT) overhangs.

127. The siRNA of any one of claims 113 to 126, wherein the siRNA is in a 19+2 format.

128. The siRNA of any one of claims 113 to 124, wherein the siRNA is an asymmetric siRNA (asiRNA) having a blunt end corresponding to the 5′ end of the antisense strand.

129. The siRNA of claim 128, wherein the asiRNA comprises a 19-24 nucleotide antisense strand and a 15-21 nucleotide sense strand.

130. The siRNA of any one of claims 113 to 129, wherein the asiRNA has an antisense strand with a nucleotide at its 5′ end that is not base paired with the sense strand.

131. The siRNA of any one of claims 113 to 130, wherein the siRNA is formulated for parenteral delivery or local delivery to the lungs.

132. The siRNA of claim 131, wherein the siRNA is encapsulated in a lipid nanoparticle, polymeric nanoparticle, or liposome.

133. The siRNA of claim 131 or 132, wherein the siRNA is delivered by inhalation, and is optionally in aerosol form.

134. An antisense oligonucleotide that is at least 10 linked nucleotides in length and having a sequence that is complementary to a nucleotide sequence selected from Table 6 (SEQ ID NOS: 45 to 115).

135. The antisense oligonucleotide of claim 134, wherein the oligonucleotide is fully complementary to a nucleotide sequence selected from Table 6 (SEQ ID NOS: 45 to 115).

136. The antisense oligonucleotide of any one of claims 134 or 135, wherein the oligonucleotide is about 12 to about 40 nucleotides in length.

137. The antisense oligonucleotide of claim 136, wherein the oligonucleotide is 12, 13, 14, 15, 16, 17, 18, 19, or 20 nucleotides in length.

138. The antisense oligonucleotide of any one of claims 134 to 137, wherein the oligonucleotide consists of a nucleotide sequence that is complementary to a sequence selected from SEQ ID NOs: 45 to 115.

139. The antisense oligonucleotide of any one of claims 134 to 138, wherein the oligonucleotide has a contiguous sequence of at least 10 DNA nucleotides sufficient to recruit RNaseH.

140. The antisense oligonucleotide of claim 139, wherein the oligonucleotide is a gapmer having a 5′ segment, a 3′ segment, and a middle segment recruiting RNaseH; each of the 5′ and 3′ segments being from 2 to 6 nucleotides or from 2 to 4 nucleotides in length, and where the 5′ and 3′ segments do not contain DNA nucleotides.

141. The antisense oligonucleotide of claim 140, wherein one or more nucleotides of the 5′ segment and the 3′ segment comprise 2′-O substituents, optionally wherein all of the nucleotides of the 5′ segment and the 3′ segment comprise 2′-O substituents.

142. The antisense oligonucleotide of any one of claims 134 to 141, wherein the oligonucleotide comprises one or more 2′ chemical modifications independently selected from 2′-Fluoro, 2′-Methyl, and 2′-Ethyl.

143. The antisense oligonucleotide of any one of claims 134 to 142, wherein the oligonucleotide comprises one or more 2′-O substituents are selected from 2′-O methyl, 2′-O ethyl, 2′-O methoxyethyl (MOE), and a bridged nucleotide having a 2′ to 4′ bridge.

144. The antisense oligonucleotide of claim 143, wherein the bridged nucleotide has a methylene bridge (LNA) or a constrained ethyl bridge (cEt).

145. The antisense oligonucleotide of any one of claims 134 to 144, wherein the oligonucleotide has a modified backbone.

146. The antisense oligonucleotide of claim 145, wherein the oligonucleotide comprises one or more phosphorothioate or phosphorodithioate nucleotides.

147. The antisense oligonucleotide of claim 146, wherein the oligonucleotide is fully phosphorothioate or phosphorodithioate linked.

148. The antisense oligonucleotide of any one of claims 134 to 147, wherein cytidine nucleobases are 5-methyl cytidine.

149. The antisense oligonucleotide of any one of claims 134 to 138, wherein the oligonucleotide has a morpholino or thiomorpholino backbone.

150. The antisense oligonucleotide of any one of claims 134 to 138, wherein the oligonucleotide has a PNA backbone.

151. The antisense oligonucleotide of any one of claims 134 to 150, wherein the Tm of the oligonucleotide hybridized to its target sequence is at least about 35° C.

152. The antisense oligonucleotide of claim 151, wherein the Tm of the oligonucleotide hybridized to its target sequence is at least about 40° C., or at least about 45° C., or at least about 50° C.

153. The antisense oligonucleotide of claim 151, wherein the Tm of the oligonucleotide hybridized to its target sequence is from about 35° C. to about 60° C., or from about 40° C. to about 60° C., or from about 50° C. to about 60° C.

154. The antisense oligonucleotide of any one of claims 134 to 153, wherein the composition further comprises a targeting or cell penetrating moiety.

155. The antisense oligonucleotide of any one of claims 134 to 154, wherein the composition is formulated for parenteral delivery or for local delivery to the lungs.

156. The antisense oligonucleotide of claim 155, wherein the antisense oligonucleotide is encapsulated in a lipid nanoparticle, polymeric nanoparticle, or liposome.

157. The antisense oligonucleotide of claim 155 or 156, wherein the siRNA is formulated for delivery by inhalation, and is optionally in aerosol form.

158. A pharmaceutical composition comprising an effective amount of an siRNA or antisense oligonucleotide of any one of claims 113 to 157 and one or more pharmaceutically acceptable excipients or carriers.

159. The pharmaceutical composition of claim 158, wherein the siRNA or antisense oligonucleotide is encapsulated in liposomes, polymeric nanoparticles, or lipid nanoparticles.

160. A method for treating a subject having Idiopathic Pulmonary Fibrosis, comprising administering an effective amount of a pharmaceutical composition of claim 158 or 159.

161. The method of claim 160, wherein the siRNA is a mimetic for miR-92a or an isoform thereof.

162. The method of 160, wherein the mimetic is an siRNA, and where the siRNA optionally comprises an antisense sequence of SEQ ID NO: 732, and optionally a sense strand of SEQ ID NO: 733.

163. An siRNA comprising an antisense sequence or strand that corresponds to an isoform of miR-92a-3p.

164. The siRNA of claim 163, wherein the isoform is listed in Table 10.

165. The siRNA of claim 164, wherein the isoform is SEQ ID NO: 739.

166. The siRNA of any one of claims 163 to 165, comprising an antisense strand and a sense strand.

167. The siRNA of claim 166, wherein the antisense strand and/or the sense strand have a 3′ overhang, which is optionally dTdT.

168. The siRNA of claim 167, wherein the antisense strand is selected from Table 10.

169. The siRNA of claim 168, wherein the sense strand is selected from Table 10.

170. The siRNA of any one of claims 163 to 169, wherein the siRNA is encapsulated in a lipid nanoparticle or polymeric nanoparticle.

171. A method for treating an inflammatory or fibrotic disorder, comprising administering a compound that mimics miR-92a-3p or an isoform thereof.

172. The method of claim 171, wherein the compound is an siRNA.

173. The method of claim 172, wherein the compound is an siRNA of any one of claims 163 to 170.

174. The method of any one of claims 171 to 173, wherein the disorder is characterized by dysregulation of the WNT/TGF-b/ITGA/FAK regulatory axis.

175. The method of any one of claims 171 to 174, wherein the disorder is IPF.

176. The method of any one of claims 171 to 174, wherein the disorder is selected from nonalcoholic steatohepatitis (NASH), heart failure, cardiomyopathy, Crohn's disease, ulcerative colitis, Alzheimer's disease, Parkinson's disease, Huntington's disease, amyotrophic lateral sclerosis (ALS), pre-eclampsia, psoriasis, Pompe's disease, sCID, breast cancer, and other cancers.


SEQUENCE LISTING (896 sequences)

SEQ ID NO: 1 - RNA, 22 nt, synthetic construct
  source 1..22: mol_type = other RNA
aacccgtagatccgaacttgtg

SEQ ID NO: 2 - RNA, 21 nt, synthetic construct
  source 1..21: mol_type = other RNA
aagctcggtctgaggcccctc

SEQ ID NO: 3 - RNA, 17 nt, synthetic construct
  source 1..17: mol_type = other RNA
aatcccggacgagcccc

SEQ ID NO: 4 - RNA, 23 nt, synthetic construct
  source 1..23: mol_type = other RNA
acctgtactgagctgccccgaga

SEQ ID NO: 5 - RNA, 23 nt, synthetic construct
  source 1..23: mol_type = other RNA
actcggcgtggcgtcggtcgtgt

SEQ ID NO: 6 - RNA, 17 nt, synthetic construct
  source 1..17: mol_type = other RNA
atccccggcacctccac

SEQ ID NO: 7 - RNA, 23 nt, synthetic construct
  source 1..23: mol_type = other RNA
atctcccaacccttgtaccagtg

SEQ ID NO: 8 - RNA, 24 nt, synthetic construct
  source 1..24: mol_type = other RNA
atgtcccacccccactcctgttaa

SEQ ID NO: 9 - RNA, 23 nt, synthetic construct
  source 1..23: mol_type = other RNA
atgtcccacccccactcctgttt

SEQ ID NO: 10 - RNA, 22 nt, synthetic construct
  source 1..22: mol_type = other RNA
cgtcccaccccccactcctgtt

SEQ ID NO: 11 - RNA, 22 nt, synthetic construct
  source 1..22: mol_type = other RNA
ctatacgacctgctgcctttcg

SEQ ID NO: 12 - RNA, 21 nt, synthetic construct
  source 1..21: mol_type = other RNA
gacctcgccgtcccgcccgcc

SEQ ID NO: 13 - RNA, 18 nt, synthetic construct
  source 1..18: mol_type = other RNA
gcacttgtctcggtctga

SEQ ID NO: 14 - RNA, 18 nt, synthetic construct
  source 1..18: mol_type = other RNA
gtagtaggttgtatagtt

SEQ ID NO: 15 - RNA, 23 nt, synthetic construct
  source 1..23: mol_type = other RNA
gtcaacacttgctggtttcctct

SEQ ID NO: 16 - RNA, 22 nt, synthetic construct
  source 1..22: mol_type = other RNA
gtcatacacggctctcctctct

SEQ ID NO: 17 - RNA, 24 nt, synthetic construct
  source 1..24: mol_type = other RNA
tattgcacttgtcccggcctgtga

SEQ ID NO: 18 - RNA, 21 nt, synthetic construct
  source 1..21: mol_type = other RNA
tccccggcatctccaccaaaa

SEQ ID NO: 19 - RNA, 23 nt, synthetic construct
  source 1..23: mol_type = other RNA
tccctgtcctccaggagctcacg

SEQ ID NO: 20 - RNA, 24 nt, synthetic construct
  source 1..24: mol_type = other RNA
tccctgtcctccaggagctcacga

SEQ ID NO: 21 - RNA, 21 nt, synthetic construct
  source 1..21: mol_type = other RNA
tccctgtcctccaggagctca

SEQ ID NO: 22 - RNA, 22 nt, synthetic construct
  source 1..22: mol_type = other RNA
tcctgtactgagctaccccgag

SEQ ID NO: 23 - RNA, 22 nt, synthetic construct
  source 1..22: mol_type = other RNA
tcctgtactgagctgccccaga

SEQ ID NO: 24 - RNA, 25 nt, synthetic construct
  source 1..25: mol_type = other RNA
tcctgtactgagctgccccgaggcc

SEQ ID NO: 25 - RNA, 22 nt, synthetic construct
  source 1..22: mol_type = other RNA
tctacagtgcacgtgtctccag

SEQ ID NO: 26 - RNA, 17 nt, synthetic construct
  source 1..17: mol_type = other RNA
tcttcgggggcgtgggt

SEQ ID NO: 27 - RNA, 22 nt, synthetic construct
  source 1..22: mol_type = other RNA
tcttgtactgagctgccccgag

SEQ ID NO: 28 - RNA, 21 nt, synthetic construct
  source 1..21: mol_type = other RNA
tgaggggcagagagcgagaaa

SEQ ID NO: 29 - RNA, 23 nt, synthetic construct
  source 1..23: mol_type = other RNA
tggcatgcacgaggccccgggtt

SEQ ID NO: 30 - RNA, 24 nt, synthetic construct
  source 1..24: mol_type = other RNA
tggcatgcacgaggccccgggttt

SEQ ID NO: 31 - RNA, 20 nt, synthetic construct
  source 1..20: mol_type = other RNA
tgggtttacgttgggagaac

SEQ ID NO: 32 - RNA, 22 nt, synthetic construct
  source 1..22: mol_type = other RNA
tggttaggattcggcgctctca

SEQ ID NO: 33 - RNA, 21 nt, synthetic construct
  source 1..21: mol_type = other RNA
tgtcccacccccactcctgtt

SEQ ID NO: 34 - RNA, 68 nt, synthetic construct
  source 1..68: mol_type = other RNA
tcaagtgatggagagcaatacccggggatgactgtgaccacattgggatgtattcgtactgtctgatg

SEQ ID NO: 35 - RNA, 51 nt, synthetic construct
  source 1..51: mol_type = other RNA
gcattggtggttcagtggtagaattctcgcctgccacgcgggaggcccggg

SEQ ID NO: 36 - RNA, 51 nt, synthetic construct
  source 1..51: mol_type = other RNA
gcatgggtggttcagtggtagaattctcgcctgccacgcgggaggcccggg

SEQ ID NO: 37 - RNA, 58 nt, synthetic construct
  source 1..58: mol_type = other RNA
gcattggtggttcagtggtagaattctcgcctgccacgcgggaggcccgggttcgttt

SEQ ID NO: 38 - RNA, 49 nt, synthetic construct
  source 1..49: mol_type = other RNA
ttggtggttcagtggtagaattctcgcctgccacgcgggaggcccgggt

SEQ ID NO: 39 - RNA, 52 nt, synthetic construct
  source 1..52: mol_type = other RNA
gcattggtggttcagtggtagaattctcgcctgccacgcgggagacccgggt

SEQ ID NO: 40 - RNA, 65 nt, synthetic construct
  source 1..65: mol_type = other RNA
gcattggtggttcagtggtagaattctcgcctgccacgcgggaggcccgggttcgtttcccggcc

SEQ ID NO: 41 - RNA, 49 nt, synthetic construct
  source 1..49: mol_type = other RNA
gtgggggagaggctgtcgctgcgcttctgggcccgcggcgggcgtgggg

SEQ ID NO: 42 - RNA, 52 nt, synthetic construct
  source 1..52: mol_type = other RNA
gcattagtggttcagtggtagaattctcgcctgccacgcgggaggcccgggt

SEQ ID NO: 43 - RNA, 53 nt, synthetic construct
  source 1..53: mol_type = other RNA
gcattggtggttcagtggtagaattctcgcctgccacgcgggaggcccgggta

SEQ ID NO: 44 - RNA, 52 nt, synthetic construct
  source 1..52: mol_type = other RNA
gcattggtggtttagtggtagaattctcgcctgccacgcgggaggcccgggt

SEQ ID NO: 45 - RNA, 23 nt, synthetic construct
  source 1..23: mol_type = other RNA
tattgcacttgtcccggcctata

SEQ ID NO: 46 - RNA, 22 nt, synthetic construct
  source 1..22: mol_type = other RNA
tattgcacttgtcccggcctgt

SEQ ID NO: 47 - RNA, 22 nt, synthetic construct
  source 1..22: mol_type = other RNA
tatggcactggtagaattcact

SEQ ID NO: 48 - RNA, 23 nt, synthetic construct
  source 1..23: mol_type = other RNA
atggcactggtagaattcactgt

SEQ ID NO: 49 - RNA, 25 nt, synthetic construct
  source 1..25: mol_type = other RNA
aatattgcacttgtcccggcctgta

SEQ ID NO: 50 - RNA, 21 nt, synthetic construct
  source 1..21: mol_type = other RNA
ccaaaactgcagttacttttg

SEQ ID NO: 51 - RNA, 22 nt, synthetic construct
  source 1..22: mol_type = other RNA
tattacacttgtcccggcctgt

SEQ ID NO: 52 - RNA, 24 nt, synthetic construct
  source 1..24: mol_type = other RNA
caaagtgctcatagtgcaggtaga

SEQ ID NO: 53 - RNA, 23 nt, synthetic construct
  source 1..23: mol_type = other RNA
gggagccaggaagtattgatgtt

SEQ ID NO: 54 - RNA, 25 nt, synthetic construct
  source 1..25: mol_type = other RNA
tattgcacttgtcccggcctgttaa

SEQ ID NO: 55 - RNA, 22 nt, synthetic construct
  source 1..22: mol_type = other RNA
gattgcacttgtcccggcctgt

SEQ ID NO: 56 - RNA, 25 nt, synthetic construct
  source 1..25: mol_type = other RNA
gtgtcagtttgtcaaataccccaat

SEQ ID NO: 57 - RNA, 26 nt, synthetic construct
  source 1..26: mol_type = other RNA
agctcggtctgaggcccctcagttat

SEQ ID NO: 58 - RNA, 24 nt, synthetic construct
  source 1..24: mol_type = other RNA
acaaaccgttaccattactgagtt

SEQ ID NO: 59 - RNA, 21 nt, synthetic construct
  source 1..21: mol_type = other RNA
cctgtactgagctgcccctag

SEQ ID NO: 60 - RNA, 20 nt, synthetic construct
  source 1..20: mol_type = other RNA
aatgcacccgggcaaggatt

SEQ ID NO: 61 - RNA, 21 nt, synthetic construct
  source 1..21: mol_type = other RNA
ttagcagcacatcatggttta

SEQ ID NO: 62 - RNA, 23 nt, synthetic construct
  source 1..23: mol_type = other RNA
aatggcgccactagggttgtgaa

SEQ ID NO: 63 - RNA, 22 nt, synthetic construct
  source 1..22: mol_type = other RNA
ttctggaattctgtgtgaggga

SEQ ID NO: 64 - RNA, 23 nt, synthetic construct
  source 1..23: mol_type = other RNA
atattgcacttgtcccggcctga

SEQ ID NO: 65 - RNA, 22 nt, synthetic construct
  source 1..22: mol_type = other RNA
aaactctacttgtccttctgag

SEQ ID NO: 66 - RNA, 22 nt, synthetic construct
  source 1..22: mol_type = other RNA
ttattgcacttgtcccggcctg

SEQ ID NO: 67 - RNA, 26 nt, synthetic construct
  source 1..26: mol_type = other RNA
ctcgtcctgtactgagctgccccgag

SEQ ID NO: 68 - RNA, 24 nt, synthetic construct
  source 1..24: mol_type = other RNA
cctgtactgagctgccccgagttt

SEQ ID NO: 69 - RNA, 22 nt, synthetic construct
  source 1..22: mol_type = other RNA
gaactcaccctctgctcccagt

SEQ ID NO: 70 - RNA, 22 nt, synthetic construct
  source 1..22: mol_type = other RNA
tggagagaaaggcagttcctgt

SEQ ID NO: 71 - RNA, 23 nt, synthetic construct
  source 1..23: mol_type = other RNA
tattgcacttgtcccggccaaaa

SEQ ID NO: 72 - RNA, 21 nt, synthetic construct
  source 1..21: mol_type = other RNA
ttttagagacggggtcttgct

SEQ ID NO: 73 - RNA, 27 nt, synthetic construct
  source 1..27: mol_type = other RNA
tttggcaatggtagaactcacactgga

SEQ ID NO: 74 - RNA, 21 nt, synthetic construct
  source 1..21: mol_type = other RNA
tgtagagcagggagcaggaag

SEQ ID NO: 75 - RNA, 23 nt, synthetic construct
  source 1..23: mol_type = other RNA
gtattgcacttgtcccggcctgt

SEQ ID NO: 76 - RNA, 22 nt, synthetic construct
  source 1..22: mol_type = other RNA
tgtgaggttggcattgttgtct

SEQ ID NO: 77 - RNA, 24 nt, synthetic construct
  source 1..24: mol_type = other RNA
gtattgcacttgtcccggcctgta

SEQ ID NO: 78 - RNA, 20 nt, synthetic construct
  source 1..20: mol_type = other RNA
tgtgaggttggcattgttgt

SEQ ID NO: 79 - RNA, 22 nt, synthetic construct
  source 1..22: mol_type = other RNA
tccactctcctggcccccagtt

SEQ ID NO: 80 - RNA, 23 nt, synthetic construct
  source 1..23: mol_type = other RNA
tcctgtactgatctgccccgagt

SEQ ID NO: 81 - RNA, 23 nt, synthetic construct
  source 1..23: mol_type = other RNA
aaacattcaacgctgtcggtgag

SEQ ID NO: 82 - RNA, 24 nt, synthetic construct
  source 1..24: mol_type = other RNA
taaagtgctgacagtgcagataga

SEQ ID NO: 83 - RNA, 25 nt, synthetic construct
  source 1..25: mol_type = other RNA
tcctgtactgagctgccccgagttc

SEQ ID NO: 84 - RNA, 23 nt, synthetic construct
  source 1..23: mol_type = other RNA
tcctgtactgagctgccccgttt

SEQ ID NO: 85 - RNA, 24 nt, synthetic construct
  source 1..24: mol_type = other RNA
tctctcggctcctcgcggctcgta

SEQ ID NO: 86 - RNA, 21 nt, synthetic construct
  source 1..21: mol_type = other RNA
cctgtactgagctgcgccgag

SEQ ID NO: 87 - RNA, 19 nt, synthetic construct
  source 1..19: mol_type = other RNA
atggcactggtagaattca

SEQ ID NO: 88 - RNA, 24 nt, synthetic construct
  source 1..24: mol_type = other RNA
atattgcacttgtcccggcctgta

SEQ ID NO: 89 - RNA, 23 nt, synthetic construct
  source 1..23: mol_type = other RNA
atattgcacttgtcccggcctgt

SEQ ID NO: 90 - RNA, 23 nt, synthetic construct
  source 1..23: mol_type = other RNA
tattgcacttgtctcggcctgta

SEQ ID NO: 91 - RNA, 24 nt, synthetic construct
  source 1..24: mol_type = other RNA
tattgcacttgtcccggcctgatt

SEQ ID NO: 92 - RNA, 22 nt, synthetic construct
  source 1..22: mol_type = other RNA
atattgcacttgtcccggcctg

SEQ ID NO: 93 - RNA, 25 nt, synthetic construct
  source 1..25: mol_type = other RNA
tgtcctgtactgagctgccccgagt

SEQ ID NO: 94 - RNA, 21 nt, synthetic construct
  source 1..21: mol_type = other RNA
tgtgtccgggaagtggaggag

SEQ ID NO: 95 - RNA, 23 nt, synthetic construct
  source 1..23: mol_type = other RNA
ttattgcacttgtcccggcctgt

SEQ ID NO: 96 - RNA, 25 nt, synthetic construct
  source 1..25: mol_type = other RNA
ctcctgtactgagctgccccgagaa

SEQ ID NO: 97 - RNA, 23 nt, synthetic construct
  source 1..23: mol_type = other RNA
aaagtgctgttcgtgcaggtagt

SEQ ID NO: 98 - RNA, 22 nt, synthetic construct
  source 1..22: mol_type = other RNA
tattgcacttatcccggcctgt

SEQ ID NO: 99 - RNA, 24 nt, synthetic construct
  source 1..24: mol_type = other RNA
tattgcacttgtcccggcctgtca

SEQ ID NO: 100 - RNA, 22 nt, synthetic construct
  source 1..22: mol_type = other RNA
tcaggctcagtcccctcccggt

SEQ ID NO: 101 - RNA, 25 nt, synthetic construct
  source 1..25: mol_type = other RNA
tattgcacttgtcccggcctgtacg

SEQ ID NO: 102 - RNA, 24 nt, synthetic construct
  source 1..24: mol_type = other RNA
ctattgcacttgtcccggcctgta

SEQ ID NO: 103 - RNA, 24 nt, synthetic construct
  source 1..24: mol_type = other RNA
cctgtactgagctgccccgagtga

SEQ ID NO: 104 - RNA, 21 nt, synthetic construct
  source 1..21: mol_type = other RNA
ctgtactgagctgccccgaag

SEQ ID NO: 105 - RNA, 21 nt, synthetic construct
  source 1..21: mol_type = other RNA
tactgccctaaatgccccttc

SEQ ID NO: 106 - RNA, 25 nt, synthetic construct
  source 1..25: mol_type = other RNA
caaagtgcttacagtgcaggtagaa

SEQ ID NO: 107 - RNA, 23 nt, synthetic construct
  source 1..23: mol_type = other RNA
tgtagagcagggagcaggaagct

SEQ ID NO: 108 - RNA, 25 nt, synthetic construct
  source 1..25: mol_type = other RNA
tgtcagtttgtcaaataccccatat

SEQ ID NO: 109 - RNA, 23 nt, synthetic construct
  source 1..23: mol_type = other RNA
ccgcactgtgggtacttgctgat

SEQ ID NO: 110 - RNA, 23 nt, synthetic construct
  source 1..23: mol_type = other RNA
atcctgtactgagctgccccgtt

SEQ ID NO: 111 - RNA, 23 nt, synthetic construct
  source 1..23: mol_type = other RNA
tcaccctgcatcccgcacccagt

SEQ ID NO: 112 - RNA, 23 nt, synthetic construct
  source 1..23: mol_type = other RNA
cattgcacttgtcccggcctgta

SEQ ID NO: 113 - RNA, 24 nt, synthetic construct
  source 1..24: mol_type = other RNA
ttggcaatggtagaactcacactg

SEQ ID NO: 114 - RNA, 27 nt, synthetic construct
  source 1..27: mol_type = other RNA
aaaccgttaccattactgagtttagta

SEQ ID NO: 115 - RNA, 25 nt, synthetic construct
  source 1..25: mol_type = other RNA
aaccgttaccattactgagtttaga

SEQ ID NO: 116 - RNA, 24 nt, synthetic construct
  source 1..24: mol_type = other RNA
aacccgtagatccgaacttgtgtt

SEQ ID NO: 117 - RNA, 24 nt, synthetic construct
  source 1..24: mol_type = other RNA
ttatgggcttctaggcttgtacac

SEQ ID NO: 118 - RNA, 23 nt, synthetic construct
  source 1..23: mol_type = other RNA
aagctcggtctgaggcccctctt

SEQ ID NO: 119 - RNA, 23 nt, synthetic construct
  source 1..23: mol_type = other RNA
ttatcgagccagactccggggag

SEQ ID NO: 120 - RNA, 25 nt, synthetic construct
  source 1..25: mol_type = other RNA
acctgtactgagctgccccgagatt

SEQ ID NO: 121 - RNA, 25 nt, synthetic construct
  source 1..25: mol_type = other RNA
ttaggacatgactcgacggggctct

SEQ ID NO: 122 - RNA, 25 nt, synthetic construct
  source 1..25: mol_type = other RNA
actcggcgtggcgtcggtcgtgttt

SEQ ID NO: 123 - RNA, 25 nt, synthetic construct
  source 1..25: mol_type = other RNA
ttagagccgcaccgcagcctgcaca

SEQ ID NO: 124 - RNA, 19 nt, synthetic construct
  source 1..19: mol_type = other RNA
atccccggcacctccactt

SEQ ID NO: 125 - RNA, 19 nt, synthetic construct
  source 1..19: mol_type = other RNA
ttaaggggccgtggaggtg

SEQ ID NO: 126 - RNA, 25 nt, synthetic construct
  source 1..25: mol_type = other RNA
atctcccaacccttgtaccagtgtt

SEQ ID NO: 127 - RNA, 25 nt, synthetic construct
  source 1..25: mol_type = other RNA
ttaagagggttgggaacatggtcac

SEQ ID NO: 128 - RNA, 26 nt, synthetic construct
  source 1..26: mol_type = other RNA
atgtcccacccccactcctgttaatt

SEQ ID NO: 129 - RNA, 26 nt, synthetic construct
  source 1..26: mol_type = other RNA
ttaacagggtgggggtgaggacaatt

SEQ ID NO: 130 - RNA, 25 nt, synthetic construct
  source 1..25: mol_type = other RNA
atgtcccacccccactcctgttttt

SEQ ID NO: 131 - RNA, 25 nt, synthetic construct
  source 1..25: mol_type = other RNA
ttaacagggtgggggtgaggtcaaa

SEQ ID NO: 132 - RNA, 24 nt, synthetic construct
  source 1..24: mol_type = other RNA
cgtcccaccccccactcctgtttt

SEQ ID NO: 133 - RNA, 24 nt, synthetic construct
  source 1..24: mol_type = other RNA
ttccagggtggggggtgaggacaa

SEQ ID NO: 134 - RNA, 24 nt, synthetic construct
  source 1..24: mol_type = other RNA
ctatacgacctgctgcctttcgtt

SEQ ID NO: 135 - RNA, 24 nt, synthetic construct
  source 1..24: mol_type = other RNA
ttcatatgctggacgacggaaagc

SEQ ID NO: 136 - RNA, 23 nt, synthetic construct
  source 1..23: mol_type = other RNA
gacctcgccgtcccgcccgcctt

SEQ ID NO: 137 - RNA, 23 nt, synthetic construct
  source 1..23: mol_type = other RNA
ttgtggagcggcagggcgggcgg

SEQ ID NO: 138 - RNA, 20 nt, synthetic construct
  source 1..20: mol_type = other RNA
gcacttgtctcggtctgatt

SEQ ID NO: 139 - RNA, 20 nt, synthetic construct
  source 1..20: mol_type = other RNA
ttggtgaacagtgccagact

SEQ ID NO: 140 - RNA, 20 nt, synthetic construct
  source 1..20: mol_type = other RNA
gtagtaggttgtatagtttt

SEQ ID NO: 141 - RNA, 20 nt, synthetic construct
  source 1..20: mol_type = other RNA
ttgatcatccaacatatcaa

SEQ ID NO: 142 - RNA, 25 nt, synthetic construct
  source 1..25: mol_type = other RNA
gtcaacacttgctggtttcctcttt

SEQ ID NO: 143 - RNA, 25 nt, synthetic construct
  source 1..25: mol_type = other RNA
ttgagttgtgaacgaccaaaggaga

SEQ ID NO: 144 - RNA, 24 nt, synthetic construct
  source 1..24: mol_type = other RNA
gtcatacacggctctcctctcttt

SEQ ID NO: 145 - RNA, 24 nt, synthetic construct
  source 1..24: mol_type = other RNA
ttgagtatgtgccgagaggagaga

SEQ ID NO: 146 - RNA, 26 nt, synthetic construct
  source 1..26: mol_type = other RNA
tattgcacttgtcccggcctgtgatt

SEQ ID NO: 147 - RNA, 26 nt, synthetic construct
  source 1..26: mol_type = other RNA
ttttaacgtgaacagggccggacact

SEQ ID NO: 148 - RNA, 23 nt, synthetic construct
  source 1..23: mol_type = other RNA
tccccggcatctccaccaaaatt

SEQ ID NO: 149 - RNA, 23 nt, synthetic construct
  source 1..23: mol_type = other RNA
tttggggccgtagaggtggtttt

SEQ ID NO: 150 - RNA, 25 nt, synthetic construct
  source 1..25: mol_type = other RNA
tccctgtcctccaggagctcacgtt

SEQ ID NO: 151 - RNA, 25 nt, synthetic construct
  source 1..25: mol_type = other RNA
tttgggacaggaggtcctcgagtgc

SEQ ID NO: 152 - RNA, 26 nt, synthetic construct
  source 1..26: mol_type = other RNA
tccctgtcctccaggagctcacgatt

SEQ ID NO: 153 - RNA, 26 nt, synthetic construct
  source 1..26: mol_type = other RNA
tttgggtctggtggtcctcgtgtgct

SEQ ID NO: 154 - RNA, 23 nt, synthetic construct
  source 1..23: mol_type = other RNA
tccctgtcctccaggagctcatt

SEQ ID NO: 155 - RNA, 23 nt, synthetic construct
  source 1..23: mol_type = other RNA
tttgggacaggaggtcctcgagt

SEQ ID NO: 156 - RNA, 24 nt, synthetic construct
  source 1..24: mol_type = other RNA
tcctgtactgagctaccccgagtt

SEQ ID NO: 157 - RNA, 24 nt, synthetic construct
  source 1..24: mol_type = other RNA
tttggacatgactcgatggggctc

SEQ ID NO: 158 - RNA, 24 nt, synthetic construct
  source 1..24: mol_type = other RNA
tcctgtactgagctgccccagatt

SEQ ID NO: 159 - RNA, 24 nt, synthetic construct
  source 1..24: mol_type = other RNA
tttggacatgactcgacggggtct

SEQ ID NO: 160 - RNA, 27 nt, synthetic construct
  source 1..27: mol_type = other RNA
tcctgtactgagctgccccgaggcctt

SEQ ID NO: 161 - RNA, 27 nt, synthetic construct
  source 1..27: mol_type = other RNA
tttggacatgactcgacggggctccgg

SEQ ID NO: 162 - RNA, 24 nt, synthetic construct
  source 1..24: mol_type = other RNA
tctacagtgcacgtgtctccagtt

SEQ ID NO: 163 - RNA, 24 nt, synthetic construct
  source 1..24: mol_type = other RNA
tttgatgtcacgtgcacagtggtc

SEQ ID NO: 164 - RNA, 19 nt, synthetic construct
  source 1..19: mol_type = other RNA
tcttcgggggcgtgggttt

SEQ ID NO: 165 - RNA, 19 nt, synthetic construct
  source 1..19: mol_type = other RNA
tttgaagcccccgcaccca

SEQ ID NO: 166 - RNA, 24 nt, synthetic construct
  source 1..24: mol_type = other RNA
tcttgtactgagctgccccgagtt

SEQ ID NO: 167 - RNA, 24 nt, synthetic construct
  source 1..24: mol_type = other RNA
tttgaacatgactcgacggggctc

SEQ ID NO: 168 - RNA, 23 nt, synthetic construct
  source 1..23: mol_type = other RNA
tgaggggcagagagcgagaaatt

SEQ ID NO: 169 - RNA, 23 nt, synthetic construct
  source 1..23: mol_type = other RNA
tttctccccgtctctcgctcttt

SEQ ID NO: 170 - RNA, 25 nt, synthetic construct
  source 1..25: mol_type = other RNA
tggcatgcacgaggccccgggtttt

SEQ ID NO: 171 - RNA, 25 nt, synthetic construct
  source 1..25: mol_type = other RNA
tttccgtacgtgctccggggcccaa

SEQ ID NO: 172 - RNA, 26 nt, synthetic construct
  source 1..26: mol_type = other RNA
tggcatgcacgaggccccgggttttt

SEQ ID NO: 173 - RNA, 26 nt, synthetic construct
  source 1..26: mol_type = other RNA
tttccgttcgtgctccggggcccaaa

SEQ ID NO: 174 - RNA, 22 nt, synthetic construct
  source 1..22: mol_type = other RNA
tgggtttacgttgggagaactt

SEQ ID NO: 175 - RNA, 22 nt, synthetic construct
  source 1..22: mol_type = other RNA
tttcccaaatgcaaccctcttg

SEQ ID NO: 176 - RNA, 24 nt, synthetic construct
  source 1..24: mol_type = other RNA
tggttaggattcggcgctctcatt

SEQ ID NO: 177 - RNA, 24 nt, synthetic construct
  source 1..24: mol_type = other RNA
tttccaatcctaagccgcgagagt

SEQ ID NO: 178 - RNA, 23 nt, synthetic construct
  source 1..23: mol_type = other RNA
tgtcccacccccactcctgtttt

SEQ ID NO: 179 - RNA, 23 nt, synthetic construct
  source 1..23: mol_type = other RNA
tttcagggtgggggtgaggacaa

SEQ ID NO: 180 - RNA, 70 nt, synthetic construct
  source 1..70: mol_type = other RNA
tcaagtgatggagagcaatacccggggatgactgtgaccacattgggatgtattcgtactgtctgatgtt

SEQ ID NO: 181 - RNA, 70 nt, synthetic construct
  source 1..70: mol_type = other RNA
tttgttcactacctctcgttatgggcccctactgacactggtgtaaccctacataagcatgacagactac

SEQ ID NO: 182 - RNA, 53 nt, synthetic construct
  source 1..53: mol_type = other RNA
ttggtaaccaccaagtcaccatcttaagagcggacggtgcgccctccgggccc

SEQ ID NO: 183 - RNA, 53 nt, synthetic construct
  source 1..53: mol_type = other RNA
gcatgggtggttcagtggtagaattctcgcctgccacgcgggaggcccgggtt

SEQ ID NO: 184 - RNA, 53 nt, synthetic construct
  source 1..53: mol_type = other RNA
ttggtacccaccaagtcaccatcttaagagcggacggtgcgccctccgggccc

SEQ ID NO: 185 - RNA, 60 nt, synthetic construct
  source 1..60: mol_type = other RNA
gcattggtggttcagtggtagaattctcgcctgccacgcgggaggcccgggttcgttttt

SEQ ID NO: 186 - RNA, 60 nt, synthetic construct
  source 1..60: mol_type = other RNA
ttggtaaccaccaagtcaccatcttaagagcggacggtgcgccctccgggcccaagcaaa

SEQ ID NO: 187 - RNA, 51 nt, synthetic construct
  source 1..51: mol_type = other RNA
ttggtggttcagtggtagaattctcgcctgccacgcgggaggcccgggttt

SEQ ID NO: 188 - RNA, 51 nt, synthetic construct
  source 1..51: mol_type = other RNA
tttaccaccaagtcaccatcttaagagcggacggtgcgccctccgggccca

SEQ ID NO: 189 - RNA, 54 nt, synthetic construct
  source 1..54: mol_type = other RNA
gcattggtggttcagtggtagaattctcgcctgccacgcgggagacccgggttt

SEQ ID NO: 190 - RNA, 54 nt, synthetic construct
  source 1..54: mol_type = other RNA
ttggtaaccaccaagtcaccatcttaagagcggacggtgcgccctctgggccca

SEQ ID NO: 191 - RNA, 67 nt, synthetic construct
  source 1..67: mol_type = other RNA
gcattggtggttcagtggtagaattctcgcctgccacgcgggaggcccgggttcgtttcccggcctt

SEQ ID NO: 192 - RNA, 67 nt, synthetic construct
  source 1..67: mol_type = other RNA
ttggtaaccaccaagtcaccatcttaagagcggacggtgcgccctccgggcccaagcaaagggccgg

SEQ ID NO: 193 - RNA, 51 nt, synthetic construct
  source 1..51: mol_type = other RNA
gtgggggagaggctgtcgctgcgcttctgggcccgcggcgggcgtggggtt

SEQ ID NO: 194 - RNA, 51 nt, synthetic construct
  source 1..51: mol_type = other RNA
ttgaccccctctccgacagcgacgcgaagacccgggcgccgcccgcacccc

SEQ ID NO: 195 - RNA, 54 nt, synthetic construct
  source 1..54: mol_type = other RNA
gcattagtggttcagtggtagaattctcgcctgccacgcgggaggcccgggttt

SEQ ID NO: 196 - RNA, 54 nt, synthetic construct
  source 1..54: mol_type = other RNA
ttggtaatcaccaagtcaccatcttaagagcggacggtgcgccctccgggccca

SEQ ID NO: 197 - RNA, 55 nt, synthetic construct
  source 1..55: mol_type = other RNA
gcattggtggttcagtggtagaattctcgcctgccacgcgggaggcccgggtatt

SEQ ID NO: 198 - RNA, 55 nt, synthetic construct
  source 1..55: mol_type = other RNA
ttggtaacctccaagtctccatcttaagagcggacggtgcgccctccgggcccat

SEQ ID NO: 199 - RNA, 54 nt, synthetic construct
  source 1..54: mol_type = other RNA
gcattggtggtttagtggtagaattctcgcctgccacgcgggaggcccgggttt

SEQ ID NO: 200 - RNA, 54 nt, synthetic construct
  source 1..54: mol_type = other RNA
ttggtaaccaccaaatctccatcttaagagcggacggtgcgccctccgggccca

SEQ ID NO: 201 - RNA, 25 nt, synthetic construct
  source 1..25: mol_type = other RNA
ttttaacgtgaacagggccggatat

SEQ ID NO: 202 - RNA, 24 nt, synthetic construct
  source 1..24: mol_type = other RNA
tattgcacttgtcccggcctgttt

SEQ ID NO: 203 - RNA, 24 nt, synthetic construct
  source 1..24: mol_type = other RNA
ttttaacgtgaacagggccggaca

SEQ ID NO: 204 - RNA, 24 nt, synthetic construct
  source 1..24: mol_type = other RNA
tatggcactggtagaattcacttt

SEQ ID NO: 205 - RNA, 24 nt, synthetic construct
  source 1..24: mol_type = other RNA
ttttaccgtgaccatcttaagtga

SEQ ID NO: 206 - RNA, 25 nt, synthetic construct
  source 1..25: mol_type = other RNA
atggcactggtagaattcactgttt

SEQ ID NO: 207 - RNA, 25 nt, synthetic construct
  source 1..25: mol_type = other RNA
ttaaccgtgaccatcttaagtgaca

SEQ ID NO: 208 - RNA, 27 nt, synthetic construct
  source 1..27: mol_type = other RNA
aatattgcacttgtcccggcctgtatt

SEQ ID NO: 209 - RNA, 27 nt, synthetic construct
  source 1..27: mol_type = other RNA
ttatataacgtgaacagggccggacat

SEQ ID NO: 210 - RNA, 23 nt, synthetic construct
  source 1..23: mol_type = other RNA
ccaaaactgcagttacttttgtt

SEQ ID NO: 211 - RNA, 23 nt, synthetic construct
  source 1..23: mol_type = other RNA
ttcgttttgacgtcaatgaaaac

SEQ ID NO: 212 - RNA, 24 nt, synthetic construct
  source 1..24: mol_type = other RNA
tattacacttgtcccggcctgttt

SEQ ID NO: 213 - RNA, 24 nt, synthetic construct
  source 1..24: mol_type = other RNA
ttttaatgtgaacagggccggaca

SEQ ID NO: 214 - RNA, 26 nt, synthetic construct
  source 1..26: mol_type = other RNA
caaagtgctcatagtgcaggtagatt

SEQ ID NO: 215 - RNA, 26 nt, synthetic construct
  source 1..26: mol_type = other RNA
ttctttcacgagtatcacgtccatct

SEQ ID NO: 216 - RNA, 25 nt, synthetic construct
  source 1..25: mol_type = other RNA
gggagccaggaagtattgatgtttt

SEQ ID NO: 217 - RNA, 25 nt, synthetic construct
  source 1..25: mol_type = other RNA
ttgcctcggtccttcataactacaa

SEQ ID NO: 218 - RNA, 27 nt, synthetic construct
  source 1..27: mol_type = other RNA
tattgcacttgtcccggcctgttaatt

SEQ ID NO: 219 - RNA, 27 nt, synthetic construct
  source 1..27: mol_type = other RNA
ttttaacgtgaacagggccggacaatt

SEQ ID NO: 220 - RNA, 24 nt, synthetic construct
  source 1..24: mol_type = other RNA
gattgcacttgtcccggcctgttt

SEQ ID NO: 221 - RNA, 24 nt, synthetic construct
  source 1..24: mol_type = other RNA
ttgtaacgtgaacagggccggaca

SEQ ID NO: 222 - RNA, 27 nt, synthetic construct
  source 1..27: mol_type = other RNA
gtgtcagtttgtcaaataccccaattt

SEQ ID NO: 223 - RNA, 27 nt, synthetic construct
  source 1..27: mol_type = other RNA
ttgacagtcaaacagtttatggggtta

SEQ ID NO: 224 - RNA, 28 nt, synthetic construct
  source 1..28: mol_type = other RNA
agctcggtctgaggcccctcagttattt

SEQ ID NO: 225 - RNA, 28 nt, synthetic construct
  source 1..28: mol_type = other RNA
ttacgagccagactccggggagtcaata

SEQ ID NO: 226 - RNA, 26 nt, synthetic construct
  source 1..26: mol_type = other RNA
acaaaccgttaccattactgagtttt

SEQ ID NO: 227 - RNA, 26 nt, synthetic construct
  source 1..26: mol_type = other RNA
ttagtttggcaatggtaatgactcaa

SEQ ID NO: 228 - RNA, 23 nt, synthetic construct
  source 1..23: mol_type = other RNA
cctgtactgagctgcccctagtt

SEQ ID NO: 229 - RNA, 23 nt, synthetic construct
  source 1..23: mol_type = other RNA
ttcgacatgactcgacggggatc

SEQ ID NO: 230 - RNA, 22 nt, synthetic construct
  source 1..22: mol_type = other RNA
aatgcacccgggcaaggatttt

SEQ ID NO: 231 - RNA, 22 nt, synthetic construct
  source 1..22: mol_type = other RNA
ttatacgtgggcccgttcctaa

SEQ ID NO: 232 - RNA, 23 nt, synthetic construct
  source 1..23: mol_type = other RNA
ttagcagcacatcatggtttatt

SEQ ID NO: 233 - RNA, 23 nt, synthetic construct
  source 1..23: mol_type = other RNA
tttatcgtcgtgtagtaccaaat

SEQ ID NO: 234 - RNA, 25 nt, synthetic construct
  source 1..25: mol_type = other RNA
aatggcgccactagggttgtgaatt

SEQ ID NO: 235 - RNA, 25 nt, synthetic construct
  source 1..25: mol_type = other RNA
ttataccgcggtgatcccaacactt

SEQ ID NO: 236 - RNA, 24 nt, synthetic construct
  source 1..24: mol_type = other RNA
ttctggaattctgtgtgagggatt

SEQ ID NO: 237 - RNA, 24 nt, synthetic construct
  source 1..24: mol_type = other RNA
tttagaccttaagacacactccct

SEQ ID NO: 238 - RNA, 25 nt, synthetic construct
  source 1..25: mol_type = other RNA
atattgcacttgtcccggcctgatt

SEQ ID NO: 239 - RNA, 25 nt, synthetic construct
  source 1..25: mol_type = other RNA
ttaataacgtgaacagggccggact

SEQ ID NO: 240 - RNA, 24 nt, synthetic construct
  source 1..24: mol_type = other RNA
aaactctacttgtccttctgagtt

SEQ ID NO: 241 - RNA, 24 nt, synthetic construct
  source 1..24: mol_type = other RNA
ttattgagatgaacaggaagactc

SEQ ID NO: 242 - RNA, 24 nt, synthetic construct
  source 1..24: mol_type = other RNA
ttattgcacttgtcccggcctgtt

SEQ ID NO: 243 - RNA, 24 nt, synthetic construct
  source 1..24: mol_type = other RNA
tttataacgtgaacagggccggac

SEQ ID NO: 244 - RNA, 28 nt, synthetic construct
  source 1..28: mol_type = other RNA
ctcgtcctgtactgagctgccccgagtt

SEQ ID NO: 245 - RNA, 28 nt, synthetic construct
  source 1..28: mol_type = other RNA
ttcagcaggacatgactcgacggggctc

SEQ ID NO: 246 - RNA, 26 nt, synthetic construct
  source 1..26: mol_type = other RNA
cctgtactgagctgccccgagttttt

SEQ ID NO: 247 - RNA, 26 nt, synthetic construct
  source 1..26: mol_type = other RNA
ttcgacatgactcgacggggctcaaa

SEQ ID NO: 248 - RNA, 24 nt, synthetic construct
  source 1..24: mol_type = other RNA
gaactcaccctctgctcccagttt

SEQ ID NO: 249 - RNA, 24 nt, synthetic construct
  source 1..24: mol_type = other RNA
ttgttgagtgggagacgagggtca

SEQ ID NO: 250 - RNA, 24 nt, synthetic construct
  source 1..24: mol_type = other RNA
tggagagaaaggcagttcctgttt

SEQ ID NO: 251 - RNA, 24 nt, synthetic construct
  source 1..24: mol_type = other RNA
tttcctctctttccgtcaaggaca

SEQ ID NO: 252 - RNA, 25 nt, synthetic construct
  source 1..25: mol_type = other RNA
tattgcacttgtcccggccaaaatt

SEQ ID NO: 253 - RNA, 25 nt, synthetic construct
  source 1..25: mol_type = other RNA
ttttaacgtgaacagggccggtttt

SEQ ID NO: 254 - RNA, 23 nt, synthetic construct
  source 1..23: mol_type = other RNA
ttttagagacggggtcttgcttt

SEQ ID NO: 255 - RNA, 23 nt, synthetic construct
  source 1..23: mol_type = other RNA
tttaaatctctgccccagaacga

SEQ ID NO: 256 - RNA, 29 nt, synthetic construct
  source 1..29: mol_type = other RNA
tttggcaatggtagaactcacactggatt

SEQ ID NO: 257 - RNA, 29 nt, synthetic construct
  source 1..29: mol_type = other RNA
tttaaccgttaccatcttgagtgtgacct

SEQ ID NO: 258 - RNA, 23 nt, synthetic construct
  source 1..23: mol_type = other RNA
tgtagagcagggagcaggaagtt

SEQ ID NO: 259 - RNA, 23 nt, synthetic construct
  source 1..23: mol_type = other RNA
tttcatctcgtccctcgtccttc

SEQ ID NO: 260 - RNA, 25 nt, synthetic construct
  source 1..25: mol_type = other RNA
gtattgcacttgtcccggcctgttt

SEQ ID NO: 261 - RNA, 25 nt, synthetic construct
  source 1..25: mol_type = other RNA
ttgataacgtgaacagggccggaca

SEQ ID NO: 262 - RNA, 24 nt, synthetic construct
  source 1..24: mol_type = other RNA
tgtgaggttggcattgttgtcttt

SEQ ID NO: 263 - RNA, 24 nt, synthetic construct
  source 1..24: mol_type = other RNA
tttcactccaaccgtaacaacaga

SEQ ID NO: 264 - RNA, 26 nt, synthetic construct
  source 1..26: mol_type = other RNA
gtattgcacttgtcccggcctgtatt

SEQ ID NO: 265 - RNA, 26 nt, synthetic construct
  source 1..26: mol_type = other RNA
ttgataacgtgaacagggccggacat

SEQ ID NO: 266 - RNA, 22 nt, synthetic construct
  source 1..22: mol_type = other RNA
tgtgaggttggcattgttgttt

SEQ ID NO: 267 - RNA, 22 nt, synthetic construct
  source 1..22: mol_type = other RNA
tttcactccaaccgtaacaaca

SEQ ID NO: 268 - RNA, 24 nt, synthetic construct
  source 1..24: mol_type = other RNA
tccactctcctggcccccagtttt

SEQ ID NO: 269 - RNA, 24 nt, synthetic construct
  source 1..24: mol_type = other RNA
tttggtgagaggaccgggggtcaa

SEQ ID NO: 270 - RNA, 25 nt, synthetic construct
  source 1..25: mol_type = other RNA
tcctgtactgatctgccccgagttt

SEQ ID NO: 271 - RNA, 25 nt, synthetic construct
  source 1..25: mol_type = other RNA
tttggacatgactagacggggctca

SEQ ID NO: 272 - RNA, 25 nt, synthetic construct
  source 1..25: mol_type = other RNA
aaacattcaacgctgtcggtgagtt

SEQ ID NO: 273 - RNA, 25 nt, synthetic construct
  source 1..25: mol_type = other RNA
ttattgtaagttgcgacagccactc

SEQ ID NO: 274 - RNA, 26 nt, synthetic construct
  source 1..26: mol_type = other RNA
taaagtgctgacagtgcagatagatt

SEQ ID NO: 275 - RNA, 26 nt, synthetic construct
  source 1..26: mol_type = other RNA
ttttttcacgactgtcacgtctatct

SEQ ID NO: 276 - RNA, 27 nt, synthetic construct
  source 1..27: mol_type = other RNA
tcctgtactgagctgccccgagttctt

SEQ ID NO: 277 - RNA, 27 nt, synthetic construct
  source 1..27: mol_type = other RNA
tttggacatgactcgacggggctcaag

SEQ ID NO: 278 - RNA, 25 nt, synthetic construct
  source 1..25: mol_type = other RNA
tcctgtactgagctgccccgttttt

SEQ ID NO: 279 - RNA, 25 nt, synthetic construct
  source 1..25: mol_type = other RNA
tttggacatgactcgacggggcaaa

SEQ ID NO: 280 - RNA, 26 nt, synthetic construct
  source 1..26: mol_type = other RNA
tctctcggctcctcgcggctcgtatt

SEQ ID NO: 281 - RNA, 26 nt, synthetic construct
  source 1..26: mol_type = other RNA
tttgagagccgaggagcgccgagcat

SEQ ID NO: 282 - RNA, 23 nt, synthetic construct
  source 1..23: mol_type = other RNA
cctgtactgagctgcgccgagtt

SEQ ID NO: 283 - RNA, 23 nt, synthetic construct
  source 1..23: mol_type = other RNA
ttcgacatgactcgacgcggctc

SEQ ID NO: 284 - RNA, 21 nt, synthetic construct
  source 1..21: mol_type = other RNA
atggcactggtagaattcatt

SEQ ID NO: 285 - RNA, 21 nt, synthetic construct
  source 1..21: mol_type = other RNA
ttaaccgtgaccatcttaagt

SEQ ID NO: 286 - RNA, 26 nt, synthetic construct
  source 1..26: mol_type = other RNA
atattgcacttgtcccggcctgtatt

SEQ ID NO: 287 - RNA, 26 nt, synthetic construct
  source 1..26: mol_type = other RNA
ttaataacgtgaacagggccggacat

SEQ ID NO: 288 - RNA, 25 nt, synthetic construct
  source 1..25: mol_type = other RNA
atattgcacttgtcccggcctgttt

SEQ ID NO: 289 - RNA, 25 nt, synthetic construct
  source 1..25: mol_type = other RNA
ttaataacgtgaacagggccggaca

SEQ ID NO: 290 - RNA, 25 nt, synthetic construct
  source 1..25: mol_type = other RNA
tattgcacttgtctcggcctgtatt

SEQ ID NO: 291 - RNA, 25 nt, synthetic construct
  source 1..25: mol_type = other RNA
ttttaacgtgaacagagccggacat

SEQ ID NO: 292 - RNA, 26 nt, synthetic construct
  source 1..26: mol_type = other RNA
tattgcacttgtcccggcctgatttt

SEQ ID NO: 293 - RNA, 26 nt, synthetic construct
  source 1..26: mol_type = other RNA
ttttaacgtgaacagggccggactaa

SEQ ID NO: 294 - RNA, 24 nt, synthetic construct
  source 1..24: mol_type = other RNA
atattgcacttgtcccggcctgtt

SEQ ID NO: 295 - RNA, 24 nt, synthetic construct
  source 1..24: mol_type = other RNA
ttaataacgtgaacagggccggac

SEQ ID NO: 296 - RNA, 27 nt, synthetic construct
  source 1..27: mol_type = other RNA
tgtcctgtactgagctgccccgagttt

SEQ ID NO: 297 - RNA, 27 nt, synthetic construct
  source 1..27: mol_type = other RNA
tttcaggacatgactcgacggggctca

SEQ ID NO: 298 - RNA, 23 nt, synthetic construct
  source 1..23: mol_type = other RNA
tgtgtccgggaagtggaggagtt

SEQ ID NO: 299 - RNA, 23 nt, synthetic construct
  source 1..23: mol_type = other RNA
tttcacaggcccttcacctcctc

SEQ ID NO: 300 - RNA, 25 nt, synthetic construct
  source 1..25: mol_type = other RNA
ttattgcacttgtcccggcctgttt

SEQ ID NO: 301 - RNA, 25 nt, synthetic construct
  source 1..25: mol_type = other RNA
tttataacgtgaacagggccggaca

SEQ ID NO: 302 - RNA, 27 nt, synthetic construct
  source 1..27: mol_type = other RNA
ctcctgtactgagctgccccgagaatt

SEQ ID NO: 303 - RNA, 27 nt, synthetic construct
  source 1..27: mol_type = other RNA
ttcaggacatgactcgacggggctctt

SEQ ID NO: 304 - RNA, 25 nt, synthetic construct
  source 1..25: mol_type = other RNA
aaagtgctgttcgtgcaggtagttt

SEQ ID NO: 305 - RNA, 25 nt, synthetic construct
  source 1..25: mol_type = other RNA
ttattcacgacaagcacgtccatca

SEQ ID NO: 306 - RNA, 24 nt, synthetic construct
  source 1..24: mol_type = other RNA
tattgcacttatcccggcctgttt

SEQ ID NO: 307 - RNA, 24 nt, synthetic construct
  source 1..24: mol_type = other RNA
ttttaacgtgaatagggccggaca

SEQ ID NO: 308 - RNA, 26 nt, synthetic construct
  source 1..26: mol_type = other RNA
tattgcacttgtcccggcctgtcatt

SEQ ID NO: 309 - RNA, 26 nt, synthetic construct
  source 1..26: mol_type = other RNA
ttttaacgtgaacagggccggacagt

SEQ ID NO: 310 - RNA, 24 nt, synthetic construct
  source 1..24: mol_type = other RNA
tcaggctcagtcccctcccggttt

SEQ ID NO: 311 - RNA, 24 nt, synthetic construct
  source 1..24: mol_type = other RNA
tttgtccgagtcaggggagggcca

SEQ ID NO: 312 - RNA, 27 nt, synthetic construct
  source 1..27: mol_type = other RNA
tattgcacttgtcccggcctgtacgtt

SEQ ID NO: 313 - RNA, 27 nt, synthetic construct
  source 1..27: mol_type = other RNA
ttttaacgtgaacagggccggacatgc

SEQ ID NO: 314 - RNA, 26 nt, synthetic construct
  source 1..26: mol_type = other RNA
ctattgcacttgtcccggcctgtatt

SEQ ID NO: 315 - RNA, 26 nt, synthetic construct
  source 1..26: mol_type = other RNA
ttcataacgtgaacagggccggacat

SEQ ID NO: 316 - RNA, 26 nt, synthetic construct
  source 1..26: mol_type = other RNA
cctgtactgagctgccccgagtgatt

SEQ ID NO: 317 - RNA, 26 nt, synthetic construct
  source 1..26: mol_type = other RNA
ttcgacatgactcgacggggctcact

SEQ ID NO: 318 - RNA, 23 nt, synthetic construct
  source 1..23: mol_type = other RNA
ctgtactgagctgccccgaagtt

SEQ ID NO: 319 - RNA, 23 nt, synthetic construct
  source 1..23: mol_type = other RNA
ttcacatgactcgacggggcttc

SEQ ID NO: 320 - RNA, 23 nt, synthetic construct
  source 1..23: mol_type = other RNA
tactgccctaaatgccccttctt

SEQ ID NO: 321 - RNA, 23 nt, synthetic construct
  source 1..23: mol_type = other RNA
ttttgacgggatttacggggaag

SEQ ID NO: 322 - RNA, 27 nt, synthetic construct
  source 1..27: mol_type = other RNA
caaagtgcttacagtgcaggtagaatt

SEQ ID NO: 323 - RNA, 27 nt, synthetic construct
  source 1..27: mol_type = other RNA
ttctttcacgaatgtcacgtccatctt

SEQ ID NO: 324 - RNA, 25 nt, synthetic construct
  source 1..25: mol_type = other RNA
tgtagagcagggagcaggaagcttt

SEQ ID NO: 325 - RNA, 25 nt, synthetic construct
  source 1..25: mol_type = other RNA
tttcatctcgtccctcgtccttcga

SEQ ID NO: 326 - RNA, 27 nt, synthetic construct
  source 1..27: mol_type = other RNA
tgtcagtttgtcaaataccccatattt

SEQ ID NO: 327 - RNA, 27 nt, synthetic construct
  source 1..27: mol_type = other RNA
tttcagtcaaacagtttatggggtata

SEQ ID NO: 328 - RNA, 25 nt, synthetic construct
  source 1..25: mol_type = other RNA
ccgcactgtgggtacttgctgattt

SEQ ID NO: 329 - RNA, 25 nt, synthetic construct
  source 1..25: mol_type = other RNA
ttcgcgtgacacccatgaacgacta

SEQ ID NO: 330 - RNA, 25 nt, synthetic construct
  source 1..25: mol_type = other RNA
atcctgtactgagctgccccgtttt

SEQ ID NO: 331 - RNA, 25 nt, synthetic construct
  source 1..25: mol_type = other RNA
ttaaggacatgactcgacggggcaa

SEQ ID NO: 332 - RNA, 25 nt, synthetic construct
  source 1..25: mol_type = other RNA
tcaccctgcatcccgcacccagttt

SEQ ID NO: 333 - RNA, 25 nt, synthetic construct
  source 1..25: mol_type = other RNA
tttgtgggacgtagggcgtgggtca

SEQ ID NO: 334 - RNA, 25 nt, synthetic construct
  source 1..25: mol_type = other RNA
cattgcacttgtcccggcctgtatt

SEQ ID NO: 335 - RNA, 25 nt, synthetic construct
  source 1..25: mol_type = other RNA
ttctaacgtgaacagggccggacat

SEQ ID NO: 336 - RNA, 26 nt, synthetic construct
  source 1..26: mol_type = other RNA
ttggcaatggtagaactcacactgtt

SEQ ID NO: 337 - RNA, 26 nt, synthetic construct
  source 1..26: mol_type = other RNA
tttaccgttaccatcttgagtgtgac

SEQ ID NO: 338 - RNA, 29 nt, synthetic construct
  source 1..29: mol_type = other RNA
aaaccgttaccattactgagtttagtatt

SEQ ID NO: 339 - RNA, 29 nt, synthetic construct
  source 1..29: mol_type = other RNA
ttattggcaatggtaatgactcaaatcat

SEQ ID NO: 340 - RNA, 27 nt, synthetic construct
  source 1..27: mol_type = other RNA
aaccgttaccattactgagtttagatt

SEQ ID NO: 341 - RNA, 27 nt, synthetic construct
  source 1..27: mol_type = other RNA
ttatggcaatggtaatgactcaaatct

SEQ ID NO: 342 - RNA, 50 nt, synthetic construct
  source 1..50: mol_type = other RNA
gtcgtatccagtgcagggtccgaggtattcgcactggatacgaccacaag

SEQ ID NO: 343 - RNA, 50 nt, synthetic construct
  source 1..50: mol_type = other RNA
gtcgtatccagtgcagggtccgaggtattcgcactggatacgacgagggg

SEQ ID NO: 344 - RNA, 50 nt, synthetic construct
  source 1..50: mol_type = other RNA
gtcgtatccagtgcagggtccgaggtattcgcactggatacgacggggct

SEQ ID NO: 345 - RNA, 50 nt, synthetic construct
  source 1..50: mol_type = other RNA
gtcgtatccagtgcagggtccgaggtattcgcactggatacgactctcgg

SEQ ID NO: 346 - RNA, 50 nt, synthetic construct
  source 1..50: mol_type = other RNA
gtcgtatccagtgcagggtccgaggtattcgcactggatacgacacacga

SEQ ID NO: 347 - RNA, 50 nt, synthetic construct
  source 1..50: mol_type = other RNA
gtcgtatccagtgcagggtccgaggtattcgcactggatacgacgtggag

SEQ ID NO: 348 - RNA, 50 nt, synthetic construct
  source 1..50: mol_type = other RNA
gtcgtatccagtgcagggtccgaggtattcgcactggatacgaccactgg

SEQ ID NO: 349 - RNA, 50 nt, synthetic construct
  source 1..50: mol_type = other RNA
gtcgtatccagtgcagggtccgaggtattcgcactggatacgacttaaca

SEQ ID NO: 350 - RNA, 50 nt, synthetic construct
  source 1..50: mol_type = other RNA
gtcgtatccagtgcagggtccgaggtattcgcactggatacgacaaacag

SEQ ID NO: 351 - RNA, 50 nt, synthetic construct
  source 1..50: mol_type = other RNA
gtcgtatccagtgcagggtccgaggtattcgcactggatacgacaacagg

SEQ ID NO: 352 - RNA, 50 nt, synthetic construct
  source 1..50: mol_type = other RNA
gtcgtatccagtgcagggtccgaggtattcgcactggatacgaccgaaag

SEQ ID NO: 353 - RNA, 50 nt, synthetic construct
  source 1..50: mol_type = other RNA
gtcgtatccagtgcagggtccgaggtattcgcactggatacgacggcggg

SEQ ID NO: 354 - RNA, 50 nt, synthetic construct
  source 1..50: mol_type = other RNA
gtcgtatccagtgcagggtccgaggtattcgcactggatacgactcagac

SEQ ID NO: 355 - RNA, 50 nt, synthetic construct
  source 1..50: mol_type = other RNA
gtcgtatccagtgcagggtccgaggtattcgcactggatacgacaactat

SEQ ID NO: 356 - RNA, 50 nt, synthetic construct
  source 1..50: mol_type = other RNA
gtcgtatccagtgcagggtccgaggtattcgcactggatacgacagagga

SEQ ID NO: 357 - RNA, 50 nt, synthetic construct
  source 1..50: mol_type = other RNA
gtcgtatccagtgcagggtccgaggtattcgcactggatacgacagagag

SEQ ID NO: 358 - RNA, 50 nt, synthetic construct
  source 1..50: mol_type = other RNA
gtcgtatccagtgcagggtccgaggtattcgcactggatacgactcacag

SEQ ID NO: 359 - RNA, 50 nt, synthetic construct
  source 1..50: mol_type = other RNA
gtcgtatccagtgcagggtccgaggtattcgcactggatacgacttttgg

SEQ ID NO: 360 - RNA, 50 nt, synthetic construct
  source 1..50: mol_type = other RNA
gtcgtatccagtgcagggtccgaggtattcgcactggatacgaccgtgag

SEQ ID NO: 361 - RNA, 50 nt, synthetic construct
  source 1..50: mol_type = other RNA
gtcgtatccagtgcagggtccgaggtattcgcactggatacgactcgtga

SEQ ID NO: 362 - RNA, 50 nt, synthetic construct
  source 1..50: mol_type = other RNA
gtcgtatccagtgcagggtccgaggtattcgcactggatacgactgagct

SEQ ID NO: 363 - RNA, 50 nt, synthetic construct
  source 1..50: mol_type = other RNA
gtcgtatccagtgcagggtccgaggtattcgcactggatacgacctcggg

SEQ ID NO: 364 - RNA, 50 nt, synthetic construct
  source 1..50: mol_type = other RNA
gtcgtatccagtgcagggtccgaggtattcgcactggatacgactctggg

SEQ ID NO: 365 - RNA, 50 nt, synthetic construct
  source 1..50: mol_type = other RNA
gtcgtatccagtgcagggtccgaggtattcgcactggatacgacggcctc

SEQ ID NO: 366 - RNA, 50 nt, synthetic construct
  source 1..50: mol_type = other RNA
gtcgtatccagtgcagggtccgaggtattcgcactggatacgacctggag

SEQ ID NO: 367 - RNA, 50 nt, synthetic construct
  source 1..50: mol_type = other RNA
gtcgtatccagtgcagggtccgaggtattcgcactggatacgacacccac

SEQ ID NO: 368 - RNA, 50 nt, synthetic construct; identical to SEQ ID NO: 363
  source 1..50: mol_type = other RNA

SEQ ID NO: 369 - RNA, 50 nt, synthetic construct
  source 1..50: mol_type = other RNA
gtcgtatccagtgcagggtccgaggtattcgcactggatacgactttctc

SEQ ID NO: 370 - RNA, 50 nt, synthetic construct
  source 1..50: mol_type = other RNA
gtcgtatccagtgcagggtccgaggtattcgcactggatacgacaacccg

SEQ ID NO: 371 - RNA, 50 nt, synthetic construct
  source 1..50: mol_type = other RNA
gtcgtatccagtgcagggtccgaggtattcgcactggatacgacaaaccc

SEQ ID NO: 372 - RNA, 50 nt, synthetic construct
  source 1..50: mol_type = other RNA
gtcgtatccagtgcagggtccgaggtattcgcactggatacgacgttctc

SEQ ID NO: 373 - RNA, 50 nt, synthetic construct
  source 1..50: mol_type = other RNA
gtcgtatccagtgcagggtccgaggtattcgcactggatacgactgagag

SEQ ID NO: 374 - RNA, 50 nt, synthetic construct; identical to SEQ ID NO: 351
  source 1..50: mol_type = other RNA

SEQ ID NO: 375 - RNA, 50 nt, synthetic construct
  source 1..50: mol_type = other RNA
gtcgtatccagtgcagggtccgaggtattcgcactggatacgaccatcag

SEQ ID NO: 376 - RNA, 50 nt, synthetic construct
  source 1..50: mol_type = other RNA
gtcgtatccagtgcagggtccgaggtattcgcactggatacgaccccggg

SEQ ID NO: 377 - RNA, 50 nt, synthetic construct; identical to SEQ ID NO: 376
  source 1..50: mol_type = other RNA

SEQ ID NO: 378 - RNA, 50 nt, synthetic construct
  source 1..50: mol_type = other RNA
gtcgtatccagtgcagggtccgaggtattcgcactggatacgacaaacga

SEQ ID NO: 379 - RNA, 50 nt, synthetic construct
  source 1..50: mol_type = other RNA
gtcgtatccagtgcagggtccgaggtattcgcactggatacgacacccgg

SEQ ID NO: 380 - RNA, 50 nt, synthetic construct; identical to SEQ ID NO: 379
  source 1..50: mol_type = other RNA

SEQ ID NO: 381 - RNA, 50 nt, synthetic construct
  source 1..50: mol_type = other RNA
gtcgtatccagtgcagggtccgaggtattcgcactggatacgacggccgg

SEQ ID NO: 382 - RNA, 50 nt, synthetic construct
  source 1..50: mol_type = other RNA
gtcgtatccagtgcagggtccgaggtattcgcactggatacgacccccac

SEQ ID NO: 383 - RNA, 50 nt, synthetic construct; identical to SEQ ID NO: 379
  source 1..50: mol_type = other RNA

SEQ ID NO: 384 - RNA, 50 nt, synthetic construct
  source 1..50: mol_type = other RNA
gtcgtatccagtgcagggtccgaggtattcgcactggatacgactacccg

SEQ ID NO: 385 - RNA, 50 nt, synthetic construct; identical to SEQ ID NO: 379
  source 1..50: mol_type = other RNA

SEQ ID NO: 386 - RNA, 20 nt, synthetic construct
  source 1..20: mol_type = other RNA
aacccgtagatccgaacttg

SEQ ID NO: 387 - RNA, 20 nt, synthetic construct
  source 1..20: mol_type = other RNA
aagctcggtctgaggcccct

SEQ ID NO: 388 - RNA, 17 nt, synthetic construct
  source 1..17: mol_type = other RNA
aatcccggacgagcccc

SEQ ID NO: 389 - RNA, 20 nt, synthetic construct
  source 1..20: mol_type = other RNA
acctgtactgagctgccccg

SEQ ID NO: 390 - RNA, 20 nt, synthetic construct
  source 1..20: mol_type = other RNA
actcggcgtggcgtcggtcg

SEQ ID NO: 391 - RNA, 17 nt, synthetic construct
  source 1..17: mol_type = other RNA
atccccggcacctccac

SEQ ID NO: 392 - RNA, 20 nt, synthetic construct
  source 1..20: mol_type = other RNA
atctcccaacccttgtacca

SEQ ID NO: 393 - RNA, 20 nt, synthetic construct
  source 1..20: mol_type = other RNA
atgtcccacccccactcctg

SEQ ID NO: 394 - RNA, 20 nt, synthetic construct
  source 1..20: mol_type = other RNA
atgtcccacccccactcctg

SEQ ID NO: 395 - RNA, 20 nt, synthetic construct
  source 1..20: mol_type = other RNA
cgtcccaccccccactcctg

SEQ ID NO: 396 - RNA, 20 nt, synthetic construct
  source 1..20: mol_type = other RNA
ctatacgacctgctgccttt

SEQ ID NO: 397 - RNA, 20 nt, synthetic construct
  source 1..20: mol_type = other RNA
gacctcgccgtcccgcccgc

SEQ ID NO: 398 - RNA, 18 nt, synthetic construct
  source 1..18: mol_type = other RNA
gcacttgtctcggtctga

SEQ ID NO: 399 - RNA, 18 nt, synthetic construct
  source 1..18: mol_type = other RNA
gtagtaggttgtatagtt

SEQ ID NO: 400 - RNA, 20 nt, synthetic construct
  source 1..20: mol_type = other RNA
gtcaacacttgctggtttcc

SEQ ID NO: 401 - RNA, 20 nt, synthetic construct
  source 1..20: mol_type = other RNA
gtcatacacggctctcctct

SEQ ID NO: 402 - RNA, 20 nt, synthetic construct
  source 1..20: mol_type = other RNA
tattgcacttgtcccggcct

SEQ ID NO: 403 - RNA, 20 nt, synthetic construct
  source 1..20: mol_type = other RNA
tccccggcatctccaccaaa

SEQ ID NO: 404 - RNA, 20 nt, synthetic construct
  source 1..20: mol_type = other RNA
tccctgtcctccaggagctc

SEQ ID NO: 405 - RNA, 20 nt, synthetic construct
  source 1..20: mol_type = other RNA
tccctgtcctccaggagctc

SEQ ID NO: 406 - RNA, 20 nt, synthetic construct
  source 1..20: mol_type = other RNA
tccctgtcctccaggagctc

SEQ ID NO: 407 - RNA, 20 nt, synthetic construct
  source 1..20: mol_type = other RNA
tcctgtactgagctaccccg

SEQ ID NO: 408 - RNA, 20 nt, synthetic construct
  source 1..20: mol_type = other RNA
tcctgtactgagctgcccca

SEQ ID NO: 409 - RNA, 20 nt, synthetic construct
  source 1..20: mol_type = other RNA
tcctgtactgagctgccccg

SEQ ID NO: 410 - RNA, 20 nt, synthetic construct
  source 1..20: mol_type = other RNA
tctacagtgcacgtgtctcc

SEQ ID NO: 411 - RNA, 17 nt, synthetic construct
  source 1..17: mol_type = other RNA
tcttcgggggcgtgggt

SEQ ID NO: 412 - RNA, 20 nt, synthetic construct
  source 1..20: mol_type = other RNA
tcttgtactgagctgccccg

SEQ ID NO: 413 - RNA, 20 nt, synthetic construct
  source 1..20: mol_type = other RNA
tgaggggcagagagcgagaa

SEQ ID NO: 414 - RNA, 20 nt, synthetic construct
  source 1..20: mol_type = other RNA
tggcatgcacgaggccccgg

SEQ ID NO: 415 - RNA, 20 nt, synthetic construct
  source 1..20: mol_type = other RNA
tggcatgcacgaggccccgg

SEQ ID NO: 416 - RNA, 20 nt, synthetic construct
  source 1..20: mol_type = other RNA
tgggtttacgttgggagaac

SEQ ID NO: 417 - RNA, 20 nt, synthetic construct
  source 1..20: mol_type = other RNA
tggttaggattcggcgctct

SEQ ID NO: 418 - RNA, 20 nt, synthetic construct
  source 1..20: mol_type = other RNA
tgtcccacccccactcctgt

SEQ ID NO: 419 - RNA, 20 nt, synthetic construct
  source 1..20: mol_type = other RNA
tcaagtgatggagagcaata

SEQ ID NO: 420 - RNA, 20 nt, synthetic construct
  source 1..20: mol_type = other RNA
gcattggtggttcagtggta

SEQ ID NO: 421 - RNA, 20 nt, synthetic construct
  source 1..20: mol_type = other RNA
gcatgggtggttcagtggta

SEQ ID NO: 422 - RNA, 20 nt, synthetic construct
  source 1..20: mol_type = other RNA
gcattggtggttcagtggta

SEQ ID NO: 423 - RNA, 20 nt, synthetic construct
  source 1..20: mol_type = other RNA
ttggtggttcagtggtagaa

SEQ ID NO: 424 - RNA, 20 nt, synthetic construct
  source 1..20: mol_type = other RNA
gcattggtggttcagtggta

SEQ ID NO: 425 - RNA, 20 nt, synthetic construct
  source 1..20: mol_type = other RNA
gcattggtggttcagtggta

SEQ ID NO: 426 - RNA, 20 nt, synthetic construct
  source 1..20: mol_type = other RNA
gtgggggagaggctgtcgct

SEQ ID NO: 427 - RNA, 20 nt, synthetic construct
  source 1..20: mol_type = other RNA
gcattagtggttcagtggta

SEQ ID NO: 428 - RNA, 20 nt, synthetic construct
  source 1..20: mol_type = other RNA
gcattggtggttcagtggta

SEQ ID NO: 429 - RNA, 20 nt, synthetic construct
  source 1..20: mol_type = other RNA
gcattggtggtttagtggta

SEQ ID NO: 430 - RNA, 16 nt, synthetic construct
  source 1..16: mol_type = other RNA
gtgcagggtccgaggt

SEQ ID NO: 431 - RNA, 15 nt, synthetic construct
  source 1..15: mol_type = other RNA
cttgaacaccagcat

SEQ ID NO: 432 - RNA, 15 nt, synthetic construct
  source 1..15: mol_type = other RNA
tccggggagcagcat

SEQ ID NO: 433 - RNA, 15 nt, synthetic construct
  source 1..15: mol_type = other RNA
tgctcggggcagcat

SEQ ID NO: 434 - RNA, 15 nt, synthetic construct
  source 1..15: mol_type = other RNA
cggggctctcagcat

SEQ ID NO: 435 - RNA, 15 nt, synthetic construct
  source 1..15: mol_type = other RNA
gccagcacacagcat

SEQ ID NO: 436 - RNA, 15 nt, synthetic construct
  source 1..15: mol_type = other RNA
gtggaggtgcagcat

SEQ ID NO: 437 - RNA, 15 nt, synthetic construct
  source 1..15: mol_type = other RNA
catggtcaccagcat

SEQ ID NO: 438 - RNA, 15 nt, synthetic construct
  source 1..15: mol_type = other RNA
aggacaattcagcat

SEQ ID NO: 439 - RNA, 15 nt, synthetic construct
  source 1..15: mol_type = other RNA
gaggacaaacagcat

SEQ ID NO: 440 - RNA, 15 nt, synthetic construct
  source 1..15: mol_type = other RNA
tgaggacaacagcat

SEQ ID NO: 441 - RNA, 15 nt, synthetic construct
  source 1..15: mol_type = other RNA
acggaaagccagcat

SEQ ID NO: 442 - RNA, 15 nt, synthetic construct
  source 1..15: mol_type = other RNA
ggcgggcggcagcat

SEQ ID NO: 443 - RNA, 15 nt, synthetic construct
  source 1..15: mol_type = other RNA
agccagactcagcat

SEQ ID NO: 444 - RNA, 15 nt, synthetic construct
  source 1..15: mol_type = other RNA
acatatcaacagcat

SEQ ID NO: 445 - RNA, 15 nt, synthetic construct
  source 1..15: mol_type = other RNA
caaaggagacagcat

SEQ ID NO: 446 - RNA, 15 nt, synthetic construct
  source 1..15: mol_type = other RNA
gaggagagacagcat

SEQ ID NO: 447 - RNA, 15 nt, synthetic construct
  source 1..15: mol_type = other RNA
ccggacactcagcat

SEQ ID NO: 448 - RNA, 15 nt, synthetic construct
  source 1..15: mol_type = other RNA
ggtggttttcagcat

SEQ ID NO: 449 - RNA, 15 nt, synthetic construct
  source 1..15: mol_type = other RNA
ctcgagtgccagcat

SEQ ID NO: 450 - RNA, 15 nt, synthetic construct
  source 1..15: mol_type = other RNA
tcgagtgctcagcat

SEQ ID NO: 451 - RNA, 15 nt, synthetic construct
  source 1..15: mol_type = other RNA
tcctcgagtcagcat

SEQ ID NO: 452 - RNA, 15 nt, synthetic construct
  source 1..15: mol_type = other RNA
atggggctccagcat

SEQ ID NO: 453 - RNA, 15 nt, synthetic construct
  source 1..15: mol_type = other RNA
acggggtctcagcat

SEQ ID NO: 454 - RNA, 15 nt, synthetic construct
  source 1..15: mol_type = other RNA
gggctccggcagcat

SEQ ID NO: 455 - RNA, 15 nt, synthetic construct
  source 1..15: mol_type = other RNA
acagaggtccagcat

SEQ ID NO: 456 - RNA, 15 nt, synthetic construct
  source 1..15: mol_type = other RNA
ccgcacccacagcat

SEQ ID NO: 457 - RNA, 15 nt, synthetic construct
  source 1..15: mol_type = other RNA
acggggctccagcat

SEQ ID NO: 458 - RNA, 15 nt, synthetic construct
  source 1..15: mol_type = other RNA
tcgctctttcagcat

SEQ ID NO: 459 - RNA, 15 nt, synthetic construct
  source 1..15: mol_type = other RNA
ggggcccaacagcat

SEQ ID NO: 460 - RNA, 15 nt, synthetic construct
  source 1..15: mol_type = other RNA
gggcccaaacagcat

SEQ ID NO: 461 - RNA, 15 nt, synthetic construct
  source 1..15: mol_type = other RNA
accctcttgcagcat

SEQ ID NO: 462 - RNA, 15 nt, synthetic construct
  source 1..15: mol_type = other RNA
cgcgagagtcagcat

SEQ ID NO: 463 - RNA, 15 nt, synthetic construct
  source 1..15: mol_type = other RNA
tgaggacaacagcat

SEQ ID NO: 464 - RNA, 15 nt, synthetic construct
  source 1..15: mol_type = other RNA
acagactaccagcat

SEQ ID NO: 465 - RNA, 15 nt, synthetic construct
  source 1..15: mol_type = other RNA
tccgggccccagcat

SEQ ID NO: 466 - RNA, 15 nt, synthetic construct
  source 1..15: mol_type = other RNA
tccgggccccagcat

SEQ ID NO: 467 - RNA, 15 nt, synthetic construct
  source 1..15: mol_type = other RNA
ccaagcaaacagcat

SEQ ID NO: 468 - RNA, 15 nt, synthetic construct
  source 1..15: mol_type = other RNA
ccgggcccacagcat

SEQ ID NO: 469 - RNA, 15 nt, synthetic construct
  source 1..15: mol_type = other RNA
ctgggcccacagcat

SEQ ID NO: 470 - RNA, 15 nt, synthetic construct
  source 1..15: mol_type = other RNA
aagggccggcagcat

SEQ ID NO: 471 - RNA, 15 nt, synthetic construct
  source 1..15: mol_type = other RNA
ccgcacccccagcat

SEQ ID NO: 472 - RNA, 15 nt, synthetic construct
  source 1..15: mol_type = other RNA
ccgggcccacagcat

SEQ ID NO: 473 - RNA, 15 nt, synthetic construct
  source 1..15: mol_type = other RNA
cgggcccatcagcat

SEQ ID NO: 474 - RNA, 15 nt, synthetic construct
  source 1..15: mol_type = other RNA
ccgggcccacagcat

SEQ ID NO: 475 - RNA, 22 nt, synthetic construct
  source 1..22: mol_type = other RNA
cacaagttcggatctacgggtt

SEQ ID NO: 476 - RNA, 21 nt, synthetic construct
  source 1..21: mol_type = other RNA
gaggggcctcagaccgagctt

SEQ ID NO: 477 - RNA, 17 nt, synthetic construct
  source 1..17: mol_type = other RNA
ggggctcgtccgggatt

SEQ ID NO: 478 - RNA, 23 nt, synthetic construct
  source 1..23: mol_type = other RNA
tctcggggcagctcagtacaggt

SEQ ID NO: 479 - RNA, 23 nt, synthetic construct
  source 1..23: mol_type = other RNA
acacgaccgacgccacgccgagt

SEQ ID NO: 480 - RNA, 17 nt, synthetic construct
  source 1..17: mol_type = other RNA
gtggaggtgccggggat

SEQ ID NO: 481 - RNA, 23 nt, synthetic construct
  source 1..23: mol_type = other RNA
cactggtacaagggttgggagat

SEQ ID NO: 482 - RNA, 24 nt, synthetic construct
  source 1..24: mol_type = other RNA
ttaacaggagtgggggtgggacat

SEQ ID NO: 483 - RNA, 23 nt, synthetic construct
  source 1..23: mol_type = other RNA
aaacaggagtgggggtgggacat

SEQ ID NO: 484 - RNA, 22 nt, synthetic construct
  source 1..22: mol_type = other RNA
aacaggagtggggggtgggacg

SEQ ID NO: 485 - RNA, 22 nt, synthetic construct
  source 1..22: mol_type = other RNA
cgaaaggcagcaggtcgtatag

SEQ ID NO: 486 - RNA, 21 nt, synthetic construct
  source 1..21: mol_type = other RNA
ggcgggcgggacggcgaggtc

SEQ ID NO: 487 - RNA, 18 nt, synthetic construct
  source 1..18: mol_type = other RNA
tcagaccgagacaagtgc

SEQ ID NO: 488 - RNA, 18 nt, synthetic construct
  source 1..18: mol_type = other RNA
aactatacaacctactac

SEQ ID NO: 489 - RNA, 23 nt, synthetic construct
  source 1..23: mol_type = other RNA
agaggaaaccagcaagtgttgac

SEQ ID NO: 490 - RNA, 22 nt, synthetic construct
  source 1..22: mol_type = other RNA
agagaggagagccgtgtatgac

SEQ ID NO: 491 - RNA, 24 nt, synthetic construct
  source 1..24: mol_type = other RNA
tcacaggccgggacaagtgcaata

SEQ ID NO: 492 - RNA, 21 nt, synthetic construct
  source 1..21: mol_type = other RNA
ttttggtggagatgccgggga

SEQ ID NO: 493 - RNA, 23 nt, synthetic construct
  source 1..23: mol_type = other RNA
cgtgagctcctggaggacaggga

SEQ ID NO: 494 - RNA, 24 nt, synthetic construct
  source 1..24: mol_type = other RNA
tcgtgagctcctggaggacaggga

SEQ ID NO: 495 - RNA, 21 nt, synthetic construct
  source 1..21: mol_type = other RNA
tgagctcctggaggacaggga

SEQ ID NO: 496 - RNA, 22 nt, synthetic construct
  source 1..22: mol_type = other RNA
ctcggggtagctcagtacagga

SEQ ID NO: 497 - RNA, 22 nt, synthetic construct
  source 1..22: mol_type = other RNA
tctggggcagctcagtacagga

SEQ ID NO: 498 - RNA, 25 nt, synthetic construct
  source 1..25: mol_type = other RNA
ggcctcggggcagctcagtacagga

SEQ ID NO: 499 - RNA, 22 nt, synthetic construct
  source 1..22: mol_type = other RNA
ctggagacacgtgcactgtaga

SEQ ID NO: 500 - RNA, 17 nt, synthetic construct
  source 1..17: mol_type = other RNA
acccacgcccccgaaga

SEQ ID NO: 501 - RNA, 22 nt, synthetic construct
  source 1..22: mol_type = other RNA
ctcggggcagctcagtacaaga

SEQ ID NO: 502 - RNA, 21 nt, synthetic construct
  source 1..21: mol_type = other RNA
tttctcgctctctgcccctca

SEQ ID NO: 503 - RNA, 23 nt, synthetic construct
  source 1..23: mol_type = other RNA
aacccggggcctcgtgcatgcca

SEQ ID NO: 504 - RNA, 24 nt, synthetic construct
  source 1..24: mol_type = other RNA
aaacccggggcctcgtgcatgcca

SEQ ID NO: 505 - RNA, 20 nt, synthetic construct
  source 1..20: mol_type = other RNA
gttctcccaacgtaaaccca

SEQ ID NO: 506 - RNA, 22 nt, synthetic construct
  source 1..22: mol_type = other RNA
tgagagcgccgaatcctaacca

SEQ ID NO: 507 - RNA, 21 nt, synthetic construct
  source 1..21: mol_type = other RNA
aacaggagtgggggtgggaca

SEQ ID NO: 508 - RNA, 68 nt, synthetic construct
  source 1..68: mol_type = other RNA
catcagacagtacgaatacatcccaatgtggtcacagtcatccccgggtattgctctccatcacttga

SEQ ID NO: 509 - RNA, 51 nt, synthetic construct
  source 1..51: mol_type = other RNA
cccgggcctcccgcgtggcaggcgagaattctaccactgaaccaccaatgc

SEQ ID NO: 510 - RNA, 51 nt, synthetic construct
  source 1..51: mol_type = other RNA
cccgggcctcccgcgtggcaggcgagaattctaccactgaaccacccatgc

SEQ ID NO: 511 - RNA, 58 nt, synthetic construct
  source 1..58: mol_type = other RNA
aaacgaacccgggcctcccgcgtggcaggcgagaattctaccactgaaccaccaatgc

SEQ ID NO: 512 - RNA, 49 nt, synthetic construct
  source 1..49: mol_type = other RNA
acccgggcctcccgcgtggcaggcgagaattctaccactgaaccaccaa

SEQ ID NO: 513 - RNA, 52 nt, synthetic construct
  source 1..52: mol_type = other RNA
acccgggtctcccgcgtggcaggcgagaattctaccactgaaccaccaatgc

SEQ ID NO: 514 - RNA, 65 nt, synthetic construct
  source 1..65: mol_type = other RNA
ggccgggaaacgaacccgggcctcccgcgtggcaggcgagaattctaccactgaaccaccaatgc

SEQ ID NO: 515 - RNA, 49 nt, synthetic construct
  source 1..49: mol_type = other RNA
ccccacgcccgccgcgggcccagaagcgcagcgacagcctctcccccac

SEQ ID NO: 516 - RNA, 52 nt, synthetic construct
  source 1..52: mol_type = other RNA
acccgggcctcccgcgtggcaggcgagaattctaccactgaaccactaatgc

SEQ ID NO: 517 - RNA, 53 nt, synthetic construct
  source 1..53: mol_type = other RNA
tacccgggcctcccgcgtggcaggcgagaattctaccactgaaccaccaatgc

SEQ ID NO: 518 - RNA, 52 nt, synthetic construct
  source 1..52: mol_type = other RNA
acccgggcctcccgcgtggcaggcgagaattctaccactaaaccaccaatgc

SEQ ID NO: 519 - RNA, 50 nt, synthetic construct
  source 1..50: mol_type = other RNA
gtcgtatccagtgcagggtccgaggtattcgcactggatacgactatagg

SEQ ID NO: 520 - RNA, 50 nt, synthetic construct
  source 1..50: mol_type = other RNA
gtcgtatccagtgcagggtccgaggtattcgcactggatacgacacaggc

SEQ ID NO: 521 - RNA, 50 nt, synthetic construct
  source 1..50: mol_type = other RNA
gtcgtatccagtgcagggtccgaggtattcgcactggatacgacagtgaa

SEQ ID NO: 522 - RNA, 50 nt, synthetic construct
  source 1..50: mol_type = other RNA
gtcgtatccagtgcagggtccgaggtattcgcactggatacgacacagtg

SEQ ID NO: 523 - RNA, 50 nt, synthetic construct
  source 1..50: mol_type = other RNA
gtcgtatccagtgcagggtccgaggtattcgcactggatacgactacagg

SEQ ID NO: 524 - RNA, 50 nt, synthetic construct
  source 1..50: mol_type = other RNA
gtcgtatccagtgcagggtccgaggtattcgcactggatacgaccaaaag

SEQ ID NO: 525 - RNA, 50 nt, synthetic construct; identical to SEQ ID NO: 520
  source 1..50: mol_type = other RNA

SEQ ID NO: 526 - RNA, 50 nt, synthetic construct
  source 1..50: mol_type = other RNA
gtcgtatccagtgcagggtccgaggtattcgcactggatacgactctacc

SEQ ID NO: 527 - RNA, 50 nt, synthetic construct
  source 1..50: mol_type = other RNA
gtcgtatccagtgcagggtccgaggtattcgcactggatacgacaacatc

SEQ ID NO: 528 - RNA, 50 nt, synthetic construct; identical to SEQ ID NO: 349
  source 1..50: mol_type = other RNA

SEQ ID NO: 529 - RNA, 50 nt, synthetic construct; identical to SEQ ID NO: 520
  source 1..50: mol_type = other RNA

SEQ ID NO: 530 - RNA, 50 nt, synthetic construct
  source 1..50: mol_type = other RNA
gtcgtatccagtgcagggtccgaggtattcgcactggatacgacattggg

SEQ ID NO: 531 - RNA, 50 nt, synthetic construct
  source 1..50: mol_type = other RNA
gtcgtatccagtgcagggtccgaggtattcgcactggatacgacataact

SEQ ID NO: 532 - RNA, 50 nt, synthetic construct
  source 1..50: mol_type = other RNA
gtcgtatccagtgcagggtccgaggtattcgcactggatacgacaactca

SEQ ID NO: 533 - RNA, 50 nt, synthetic construct
  source 1..50: mol_type = other RNA
gtcgtatccagtgcagggtccgaggtattcgcactggatacgacctaggg

SEQ ID NO: 534 - RNA, 50 nt, synthetic construct
  source 1..50: mol_type = other RNA
gtcgtatccagtgcagggtccgaggtattcgcactggatacgacaatcct

SEQ ID NO: 535 - RNA, 50 nt, synthetic construct
  source 1..50: mol_type = other RNA
gtcgtatccagtgcagggtccgaggtattcgcactggatacgactaaacc

SEQ ID NO: 536 - RNA, 50 nt, synthetic construct
  source 1..50: mol_type = other RNA
gtcgtatccagtgcagggtccgaggtattcgcactggatacgacttcaca

SEQ ID NO: 537 - RNA, 50 nt, synthetic construct
  source 1..50: mol_type = other RNA
gtcgtatccagtgcagggtccgaggtattcgcactggatacgactccctc

SEQ ID NO: 538 - RNA, 50 nt, synthetic construct
  source 1..50: mol_type = other RNA
gtcgtatccagtgcagggtccgaggtattcgcactggatacgactcaggc

SEQ ID NO: 539 - RNA, 50 nt, synthetic construct
  source 1..50: mol_type = other RNA
gtcgtatccagtgcagggtccgaggtattcgcactggatacgacctcaga

SEQ ID NO: 540 - RNA, 50 nt, synthetic construct
  source 1..50: mol_type = other RNA
gtcgtatccagtgcagggtccgaggtattcgcactggatacgaccaggcc

SEQ ID NO: 541 - RNA, 50 nt, synthetic construct; identical to SEQ ID NO: 363
  source 1..50: mol_type = other RNA

SEQ ID NO: 542 - RNA, 50 nt, synthetic construct
  source 1..50: mol_type = other RNA
gtcgtatccagtgcagggtccgaggtattcgcactggatacgacaaactc

SEQ ID NO: 543 - RNA, 50 nt, synthetic construct
  source 1..50: mol_type = other RNA
gtcgtatccagtgcagggtccgaggtattcgcactggatacgacactggg

SEQ ID NO: 544 - RNA, 50 nt, synthetic construct
  source 1..50: mol_type = other RNA
gtcgtatccagtgcagggtccgaggtattcgcactggatacgacacagga

SEQ ID NO: 545 - RNA, 50 nt, synthetic construct; identical to SEQ ID NO: 359
  source 1..50: mol_type = other RNA

SEQ ID NO: 546 - RNA, 50 nt, synthetic construct
  source 1..50: mol_type = other RNA
gtcgtatccagtgcagggtccgaggtattcgcactggatacgacagcaag

SEQ ID NO: 547 - RNA, 50 nt, synthetic construct
  source 1..50: mol_type = other RNA
gtcgtatccagtgcagggtccgaggtattcgcactggatacgactccagt

SEQ ID NO: 548 - RNA, 50 nt, synthetic construct
  source 1..50: mol_type = other RNA
gtcgtatccagtgcagggtccgaggtattcgcactggatacgaccttcct

SEQ ID NO: 549 - RNA, 50 nt, synthetic construct; identical to SEQ ID NO: 520
  source 1..50: mol_type = other RNA

SEQ ID NO: 550 - RNA, 50 nt, synthetic construct
  source 1..50: mol_type = other RNA
gtcgtatccagtgcagggtccgaggtattcgcactggatacgacagacaa

SEQ ID NO: 551 - RNA, 50 nt, synthetic construct; identical to SEQ ID NO: 523
  source 1..50: mol_type = other RNA

SEQ ID NO: 552 - RNA, 50 nt, synthetic construct
  source 1..50: mol_type = other RNA
gtcgtatccagtgcagggtccgaggtattcgcactggatacgacacaaca

SEQ ID NO: 553 - RNA, 50 nt, synthetic construct
  source 1..50: mol_type = other RNA
gtcgtatccagtgcagggtccgaggtattcgcactggatacgacaactgg

SEQ ID NO: 554 - RNA, 50 nt, synthetic construct
  source 1..50: mol_type = other RNA
gtcgtatccagtgcagggtccgaggtattcgcactggatacgacactcgg

SEQ ID NO: 555 - RNA, 50 nt, synthetic construct
  source 1..50: mol_type = other RNA
gtcgtatccagtgcagggtccgaggtattcgcactggatacgacctcacc

SEQ ID NO: 556 - RNA, 50 nt, synthetic construct
  source 1..50: mol_type = other RNA
gtcgtatccagtgcagggtccgaggtattcgcactggatacgactctatc

SEQ ID NO: 557 - RNA, 50 nt, synthetic construct
  source 1..50: mol_type = other RNA
gtcgtatccagtgcagggtccgaggtattcgcactggatacgacgaactc

SEQ ID NO: 558 - RNA, 50 nt, synthetic construct
  source 1..50: mol_type = other RNA
gtcgtatccagtgcagggtccgaggtattcgcactggatacgacaaacgg

SEQ ID NO: 559 - RNA, 50 nt, synthetic construct
  source 1..50: mol_type = other RNA
gtcgtatccagtgcagggtccgaggtattcgcactggatacgactacgag

SEQ ID NO: 560 - RNA, 50 nt, synthetic construct
  source 1..50: mol_type = other RNA
gtcgtatccagtgcagggtccgaggtattcgcactggatacgacctcggc

SEQ ID NO: 561 - RNA, 50 nt, synthetic construct
  source 1..50: mol_type = other RNA
gtcgtatccagtgcagggtccgaggtattcgcactggatacgactgaatt

SEQ ID NO: 562 - RNA, 50 nt, synthetic construct; identical to SEQ ID NO: 523
  source 1..50: mol_type = other RNA

SEQ ID NO: 563 - RNA, 50 nt, synthetic construct; identical to SEQ ID NO: 520
  source 1..50: mol_type = other RNA

SEQ ID NO: 564 - RNA, 50 nt, synthetic construct; identical to SEQ ID NO: 523
  source 1..50: mol_type = other RNA

SEQ ID NO: 565 - RNA, 50 nt, synthetic construct
  source 1..50: mol_type = other RNA
gtcgtatccagtgcagggtccgaggtattcgcactggatacgacaatcag

SEQ ID NO: 566 - RNA, 50 nt, synthetic construct; identical to SEQ ID NO: 540
  source 1..50: mol_type = other RNA

SEQ ID NO: 567 - RNA, 50 nt, synthetic construct; identical to SEQ ID NO: 554
  source 1..50: mol_type = other RNA

SEQ ID NO: 568 - RNA, 50 nt, synthetic construct
  source 1..50: mol_type = other RNA
gtcgtatccagtgcagggtccgaggtattcgcactggatacgacctcctc

SEQ ID NO: 569 - RNA, 50 nt, synthetic construct; identical to SEQ ID NO: 520
  source 1..50: mol_type = other RNA

SEQ ID NO: 570 - RNA, 50 nt, synthetic construct
  source 1..50: mol_type = other RNA
gtcgtatccagtgcagggtccgaggtattcgcactggatacgacttctcg

SEQ ID NO: 571 - RNA, 50 nt, synthetic construct
  source 1..50: mol_type = other RNA
gtcgtatccagtgcagggtccgaggtattcgcactggatacgacactacc

SEQ ID NO: 572 - RNA, 50 nt, synthetic construct; identical to SEQ ID NO: 520
  source 1..50: mol_type = other RNA

SEQ ID NO: 573 - RNA, 50 nt, synthetic construct
  source 1..50: mol_type = other RNA
gtcgtatccagtgcagggtccgaggtattcgcactggatacgactgacag

SEQ ID NO: 574 - RNA, 50 nt, synthetic construct
  source 1..50: mol_type = other RNA
gtcgtatccagtgcagggtccgaggtattcgcactggatacgacaccggg

SEQ ID NO: 575 - RNA, 50 nt, synthetic construct
  source 1..50: mol_type = other RNA
gtcgtatccagtgcagggtccgaggtattcgcactggatacgaccgtaca

SEQ ID NO: 576 - RNA, 50 nt, synthetic construct; identical to SEQ ID NO: 523
  source 1..50: mol_type = other RNA

SEQ ID NO: 577 - RNA, 50 nt, synthetic construct
  source 1..50: mol_type = other RNA
gtcgtatccagtgcagggtccgaggtattcgcactggatacgactcactc

SEQ ID NO: 578 - RNA, 50 nt, synthetic construct
  source 1..50: mol_type = other RNA
gtcgtatccagtgcagggtccgaggtattcgcactggatacgaccttcgg

SEQ ID NO: 579 - RNA, 50 nt, synthetic construct
  source 1..50: mol_type = other RNA
gtcgtatccagtgcagggtccgaggtattcgcactggatacgacgaaggg

SEQ ID NO: 580 - RNA, 50 nt, synthetic construct
  source 1..50: mol_type = other RNA
gtcgtatccagtgcagggtccgaggtattcgcactggatacgacttctac

SEQ ID NO: 581 - RNA, 50 nt, synthetic construct
  source 1..50: mol_type = other RNA
gtcgtatccagtgcagggtccgaggtattcgcactggatacgacagcttc

SEQ ID NO: 582 - RNA, 50 nt, synthetic construct
  source 1..50: mol_type = other RNA
gtcgtatccagtgcagggtccgaggtattcgcactggatacgacatatgg

SEQ ID NO: 583 - RNA, 50 nt, synthetic construct
  source 1..50: mol_type = other RNA
gtcgtatccagtgcagggtccgaggtattcgcactggatacgacatcagc

SEQ ID NO: 584 - RNA, 50 nt, synthetic construct
  source 1..50: mol_type = other RNA
gtcgtatccagtgcagggtccgaggtattcgcactggatacgacaacggg

SEQ ID NO: 585 - RNA, 50 nt, synthetic construct; identical to SEQ ID NO: 543
  source 1..50: mol_type = other RNA

SEQ ID NO: 586 - RNA, 50 nt, synthetic construct; identical to SEQ ID NO: 523
  source 1..50: mol_type = other RNA

SEQ ID NO: 587 - RNA, 50 nt, synthetic construct
  source 1..50: mol_type = other RNA
gtcgtatccagtgcagggtccgaggtattcgcactggatacgaccagtgt

SEQ ID NO: 588 - RNA, 50 nt, synthetic construct
  source 1..50: mol_type = other RNA
gtcgtatccagtgcagggtccgaggtattcgcactggatacgactactaa

SEQ ID NO: 589 - RNA, 50 nt, synthetic construct
  source 1..50: mol_type = other RNA
gtcgtatccagtgcagggtccgaggtattcgcactggatacgactctaaa

SEQ ID NO: 590 - RNA, 20 nt, synthetic construct
  source 1..20: mol_type = other RNA
tattgcacttgtcccggcct

SEQ ID NO: 591 - RNA, 20 nt, synthetic construct
  source 1..20: mol_type = other RNA
tattgcacttgtcccggcct

SEQ ID NO: 592 - RNA, 20 nt, synthetic construct
  source 1..20: mol_type = other RNA
tatggcactggtagaattca

SEQ ID NO: 593 - RNA, 20 nt, synthetic construct
  source 1..20: mol_type = other RNA
atggcactggtagaattcac

SEQ ID NO: 594 - RNA, 20 nt, synthetic construct
  source 1..20: mol_type = other RNA
aatattgcacttgtcccggc

SEQ ID NO: 595 - RNA, 20 nt, synthetic construct
  source 1..20: mol_type = other RNA
ccaaaactgcagttactttt

SEQ ID NO: 596 - RNA, 20 nt, synthetic construct
  source 1..20: mol_type = other RNA
tattacacttgtcccggcct

SEQ ID NO: 597 - RNA, 20 nt, synthetic construct
  source 1..20: mol_type = other RNA
caaagtgctcatagtgcagg

SEQ ID NO: 598 - RNA, 20 nt, synthetic construct
  source 1..20: mol_type = other RNA
gggagccaggaagtattgat

SEQ ID NO: 599 - RNA, 20 nt, synthetic construct
  source 1..20: mol_type = other RNA
tattgcacttgtcccggcct

SEQ ID NO: 600 - RNA, 20 nt, synthetic construct
  source 1..20: mol_type = other RNA
gattgcacttgtcccggcct

SEQ ID NO: 601 - RNA, 20 nt, synthetic construct
  source 1..20: mol_type = other RNA
gtgtcagtttgtcaaatacc

SEQ ID NO: 602 - RNA, 20 nt, synthetic construct
  source 1..20: mol_type = other RNA
agctcggtctgaggcccctc

SEQ ID NO: 603 - RNA, 20 nt, synthetic construct
  source 1..20: mol_type = other RNA
acaaaccgttaccattactg

SEQ ID NO: 604 - RNA, 20 nt, synthetic construct
  source 1..20: mol_type = other RNA
cctgtactgagctgccccta

SEQ ID NO: 605 - RNA, 20 nt, synthetic construct
  source 1..20: mol_type = other RNA
aatgcacccgggcaaggatt

SEQ ID NO: 606 - RNA, 20 nt, synthetic construct
  source 1..20: mol_type = other RNA
ttagcagcacatcatggttt

SEQ ID NO: 607 - RNA, 20 nt, synthetic construct
  source 1..20: mol_type = other RNA
aatggcgccactagggttgt

SEQ ID NO: 608 - RNA, 20 nt, synthetic construct
  source 1..20: mol_type = other RNA
ttctggaattctgtgtgagg

SEQ ID NO: 609 - RNA, 20 nt, synthetic construct
  source 1..20: mol_type = other RNA
atattgcacttgtcccggcc

SEQ ID NO: 610 - RNA, 20 nt, synthetic construct
  source 1..20: mol_type = other RNA
aaactctacttgtccttctg

SEQ ID NO: 611 - RNA, 20 nt, synthetic construct
  source 1..20: mol_type = other RNA
ttattgcacttgtcccggcc

SEQ ID NO: 612 - RNA, 20 nt, synthetic construct
  source 1..20: mol_type = other RNA
ctcgtcctgtactgagctgc

SEQ ID NO: 613 - RNA, 20 nt, synthetic construct
  source 1..20: mol_type = other RNA
cctgtactgagctgccccga

SEQ ID NO: 614 - RNA, 20 nt, synthetic construct
  source 1..20: mol_type = other RNA
gaactcaccctctgctccca

SEQ ID NO: 615 - RNA, 20 nt, synthetic construct
  source 1..20: mol_type = other RNA
tggagagaaaggcagttcct

SEQ ID NO: 616 - RNA, 20 nt, synthetic construct
  source 1..20: mol_type = other RNA
tattgcacttgtcccggcca

SEQ ID NO: 617 - RNA, 20 nt, synthetic construct
  source 1..20: mol_type = other RNA
ttttagagacggggtcttgc

SEQ ID NO: 618 - RNA, 20 nt, synthetic construct
  source 1..20: mol_type = other RNA
tttggcaatggtagaactca

SEQ ID NO: 619 - RNA, 20 nt, synthetic construct
  source 1..20: mol_type = other RNA
tgtagagcagggagcaggaa

SEQ ID NO: 620 - RNA, 20 nt, synthetic construct
  source 1..20: mol_type = other RNA
gtattgcacttgtcccggcc

SEQ ID NO: 621 - RNA, 20 nt, synthetic construct
  source 1..20: mol_type = other RNA
tgtgaggttggcattgttgt

SEQ ID NO: 622 - RNA, 20 nt, synthetic construct
  source 1..20: mol_type = other RNA
gtattgcacttgtcccggcc

SEQ ID NO: 623 - RNA, 20 nt, synthetic construct
  source 1..20: mol_type = other RNA
tgtgaggttggcattgttgt

SEQ ID NO: 624 - RNA, 20 nt, synthetic construct
  source 1..20: mol_type = other RNA
tccactctcctggcccccag

SEQ ID NO: 625 - RNA, 20 nt, synthetic construct
  source 1..20: mol_type = other RNA
tcctgtactgatctgccccg

SEQ ID NO: 626 - RNA, 20 nt, synthetic construct
  source 1..20: mol_type = other RNA
aaacattcaacgctgtcggt

SEQ ID NO: 627 - RNA, 20 nt, synthetic construct
  source 1..20: mol_type = other RNA
taaagtgctgacagtgcaga

SEQ ID NO: 628 - RNA, 20 nt, synthetic construct
  source 1..20: mol_type = other RNA
tcctgtactgagctgccccg

SEQ ID NO: 629 - RNA, 20 nt, synthetic construct
  source 1..20: mol_type = other RNA
tcctgtactgagctgccccg

SEQ ID NO: 630 - RNA, 20 nt, synthetic construct
  source 1..20: mol_type = other RNA
tctctcggctcctcgcggct

SEQ ID NO: 631 - RNA, 20 nt, synthetic construct
  source 1..20: mol_type = other RNA
cctgtactgagctgcgccga

SEQ ID NO: 632 - RNA, 19 nt, synthetic construct
  source 1..19: mol_type = other RNA
atggcactggtagaattca

SEQ ID NO: 633 - RNA, 20 nt, synthetic construct
  source 1..20: mol_type = other RNA
atattgcacttgtcccggcc

SEQ ID NO: 634 - RNA, 20 nt, synthetic construct
  source 1..20: mol_type = other RNA
atattgcacttgtcccggcc

SEQ ID NO: 635 - RNA, 20 nt, synthetic construct
  source 1..20: mol_type = other RNA
tattgcacttgtctcggcct

SEQ ID NO: 636 - RNA, 20 nt, synthetic construct
  source 1..20: mol_type = other RNA
tattgcacttgtcccggcct

SEQ ID NO: 637 - RNA, 20 nt, synthetic construct
  source 1..20: mol_type = other RNA
atattgcacttgtcccggcc

SEQ ID NO: 638 - RNA, 20 nt, synthetic construct
  source 1..20: mol_type = other RNA
tgtcctgtactgagctgccc

SEQ ID NO: 639 - RNA, 20 nt, synthetic construct
  source 1..20: mol_type = other RNA
tgtgtccgggaagtggagga

SEQ ID NO: 640 - RNA, 20 nt, synthetic construct
  source 1..20: mol_type = other RNA
ttattgcacttgtcccggcc

SEQ ID NO: 641 - RNA, 20 nt, synthetic construct
  source 1..20: mol_type = other RNA
ctcctgtactgagctgcccc

SEQ ID NO: 642 - RNA, 20 nt, synthetic construct
  source 1..20: mol_type = other RNA
aaagtgctgttcgtgcaggt

SEQ ID NO: 643 - RNA, 20 nt, synthetic construct
  source 1..20: mol_type = other RNA
tattgcacttatcccggcct

SEQ ID NO: 644 - RNA, 20 nt, synthetic construct
  source 1..20: mol_type = other RNA
tattgcacttgtcccggcct

SEQ ID NO: 645 - RNA, 20 nt, synthetic construct
  source 1..20: mol_type = other RNA
tcaggctcagtcccctcccg

SEQ ID NO: 646 - RNA, 20 nt, synthetic construct
  source 1..20: mol_type = other RNA
tattgcacttgtcccggcct

SEQ ID NO: 647 - RNA, 20 nt, synthetic construct
  source 1..20: mol_type = other RNA
ctattgcacttgtcccggcc

SEQ ID NO: 648 - RNA, 20 nt, synthetic construct
  source 1..20: mol_type = other RNA
cctgtactgagctgccccga

SEQ ID NO: 649 - RNA, 20 nt, synthetic construct
  source 1..20: mol_type = other RNA
ctgtactgagctgccccgaa

SEQ ID NO: 650 - RNA, 20 nt, synthetic construct
  source 1..20: mol_type = other RNA
tactgccctaaatgcccctt

SEQ ID NO: 651 - RNA, 20 nt, synthetic construct
  source 1..20: mol_type = other RNA
caaagtgcttacagtgcagg

SEQ ID NO: 652 - RNA, 20 nt, synthetic construct
  source 1..20: mol_type = other RNA
tgtagagcagggagcaggaa

SEQ ID NO: 653 - RNA, 20 nt, synthetic construct
  source 1..20: mol_type = other RNA
tgtcagtttgtcaaataccc

SEQ ID NO: 654 - RNA, 20 nt, synthetic construct
  source 1..20: mol_type = other RNA
ccgcactgtgggtacttgct

SEQ ID NO: 655 - RNA, 20 nt, synthetic construct
  source 1..20: mol_type = other RNA
atcctgtactgagctgcccc

SEQ ID NO: 656 - RNA, 20 nt, synthetic construct
  source 1..20: mol_type = other RNA
tcaccctgcatcccgcaccc

SEQ ID NO: 657 - RNA, 20 nt, synthetic construct
  source 1..20: mol_type = other RNA
cattgcacttgtcccggcct

SEQ ID NO: 658 - RNA, 20 nt, synthetic construct
  source 1..20: mol_type = other RNA
ttggcaatggtagaactcac

SEQ ID NO: 659 - RNA, 20 nt, synthetic construct
  source 1..20: mol_type = other RNA
aaaccgttaccattactgag

SEQ ID NO: 660 - RNA, 20 nt, synthetic construct
  source 1..20: mol_type = other RNA
aaccgttaccattactgagt

SEQ ID NO: 661 - RNA, 16 nt, synthetic construct
  source 1..16: mol_type = other RNA
gtgcagggtccgaggt

SEQ ID NO: 662 - RNA, 15 nt, synthetic construct
  source 1..15: mol_type = other RNA
gccggatatcagcat

SEQ ID NO: 663 - RNA, 15 nt, synthetic construct
  source 1..15: mol_type = other RNA
ggccggacacagcat

SEQ ID NO: 664 - RNA, 15 nt, synthetic construct
  source 1..15: mol_type = other RNA
cttaagtgacagcat

SEQ ID NO: 665 - RNA, 15 nt, synthetic construct
  source 1..15: mol_type = other RNA
taagtgacacagcat

SEQ ID NO: 666 - RNA, 15 nt, synthetic construct
  source 1..15: mol_type = other RNA
gccggacatcagcat

SEQ ID NO: 667 - RNA, 15 nt, synthetic construct
  source 1..15: mol_type = other RNA
aatgaaaaccagcat

SEQ ID NO: 668 - RNA, 15 nt, synthetic construct
  source 1..15: mol_type = other RNA
ggccggacacagcat

SEQ ID NO: 669 - RNA, 15 nt, synthetic construct
  source 1..15: mol_type = other RNA
cgtccatctcagcat

SEQ ID NO: 670 - RNA, 15 nt, synthetic construct
  source 1..15: mol_type = other RNA
taactacaacagcat

SEQ ID NO: 671 - RNA, 15 nt, synthetic construct
  source 1..15: mol_type = other RNA
cggacaattcagcat

SEQ ID NO: 672 - RNA, 15 nt, synthetic construct
  source 1..15: mol_type = other RNA
ggccggacacagcat

SEQ ID NO: 673 - RNA, 15 nt, synthetic construct
  source 1..15: mol_type = other RNA
atggggttacagcat

SEQ ID NO: 674 - RNA, 15 nt, synthetic construct
  source 1..15: mol_type = other RNA
gagtcaatacagcat

SEQ ID NO: 675 - RNA, 15 nt, synthetic construct
  source 1..15: mol_type = other RNA
atgactcaacagcat

SEQ ID NO: 676 - RNA, 15 nt, synthetic construct
  source 1..15: mol_type = other RNA
acggggatccagcat

SEQ ID NO: 677 - RNA, 15 nt, synthetic construct
  source 1..15: mol_type = other RNA
cgttcctaacagcat

SEQ ID NO: 678 - RNA, 15 nt, synthetic construct
  source 1..15: mol_type = other RNA
gtaccaaatcagcat

SEQ ID NO: 679 - RNA, 15 nt, synthetic construct
  source 1..15: mol_type = other RNA
ccaacacttcagcat

SEQ ID NO: 680 - RNA, 15 nt, synthetic construct
  source 1..15: mol_type = other RNA
acactccctcagcat

SEQ ID NO: 681 - RNA, 15 nt, synthetic construct
  source 1..15: mol_type = other RNA
ggccggactcagcat

SEQ ID NO: 682 - RNA, 15 nt, synthetic construct
  source 1..15: mol_type = other RNA
ggaagactccagcat

SEQ ID NO: 683 - RNA, 15 nt, synthetic construct
  source 1..15: mol_type = other RNA
gggccggaccagcat

SEQ ID NO: 684 - RNA, 15 nt, synthetic construct
  source 1..15: mol_type = other RNA
acggggctccagcat

SEQ ID NO: 685 - RNA, 15 nt, synthetic construct
  source 1..15: mol_type = other RNA
gggctcaaacagcat

SEQ ID NO: 686 - RNA, 15 nt, synthetic construct
  source 1..15: mol_type = other RNA
cgagggtcacagcat

SEQ ID NO: 687 - RNA, 15 nt, synthetic construct
  source 1..15: mol_type = other RNA
tcaaggacacagcat

SEQ ID NO: 688 - RNA, 15 nt, synthetic construct
  source 1..15: mol_type = other RNA
gccggttttcagcat

SEQ ID NO: 689 - RNA, 15 nt, synthetic construct
  source 1..15: mol_type = other RNA
ccagaacgacagcat

SEQ ID NO: 690 - RNA, 15 nt, synthetic construct
  source 1..15: mol_type = other RNA
gtgtgacctcagcat

SEQ ID NO: 691 - RNA, 15 nt, synthetic construct
  source 1..15: mol_type = other RNA
tcgtccttccagcat

SEQ ID NO: 692 - RNA, 15 nt, synthetic construct
  source 1..15: mol_type = other RNA
ggccggacacagcat

SEQ ID NO: 693 - RNA, 15 nt, synthetic construct
  source 1..15: mol_type = other RNA
aacaacagacagcat

SEQ ID NO: 694 - RNA, 15 nt, synthetic construct
  source 1..15: mol_type = other RNA
gccggacatcagcat

SEQ ID NO: 695 - RNA, 15 nt, synthetic construct
  source 1..15: mol_type = other RNA
gtaacaacacagcat

SEQ ID NO: 696 - RNA, 15 nt, synthetic construct
  source 1..15: mol_type = other RNA
gggggtcaacagcat

SEQ ID NO: 697 - RNA, 15 nt, synthetic construct
  source 1..15: mol_type = other RNA
cggggctcacagcat

SEQ ID NO: 698 - RNA, 15 nt, synthetic construct
  source 1..15: mol_type = other RNA
cagccactccagcat

SEQ ID NO: 699 - RNA, 15 nt, synthetic construct
  source 1..15: mol_type = other RNA
cgtctatctcagcat

SEQ ID NO: 700 - RNA, 15 nt, synthetic construct
  source 1..15: mol_type = other RNA
gggctcaagcagcat

SEQ ID NO: 701 - RNA, 15 nt, synthetic construct
  source 1..15: mol_type = other RNA
cggggcaaacagcat

SEQ ID NO: 702 - RNA, 15 nt, synthetic construct
  source 1..15: mol_type = other RNA
gccgagcatcagcat

SEQ ID NO: 703 - RNA, 15 nt, synthetic construct
  source 1..15: mol_type = other RNA
acgcggctccagcat

SEQ ID NO: 704 - RNA, 15 nt, synthetic construct
  source 1..15: mol_type = other RNA
atcttaagtcagcat

SEQ ID NO: 705 - RNA, 15 nt, synthetic construct
  source 1..15: mol_type = other RNA
gccggacatcagcat

SEQ ID NO: 706 - RNA, 15 nt, synthetic construct
  source 1..15: mol_type = other RNA
ggccggacacagcat

SEQ ID NO: 707 - RNA, 15 nt, synthetic construct
  source 1..15: mol_type = other RNA
gccggacatcagcat

SEQ ID NO: 708 - RNA, 15 nt, synthetic construct
  source 1..15: mol_type = other RNA
ccggactaacagcat

SEQ ID NO: 709 - RNA, 15 nt, synthetic construct
  source 1..15: mol_type = other RNA
gggccggaccagcat

SEQ ID NO: 710 - RNA, 15 nt, synthetic construct
  source 1..15: mol_type = other RNA
cggggctcacagcat

SEQ ID NO: 711 - RNA, 15 nt, synthetic construct
  source 1..15: mol_type = other RNA
cacctcctccagcat

SEQ ID NO: 712 - RNA, 15 nt, synthetic construct
  source 1..15: mol_type = other RNA
ggccggacacagcat

SEQ ID NO: 713 - RNA, 15 nt, synthetic construct
  source 1..15: mol_type = other RNA
ggggctcttcagcat

SEQ ID NO: 714 - RNA, 15 nt, synthetic construct
  source 1..15: mol_type = other RNA
cgtccatcacagcat

SEQ ID NO: 715 - RNA, 15 nt, synthetic construct
  source 1..15: mol_type = other RNA
ggccggacacagcat

SEQ ID NO: 716 - RNA, 15 nt, synthetic construct
  source 1..15: mol_type = other RNA
ccggacagtcagcat

SEQ ID NO: 717 - RNA, 15 nt, synthetic construct
  source 1..15: mol_type = other RNA
ggagggccacagcat

SEQ ID NO: 718 - RNA, 15 nt, synthetic construct
  source 1..15: mol_type = other RNA
cggacatgccagcat

SEQ ID NO: 719 - RNA, 15 nt, synthetic construct
  source 1..15: mol_type = other RNA
gccggacatcagcat

SEQ ID NO: 720 - RNA, 15 nt, synthetic construct
  source 1..15: mol_type = other RNA
gggctcactcagcat

SEQ ID NO: 721 - RNA, 15 nt, synthetic construct
  source 1..15: mol_type = other RNA
cggggcttccagcat

SEQ ID NO: 722 - RNA, 15 nt, synthetic construct
  source 1..15: mol_type = other RNA
acggggaagcagcat

SEQ ID NO: 723 - RNA, 15 nt, synthetic construct
  source 1..15: mol_type = other RNA
gtccatcttcagcat

SEQ ID NO: 724 - RNA, 15 nt, synthetic construct
  source 1..15: mol_type = other RNA
gtccttcgacagcat

SEQ ID NO: 725 - RNA, 15 nt, synthetic construct
  source 1..15: mol_type = other RNA
tggggtatacagcat

SEQ ID NO: 726 - RNA, 15 nt, synthetic construct
  source 1..15: mol_type = other RNA
gaacgactacagcat

SEQ ID NO: 727 - RNA, 15 nt, synthetic construct
  source 1..15: mol_type = other RNA
acggggcaacagcat

SEQ ID NO: 728 - RNA, 15 nt, synthetic construct
  source 1..15: mol_type = other RNA
cgtgggtcacagcat

SEQ ID NO: 729 - RNA, 15 nt, synthetic construct
  source 1..15: mol_type = other RNA
gccggacatcagcat

SEQ ID NO: 730 - RNA, 15 nt, synthetic construct
  source 1..15: mol_type = other RNA
gagtgtgaccagcat

SEQ ID NO: 731 - RNA, 15 nt, synthetic construct
  source 1..15: mol_type = other RNA
tcaaatcatcagcat

SEQ ID NO: 732 - RNA, 15 nt, synthetic construct
  source 1..15: mol_type = other RNA
ctcaaatctcagcat

SEQ ID NO: 733 - RNA, 24 nt, synthetic construct
  source 1..24: mol_type = other RNA
ttcataacgtgaacagggccggac

SEQ ID NO: 734 - RNA, 25 nt, synthetic construct
  source 1..25: mol_type = other RNA
aatattgcacttgtcccggcctgta

SEQ ID NO: 735 - RNA, 23 nt, synthetic construct
  source 1..23: mol_type = other RNA
atattgcacttgtcccggcctga

SEQ ID NO: 736 - RNA, 24 nt, synthetic construct
  source 1..24: mol_type = other RNA
atattgcacttgtcccggcctgta

SEQ ID NO: 737 - RNA, 22 nt, synthetic construct
  source 1..22: mol_type = other RNA
atattgcacttgtcccggcctg

SEQ ID NO: 738 - RNA, 23 nt, synthetic construct
  source 1..23: mol_type = other RNA
atattgcacttgtcccggcctgt

SEQ ID NO: 739 - RNA, 23 nt, synthetic construct
  source 1..23: mol_type = other RNA
cattgcacttgtcccggcctgta

SEQ ID NO: 740 - RNA, 24 nt, synthetic construct
  source 1..24: mol_type = other RNA
ctattgcacttgtcccggcctgta

SEQ ID NO: 741 - RNA, 22 nt, synthetic construct
  source 1..22: mol_type = other RNA
gattgcacttgtcccggcctgt

SEQ ID NO: 742 - RNA, 24 nt, synthetic construct
  source 1..24: mol_type = other RNA
gtattgcacttgtcccggcctgta

SEQ ID NO: 743 - RNA, 23 nt, synthetic construct
  source 1..23: mol_type = other RNA
gtattgcacttgtcccggcctgt

SEQ ID NO: 744 - RNA, 22 nt, synthetic construct
  source 1..22: mol_type = other RNA
tattacacttgtcccggcctgt

SEQ ID NO: 745 - RNA, 23 nt, synthetic construct
  source 1..23: mol_type = other RNA
tattgcacttgtcccggcctata

SEQ ID NO: 746 - RNA, 25 nt, synthetic construct
  source 1..25: mol_type = other RNA
tattgcacttgtcccggcctgtacg

SEQ ID NO: 747 - RNA, 23 nt, synthetic construct
  source 1..23: mol_type = other RNA
tattgcacttgtcccggccaaaa

SEQ ID NO: 748 - RNA, 24 nt, synthetic construct
  source 1..24: mol_type = other RNA
tattgcacttgtcccggcctgatt

SEQ ID NO: 749 - RNA, 22 nt, synthetic construct
  source 1..22: mol_type = other RNA
tattgcacttatcccggcctgt

SEQ ID NO: 750 - RNA, 24 nt, synthetic construct
  source 1..24: mol_type = other RNA
tattgcacttgtcccggcctgtca

SEQ ID NO: 751 - RNA, 23 nt, synthetic construct
  source 1..23: mol_type = other RNA
tattgcacttgtctcggcctgta

SEQ ID NO: 752 - RNA, 22 nt, synthetic construct
  source 1..22: mol_type = other RNA
tattgcacttgtcccggcctgt

SEQ ID NO: 753 - RNA, 25 nt, synthetic construct
  source 1..25: mol_type = other RNA
tattgcacttgtcccggcctgttaa

SEQ ID NO: 754 - RNA, 23 nt, synthetic construct
  source 1..23: mol_type = other RNA
ttattgcacttgtcccggcctgt

SEQ ID NO: 755 - RNA, 22 nt, synthetic construct
  source 1..22: mol_type = other RNA
ttattgcacttgtcccggcctg

SEQ ID NO: 756 - RNA, 23 nt, synthetic construct
  source 1..23: mol_type = other RNA
cattgcacttgtcccggcctgta

SEQ ID NO: 757 - RNA, 22 nt, synthetic construct
  source 1..22: mol_type = other RNA
gattgcacttgtcccggcctgt

SEQ ID NO: 758 - RNA, 25 nt, synthetic construct
  source 1..25: mol_type = other RNA
tattgcacttgtcccggcctgtacg

SEQ ID NO: 759 - RNA, 22 nt, synthetic construct
  source 1..22: mol_type = other RNA
tattgcacttatcccggcctgt

SEQ ID NO: 760 - RNA, 23 nt, synthetic construct
  source 1..23: mol_type = other RNA
tattgcacttgtctcggcctgta

SEQ ID NO: 761 - RNA, 22 nt, synthetic construct
  source 1..22: mol_type = other RNA
tattgcacttgtcccggcctgt

SEQ ID NO: 762 - RNA, 22 nt, synthetic construct
  source 1..22: mol_type = other RNA
tattgcacttgtcccggcctgt

SEQ ID NO: 763 - RNA, 27 nt, synthetic construct
  source 1..27: mol_type = other RNA
aatattgcacttgtcccggcctgtatt

SEQ ID NO: 764 - RNA, 25 nt, synthetic construct
  source 1..25: mol_type = other RNA
atattgcacttgtcccggcctgatt

SEQ ID NO: 765 - RNA, 26 nt, synthetic construct
  source 1..26: mol_type = other RNA
atattgcacttgtcccggcctgtatt

SEQ ID NO: 766 - RNA, 24 nt, synthetic construct
  source 1..24: mol_type = other RNA
atattgcacttgtcccggcctgtt

SEQ ID NO: 767 - RNA, 25 nt, synthetic construct
  source 1..25: mol_type = other RNA
atattgcacttgtcccggcctgttt

SEQ ID NO: 768 - RNA, 25 nt, synthetic construct
  source 1..25: mol_type = other RNA
cattgcacttgtcccggcctgtatt

SEQ ID NO: 769 - RNA, 26 nt, synthetic construct
  source 1..26: mol_type = other RNA
ctattgcacttgtcccggcctgtatt

SEQ ID NO: 770 - RNA, 24 nt, synthetic construct
  source 1..24: mol_type = other RNA
gattgcacttgtcccggcctgttt

SEQ ID NO: 771 - RNA, 26 nt, synthetic construct
  source 1..26: mol_type = other RNA
gtattgcacttgtcccggcctgtatt

SEQ ID NO: 772 - RNA, 25 nt, synthetic construct
  source 1..25: mol_type = other RNA
gtattgcacttgtcccggcctgttt

SEQ ID NO: 773 - RNA, 24 nt, synthetic construct
  source 1..24: mol_type = other RNA
tattacacttgtcccggcctgttt

SEQ ID NO: 774 - RNA, 25 nt, synthetic construct
  source 1..25: mol_type = other RNA
tattgcacttgtcccggcctatatt

SEQ ID NO: 775 - RNA, 27 nt, synthetic construct
  source 1..27: mol_type = other RNA
tattgcacttgtcccggcctgtacgtt

SEQ ID NO: 776 - RNA, 25 nt, synthetic construct
  source 1..25: mol_type = other RNA
tattgcacttgtcccggccaaaatt

SEQ ID NO: 777 - RNA, 26 nt, synthetic construct
  source 1..26: mol_type = other RNA
tattgcacttgtcccggcctgatttt

SEQ ID NO: 778 - RNA, 24 nt, synthetic construct
  source 1..24: mol_type = other RNA
tattgcacttatcccggcctgttt

SEQ ID NO: 779 - RNA, 26 nt, synthetic construct
  source 1..26: mol_type = other RNA
tattgcacttgtcccggcctgtcatt

SEQ ID NO: 780 - RNA, 25 nt, synthetic construct
  source 1..25: mol_type = other RNA
tattgcacttgtctcggcctgtatt

SEQ ID NO: 781 - RNA, 24 nt, synthetic construct
  source 1..24: mol_type = other RNA
tattgcacttgtcccggcctgttt

SEQ ID NO: 782 - RNA, 27 nt, synthetic construct
  source 1..27: mol_type = other RNA
tattgcacttgtcccggcctgttaatt

SEQ ID NO: 783 - RNA, 25 nt, synthetic construct
  source 1..25: mol_type = other RNA
ttattgcacttgtcccggcctgttt

SEQ ID NO: 784 - RNA, 24 nt, synthetic construct
  source 1..24: mol_type = other RNA
ttattgcacttgtcccggcctgtt

SEQ ID NO: 785 - RNA, 25 nt, synthetic construct
  source 1..25: mol_type = other RNA
cattgcacttgtcccggcctgtatt

SEQ ID NO: 786 - RNA, 24 nt, synthetic construct
  source 1..24: mol_type = other RNA
gattgcacttgtcccggcctgttt

SEQ ID NO: 787 - RNA, 27 nt, synthetic construct
  source 1..27: mol_type = other RNA
tattgcacttgtcccggcctgtacgtt

SEQ ID NO: 788 - RNA, 24 nt, synthetic construct
  source 1..24: mol_type = other RNA
tattgcacttatcccggcctgttt

SEQ ID NO: 789 - RNA, 25 nt, synthetic construct
  source 1..25: mol_type = other RNA
tattgcacttgtctcggcctgtatt

SEQ ID NO: 790 - RNA, 24 nt, synthetic construct
  source 1..24: mol_type = other RNA
tattgcacttgtcccggcctgttt

SEQ ID NO: 791 - RNA, 24 nt, synthetic construct
  source 1..24: mol_type = other RNA
tattgcacttgtcccggcctgttt

SEQ ID NO: 792 - RNA, 27 nt, synthetic construct
  source 1..27: mol_type = other RNA
tacaggccgggacaagtgcaatatatt

SEQ ID NO: 793 - RNA, 25 nt, synthetic construct
  source 1..25: mol_type = other RNA
tcaggccgggacaagtgcaataatt

SEQ ID NO: 794 - RNA, 26 nt, synthetic construct
  source 1..26: mol_type = other RNA
tacaggccgggacaagtgcaataatt

SEQ ID NO: 795 - RNA, 24 nt, synthetic construct
  source 1..24: mol_type = other RNA
caggccgggacaagtgcaataatt

SEQ ID NO: 796 - RNA, 25 nt, synthetic construct
  source 1..25: mol_type = other RNA
acaggccgggacaagtgcaataatt

SEQ ID NO: 797 - RNA, 25 nt, synthetic construct
  source 1..25: mol_type = other RNA
tacaggccgggacaagtgcaatctt

SEQ ID NO: 798 - RNA, 26 nt, synthetic construct
  source 1..26: mol_type = other RNA
tacaggccgggacaagtgcaatactt

SEQ ID NO: 799 - RNA, 24 nt, synthetic construct
  source 1..24: mol_type = other RNA
acaggccgggacaagtgcaatgtt

SEQ ID NO: 800 - RNA, 26 nt, synthetic construct
  source 1..26: mol_type = other RNA
tacaggccgggacaagtgcaatagtt

SEQ ID NO: 801 - RNA, 25 nt, synthetic construct
  source 1..25: mol_type = other RNA
acaggccgggacaagtgcaatagtt

SEQ ID NO: 802 - RNA, 24 nt, synthetic construct
  source 1..24: mol_type = other RNA
acaggccgggacaagtgtaatttt

SEQ ID NO: 803 - RNA, 25 nt, synthetic construct
  source 1..25: mol_type = other RNA
tataggccgggacaagtgcaatttt

SEQ ID NO: 804 - RNA, 27 nt, synthetic construct
  source 1..27: mol_type = other RNA
cgtacaggccgggacaagtgcaatttt

SEQ ID NO: 805 - RNA, 25 nt, synthetic construct
  source 1..25: mol_type = other RNA
ttttggccgggacaagtgcaatttt

SEQ ID NO: 806 - RNA, 26 nt, synthetic construct
  source 1..26: mol_type = other RNA
aatcaggccgggacaagtgcaatttt

SEQ ID NO: 807 - RNA, 24 nt, synthetic construct
  source 1..24: mol_type = other RNA
acaggccgggataagtgcaatttt

SEQ ID NO: 808 - RNA, 26 nt, synthetic construct
  source 1..26: mol_type = other RNA
tgacaggccgggacaagtgcaatttt

SEQ ID NO: 809 - RNA, 25 nt, synthetic construct
  source 1..25: mol_type = other RNA
tacaggccgagacaagtgcaatttt

SEQ ID NO: 810 - RNA, 24 nt, synthetic construct
  source 1..24: mol_type = other RNA
acaggccgggacaagtgcaatttt

SEQ ID NO: 811 - RNA, 27 nt, synthetic construct
  source 1..27: mol_type = other RNA
ttaacaggccgggacaagtgcaatttt

SEQ ID NO: 812 - RNA, 25 nt, synthetic construct
  source 1..25: mol_type = other RNA
acaggccgggacaagtgcaatattt

SEQ ID NO: 813 - RNA, 24 nt, synthetic construct
  source 1..24: mol_type = other RNA
caggccgggacaagtgcaatattt

SEQ ID NO: 814 - RNA, 25 nt, synthetic construct
  source 1..25: mol_type = other RNA
tacaggccgggacaagtgcaatctt

SEQ ID NO: 815 - RNA, 24 nt, synthetic construct
  source 1..24: mol_type = other RNA
acaggccgggacaagtgcaatgtt

SEQ ID NO: 816 - RNA, 27 nt, synthetic construct
  source 1..27: mol_type = other RNA
cgtacaggccgggacaagtgcaatttt

SEQ ID NO: 817 - RNA, 24 nt, synthetic construct
  source 1..24: mol_type = other RNA
acaggccgggataagtgcaatttt

SEQ ID NO: 818 - RNA, 25 nt, synthetic construct
  source 1..25: mol_type = other RNA
tacaggccgagacaagtgcaatttt

SEQ ID NO: 819 - RNA, 24 nt, synthetic construct
  source 1..24: mol_type = other RNA
acaggccgggacaagtgcaatttt

SEQ ID NO: 820 - RNA, 24 nt, synthetic construct
  source 1..24: mol_type = other RNA
acaggccgggacaagtgcaatttt

SEQ ID NO: 821 - RNA, 25 nt, synthetic construct
  source 1..25: mol_type = other RNA
tattgcacttgtcccggcctatatt

SEQ ID NO: 822 - RNA, 19 nt, synthetic construct
  source 1..19: mol_type = other RNA
aatcccggacgagcccctt

SEQ ID NO: 823 - RNA, 19 nt, synthetic construct
  source 1..19: mol_type = other RNA
ttatagggcctgctcgggg

SEQ ID NO: 824 - RNA, 24 nt, synthetic construct
  source 1..24: mol_type = other RNA
atattgcacttgtcccggcctgtt

SEQ ID NO: 825 - RNA, 23 nt, synthetic construct
  source 1..23: mol_type = other RNA
tataggccgggacaagtgcaata

SEQ ID NO: 826 - RNA, 22 nt, synthetic construct
  source 1..22: mol_type = other RNA
acaggccgggacaagtgcaata

SEQ ID NO: 827 - RNA, 22 nt, synthetic construct
  source 1..22: mol_type = other RNA
agtgaattctaccagtgccata

SEQ ID NO: 828 - RNA, 23 nt, synthetic construct
  source 1..23: mol_type = other RNA
acagtgaattctaccagtgccat

SEQ ID NO: 829 - RNA, 25 nt, synthetic construct
  source 1..25: mol_type = other RNA
tacaggccgggacaagtgcaatatt

SEQ ID NO: 830 - RNA, 21 nt, synthetic construct
  source 1..21: mol_type = other RNA
caaaagtaactgcagttttgg

SEQ ID NO: 831 - RNA, 22 nt, synthetic construct
  source 1..22: mol_type = other RNA
acaggccgggacaagtgtaata

SEQ ID NO: 832 - RNA, 24 nt, synthetic construct
  source 1..24: mol_type = other RNA
tctacctgcactatgagcactttg

SEQ ID NO: 833 - RNA, 23 nt, synthetic construct
  source 1..23: mol_type = other RNA
aacatcaatacttcctggctccc

SEQ ID NO: 834 - RNA, 25 nt, synthetic construct
  source 1..25: mol_type = other RNA
ttaacaggccgggacaagtgcaata

SEQ ID NO: 835 - RNA, 22 nt, synthetic construct
  source 1..22: mol_type = other RNA
acaggccgggacaagtgcaatc

SEQ ID NO: 836 - RNA, 25 nt, synthetic construct
  source 1..25: mol_type = other RNA
attggggtatttgacaaactgacac

SEQ ID NO: 837 - RNA, 26 nt, synthetic construct
  source 1..26: mol_type = other RNA
ataactgaggggcctcagaccgagct

SEQ ID NO: 838 - RNA, 24 nt, synthetic construct
  source 1..24: mol_type = other RNA
aactcagtaatggtaacggtttgt

SEQ ID NO: 839 - RNA, 21 nt, synthetic construct
  source 1..21: mol_type = other RNA
ctaggggcagctcagtacagg

SEQ ID NO: 840 - RNA, 20 nt, synthetic construct
  source 1..20: mol_type = other RNA
aatccttgcccgggtgcatt

SEQ ID NO: 841 - RNA, 21 nt, synthetic construct
  source 1..21: mol_type = other RNA
taaaccatgatgtgctgctaa

SEQ ID NO: 842 - RNA, 46 nt, synthetic construct
  source 1..46: mol_type = other RNA
ttcacaaccctagtggcgccattttcacaaccctagtggcgccatt

SEQ ID NO: 843 - RNA, 22 nt, synthetic construct
  source 1..22: mol_type = other RNA
tccctcacacagaattccagaa

SEQ ID NO: 844 - RNA, 23 nt, synthetic construct
  source 1..23: mol_type = other RNA
tcaggccgggacaagtgcaatat

SEQ ID NO: 845 - RNA, 22 nt, synthetic construct
  source 1..22: mol_type = other RNA
ctcagaaggacaagtagagttt

SEQ ID NO: 846 - RNA, 22 nt, synthetic construct
  source 1..22: mol_type = other RNA
caggccgggacaagtgcaataa

SEQ ID NO: 847 - RNA, 26 nt, synthetic construct
  source 1..26: mol_type = other RNA
ctcggggcagctcagtacaggacgag

SEQ ID NO: 848 - RNA, 24 nt, synthetic construct
  source 1..24: mol_type = other RNA
aaactcggggcagctcagtacagg

SEQ ID NO: 849 - RNA, 22 nt, synthetic construct
  source 1..22: mol_type = other RNA
actgggagcagagggtgagttc

SEQ ID NO: 850 - RNA, 22 nt, synthetic construct
  source 1..22: mol_type = other RNA
acaggaactgcctttctctcca

SEQ ID NO: 851 - RNA, 23 nt, synthetic construct
  source 1..23: mol_type = other RNA
ttttggccgggacaagtgcaata

SEQ ID NO: 852 - RNA, 21 nt, synthetic construct
  source 1..21: mol_type = other RNA
agcaagaccccgtctctaaaa

SEQ ID NO: 853 - RNA, 27 nt, synthetic construct
  source 1..27: mol_type = other RNA
tccagtgtgagttctaccattgccaaa

SEQ ID NO: 854 - RNA, 21 nt, synthetic construct
  source 1..21: mol_type = other RNA
cttcctgctccctgctctaca

SEQ ID NO: 855 - RNA, 23 nt, synthetic construct
  source 1..23: mol_type = other RNA
acaggccgggacaagtgcaatac

SEQ ID NO: 856 - RNA, 22 nt, synthetic construct
  source 1..22: mol_type = other RNA
agacaacaatgccaacctcaca

SEQ ID NO: 857 - RNA, 24 nt, synthetic construct
  source 1..24: mol_type = other RNA
tacaggccgggacaagtgcaatac

SEQ ID NO: 858 - RNA, 20 nt, synthetic construct
  source 1..20: mol_type = other RNA
acaacaatgccaacctcaca

SEQ ID NO: 859 - RNA, 22 nt, synthetic construct
  source 1..22: mol_type = other RNA
aactgggggccaggagagtgga

SEQ ID NO: 860 - RNA, 23 nt, synthetic construct
  source 1..23: mol_type = other RNA
actcggggcagatcagtacagga

SEQ ID NO: 861 - RNA, 23 nt, synthetic construct
  source 1..23: mol_type = other RNA
ctcaccgacagcgttgaatgttt

SEQ ID NO: 862 - RNA, 24 nt, synthetic construct
  source 1..24: mol_type = other RNA
tctatctgcactgtcagcacttta

SEQ ID NO: 863 - RNA, 25 nt, synthetic construct
  source 1..25: mol_type = other RNA
gaactcggggcagctcagtacagga

SEQ ID NO: 864 - RNA, 23 nt, synthetic construct
  source 1..23: mol_type = other RNA
aaacggggcagctcagtacagga

SEQ ID NO: 865 - RNA, 24 nt, synthetic construct
  source 1..24: mol_type = other RNA
tacgagccgcgaggagccgagaga

SEQ ID NO: 866 - RNA, 21 nt, synthetic construct
  source 1..21: mol_type = other RNA
ctcggcgcagctcagtacagg

SEQ ID NO: 867 - RNA, 19 nt, synthetic construct
  source 1..19: mol_type = other RNA
tgaattctaccagtgccat

SEQ ID NO: 868 - RNA, 24 nt, synthetic construct
  source 1..24: mol_type = other RNA
tacaggccgggacaagtgcaatat

SEQ ID NO: 869 - RNA, 23 nt, synthetic construct
  source 1..23: mol_type = other RNA
acaggccgggacaagtgcaatat

SEQ ID NO: 870 - RNA, 23 nt, synthetic construct
  source 1..23: mol_type = other RNA
tacaggccgagacaagtgcaata

SEQ ID NO: 871 - RNA, 24 nt, synthetic construct
  source 1..24: mol_type = other RNA
aatcaggccgggacaagtgcaata

SEQ ID NO: 872 - RNA, 22 nt, synthetic construct
  source 1..22: mol_type = other RNA
caggccgggacaagtgcaatat

SEQ ID NO: 873 - RNA, 25 nt, synthetic construct
  source 1..25: mol_type = other RNA
actcggggcagctcagtacaggaca

SEQ ID NO: 874 - RNA, 21 nt, synthetic construct
  source 1..21: mol_type = other RNA
ctcctccacttcccggacaca

SEQ ID NO: 875 - RNA, 23 nt, synthetic construct
  source 1..23: mol_type = other RNA
acaggccgggacaagtgcaataa

SEQ ID NO: 876 - RNA, 25 nt, synthetic construct
  source 1..25: mol_type = other RNA
ttctcggggcagctcagtacaggag

SEQ ID NO: 877 - RNA, 23 nt, synthetic construct
  source 1..23: mol_type = other RNA
actacctgcacgaacagcacttt

SEQ ID NO: 878 - RNA, 22 nt, synthetic construct
  source 1..22: mol_type = other RNA
acaggccgggataagtgcaata

SEQ ID NO: 879 - RNA, 24 nt, synthetic construct
  source 1..24: mol_type = other RNA
tgacaggccgggacaagtgcaata

SEQ ID NO: 880 - RNA, 22 nt, synthetic construct
  source 1..22: mol_type = other RNA
accgggaggggactgagcctga

SEQ ID NO: 881 - RNA, 25 nt, synthetic construct
  source 1..25: mol_type = other RNA
cgtacaggccgggacaagtgcaata

SEQ ID NO: 882 - RNA, 24 nt, synthetic construct
  source 1..24: mol_type = other RNA
tacaggccgggacaagtgcaatag

SEQ ID NO: 883 - RNA, 24 nt, synthetic construct
  source 1..24: mol_type = other RNA
tcactcggggcagctcagtacagg

SEQ ID NO: 884 - RNA, 21 nt, synthetic construct
  source 1..21: mol_type = other RNA
cttcggggcagctcagtacag

SEQ ID NO: 885 - RNA, 21 nt, synthetic construct
  source 1..21: mol_type = other RNA
gaaggggcatttagggcagta

SEQ ID NO: 886 - RNA, 25 nt, synthetic construct
  source 1..25: mol_type = other RNA
ttctacctgcactgtaagcactttg

SEQ ID NO: 887 - RNA, 23 nt, synthetic construct
  source 1..23: mol_type = other RNA
agcttcctgctccctgctctaca

SEQ ID NO: 888 - RNA, 25 nt, synthetic construct
  source 1..25: mol_type = other RNA
atatggggtatttgacaaactgaca

SEQ ID NO: 889 - RNA, 23 nt, synthetic construct
  source 1..23: mol_type = other RNA
atcagcaagtacccacagtgcgg

SEQ ID NO: 890 - RNA, 23 nt, synthetic construct
  source 1..23: mol_type = other RNA
aacggggcagctcagtacaggat

SEQ ID NO: 891 - RNA, 23 nt, synthetic construct
  source 1..23: mol_type = other RNA
actgggtgcgggatgcagggtga

SEQ ID NO: 892 - RNA, 23 nt, synthetic construct
  source 1..23: mol_type = other RNA
tacaggccgggacaagtgcaatg

SEQ ID NO: 893 - RNA, 24 nt, synthetic construct
  source 1..24: mol_type = other RNA
cagtgtgagttctaccattgccaa

SEQ ID NO: 894 - RNA, 27 nt, synthetic construct
  source 1..27: mol_type = other RNA
tactaaactcagtaatggtaacggttt

SEQ ID NO: 895 - RNA, 25 nt, synthetic construct
  source 1..25: mol_type = other RNA
tctaaactcagtaatggtaacggtt

SEQ ID NO: 896 - RNA, 53 nt, synthetic construct
  source 1..53: mol_type = other RNA
gcattggtggttcagtggtagaattctcgcctgccacgcgggaggcccgggtt